(19) United States
(12) Patent Application Publication
(10) Pub. No.: US 2024/0081149 A1
(43) Pub. Date: Mar. 7, 2024

(54) COMPOUND FOR ORGANIC ELECTRONIC ELEMENT, ORGANIC ELECTRONIC ELEMENT USING THE SAME, AND AN ELECTRONIC DEVICE THEREOF

(71) Applicant: DUK SAN NEOLUX CO., LTD., Cheonan-si (KR)

(72) Inventors: Yun Suk LEE, Cheonan-si (KR); Hyun Ju SONG, Cheonan-si (KR); Junggeun LEE, Cheonan-si (KR)

(73) Assignee: DUK SAN NEOLUX CO., LTD., Cheonan-si (KR)

(21) Appl. No.: 18/355,252

(22) Filed: Jul. 19, 2023

Related U.S. Application Data
(63) Continuation of application No. 18/180,625, filed on Mar. 8, 2023.
Application No. 18/180,625 is a continuation-in-part of application No. 17/212,886, filed on Mar. 25, 2021, now Pat. No. 11,678,577.
Application No. 17/212,886 is a continuation of application No. 17/096,790, filed on Nov. 12, 2020, now Pat. No. 11,063,226.

(30) Foreign Application Priority Data
Oct. 26, 2020 (KR) 10-2020-0139441

(51) Int. Cl. H10K 85/60; C07D 251/24; C09K 11/06

(52) U.S. Cl. CPC H10K 85/6574; C07D 251/24; C09K 11/06; H10K 85/626; H10K 85/631; H10K 85/653; H10K 85/654; H10K 85/6572; H10K 85/6576; C07B 2200/05; C09K 2211/1018; H10K 50/11

(57) ABSTRACT

Provided are a compound capable of improving the light-emitting efficiency, stability, and lifespan of an element; an organic electronic element using same; and an electronic device thereof.

### BACKGROUND

### Technical Field

The present invention relates to a compound for an organic electronic element, an organic electronic element using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function. And the light emitting material can be classified into a high molecular weight type and a low molecular weight type according to the molecular weight, and according to the light emission mechanism, it can be classified into a fluorescent material derived from a singlet excited state of an electron and a phosphorescent material derived from a triplet excited state of an electron. Also, the light emitting material may be divided into blue, green, and red light emitting materials and yellow and orange light emitting materials necessary for realizing a better natural color according to the emission color.

However, when only one material is used as a light emitting material, due to intermolecular interaction, the maximum emission wavelength shifts to a longer wavelength, and there are problems in that the color purity is lowered or the device efficiency is reduced due to the emission attenuation effect, therefore in order to increase color purity and increase luminous efficiency through energy transfer, a host/dopant system may be used as a light emitting material. The principle is that when a small amount of a dopant having a smaller energy band gap than that of the host forming the emitting layer is mixed in the emitting layer, excitons generated in the emitting layer are transported to the dopant to emit light with high efficiency. At this time, since the wavelength of the host moves to the wavelength band of the dopant, light having a desired wavelength can be obtained according to the type of dopant used.

Currently, the portable display market is a large-area display, and the size thereof is increasing, and thus, more power consumption than the power consumption required for the existing portable display is required. Therefore, power consumption has become a very important factor for a portable display having a limited power supply such as a battery, and the problem of efficiency and lifespan must also be solved.

Efficiency, lifespan, and driving voltage are related to each other, and when the efficiency is increased, the driving voltage is relatively decreased, and as the driving voltage is decreased, crystallization of organic materials due to Joule heating generated during driving decreases, and consequently, the lifespan tends to increase. However, the efficiency cannot be maximized simply by improving the organic material layer. This is because, when the energy level and T1 value between each organic material layer, and the intrinsic properties (mobility, interfacial properties, etc.) of materials are optimally combined, long lifespan and high efficiency can be achieved at the same time.

Therefore, while delaying the penetration and diffusion of metal oxide from the anode electrode (ITO) into the organic layer, which is one of the causes of shortening the lifespan of the organic electronic element, it should have stable characteristics against Joule heating generated during device driving, and OLED devices are mainly formed by a deposition method, and it is necessary to develop a material that can withstand a long time during deposition, that is, a material with strong heat resistance.

That is, in order to fully exhibit the excellent characteristics of an organic electronic element, it should be preceded that the material constituting the organic material layer in the device, such as a hole injection material, a hole transport material, a light emitting material, an electron transport material, an electron injection material, etc., is supported by a stable and efficient material. But the development of a stable and efficient organic material layer material for an organic electronic device has not yet been sufficiently made. Therefore, the development of new materials is continuously required, and in particular, the development of a host material for the emitting layer is urgently required.

### DETAILED DESCRIPTION OF THE INVENTION

### Summary

In order to solve the problems of the above-mentioned background art, the present invention has revealed a compound having a novel structure, and when this compound is applied to an organic electronic element, it has been found that the luminous efficiency, stability and lifespan of the device can be significantly improved.

Accordingly, an object of the present invention is to provide a novel compound, an organic electronic element using the same, and an electronic device thereof.

### Technical Solution

The present invention provides a compound represented by Formula 3.

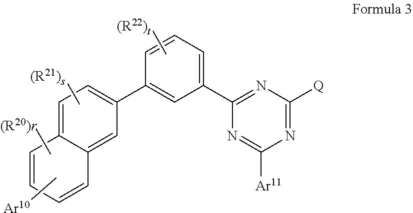

In another aspect, the present invention provides an organic electronic element comprising the compound represented by Formula 3 and an electronic device thereof.

### Effects of the Invention

By using the compound according to the present invention, high luminous efficiency, low driving voltage and high heat resistance of the element can be achieved, and color purity and lifespan of the element can be greatly improved.

### BRIEF DESCRIPTION OF THE DRAWINGS

FIG. 1 to FIG. 3 are exemplary views of an organic electroluminescent device according to the present invention.

FIG. 4 shows a Formula according to one aspect of the present invention.

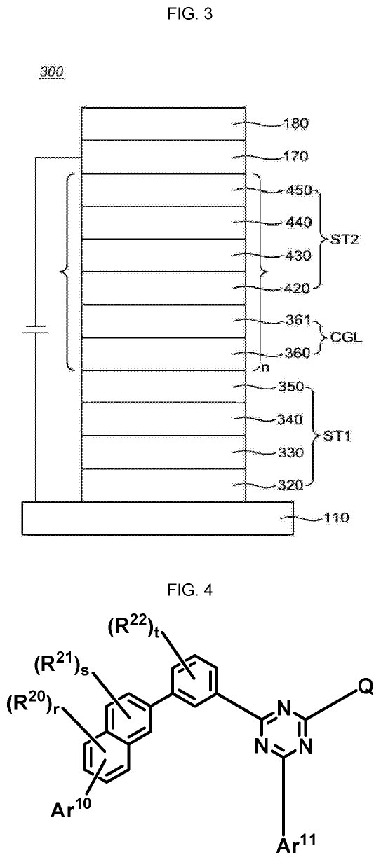

| | |
|---|---|
| 100, 200, 300: organic electronic | 110 : the first electrode |
| element | 130 : hole transport layer |
| 120 : hole injection layer | 150 : electron transport layer |
| 140 : emitting layer | 170 : second electrode |
| 160 : electron injection layer | 210 : buffer layer |
| 180 : light efficiency enhancing Layer | 320 : first hole injection layer |
| 220 : emitting auxiliary layer | 340 : first emitting layer |
| 330 : first hole transport layer | 360 : first charge generation layer |
| 350 : first electron transport layer | 420 : second hole injection layer |
| 361 : second charge generation layer | 440 : second emitting layer |
| 430 : second hole transport layer | CGL : charge generation layer |
| 450 : second electron transport layer | ST2 : second stack |
| ST1 : first stack | |

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”, “coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

The terms “aryl group” and “arylene group” used in the present invention have 6 to 60 carbon atoms, respectively, unless otherwise specified, but are not limited thereto. In the present invention, an aryl group or an arylene group means a single ring or multiple ring aromatic, and includes an aromatic ring formed by an adjacent substituent joining or participating in a reaction.

For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of a single ring or multiple ring, and may include heteroaliphadic ring and heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes the following compound.

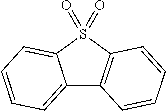 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

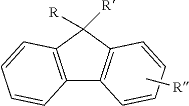

The term “spiro compound”, as used herein, has a ‘spiro union’, and a spiro union means a connection in which two rings share only one atom. At this time, atoms shared in the two rings are called ‘spiro atoms’, and these compounds are called ‘monospiro-’, ‘di-spiro-’ and ‘tri-spiro-’, respectively, depending on the number of spiro atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless there is an explicit explanation, the formula used in the present invention is the same as the definition of the substituent by the exponent definition of the following formula.

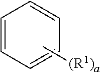

Here, when a is an integer of 0, the substituent R¹ is absent, when a is an integer of 1, the sole substituent R¹ is linked to any one of the carbon constituting the benzene ring, when a is an integer of 2 or 3, each is combined as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

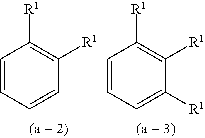

Hereinafter, a compound according to an aspect of the present invention and an organic electronic element comprising the same will be described.

The present invention provides a compound represented by Formula 2.

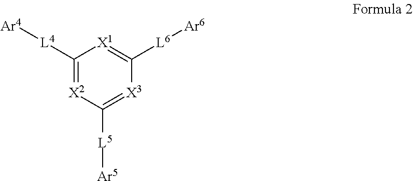

In Formula 2, each symbol may be defined as follows.

X1, X2 and X3 are each independently CR or N, provided that at least one of X1, X2 and X3 are N,

L4, L5 and L6 are each independently selected from the group consisting of a single bond; a C₆-C₆₀ arylene group; fluorenylene group; a C₂-C₆₀ heterocyclic group including at least one heteroatom of O, N, S, Si or P; a fused ring group of a C₃-C₆₀ aliphatic ring and a C₆-C₆₀ aromatic ring; C₁-C₅₀ alkylene group; C₂-C₂₀ alkenylene group; and a C₂-C₂₀ alkynylene group;

When L⁴, L⁵ and L⁶ are an arylene group, it may be preferably a C₆-C₃₀ arylene group, more preferably a C₆-C₂₄ arylene group, for example, phenylene, biphenyl, naphthalene, terphenyl, etc.,

When L⁴, L⁵ and L⁶ are a heterocyclic group, it may be preferably a C₂˜C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

When L⁴, L⁵ and L⁶ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring,

When L⁴, L⁵ and L⁶ are an alkylene group, it may be preferably a C1-C30 alkylene group, more preferably a C₁-C₂₄ alkylene group,

Ar⁴, Ar⁵ and Ar⁶ are each independently selected from the group consisting of a C₆-C₆₀ aryl group; a C₂-C₆₀ heterocyclic group including at least one heteroatom of O, N, S, Si or P; fluorenyl group; a fused ring group of a C₆-C₆₀ aromatic ring and a C₃-C₆₀ aliphatic ring; a C₁-C₅₀ alkyl group; a C₂-C₂₀ alkenyl group; a C₂-C₂₀ alkynyl group; a C₁-C₃₀ alkoxyl group; a C₆-C₃₀ arylthio group; a C₆-C₃₀ aryloxy group; and however, at least one of Ar⁴, Ar⁵ and Ar⁶ is a substituted or unsubstituted naphthyl group.

When Ar⁴, Ar⁵ and Ar⁶ are an aryl group, it may be preferably a C₆-C₃₀ aryl group, and more preferably a C₆-C₂₄ aryl group, for example, it may be phenylene, biphenyl, naphthalene, terphenyl, etc,

When Ar⁴, Ar⁵ and Ar⁶ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

When Ar⁴, Ar⁵ and Ar⁶ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

When Ar⁴, Ar⁵ and Ar⁶ are an alkyl group, it may be preferably a C₁-C₃₀ alkyl group, and more preferably a C₁-C₂₄ alkyl group.

When Ar⁴, Ar⁵ and Ar⁶ are alkoxyl groups, they may be preferably C₁˜C₂₄ alkoxyl groups.

When Ar⁴, Ar⁵ and Ar⁶ are an arylthio group, it may be preferably a C₆-C₂₄ arylthio group.

When Ar⁴, Ar⁵ and Ar⁶ are an aryloxy group, it may be preferably a C₆˜C₂₄ aryloxy group,

wherein the aryl group, arylene group, heterocyclic group, fluorenyl group, fluorenylene group, fused ring group, alkyl group, alkenyl group, alkoxy group, aryloxy group and arylthio group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, the present invention provides a compound wherein L⁴, L⁵ or L⁶ is represented by any one of the following Formulas b-1 to b-16.

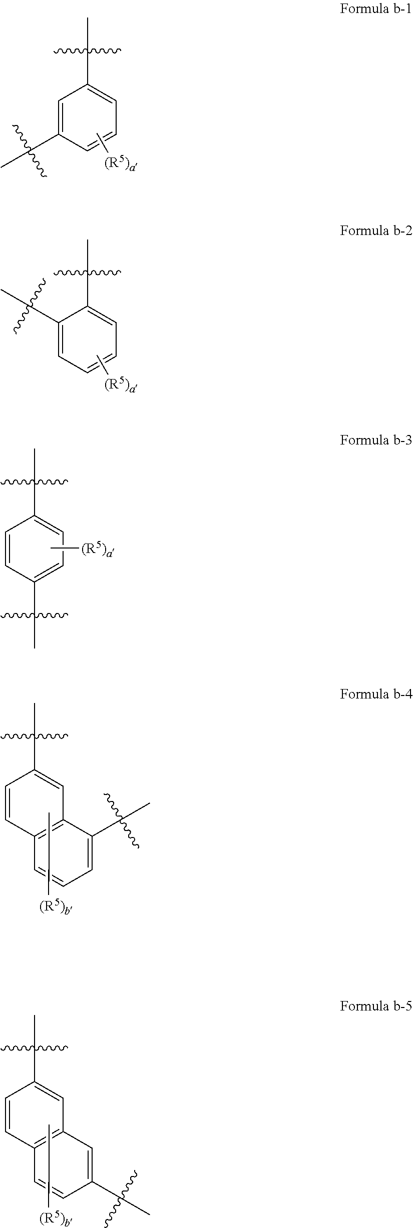

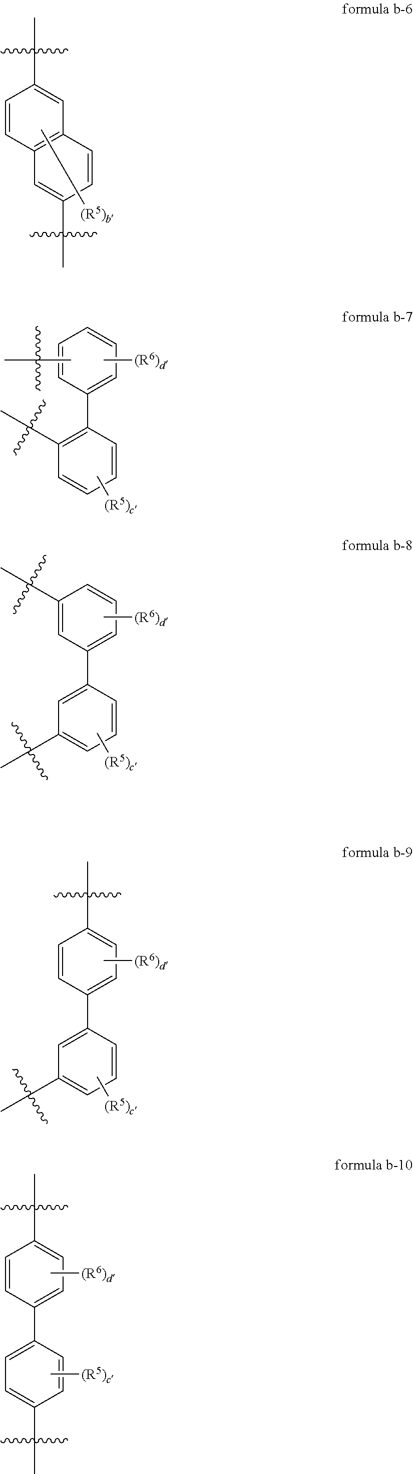

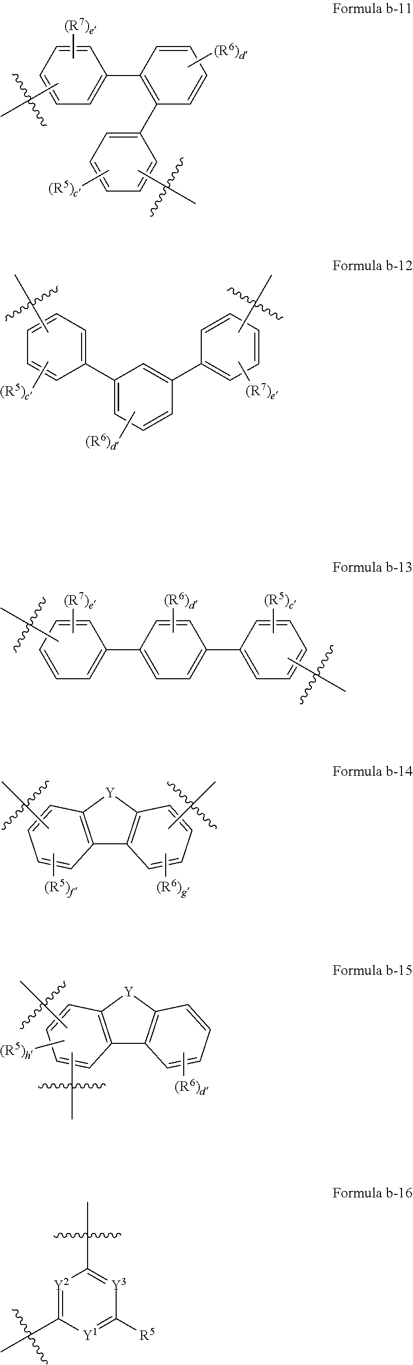

In Formula (b-1) to Formula (b-16), each symbol may be defined as follows.
1) Y is N-L⁸-Ar⁷, O, S or CR′R″;
2) L⁸ is the same as the definition of L⁴ in Formula 2,
3) Ar⁷ is the same as the definition of Ar⁴ in Formula 2,
4) R′ and R″ are each independently selected from the group consisting of a hydrogen; a C6-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(R^{a})(R^{b}); or may be bonded to each other to form a ring.

When R′ and R″ are an aryl group, it may be preferably a C₆-C₃₀ aryl group, and more preferably a C₆-C₂₄ aryl group, for example, it may be phenylene, biphenyl, naphthalene, terphenyl, etc,

When R′ and R″ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

When R′ and R″ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

When R′ and R″ are an alkyl group, it may be preferably a C₁-C₃₀ alkyl group, and more preferably a C₁-C₂₄ alkyl group.

When R′ and R″ are alkoxyl groups, they may be preferably C₁˜C₂₄ alkoxyl groups.

When R′ and R″ are an aryloxy group, it may be preferably a C₆˜C₂₄ aryloxy group,
5) a′, c′, d′ and e′ are each independently integers from 0 to 4, b′ is an integer from 0 to 6, f′ and g′ are independently integers from 0 to 3, and h′ is an integer from 0 to 2.
6) R⁵, R⁶ and R⁷ are each independently selected from the group consisting of hydrogen; deuterium; tritium; halogen; cyano group; nitro group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L^{a}-N(R^{c})(R^{d}); or in case a′, b′, c′, d′, e′ f′ g′ and h′ are 2 or more, R⁵, R⁶ and R⁷ are each in plural being the same or different, and a plurality of R⁵ or a plurality of R⁶ or a plurality of R⁷, alternatively, adjacent R⁵ and R⁶ or R⁶ and R⁷ may be bonded to each other to form an aromatic ring or a heteroaromatic ring.

Wherein R⁵, R⁶ and R⁷ are an aryl group, it may be preferably a C₆-C₃₀ aryl group, and more preferably a C₆-C₂₄ aryl group, for example, it may be phenylene, biphenyl, naphthalene, terphenyl, etc,

When R⁵, R⁶ and R⁷ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

When R⁵, R⁶ and R⁷ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

When R⁵, R⁶ and R⁷ are an alkyl group, it may be preferably a C₁-C₃₀ alkyl group, and more preferably a C₁-C₂₄ alkyl group.

When R⁵, R⁶ and R⁷ are alkoxyl groups, they may be preferably C₁˜C₂₄ alkoxyl groups.

When R⁵, R⁶ and R⁷ are an aryloxy group, it may be preferably a C₆˜C₂₄ aryloxy group,
7) L′ and L^{a} are the same as the definition of L⁴ in Formula 2,
8) R^{a}, R^{b}, R^{c} and R^{d} are each independently selected from the group consisting of a C₆-C₆₀ aryl group; fluorenyl group; a fused ring group of a C₃-C₆₀ aliphatic ring and a C₆-C₆₀ aromatic ring; a C₂-C₆₀ heterocyclic group including at least one heteroatom of O, N, S, Si or P;

Wherein R^{a}, R^{b}, R^{c} and R^{d} are an aryl group, it may be preferably a C₆-C₃₀ aryl group, and more preferably a C₆-C₂₄ aryl group, for example, it may be phenylene, biphenyl, naphthalene, terphenyl, etc,

When R^{a}, R^{b}, R^{c} and R^{d} are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

When R^{a}, R^{b}, R^{c} and R^{d} are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,
9) Y¹, Y² and Y³ are independently CR^{e} or N, provided that at least one of Y¹, Y² and Y³ is N,
10) R^{e} is selected from the group consisting of hydrogen; deuterium; tritium; halogen; cyano group; nitro group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group;

In case R^{e} is an aryl group, it may be preferably a C₆-C₃₀ aryl group, and more preferably a C₆-C₂₄ aryl group, for example, it may be phenylene, biphenyl, naphthalene, terphenyl, etc,

In case R^{e} is an a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

In case R^{e} is a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

In case R^{e} is an alkyl group, it may be preferably a C₁-C₃₀ alkyl group, and more preferably a C₁-C₂₄ alkyl group.

When R^{e} is an alkoxyl group, it may be preferably C₁˜C₂₄ alkoxyl group.

When R^{e} is an aryloxy group, it may be preferably a C₆˜C₂₄ aryloxy group,
11) Adjacent R⁵ and R^{e} may be bonded to each other to form an aromatic ring or a heteroaromatic ring,
12) [symbol] indicates the position to be bonded.

Also, at least one of Ar⁴ to Ar⁶ in Formula 2 is represented by any one of Formula c-1 to Formula c-6.

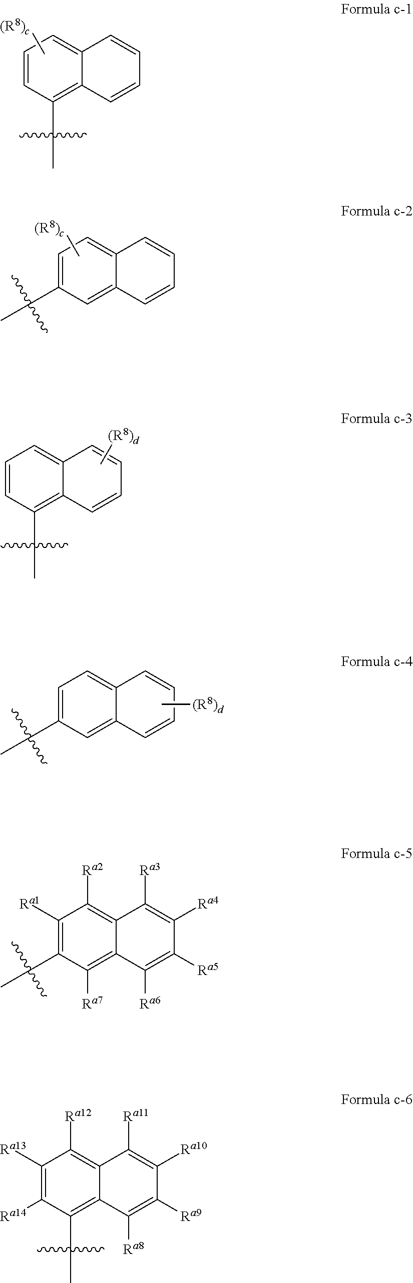

Wherein,
1) R⁸ and Ra¹, Ra², Ra³, Ra⁴, Ra⁵, Ra⁶, Ra⁷, Ra⁸, Ra⁹, Ra¹⁰, Ra¹¹, Ra¹², Ra¹³, Ra¹⁴ are each independently selected from the group consisting of a hydrogen; a C₁-C₂₀ alkyl group; a C₆-C₂₀ aryl group; a C₆-C₂₀ aryl group substituted by deuterium; fluorenyl group; and a C₂-C₂₀ heterocyclic group;
2) c is an integer from 0 to 3, d is an integer from 0 to 4.

Also, the compound represented by Formula 2 is represented by any of the following Formulas 2-1 to 2-4.

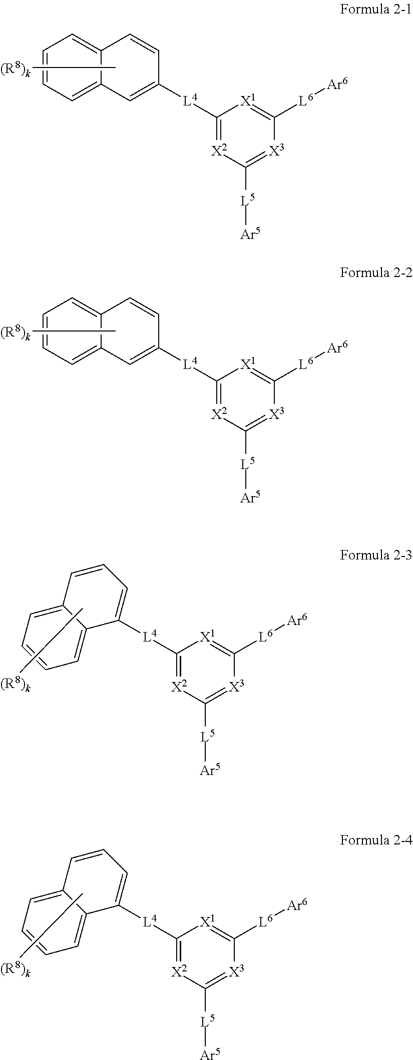

Wherein,
1) Ar⁵, Ar⁶, X¹, X², X³, L⁴, L⁵ and L⁶ are the same as defined in Formula 2,
2) R⁸ is each independently selected from the group consisting of a hydrogen; a C₁-C₂₀ alkyl group; a C₆-C₂₀ aryl group; a C₆-C₂₀ aryl group substituted by deuterium; fluorenyl group; a C₂-C₂₀ heterocyclic group;
3) k is an integer of 0 to 7.

Also, the compound represented by Formula 2 is represented by any one of Formulas 2-5 to 2-8.

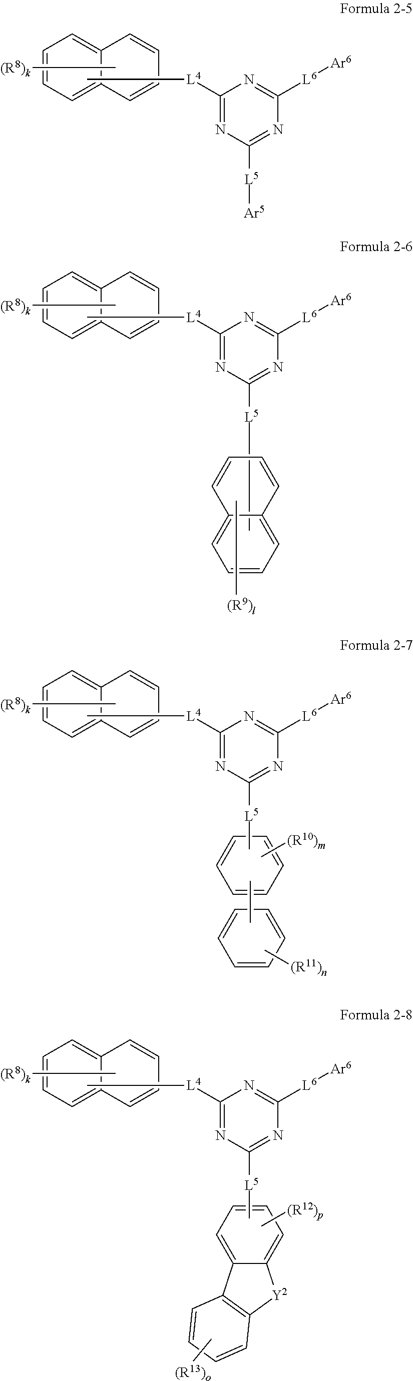

Wherein,
1) Ar⁵, Ar⁶, L⁴, L⁵ and L⁶ are the are the same as defined in Formula 2,
2) R⁸ is each independently selected from the group consisting of a hydrogen; a C₁-C₂₀ alkyl group; a C₆-C₂₀ aryl group; a C₆-C₂₀ aryl group substituted by deuterium; fluorenyl group; a C₂-C₂₀ heterocyclic group;
3) R⁹, R¹⁰, R¹¹, R¹² and R¹³ are the same as the definition of R′,
4) Y² is CR¹⁴R¹⁵, N—Ar⁷, O or S,
5) R¹⁴ and R¹⁵ are the same as the definition of R′,
6) Ar⁷ is the same as the definition of Ar⁴ in Formula 2,
7) k and l are each independently an integer of 0 to 7, m and o are each independently an integer of 0 to 4, n is an integer of 0 to 5, p is an integer of 0 to 3.

Specifically, the compound represented by Formula 2 may be any one of the following compounds.

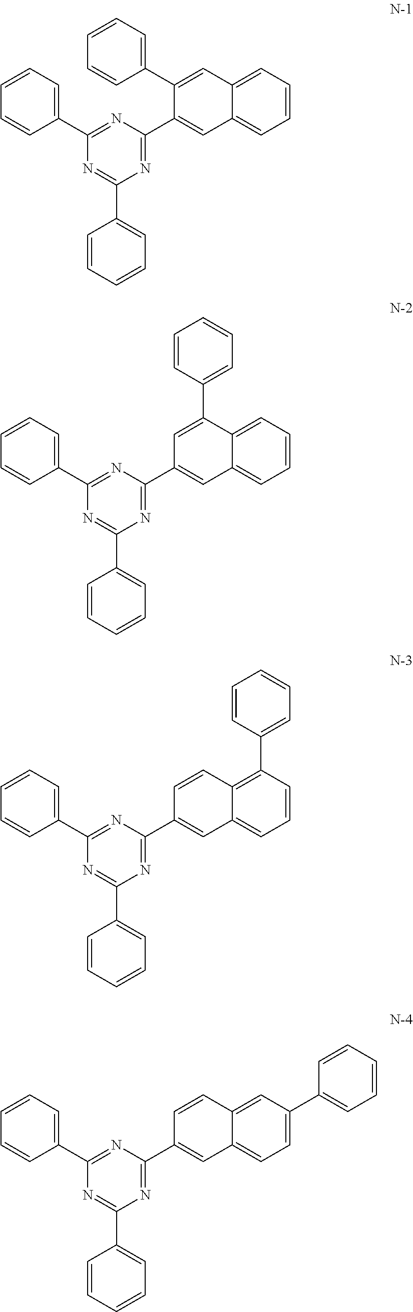 [SMILES: c1ccc(-c2nc(-c3ccccc3)nc(-c3cc4ccccc4cc3-c3ccccc3)n2)cc1 ; c1ccc(-c2nc(-c3ccccc3)nc(-c3cc(-c4ccccc4)c4ccccc4c3)n2)cc1 ; c1ccc(-c2nc(-c3ccccc3)nc(-c3ccc4c(-c5ccccc5)cccc4c3)n2)cc1 ; c1ccc(-c2ccc3cc(-c4nc(-c5ccccc5)nc(-c5ccccc5)n4)ccc3c2)cc1]

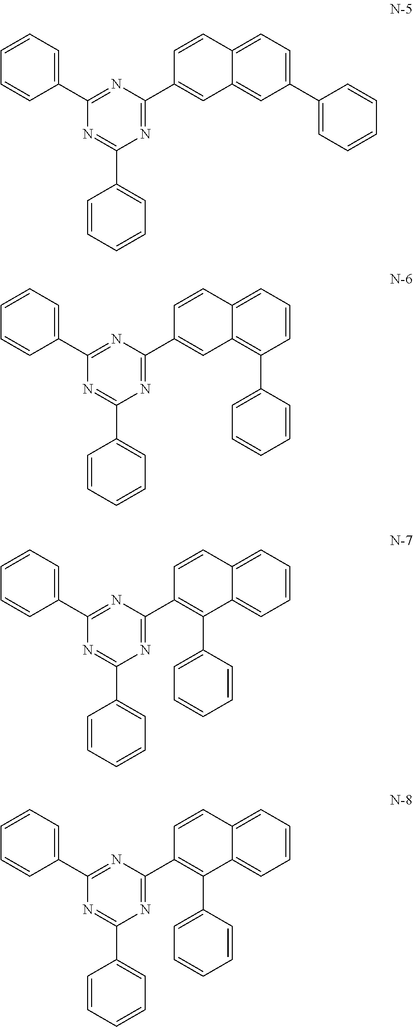 [SMILES: c1ccc(-c2ccc3ccc(-c4nc(-c5ccccc5)nc(-c5ccccc5)n4)cc3c2)cc1 ; c1ccc(-c2nc(-c3ccccc3)nc(-c3ccc4cccc(-c5ccccc5)c4c3)n2)cc1 ; c1ccc(-c2nc(-c3ccccc3)nc(-c3ccc4ccccc4c3-c3ccccc3)n2)cc1 ; c1ccc(-c2nc(-c3ccccc3)nc(-c3ccc4ccccc4c3-c3ccccc3)n2)cc1]

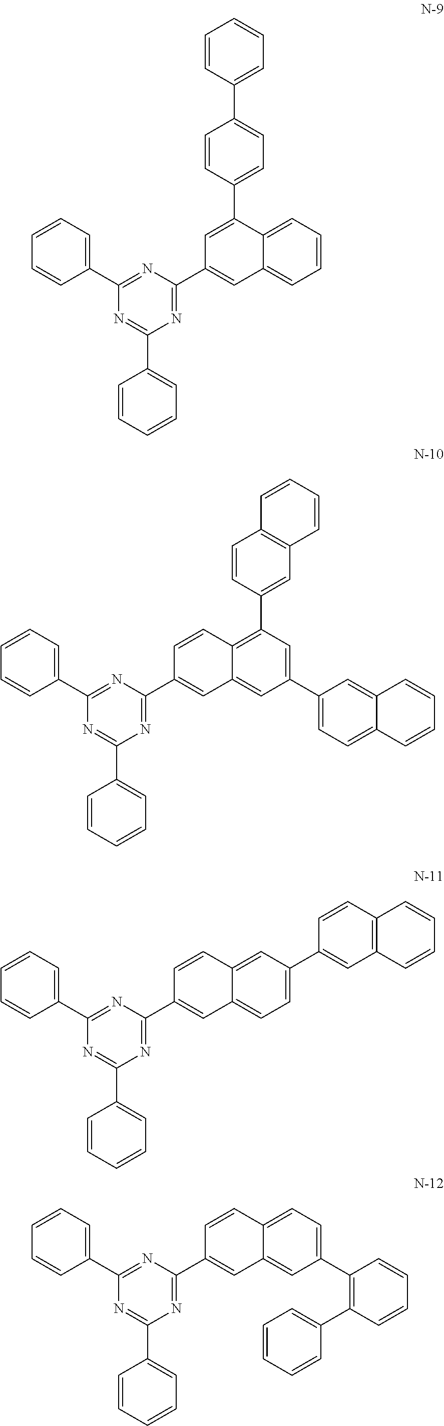 [SMILES: c1ccc(-c2ccc(-c3cc(-c4nc(-c5ccccc5)nc(-c5ccccc5)n4)cc4ccccc34)cc2)cc1 ; c1ccc(-c2nc(-c3ccccc3)nc(-c3ccc4c(-c5ccc6ccccc6c5)cc(-c5ccc6ccccc6c5)cc4c3)n2)cc1 ; c1ccc(-c2nc(-c3ccccc3)nc(-c3ccc4cc(-c5ccc6ccccc6c5)ccc4c3)n2)cc1 ; c1ccc(-c2nc(-c3ccccc3)nc(-c3ccc4ccc(-c5ccccc5-c5ccccc5)cc4c3)n2)cc1]

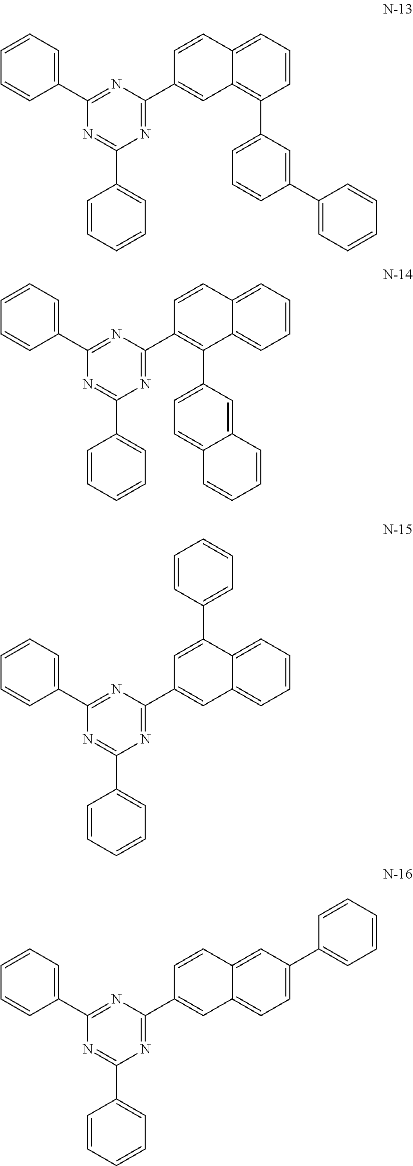 [SMILES: c1ccc(-c2cccc(-c3cccc4ccc(-c5nc(-c6ccccc6)nc(-c6ccccc6)n5)cc34)c2)cc1 ; c1ccc(-c2nc(-c3ccccc3)nc(-c3ccc4ccccc4c3-c3ccc4ccccc4c3)n2)cc1 ; c1ccc(-c2nc(-c3ccccc3)nc(-c3cc(-c4ccccc4)c4ccccc4c3)n2)cc1 ; c1ccc(-c2ccc3cc(-c4nc(-c5ccccc5)nc(-c5ccccc5)n4)ccc3c2)cc1]

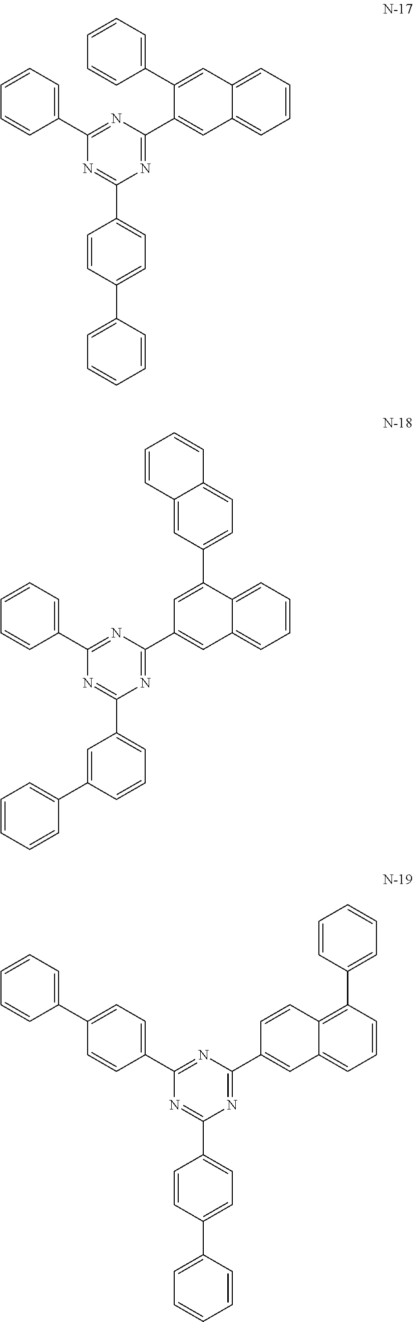 [SMILES: c1ccc(-c2ccc(-c3nc(-c4ccccc4)nc(-c4cc5ccccc5cc4-c4ccccc4)n3)cc2)cc1 ; c1ccc(-c2cccc(-c3nc(-c4ccccc4)nc(-c4cc(-c5ccc6ccccc6c5)c5ccccc5c4)n3)c2)cc1 ; c1ccc(-c2ccc(-c3nc(-c4ccc(-c5ccccc5)cc4)nc(-c4ccc5c(-c6ccccc6)cccc5c4)n3)cc2)cc1]

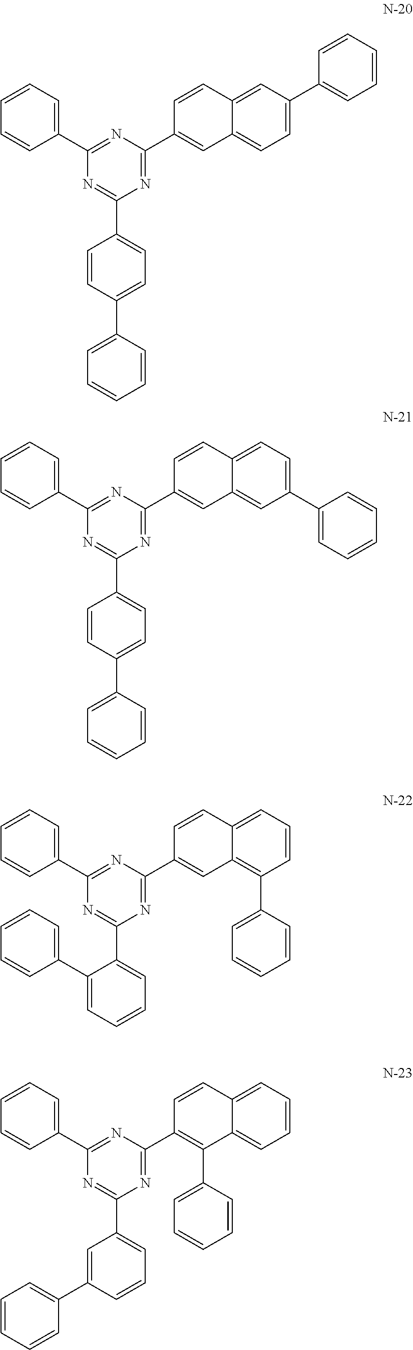 [SMILES: c1ccc(-c2ccc(-c3nc(-c4ccccc4)nc(-c4ccc5cc(-c6ccccc6)ccc5c4)n3)cc2)cc1 ; c1ccc(-c2ccc(-c3nc(-c4ccccc4)nc(-c4ccc5ccc(-c6ccccc6)cc5c4)n3)cc2)cc1 ; c1ccc(-c2nc(-c3ccc4cccc(-c5ccccc5)c4c3)nc(-c3ccccc3-c3ccccc3)n2)cc1 ; c1ccc(-c2cccc(-c3nc(-c4ccccc4)nc(-c4ccc5ccccc5c4-c4ccccc4)n3)c2)cc1]

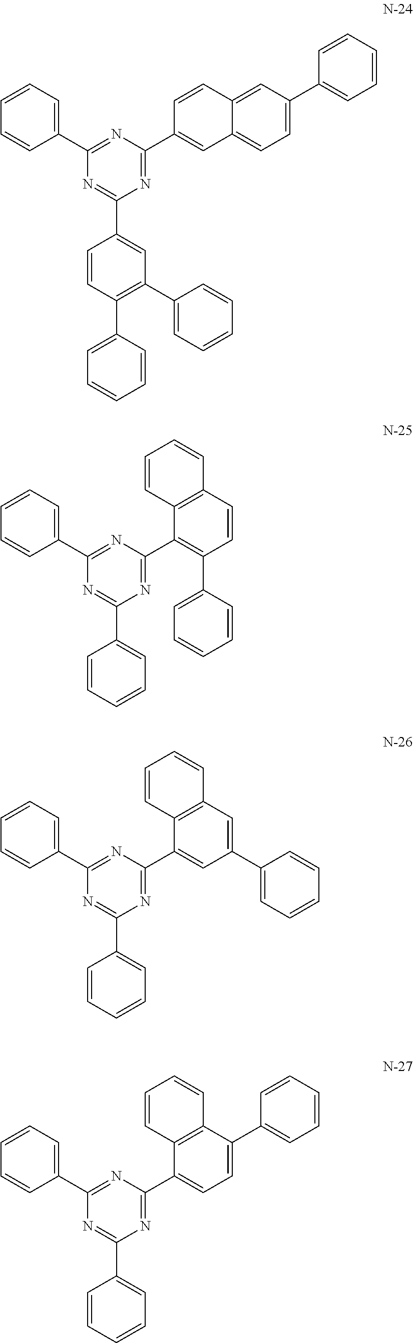 [SMILES: c1ccc(-c2ccc3cc(-c4nc(-c5ccccc5)nc(-c5ccc(-c6ccccc6)c(-c6ccccc6)c5)n4)ccc3c2)cc1 ; c1ccc(-c2nc(-c3ccccc3)nc(-c3c(-c4ccccc4)ccc4ccccc34)n2)cc1 ; c1ccc(-c2cc(-c3nc(-c4ccccc4)nc(-c4ccccc4)n3)c3ccccc3c2)cc1 ; c1ccc(-c2nc(-c3ccccc3)nc(-c3ccc(-c4ccccc4)c4ccccc34)n2)cc1]

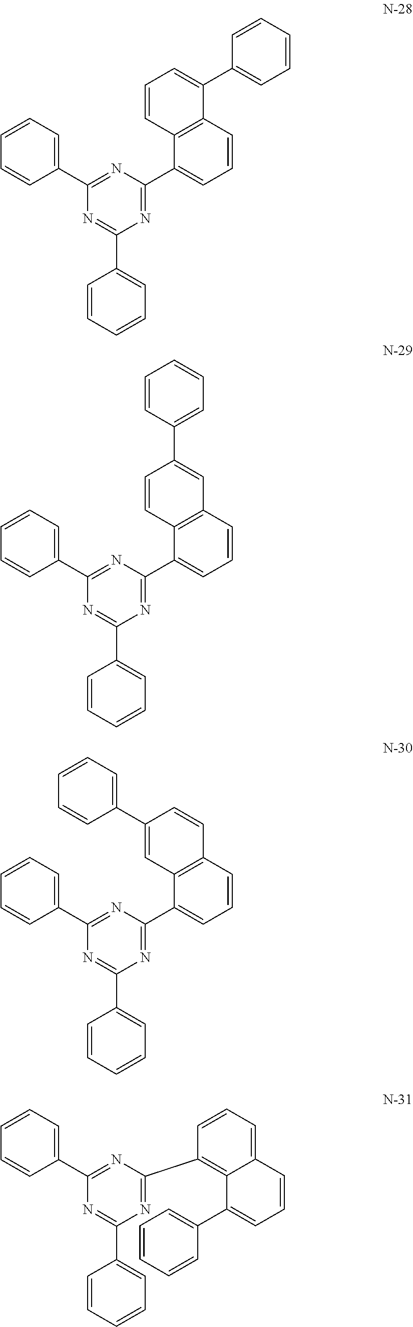

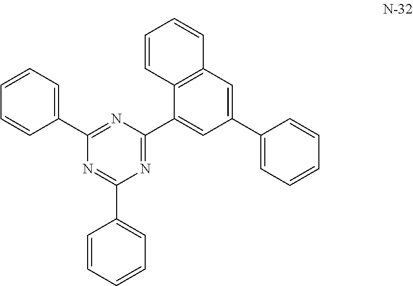 [SMILES: c1ccc(-c2cc(-c3nc(-c4ccccc4)nc(-c4ccccc4)n3)c3ccccc3c2)cc1]

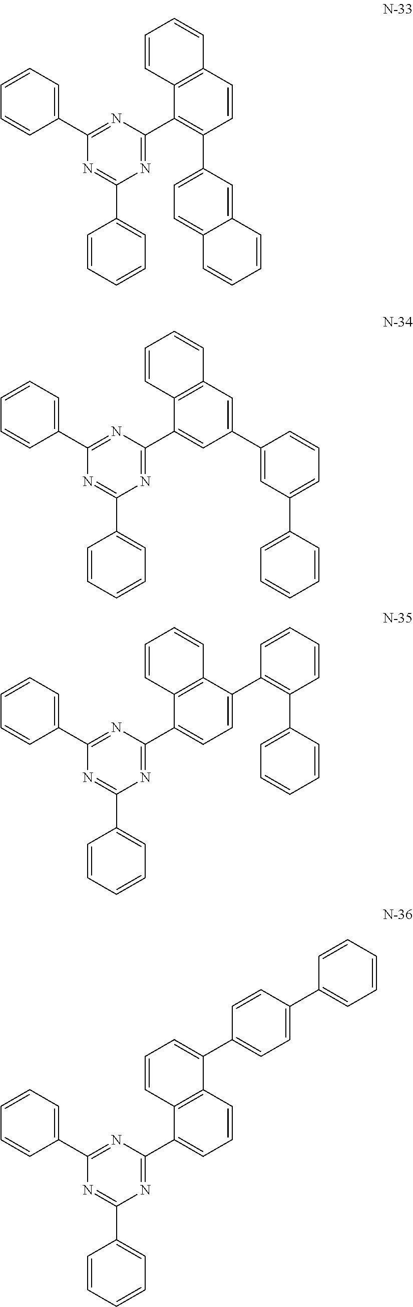

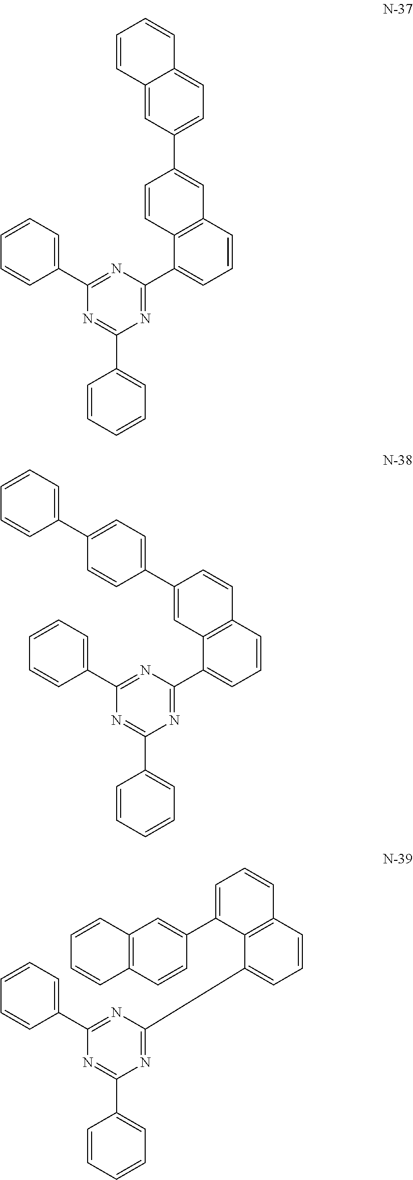 [SMILES: c1ccc(-c2nc(-c3ccccc3)nc(-c3cccc4cc(-c5ccc6ccccc6c5)ccc34)n2)cc1 ; c1ccc(-c2ccc(-c3ccc4cccc(-c5nc(-c6ccccc6)nc(-c6ccccc6)n5)c4c3)cc2)cc1 ; c1ccc(-c2nc(-c3ccccc3)nc(-c3cccc4cccc(-c5ccc6ccccc6c5)c34)n2)cc1]

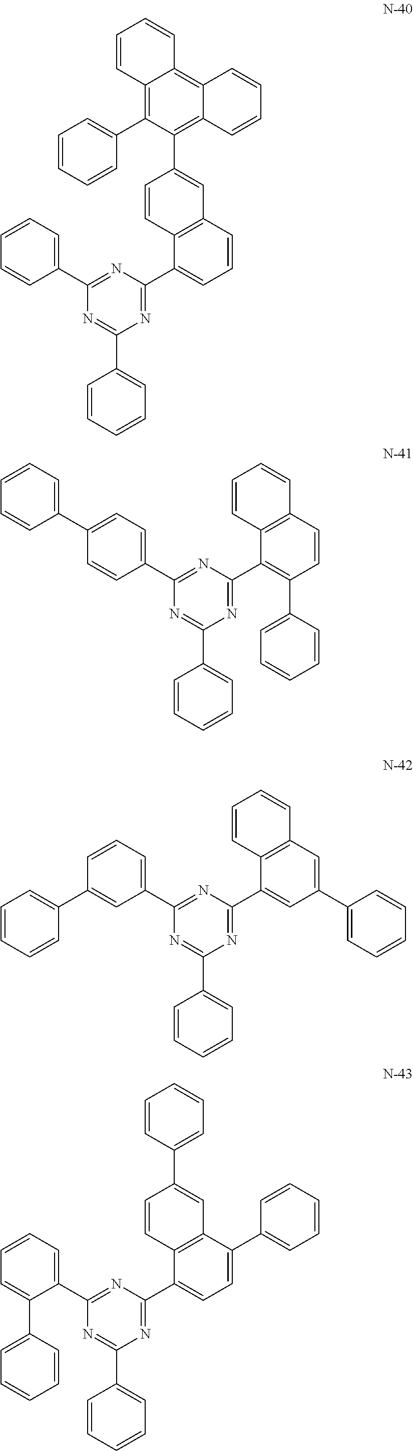

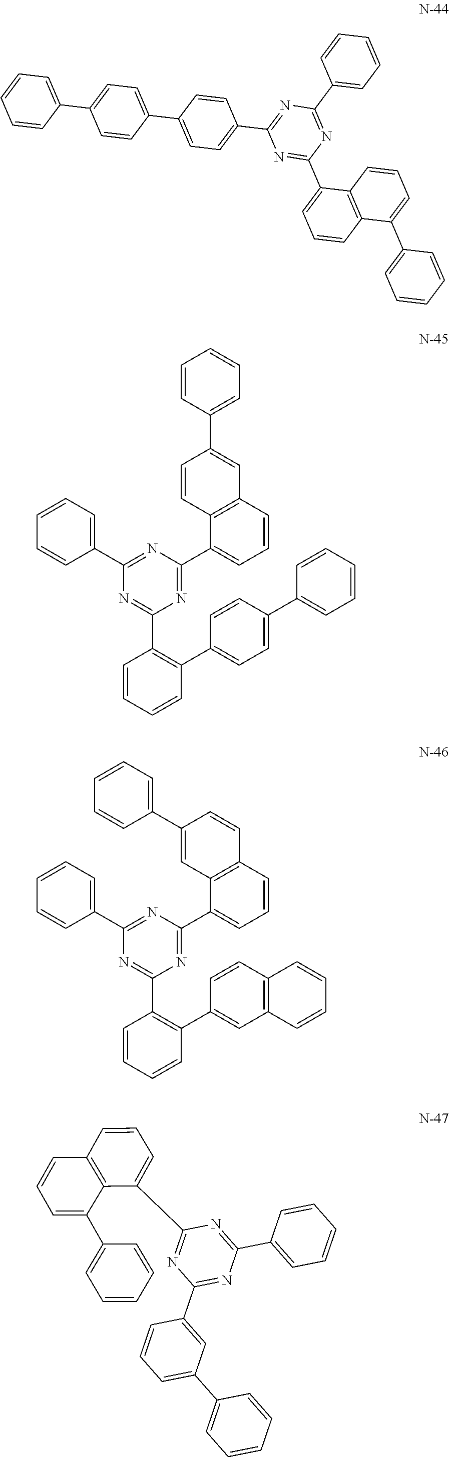 [SMILES: c1ccc(-c2ccc(-c3ccc(-c4nc(-c5ccccc5)nc(-c5cccc6c(-c7ccccc7)cccc56)n4)cc3)cc2)cc1 ; c1ccc(-c2ccc(-c3ccccc3-c3nc(-c4ccccc4)nc(-c4cccc5cc(-c6ccccc6)ccc45)n3)cc2)cc1 ; c1ccc(-c2ccc3cccc(-c4nc(-c5ccccc5)nc(-c5ccccc5-c5ccc6ccccc6c5)n4)c3c2)cc1 ; c1ccc(-c2cccc(-c3nc(-c4ccccc4)nc(-c4cccc5cccc(-c6ccccc6)c45)n3)c2)cc1]

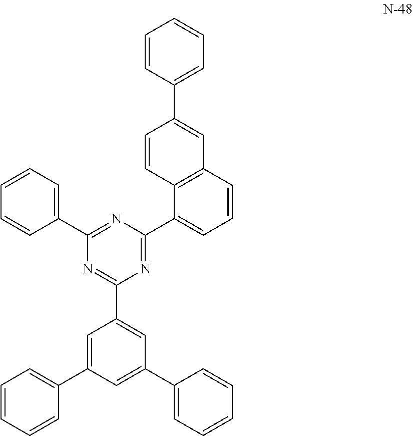 [SMILES: c1ccc(-c2cc(-c3ccccc3)cc(-c3nc(-c4ccccc4)nc(-c4cccc5cc(-c6ccccc6)ccc45)n3)c2)cc1]

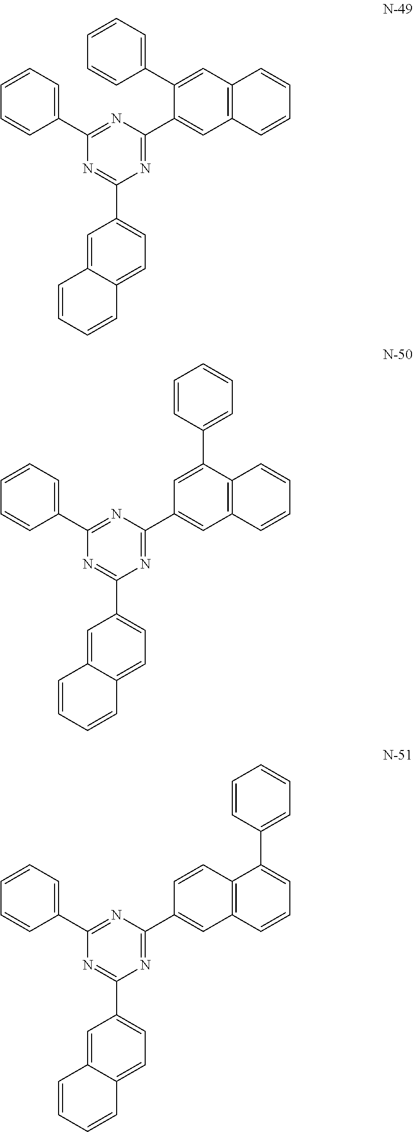 [SMILES: c1ccc(-c2nc(-c3ccc4ccccc4c3)nc(-c3cc4ccccc4cc3-c3ccccc3)n2)cc1 ; c1ccc(-c2nc(-c3ccc4ccccc4c3)nc(-c3cc(-c4ccccc4)c4ccccc4c3)n2)cc1 ; c1ccc(-c2nc(-c3ccc4ccccc4c3)nc(-c3ccc4c(-c5ccccc5)cccc4c3)n2)cc1]

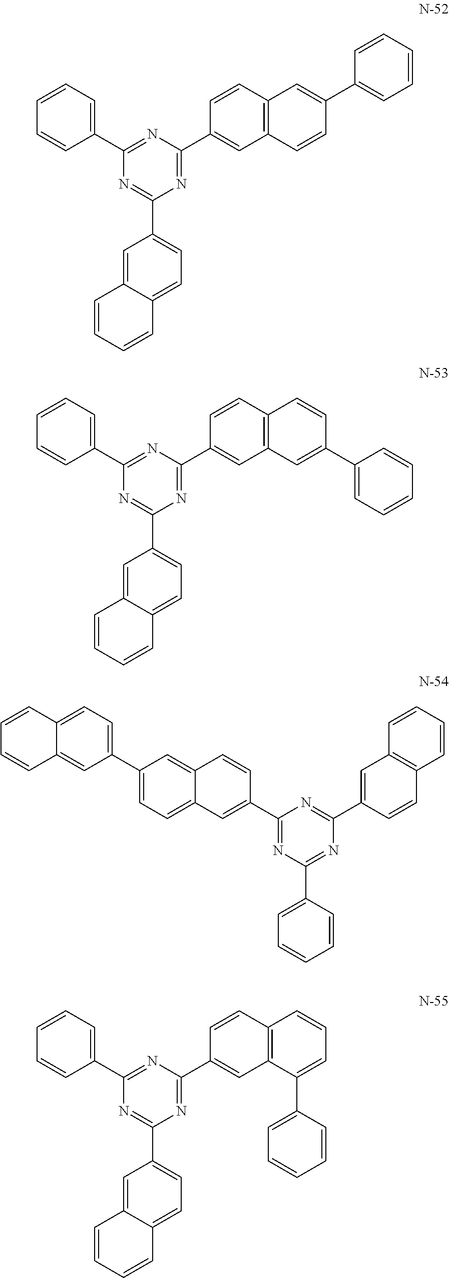 [SMILES: c1ccc(-c2ccc3cc(-c4nc(-c5ccccc5)nc(-c5ccc6ccccc6c5)n4)ccc3c2)cc1 ; c1ccc(-c2ccc3ccc(-c4nc(-c5ccccc5)nc(-c5ccc6ccccc6c5)n4)cc3c2)cc1 ; c1ccc(-c2nc(-c3ccc4ccccc4c3)nc(-c3ccc4cc(-c5ccc6ccccc6c5)ccc4c3)n2)cc1 ; c1ccc(-c2nc(-c3ccc4ccccc4c3)nc(-c3ccc4cccc(-c5ccccc5)c4c3)n2)cc1]

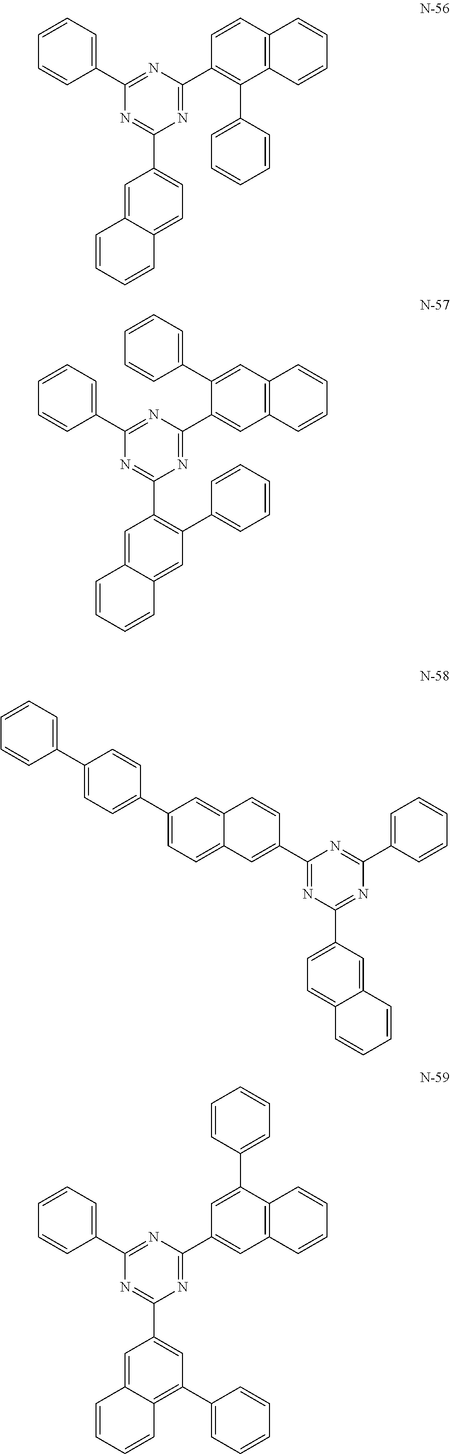

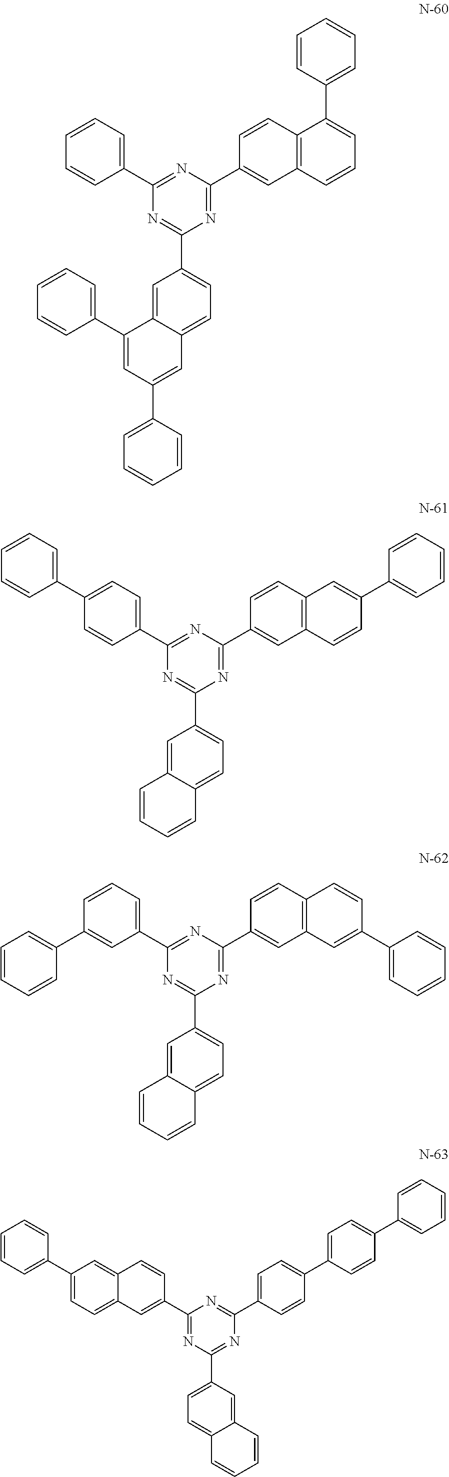 [SMILES: c1ccc(-c2cc(-c3ccccc3)c3cc(-c4nc(-c5ccccc5)nc(-c5ccc6c(-c7ccccc7)cccc6c5)n4)ccc3c2)cc1 ; c1ccc(-c2ccc(-c3nc(-c4ccc5ccccc5c4)nc(-c4ccc5cc(-c6ccccc6)ccc5c4)n3)cc2)cc1 ; c1ccc(-c2cccc(-c3nc(-c4ccc5ccccc5c4)nc(-c4ccc5ccc(-c6ccccc6)cc5c4)n3)c2)cc1 ; c1ccc(-c2ccc(-c3ccc(-c4nc(-c5ccc6ccccc6c5)nc(-c5ccc6cc(-c7ccccc7)ccc6c5)n4)cc3)cc2)cc1]

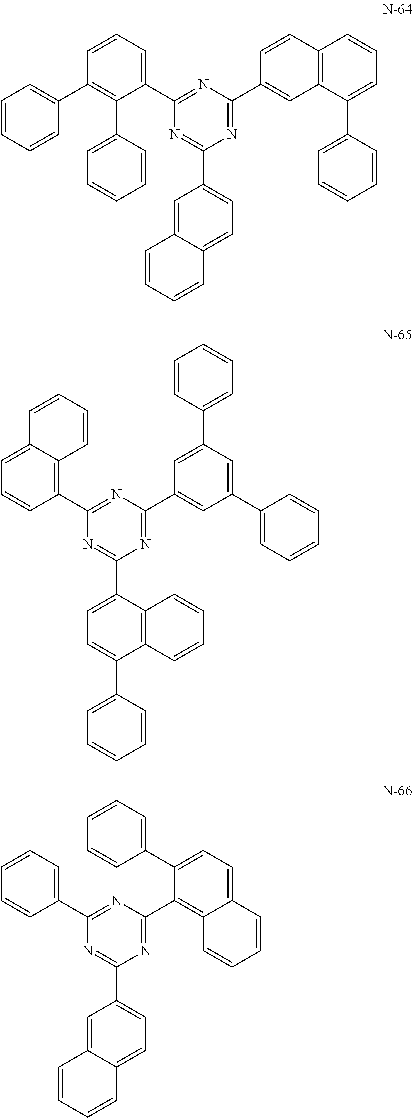

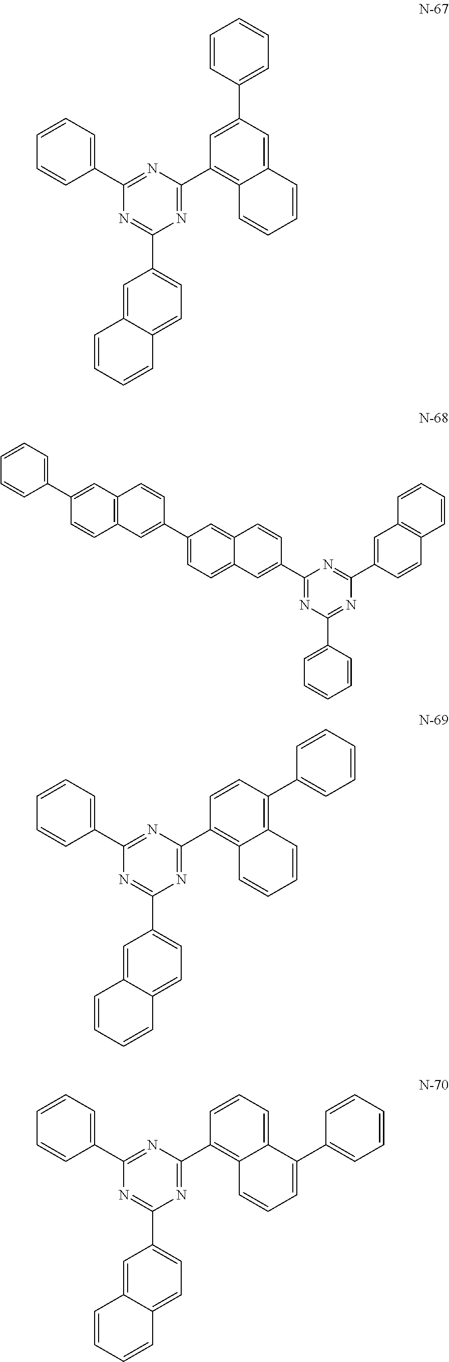

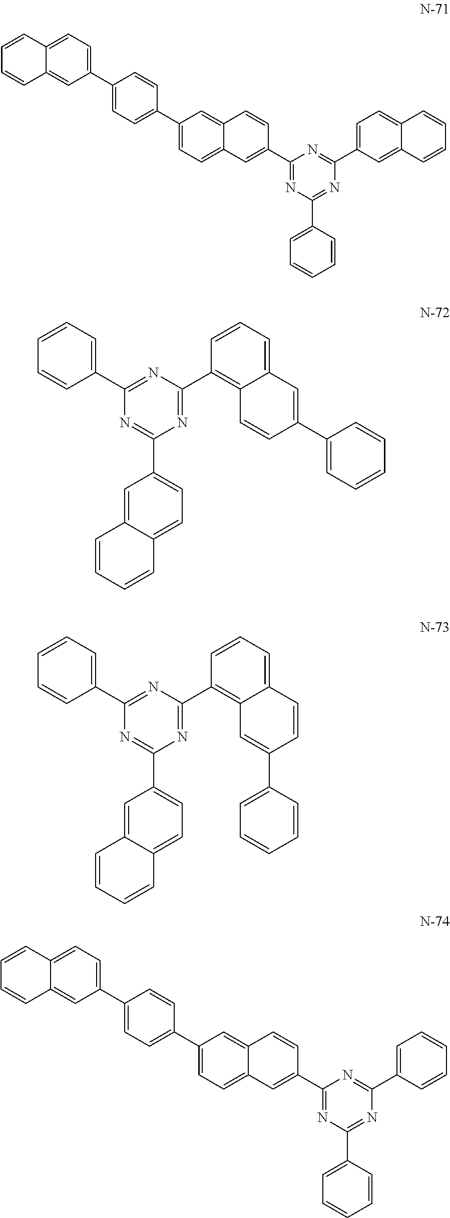 [SMILES: c1ccc(-c2nc(-c3ccc4ccccc4c3)nc(-c3ccc4cc(-c5ccc(-c6ccc7ccccc7c6)cc5)ccc4c3)n2)cc1 ; c1ccc(-c2ccc3c(-c4nc(-c5ccccc5)nc(-c5ccc6ccccc6c5)n4)cccc3c2)cc1 ; c1ccc(-c2ccc3cccc(-c4nc(-c5ccccc5)nc(-c5ccc6ccccc6c5)n4)c3c2)cc1 ; c1ccc(-c2nc(-c3ccccc3)nc(-c3ccc4cc(-c5ccc(-c6ccc7ccccc7c6)cc5)ccc4c3)n2)cc1]

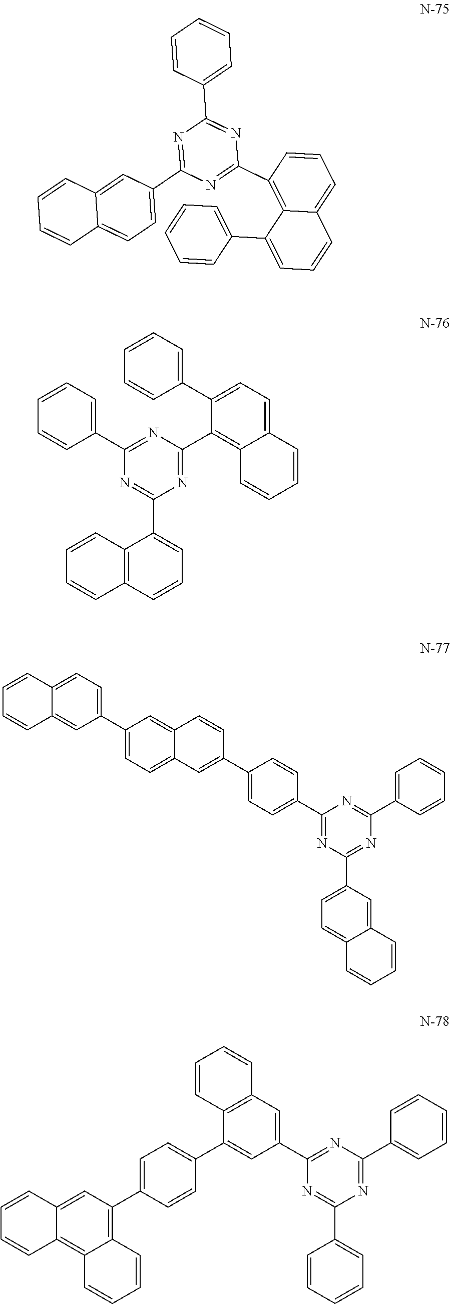 [SMILES: c1ccc(-c2nc(-c3ccc4ccccc4c3)nc(-c3cccc4cccc(-c5ccccc5)c34)n2)cc1 ; c1ccc(-c2nc(-c3cccc4ccccc34)nc(-c3c(-c4ccccc4)ccc4ccccc34)n2)cc1 ; c1ccc(-c2nc(-c3ccc(-c4ccc5cc(-c6ccc7ccccc7c6)ccc5c4)cc3)nc(-c3ccc4ccccc4c3)n2)cc1 ; c1ccc(-c2nc(-c3ccccc3)nc(-c3cc(-c4ccc(-c5cc6ccccc6c6ccccc56)cc4)c4ccccc4c3)n2)cc1]

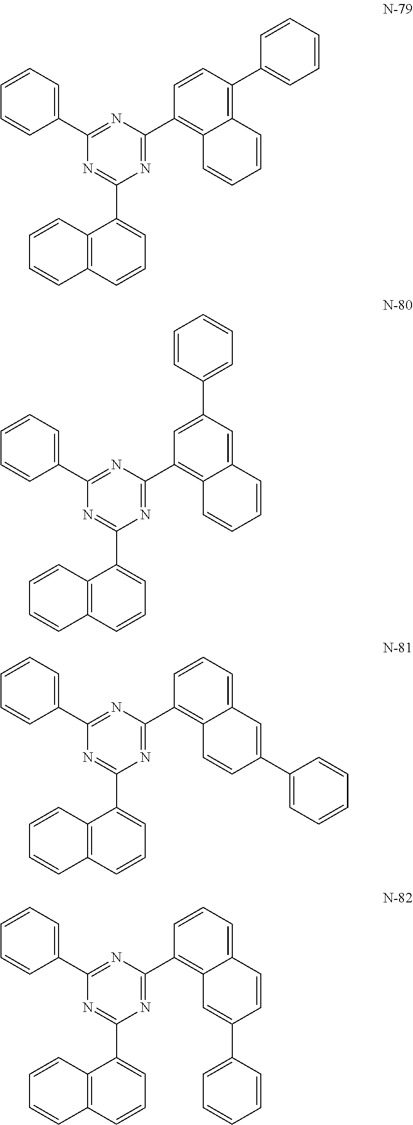

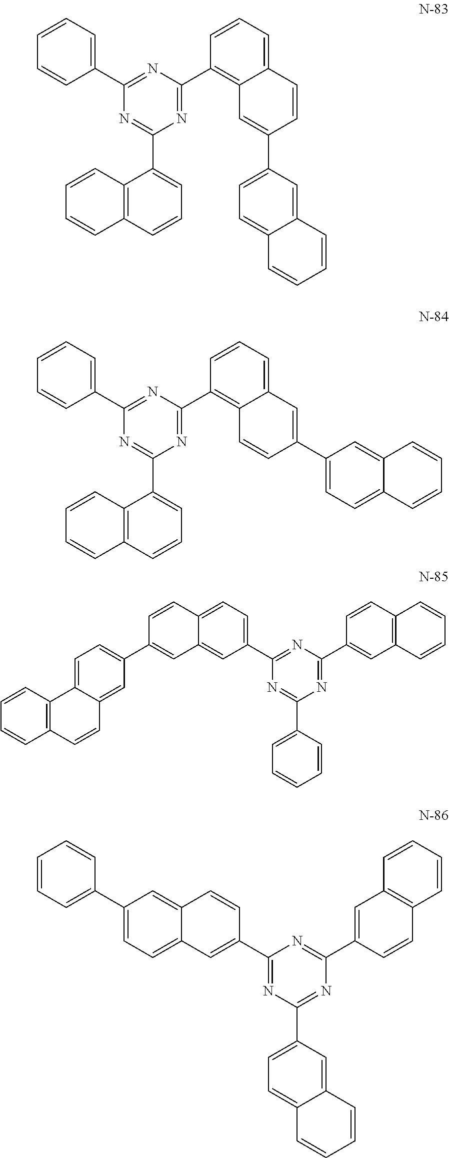 [SMILES: c1ccc(-c2nc(-c3cccc4ccccc34)nc(-c3cccc4ccc(-c5ccc6ccccc6c5)cc34)n2)cc1 ; c1ccc(-c2nc(-c3cccc4ccccc34)nc(-c3cccc4cc(-c5ccc6ccccc6c5)ccc34)n2)cc1 ; c1ccc(-c2nc(-c3ccc4ccccc4c3)nc(-c3ccc4ccc(-c5ccc6c(ccc7ccccc76)c5)cc4c3)n2)cc1 ; c1ccc(-c2ccc3cc(-c4nc(-c5ccc6ccccc6c5)nc(-c5ccc6ccccc6c5)n4)ccc3c2)cc1]

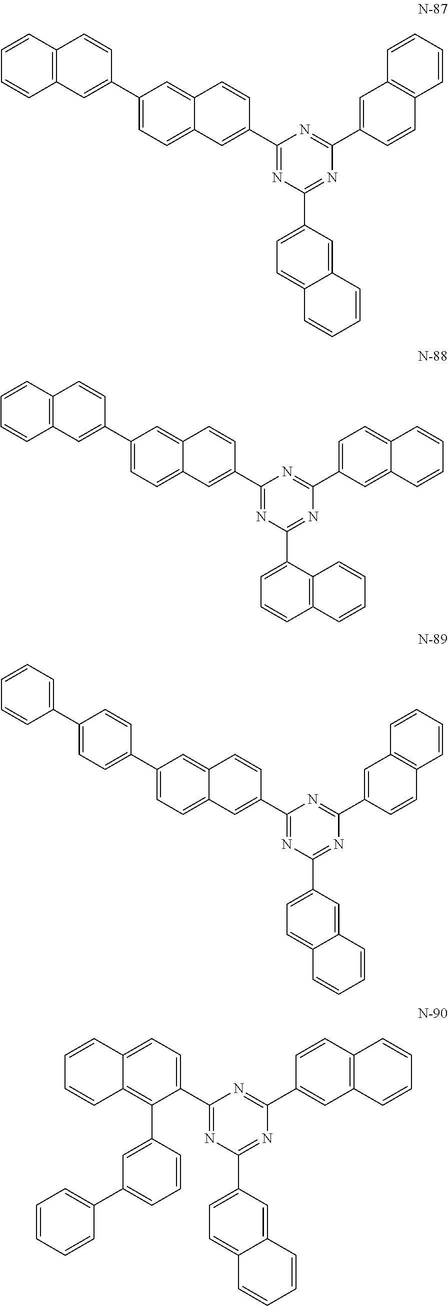 [SMILES: c1ccc2cc(-c3ccc4cc(-c5nc(-c6ccc7ccccc7c6)nc(-c6ccc7ccccc7c6)n5)ccc4c3)ccc2c1 ; c1ccc2cc(-c3ccc4cc(-c5nc(-c6ccc7ccccc7c6)nc(-c6cccc7ccccc67)n5)ccc4c3)ccc2c1 ; c1ccc(-c2ccc(-c3ccc4cc(-c5nc(-c6ccc7ccccc7c6)nc(-c6ccc7ccccc7c6)n5)ccc4c3)cc2)cc1 ; c1ccc(-c2cccc(-c3c(-c4nc(-c5ccc6ccccc6c5)nc(-c5ccc6ccccc6c5)n4)ccc4ccccc34)c2)cc1]

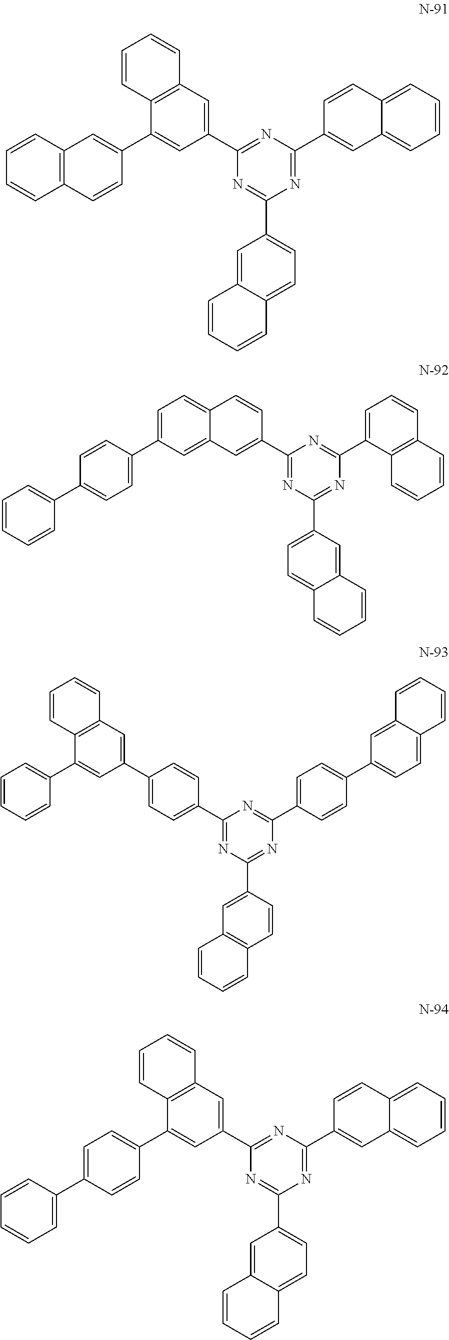 [SMILES: c1ccc2cc(-c3nc(-c4ccc5ccccc5c4)nc(-c4cc(-c5ccc6ccccc6c5)c5ccccc5c4)n3)ccc2c1 ; c1ccc(-c2ccc(-c3ccc4ccc(-c5nc(-c6ccc7ccccc7c6)nc(-c6cccc7ccccc67)n5)cc4c3)cc2)cc1 ; c1ccc(-c2cc(-c3ccc(-c4nc(-c5ccc(-c6ccc7ccccc7c6)cc5)nc(-c5ccc6ccccc6c5)n4)cc3)cc3ccccc23)cc1 ; c1ccc(-c2ccc(-c3cc(-c4nc(-c5ccc6ccccc6c5)nc(-c5ccc6ccccc6c5)n4)cc4ccccc34)cc2)cc1]

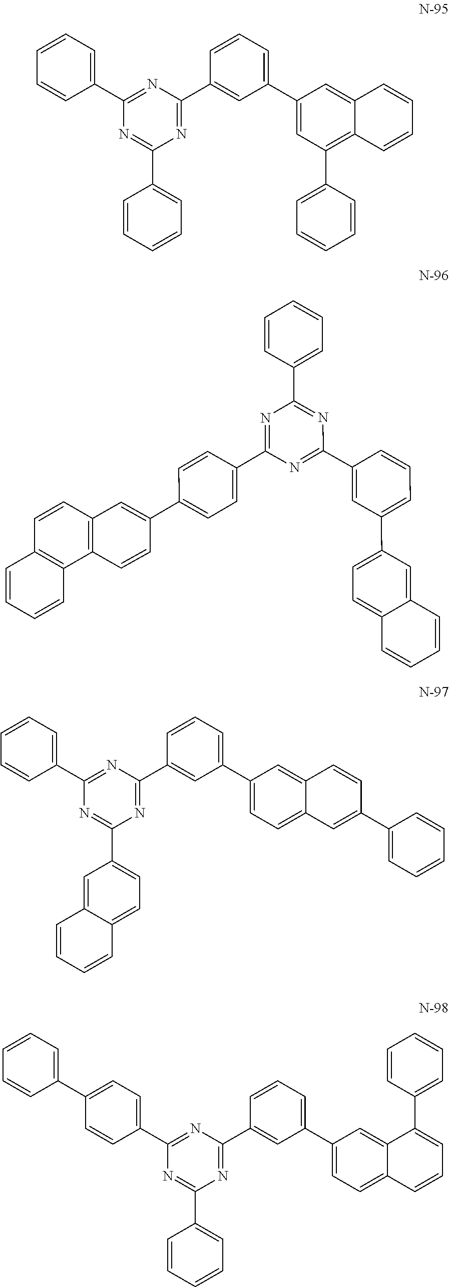 [SMILES: c1ccc(-c2nc(-c3ccccc3)nc(-c3cccc(-c4cc(-c5ccccc5)c5ccccc5c4)c3)n2)cc1 ; c1ccc(-c2nc(-c3ccc(-c4ccc5c(ccc6ccccc65)c4)cc3)nc(-c3cccc(-c4ccc5ccccc5c4)c3)n2)cc1 ; c1ccc(-c2ccc3cc(-c4cccc(-c5nc(-c6ccccc6)nc(-c6ccc7ccccc7c6)n5)c4)ccc3c2)cc1 ; c1ccc(-c2ccc(-c3nc(-c4ccccc4)nc(-c4cccc(-c5ccc6cccc(-c7ccccc7)c6c5)c4)n3)cc2)cc1]

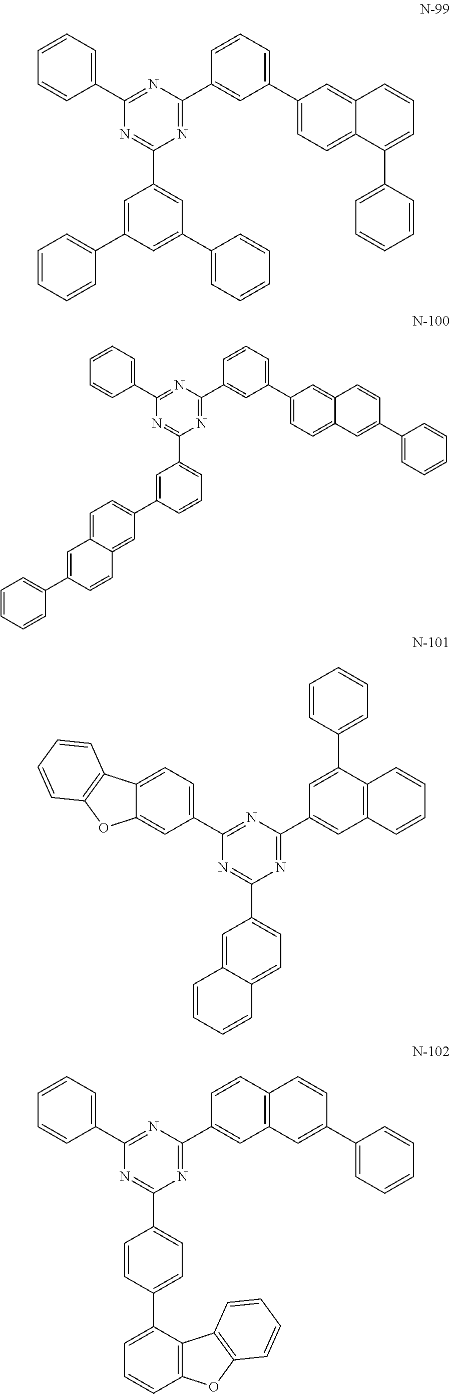

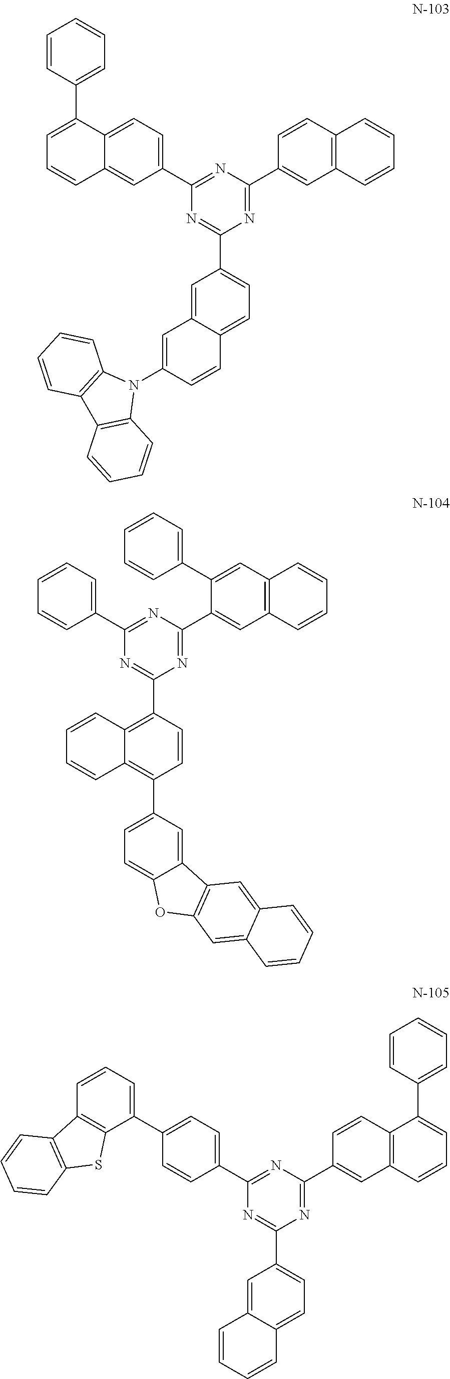

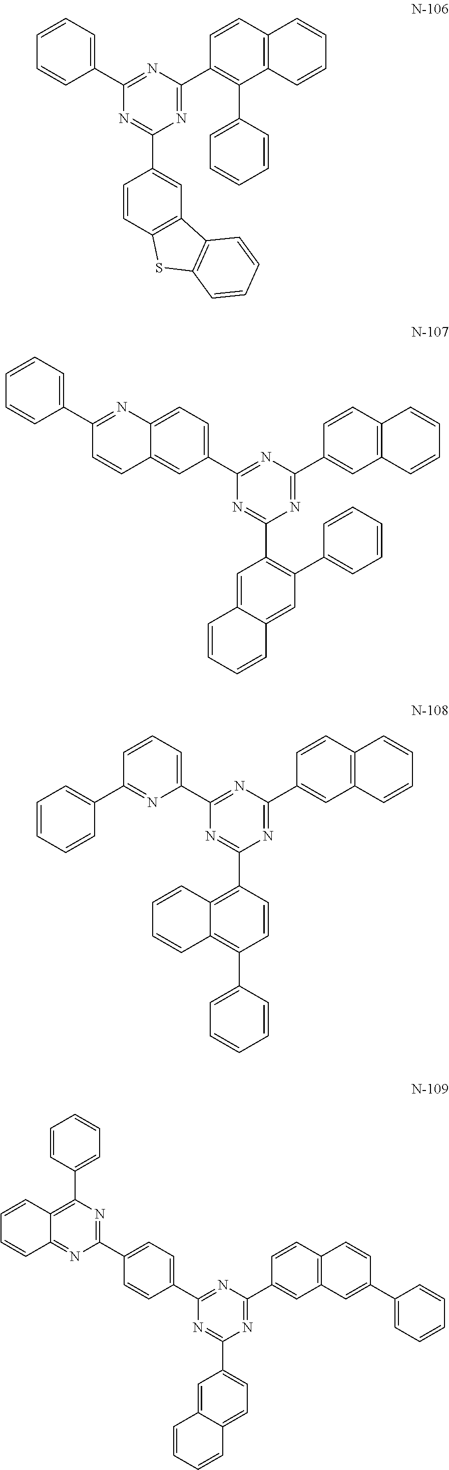 [SMILES: c1ccc(-c2nc(-c3ccc4sc5ccccc5c4c3)nc(-c3ccc4ccccc4c3-c3ccccc3)n2)cc1 ; c1ccc(-c2ccc3cc(-c4nc(-c5ccc6ccccc6c5)nc(-c5cc6ccccc6cc5-c5ccccc5)n4)ccc3n2)cc1 ; c1ccc(-c2cccc(-c3nc(-c4ccc5ccccc5c4)nc(-c4ccc(-c5ccccc5)c5ccccc45)n3)n2)cc1 ; c1ccc(-c2ccc3ccc(-c4nc(-c5ccc(-c6nc(-c7ccccc7)c7ccccc7n6)cc5)nc(-c5ccc6ccccc6c5)n4)cc3c2)cc1]

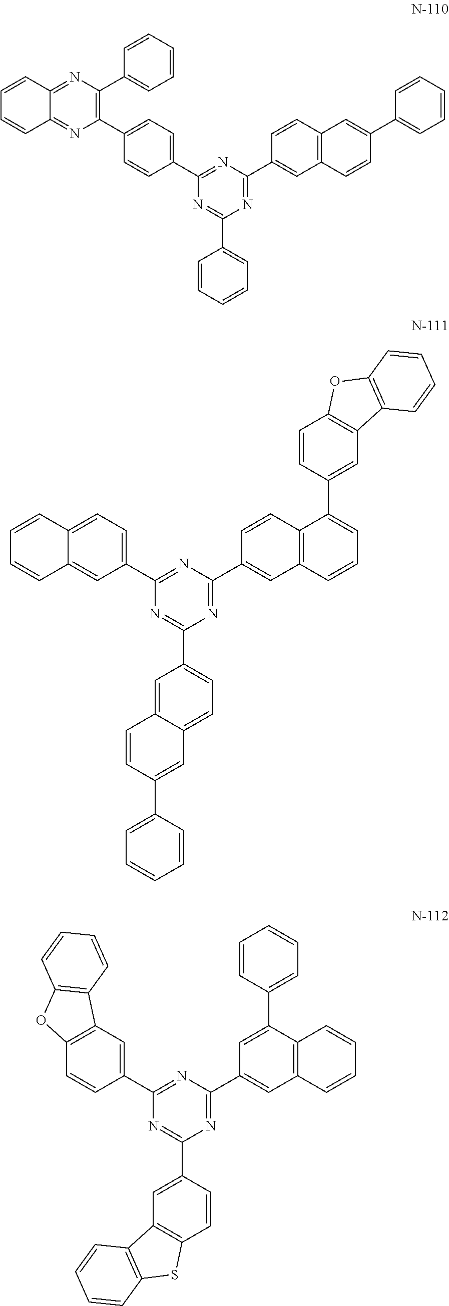 [SMILES: c1ccc(-c2ccc3cc(-c4nc(-c5ccccc5)nc(-c5ccc(-c6nc7ccccc7nc6-c6ccccc6)cc5)n4)ccc3c2)cc1 ; c1ccc(-c2ccc3cc(-c4nc(-c5ccc6ccccc6c5)nc(-c5ccc6c(-c7ccc8oc9ccccc9c8c7)cccc6c5)n4)ccc3c2)cc1 ; c1ccc(-c2cc(-c3nc(-c4ccc5oc6ccccc6c5c4)nc(-c4ccc5sc6ccccc6c5c4)n3)cc3ccccc23)cc1]

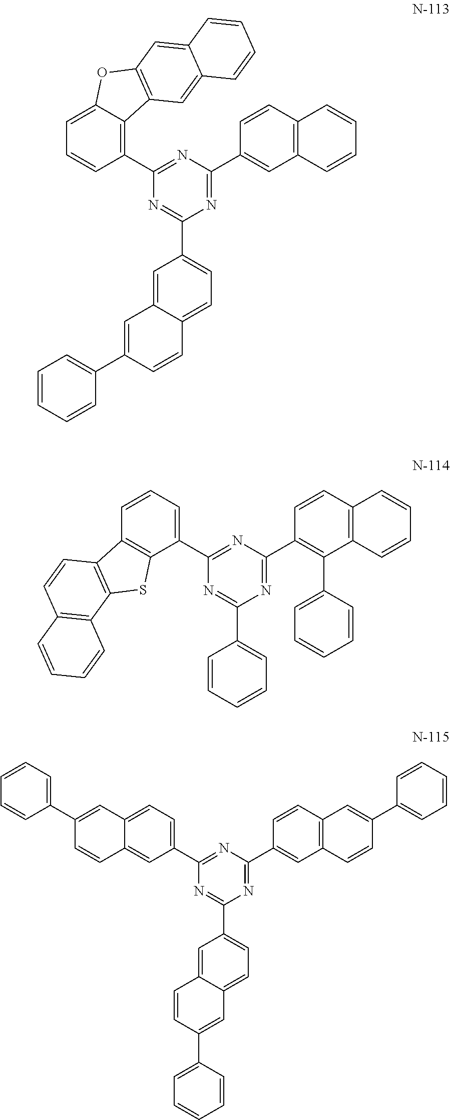 [SMILES: c1ccc(-c2ccc3ccc(-c4nc(-c5ccc6ccccc6c5)nc(-c5cccc6oc7cc8ccccc8cc7c56)n4)cc3c2)cc1 ; c1ccc(-c2nc(-c3ccc4ccccc4c3-c3ccccc3)nc(-c3cccc4c3sc3c5ccccc5ccc43)n2)cc1 ; c1ccc(-c2ccc3cc(-c4nc(-c5ccc6cc(-c7ccccc7)ccc6c5)nc(-c5ccc6cc(-c7ccccc7)ccc6c5)n4)ccc3c2)cc1]

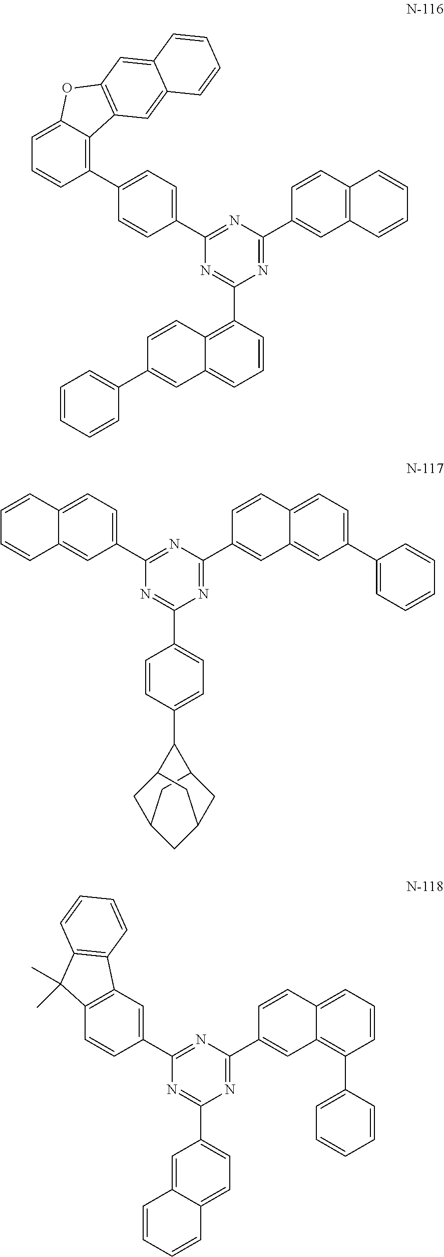 [SMILES: c1ccc(-c2ccc3c(-c4nc(-c5ccc(-c6cccc7oc8cc9ccccc9cc8c67)cc5)nc(-c5ccc6ccccc6c5)n4)cccc3c2)cc1 ; c1ccc(-c2ccc3ccc(-c4nc(-c5ccc(C6C7CC8CC(C7)CC6C8)cc5)nc(-c5ccc6ccccc6c5)n4)cc3c2)cc1 ; CC1(C)c2ccccc2-c2cc(-c3nc(-c4ccc5ccccc5c4)nc(-c4ccc5cccc(-c6ccccc6)c5c4)n3)ccc21]

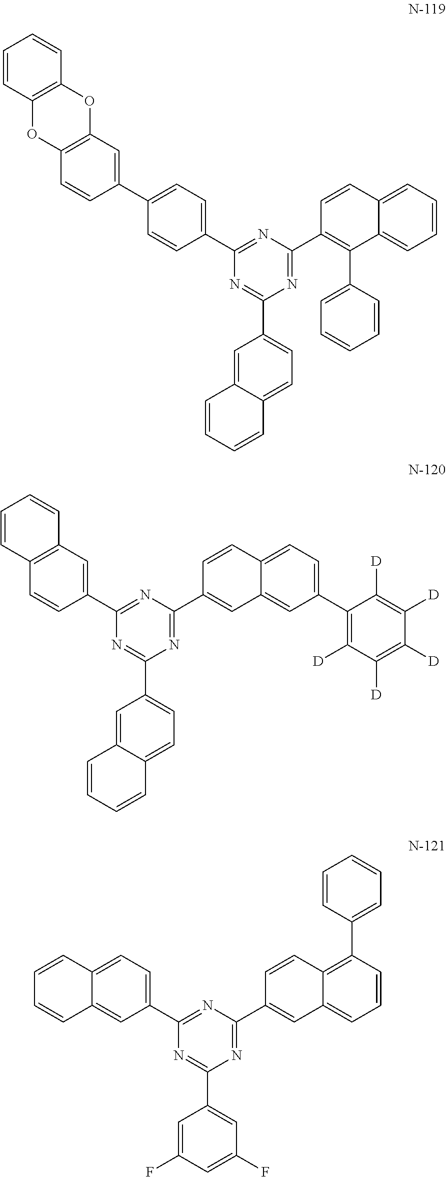 [SMILES: c1ccc(-c2c(-c3nc(-c4ccc(-c5ccc6c(c5)Oc5ccccc5O6)cc4)nc(-c4ccc5ccccc5c4)n3)ccc3ccccc23)cc1 ; [2H]c1c([2H])c([2H])c(-c2ccc3ccc(-c4nc(-c5ccc6ccccc6c5)nc(-c5ccc6ccccc6c5)n4)cc3c2)c([2H])c1[2H] ; Fc1cc(F)cc(-c2nc(-c3ccc4ccccc4c3)nc(-c3ccc4c(-c5ccccc5)cccc4c3)n2)c1]

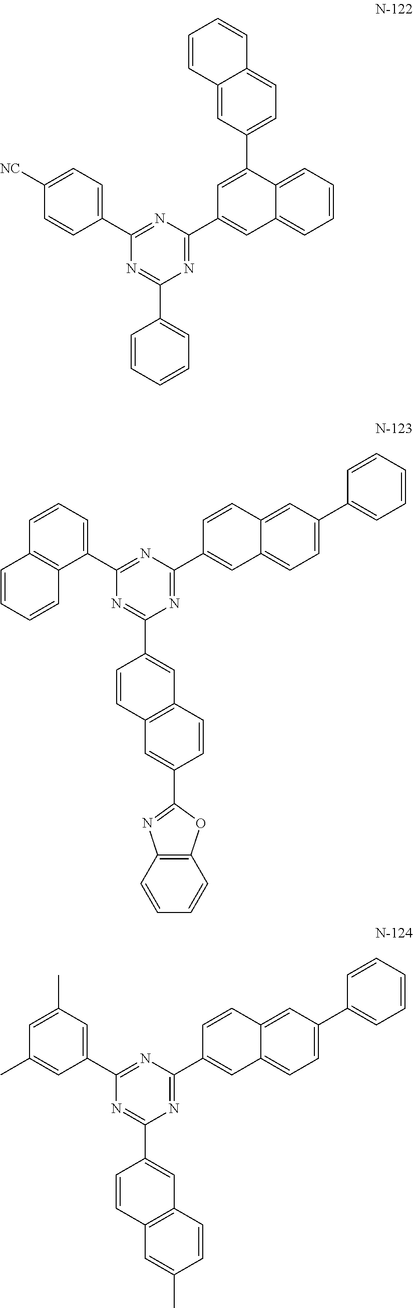 [SMILES: N#Cc1ccc(-c2nc(-c3ccccc3)nc(-c3cc(-c4ccc5ccccc5c4)c4ccccc4c3)n2)cc1 ; c1ccc(-c2ccc3cc(-c4nc(-c5ccc6cc(-c7nc8ccccc8o7)ccc6c5)nc(-c5cccc6ccccc56)n4)ccc3c2)cc1 ; Cc1cc(C)cc(-c2nc(-c3ccc4cc(C)ccc4c3)nc(-c3ccc4cc(-c5ccccc5)ccc4c3)n2)c1]

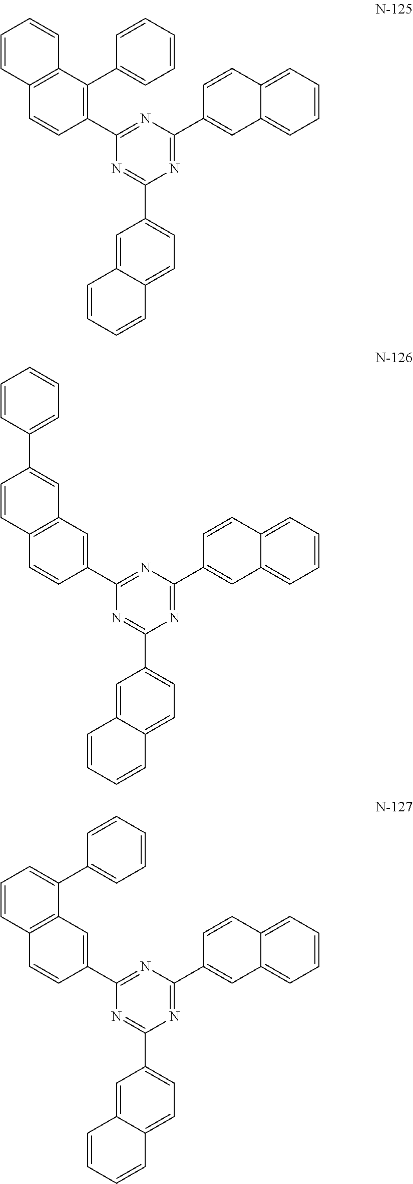

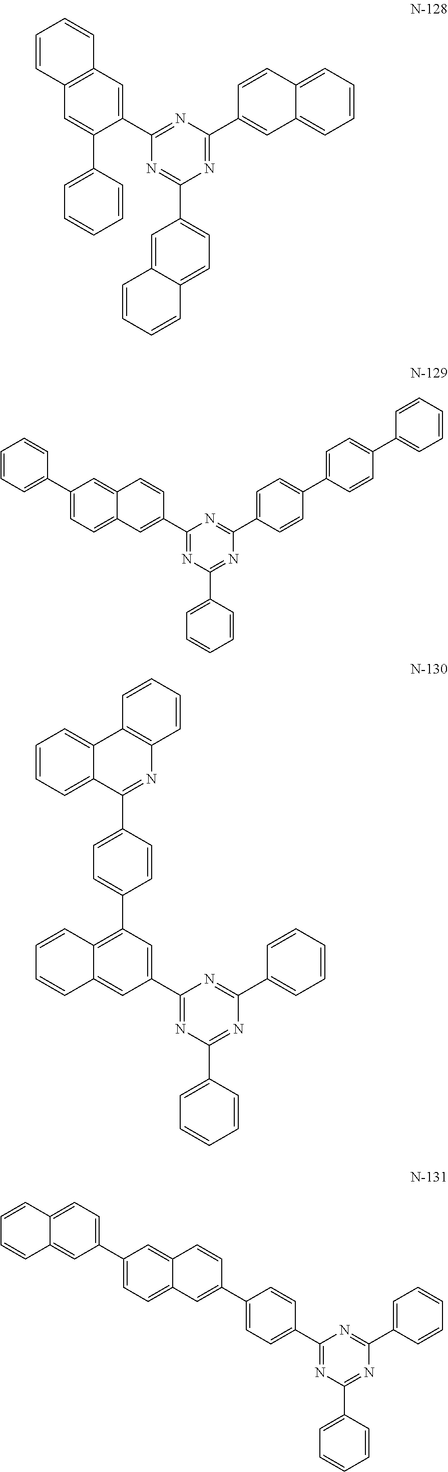 [SMILES: c1ccc(-c2cc3ccccc3cc2-c2nc(-c3ccc4ccccc4c3)nc(-c3ccc4ccccc4c3)n2)cc1 ; c1ccc(-c2ccc(-c3ccc(-c4nc(-c5ccccc5)nc(-c5ccc6cc(-c7ccccc7)ccc6c5)n4)cc3)cc2)cc1 ; c1ccc(-c2nc(-c3ccccc3)nc(-c3cc(-c4ccc(-c5nc6ccccc6c6ccccc56)cc4)c4ccccc4c3)n2)cc1 ; c1ccc(-c2nc(-c3ccccc3)nc(-c3ccc(-c4ccc5cc(-c6ccc7ccccc7c6)ccc5c4)cc3)n2)cc1]

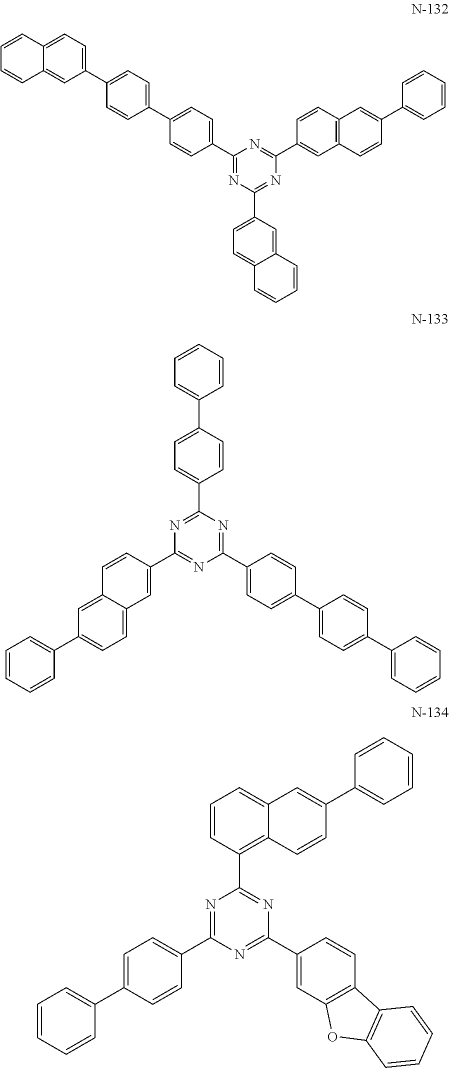 [SMILES: c1ccc(-c2ccc3cc(-c4nc(-c5ccc(-c6ccc(-c7ccc8ccccc8c7)cc6)cc5)nc(-c5ccc6ccccc6c5)n4)ccc3c2)cc1 ; c1ccc(-c2ccc(-c3ccc(-c4nc(-c5ccc(-c6ccccc6)cc5)nc(-c5ccc6cc(-c7ccccc7)ccc6c5)n4)cc3)cc2)cc1 ; c1ccc(-c2ccc(-c3nc(-c4ccc5c(c4)oc4ccccc45)nc(-c4cccc5cc(-c6ccccc6)ccc45)n3)cc2)cc1]

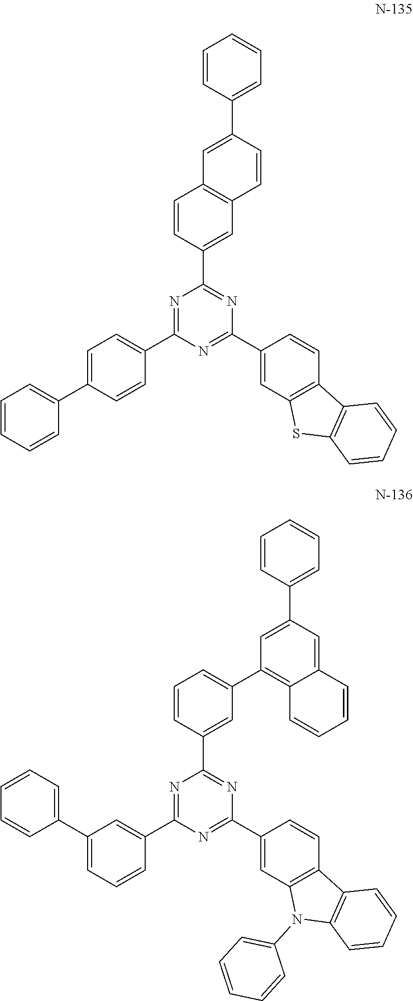 [SMILES: c1ccc(-c2ccc(-c3nc(-c4ccc5cc(-c6ccccc6)ccc5c4)nc(-c4ccc5c(c4)sc4ccccc45)n3)cc2)cc1 ; c1ccc(-c2cccc(-c3nc(-c4cccc(-c5cc(-c6ccccc6)cc6ccccc56)c4)nc(-c4ccc5c6ccccc6n(-c6ccccc6)c5c4)n3)c2)cc1]

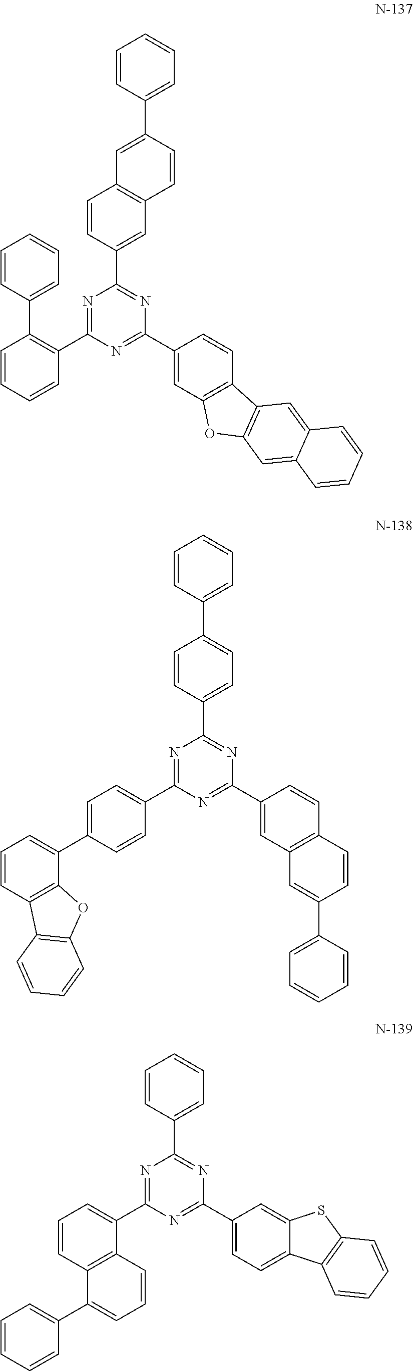 [SMILES: c1ccc(-c2ccc3cc(-c4nc(-c5ccc6c(c5)oc5cc7ccccc7cc56)nc(-c5ccccc5-c5ccccc5)n4)ccc3c2)cc1 ; c1ccc(-c2ccc(-c3nc(-c4ccc(-c5cccc6c5oc5ccccc56)cc4)nc(-c4ccc5ccc(-c6ccccc6)cc5c4)n3)cc2)cc1 ; c1ccc(-c2nc(-c3ccc4c(c3)sc3ccccc34)nc(-c3cccc4c(-c5ccccc5)cccc34)n2)cc1]

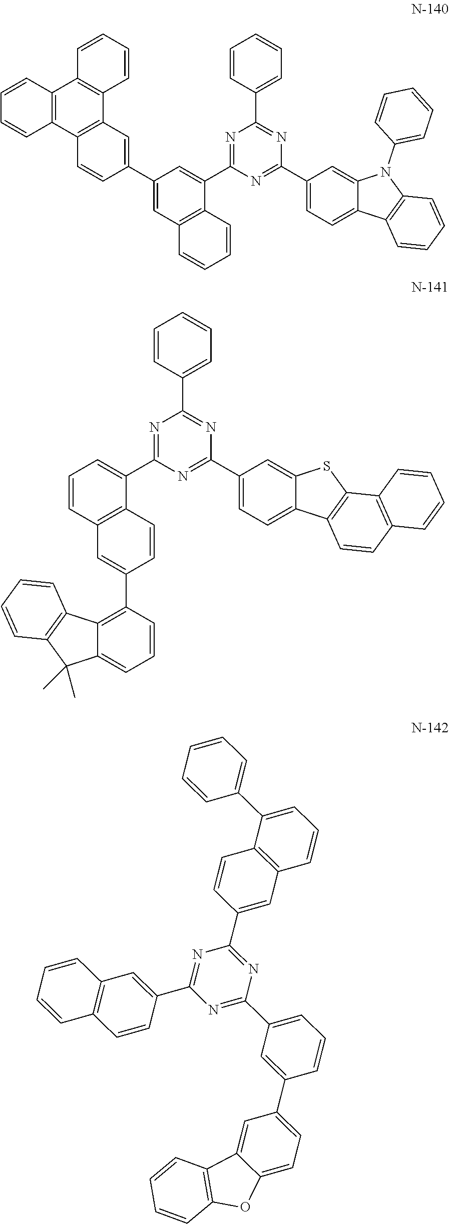 [SMILES: c1ccc(-c2nc(-c3ccc4c5ccccc5n(-c5ccccc5)c4c3)nc(-c3cc(-c4ccc5c6ccccc6c6ccccc6c5c4)cc4ccccc34)n2)cc1 ; CC1(C)c2ccccc2-c2c(-c3ccc4c(-c5nc(-c6ccccc6)nc(-c6ccc7c(c6)sc6c8ccccc8ccc76)n5)cccc4c3)cccc21 ; c1ccc(-c2cccc3cc(-c4nc(-c5cccc(-c6ccc7oc8ccccc8c7c6)c5)nc(-c5ccc6ccccc6c5)n4)ccc23)cc1]

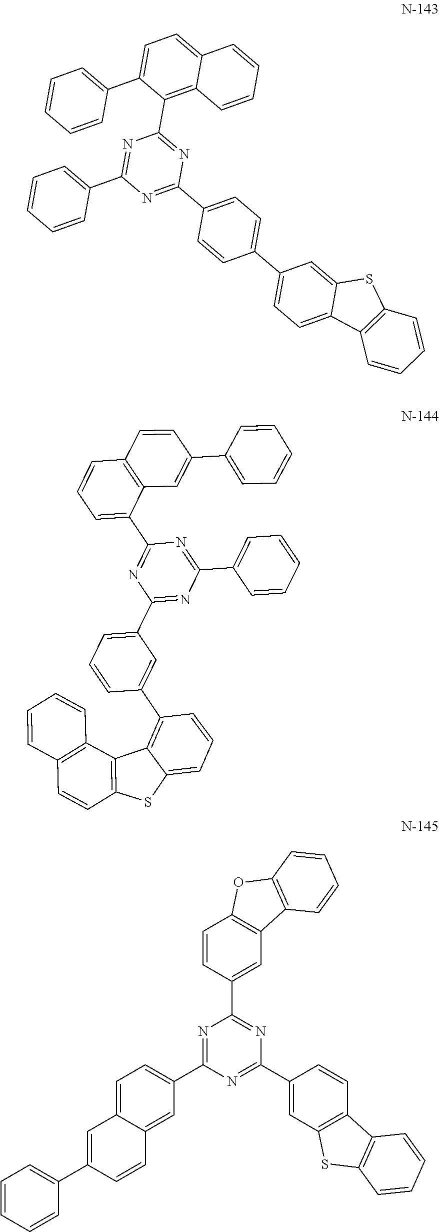 [SMILES: c1ccc(-c2nc(-c3ccc(-c4ccc5c(c4)sc4ccccc45)cc3)nc(-c3c(-c4ccccc4)ccc4ccccc34)n2)cc1 ; c1ccc(-c2ccc3cccc(-c4nc(-c5ccccc5)nc(-c5cccc(-c6cccc7sc8ccc9ccccc9c8c67)c5)n4)c3c2)cc1 ; c1ccc(-c2ccc3cc(-c4nc(-c5ccc6c(c5)sc5ccccc56)nc(-c5ccc6oc7ccccc7c6c5)n4)ccc3c2)cc1]

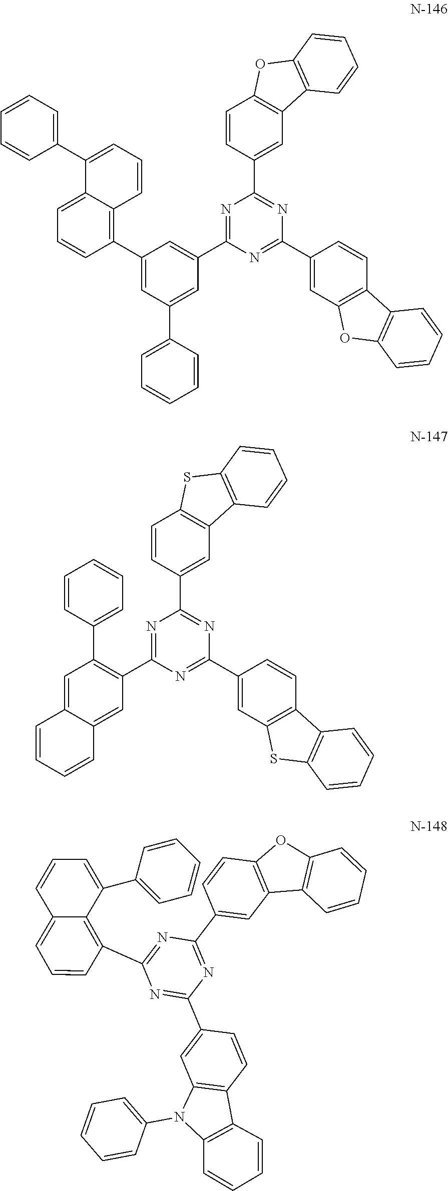 [SMILES: c1ccc(-c2cc(-c3nc(-c4ccc5c(c4)oc4ccccc45)nc(-c4ccc5oc6ccccc6c5c4)n3)cc(-c3cccc4c(-c5ccccc5)cccc34)c2)cc1 ; c1ccc(-c2cc3ccccc3cc2-c2nc(-c3ccc4c(c3)sc3ccccc34)nc(-c3ccc4sc5ccccc5c4c3)n2)cc1 ; c1ccc(-c2cccc3cccc(-c4nc(-c5ccc6oc7ccccc7c6c5)nc(-c5ccc6c7ccccc7n(-c7ccccc7)c6c5)n4)c23)cc1]

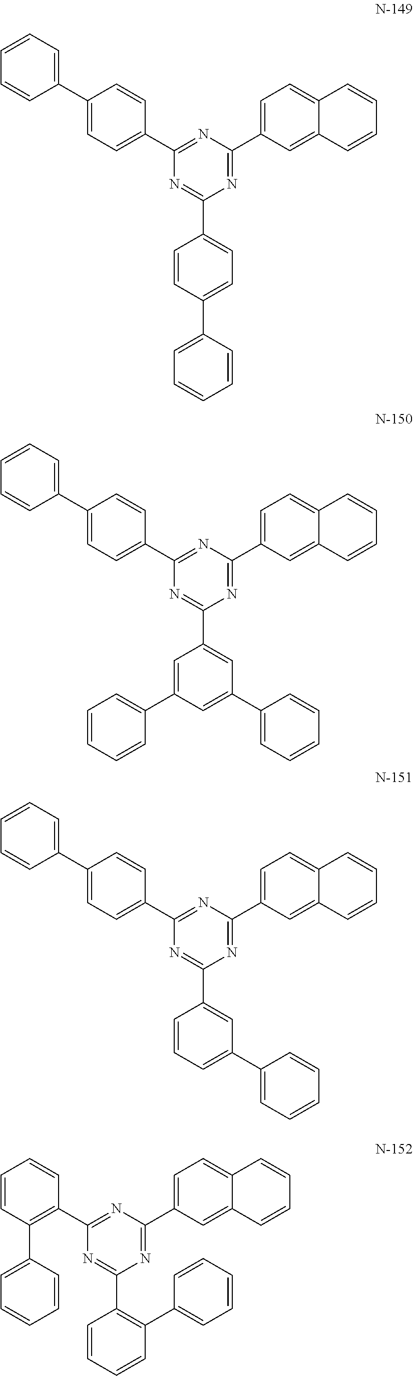 [SMILES: c1ccc(-c2ccc(-c3nc(-c4ccc(-c5ccccc5)cc4)nc(-c4ccc5ccccc5c4)n3)cc2)cc1 ; c1ccc(-c2ccc(-c3nc(-c4cc(-c5ccccc5)cc(-c5ccccc5)c4)nc(-c4ccc5ccccc5c4)n3)cc2)cc1 ; c1ccc(-c2ccc(-c3nc(-c4cccc(-c5ccccc5)c4)nc(-c4ccc5ccccc5c4)n3)cc2)cc1 ; c1ccc(-c2ccccc2-c2nc(-c3ccc4ccccc4c3)nc(-c3ccccc3-c3ccccc3)n2)cc1]

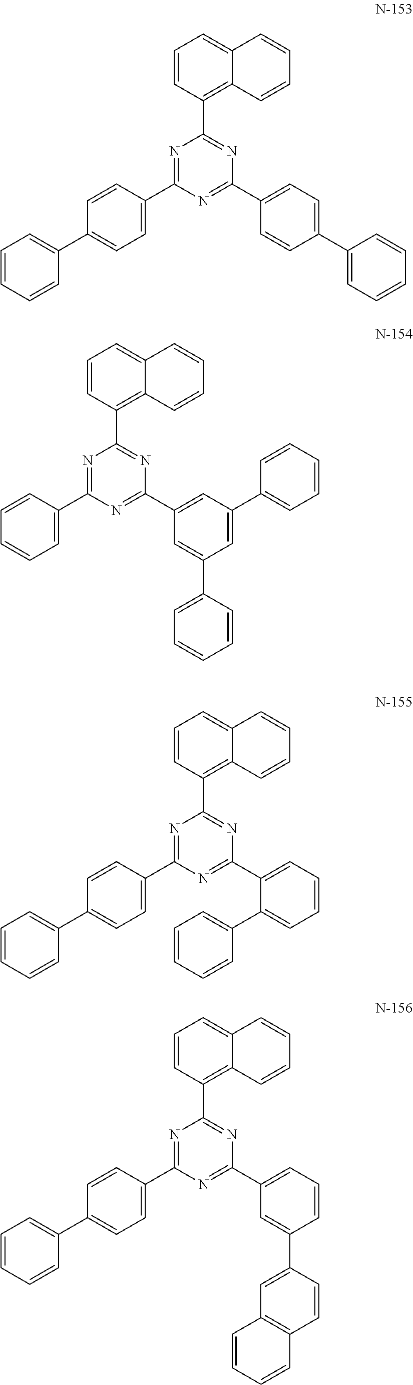

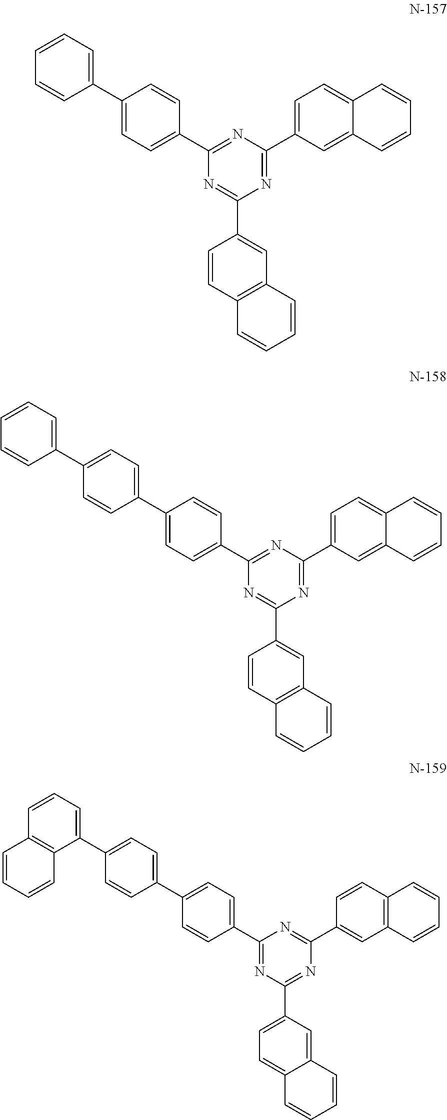 [SMILES: c1ccc(-c2ccc(-c3nc(-c4ccc5ccccc5c4)nc(-c4ccc5ccccc5c4)n3)cc2)cc1 ; c1ccc(-c2ccc(-c3ccc(-c4nc(-c5ccc6ccccc6c5)nc(-c5ccc6ccccc6c5)n4)cc3)cc2)cc1 ; c1ccc2cc(-c3nc(-c4ccc(-c5ccc(-c6cccc7ccccc67)cc5)cc4)nc(-c4ccc5ccccc5c4)n3)ccc2c1]

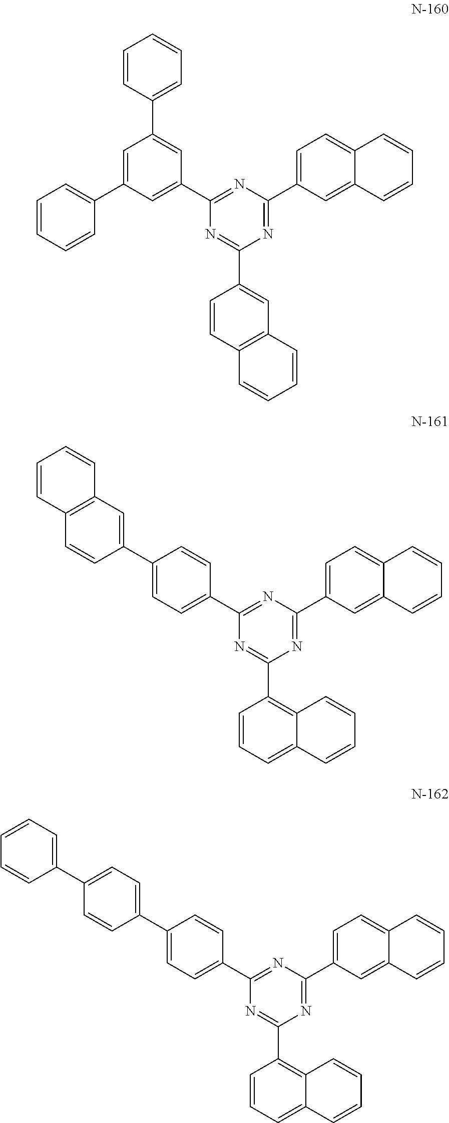 [SMILES: c1ccc(-c2cc(-c3ccccc3)cc(-c3nc(-c4ccc5ccccc5c4)nc(-c4ccc5ccccc5c4)n3)c2)cc1 ; c1ccc2cc(-c3ccc(-c4nc(-c5ccc6ccccc6c5)nc(-c5cccc6ccccc56)n4)cc3)ccc2c1 ; c1ccc(-c2ccc(-c3ccc(-c4nc(-c5ccc6ccccc6c5)nc(-c5cccc6ccccc56)n4)cc3)cc2)cc1]

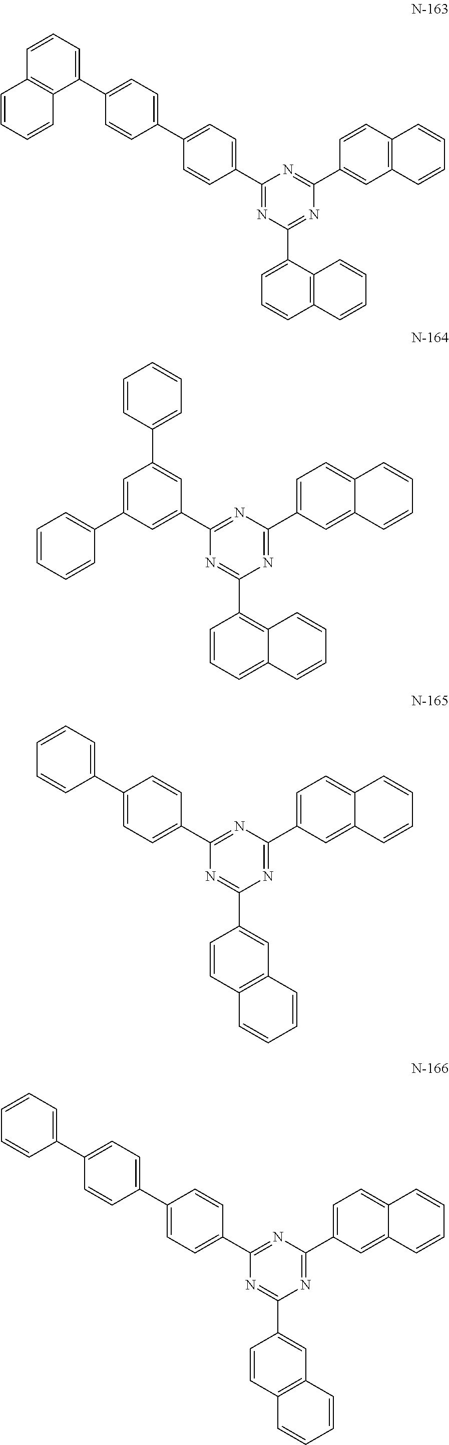 [SMILES: c1ccc2cc(-c3nc(-c4ccc(-c5ccc(-c6cccc7ccccc67)cc5)cc4)nc(-c4cccc5ccccc45)n3)ccc2c1 ; c1ccc(-c2cc(-c3ccccc3)cc(-c3nc(-c4ccc5ccccc5c4)nc(-c4cccc5ccccc45)n3)c2)cc1 ; c1ccc(-c2ccc(-c3nc(-c4ccc5ccccc5c4)nc(-c4ccc5ccccc5c4)n3)cc2)cc1 ; c1ccc(-c2ccc(-c3ccc(-c4nc(-c5ccc6ccccc6c5)nc(-c5ccc6ccccc6c5)n4)cc3)cc2)cc1]

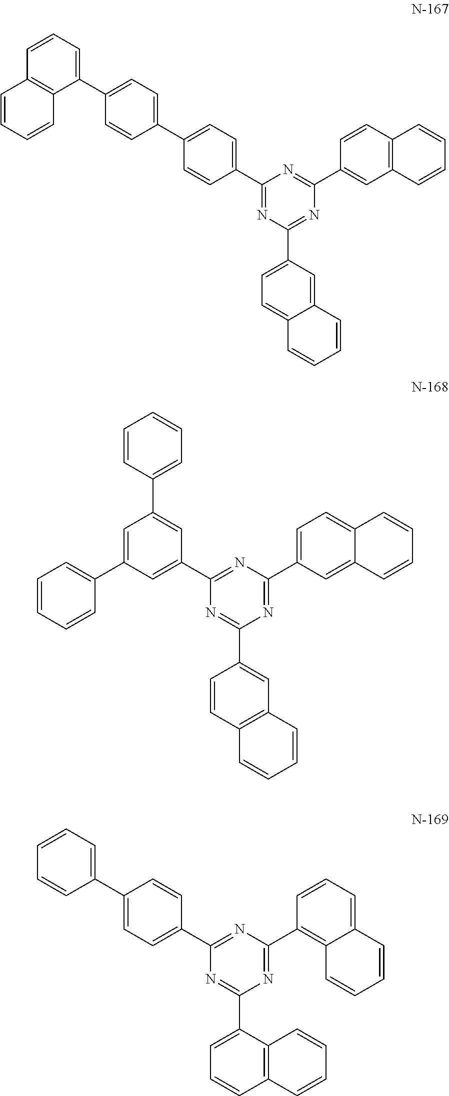 [SMILES: c1ccc2cc(-c3nc(-c4ccc(-c5ccc(-c6cccc7ccccc67)cc5)cc4)nc(-c4ccc5ccccc5c4)n3)ccc2c1 ; c1ccc(-c2cc(-c3ccccc3)cc(-c3nc(-c4ccc5ccccc5c4)nc(-c4ccc5ccccc5c4)n3)c2)cc1 ; c1ccc(-c2ccc(-c3nc(-c4cccc5ccccc45)nc(-c4cccc5ccccc45)n3)cc2)cc1]

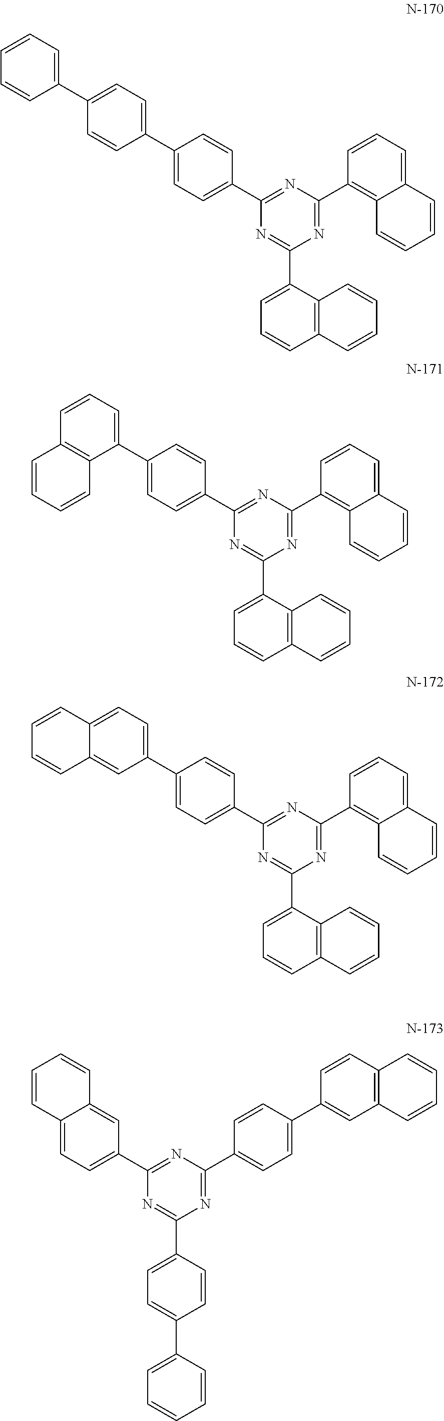 [SMILES: c1ccc(-c2ccc(-c3ccc(-c4nc(-c5cccc6ccccc56)nc(-c5cccc6ccccc56)n4)cc3)cc2)cc1 ; c1ccc2c(-c3ccc(-c4nc(-c5cccc6ccccc56)nc(-c5cccc6ccccc56)n4)cc3)cccc2c1 ; c1ccc2cc(-c3ccc(-c4nc(-c5cccc6ccccc56)nc(-c5cccc6ccccc56)n4)cc3)ccc2c1 ; c1ccc(-c2ccc(-c3nc(-c4ccc(-c5ccc6ccccc6c5)cc4)nc(-c4ccc5ccccc5c4)n3)cc2)cc1]

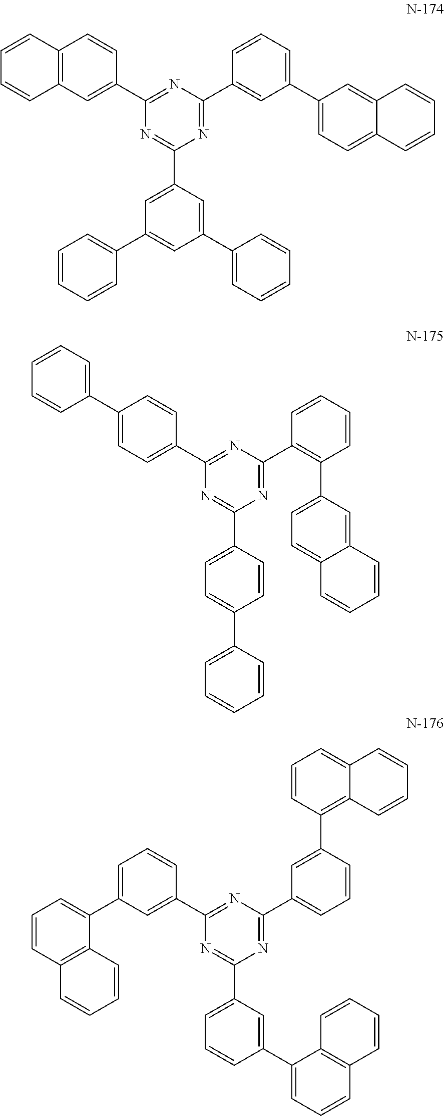 [SMILES: c1ccc(-c2cc(-c3ccccc3)cc(-c3nc(-c4cccc(-c5ccc6ccccc6c5)c4)nc(-c4ccc5ccccc5c4)n3)c2)cc1 ; c1ccc(-c2ccc(-c3nc(-c4ccc(-c5ccccc5)cc4)nc(-c4ccccc4-c4ccc5ccccc5c4)n3)cc2)cc1 ; c1cc(-c2nc(-c3cccc(-c4cccc5ccccc45)c3)nc(-c3cccc(-c4cccc5ccccc45)c3)n2)cc(-c2cccc3ccccc23)c1]

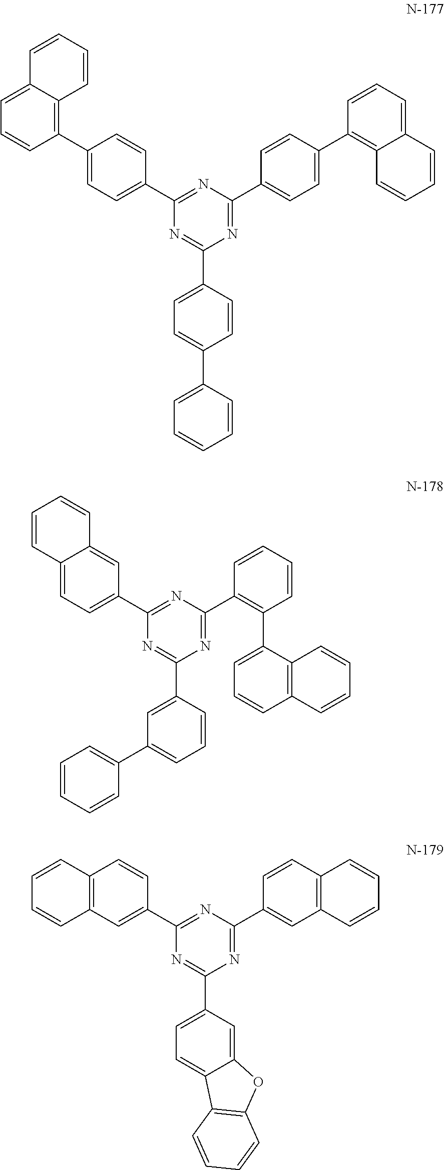 [SMILES: c1ccc(-c2ccc(-c3nc(-c4ccc(-c5cccc6ccccc56)cc4)nc(-c4ccc(-c5cccc6ccccc56)cc4)n3)cc2)cc1 ; c1ccc(-c2cccc(-c3nc(-c4ccc5ccccc5c4)nc(-c4ccccc4-c4cccc5ccccc45)n3)c2)cc1 ; c1ccc2cc(-c3nc(-c4ccc5ccccc5c4)nc(-c4ccc5c(c4)oc4ccccc45)n3)ccc2c1]

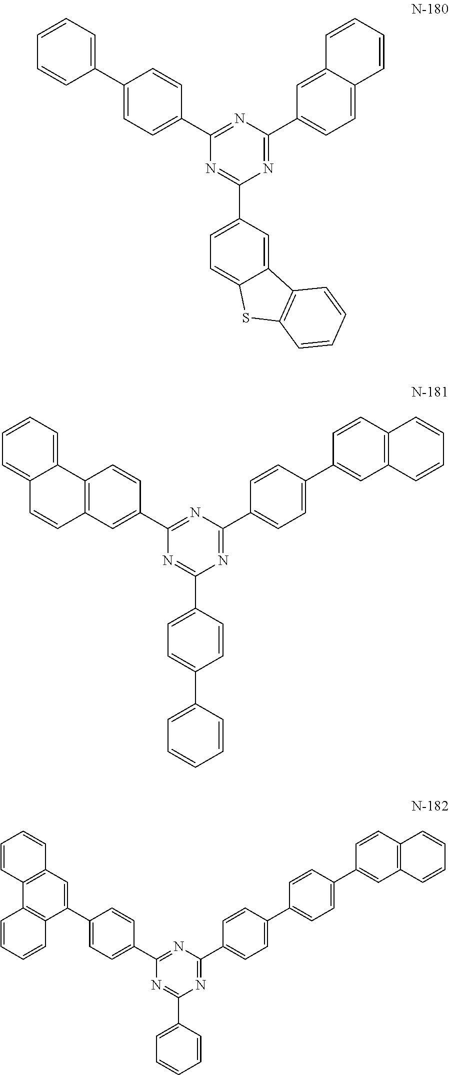 [SMILES: c1ccc(-c2ccc(-c3nc(-c4ccc5ccccc5c4)nc(-c4ccc5sc6ccccc6c5c4)n3)cc2)cc1 ; c1ccc(-c2ccc(-c3nc(-c4ccc(-c5ccc6ccccc6c5)cc4)nc(-c4ccc5c(ccc6ccccc65)c4)n3)cc2)cc1 ; c1ccc(-c2nc(-c3ccc(-c4ccc(-c5ccc6ccccc6c5)cc4)cc3)nc(-c3ccc(-c4cc5ccccc5c5ccccc45)cc3)n2)cc1]

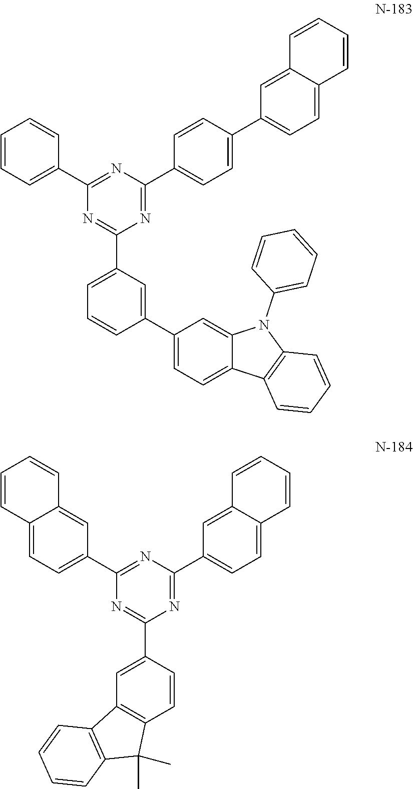 [SMILES: c1ccc(-c2nc(-c3ccc(-c4ccc5ccccc5c4)cc3)nc(-c3cccc(-c4ccc5c6ccccc6n(-c6ccccc6)c5c4)c3)n2)cc1 ; CC1(C)c2ccccc2-c2cc(-c3nc(-c4ccc5ccccc5c4)nc(-c4ccc5ccccc5c4)n3)ccc21]

Also, Formula 2 is represented by Formula 3.

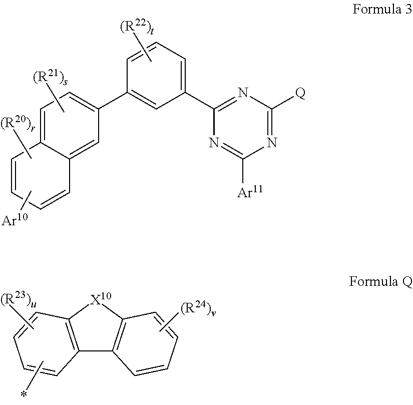

Wherein,
1) Q is a substituent represented by Formula Q,
2) Ar¹⁰ is a C₆-C₆₀ aryl group; a C₆-C₆₀ aryl group substituted by deuterium;

When Ar¹⁰ is an aryl group, it may be preferably a C₆-C₃₀ aryl group, and more preferably a C₆-C₂₅ aryl group, for example, it may be phenyl, biphenyl, naphthalene, phenanthrene, etc,
3) Ar¹¹ is a C₆-C₆₀ aryl group; or a C₂-C₆₀ heterocyclic group including at least one heteroatom of O, N, S, Si or P;

When Ar¹¹ is an aryl group, it may be preferably a C₆-C₃₀ aryl group, and more preferably a C₆-C₂₅ aryl group, for example, it may be phenyl, biphenyl, naphthalene, phenanthrene, etc,

When Ar¹¹ is are a heterocyclic group, it may be preferably a C₂˜C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, naphthobenzofuran, naphthobenzothiophene, benzothienopyrimidine, benzofuropyrimidine, phenothiazine, phenylphenothiazine etc.,
4) R²⁰, R²¹ and R²² are the same or different, and each independently hydrogen; or deuterium;
5) R²³ and R²⁴ are the same or different, and each independently selected from the group consisting of a hydrogen; deuterium; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₃-C₆₀ aliphatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; or a plurality of adjacent R²³s or a plurality of R²⁴s may be bonded to each other to form a ring,

When R²³ and R²⁴ are an aryl group, it may be preferably a C₆-C₃₀ aryl group, and more preferably a C₆-C₂₅ aryl group, for example, it may be phenyl, biphenyl, terphenyl, naphthalene, phenanthrene, etc,

When R²³ and R²⁴ are a heterocyclic group, it may be preferably a C₂˜C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, naphthobenzofuran, naphthobenzothiophene, benzothienopyrimidine, benzofuropyrimidine, phenothiazine, phenylphenothiazine etc.,

When R²³ and R²⁴ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

When R²³ and R²⁴ are an alkyl group, it may be preferably a C₁-C₃₀ alkyl group, and more preferably a C₁-C₂₄ alkyl group.

When R²³ and R²⁴ are alkoxyl groups, they may be preferably C₁˜C₂₄ alkoxyl groups.

When R²³ and R²⁴ are an aryloxy group, it may be preferably a C₆˜C₂₄ aryloxy group,
6) X¹⁰ is O or S,
7) r, s and u are each independently an integer of 0 to 3, t and v are each independently an integer of 0 to 4,
8) * means a position that binds to Formula (3),

wherein the aryl group, heterocyclic group, fluorenyl group, aliphatic ring group, fused ring group, alkyl group, alkenyl group, alkynyl group, alkoxy group, and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and also the substituents may be bonded to each other to form a saturated or unsaturated ring, wherein the term ‘ring’ means a C₃-C₆₀ aliphatic ring and a C₆-C₆₀ aromatic ring, or a C₂-C₆₀ heterocyclic group or a fused ring formed by the combination thereof.

Also, Formula 3 is represented by any one of Formulas 3-1 to 3-4.

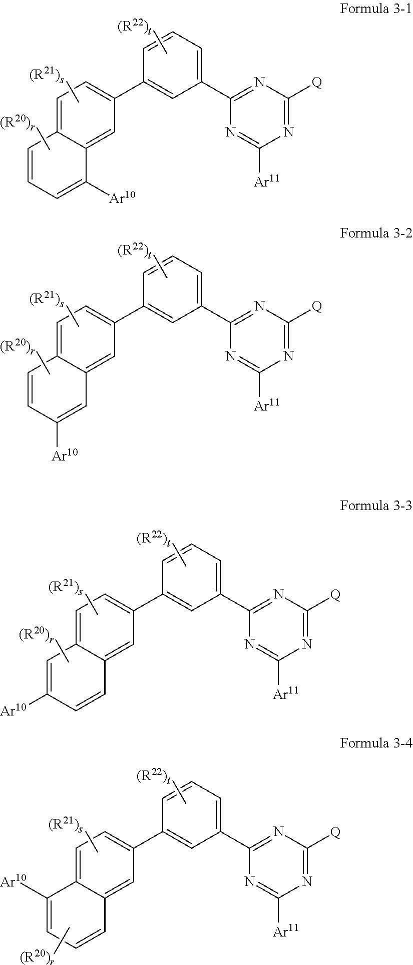

Wherein, Q, Ar¹⁰, Ar¹¹, R²⁰, R²¹, R²², r, s and t are the same as defined in Formula 3.

Also, Formula Q is represented by any one of the following Formula Q-1 to Formula Q-4.

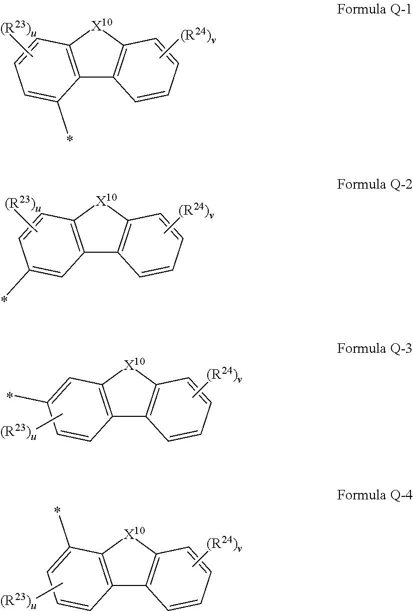

Wherein, X¹⁰, R²³, R²⁴, u, v and * are the same as defined in Formula Q.

Formula Q may be represented by any one of the following compounds, but is not limited thereto.

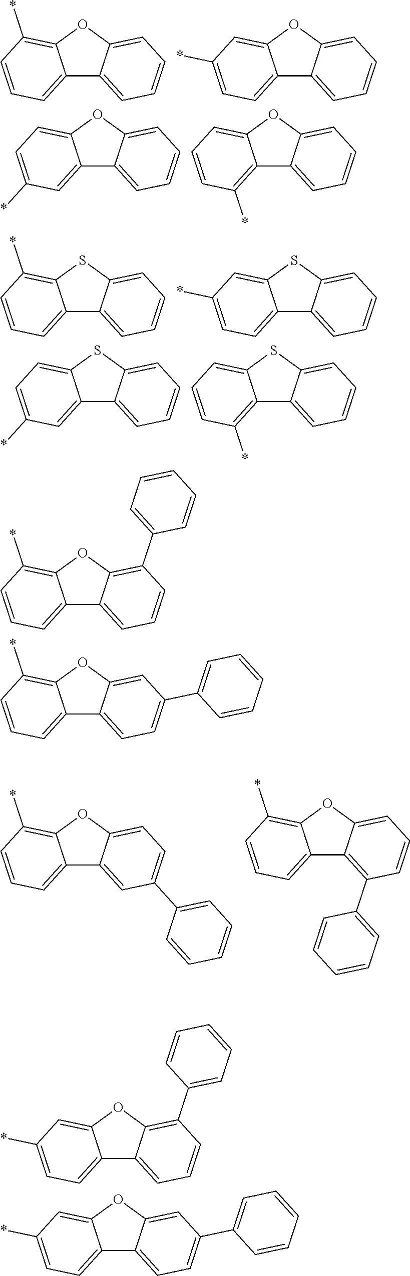

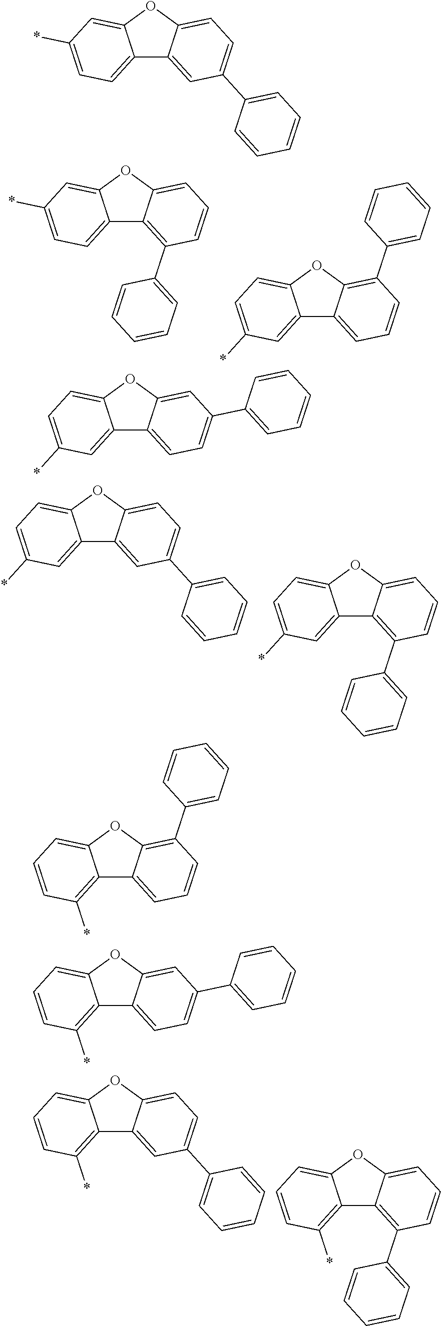

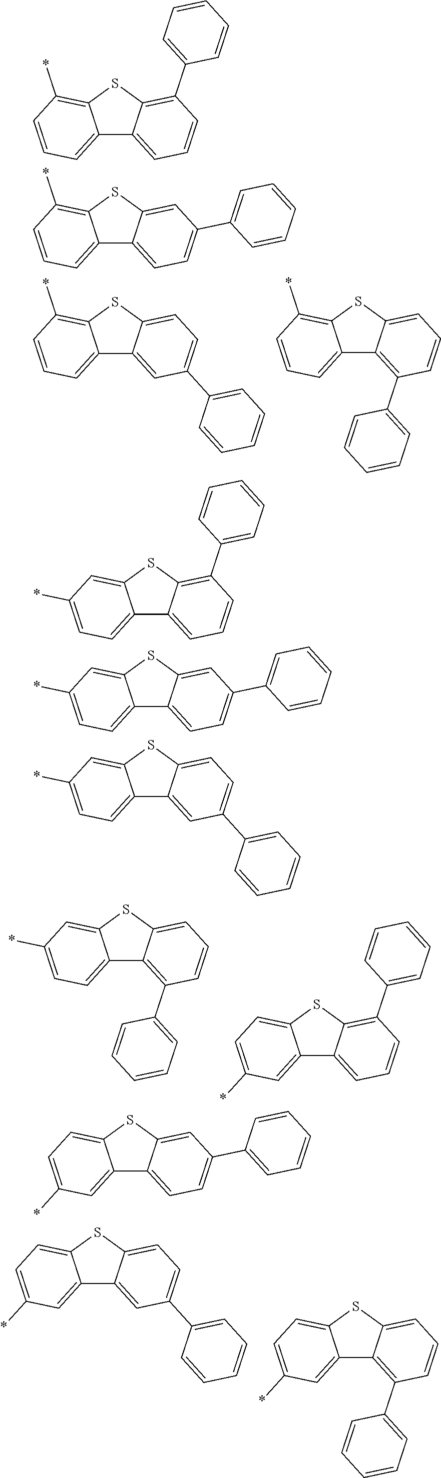

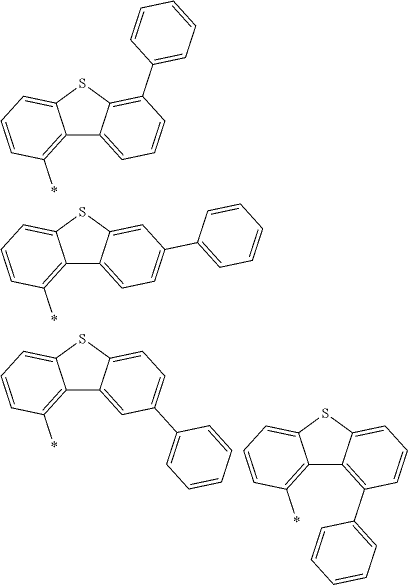

Also, Ar¹⁰ or Ar¹¹ is represented by one of Formulas Ar-1 to Ar-7.

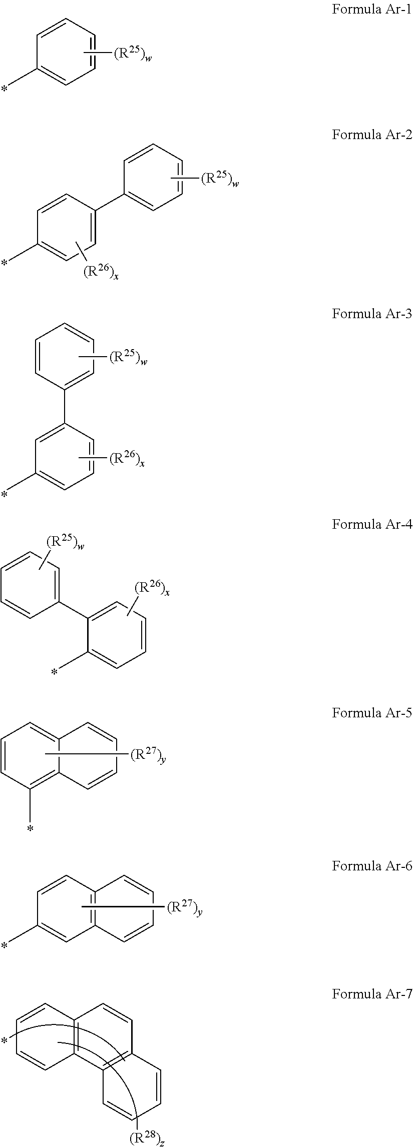

Wherein,
1) R²⁵, R²⁶, R²⁷ and R²⁸ are the same as the definition of R²³, or an adjacent plurality of R²⁵s, a plurality of R²⁶s, a plurality of R²⁷s, or a plurality of R²⁸s may be bonded to each other to form a ring,
2) w is an integer from 0 to 5, x is an integer from 0 to 4, y is an integer from 0 to 7, z is an integer from 0 to 9,
3) * means a moiety bonded to Formula 3.

Also, Ar¹¹ is represented by Formula Ar-8.

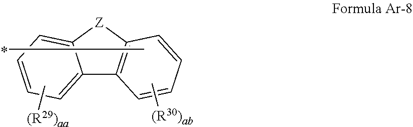

Wherein,
1) Z is O, S, C(R³¹)(R³²), NR³³ or N,
However, when Z is combined with Formula 3, it is N,
2) R²⁹, R³⁰, R³¹, R³² and R³³ are the same as definition of R²³, or an adjacent plurality of R²⁹s, a plurality of R³⁰s, a plurality of R³¹s, a plurality of R³²s may be bonded to each other to form a ring,
3) aa and ab are independently integers from 0 to 4;
4) * means a moiety bonded to Formula 3.

Specifically, Formula 3 may be any one of the following compounds N 1-1 to N 1-100, but is not limited thereto.

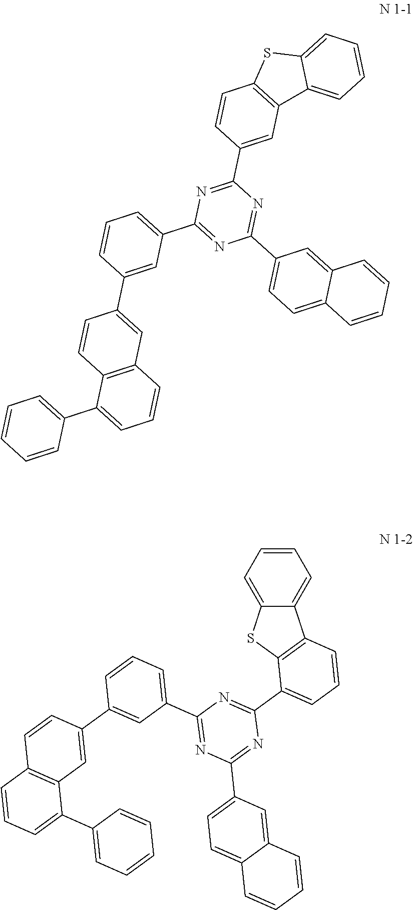 [SMILES: c1ccc(-c2cccc3cc(-c4cccc(-c5nc(-c6ccc7ccccc7c6)nc(-c6ccc7sc8ccccc8c7c6)n5)c4)ccc23)cc1 ; c1ccc(-c2cccc3ccc(-c4cccc(-c5nc(-c6ccc7ccccc7c6)nc(-c6cccc7c6sc6ccccc67)n5)c4)cc23)cc1]

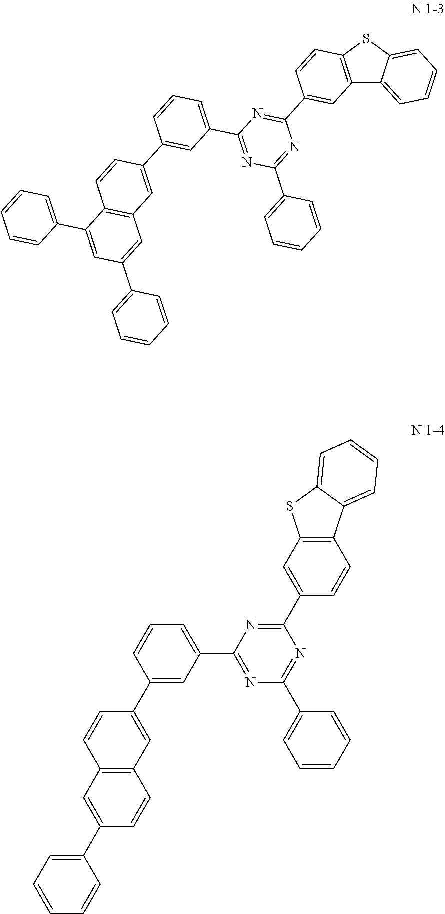 [SMILES: c1ccc(-c2cc(-c3ccccc3)c3ccc(-c4cccc(-c5nc(-c6ccccc6)nc(-c6ccc7sc8ccccc8c7c6)n5)c4)cc3c2)cc1 ; c1ccc(-c2ccc3cc(-c4cccc(-c5nc(-c6ccccc6)nc(-c6ccc7c(c6)sc6ccccc67)n5)c4)ccc3c2)cc1]

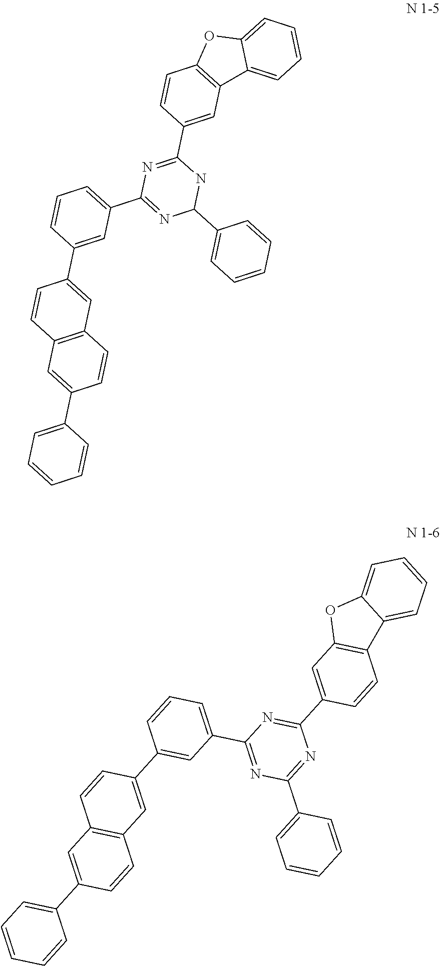

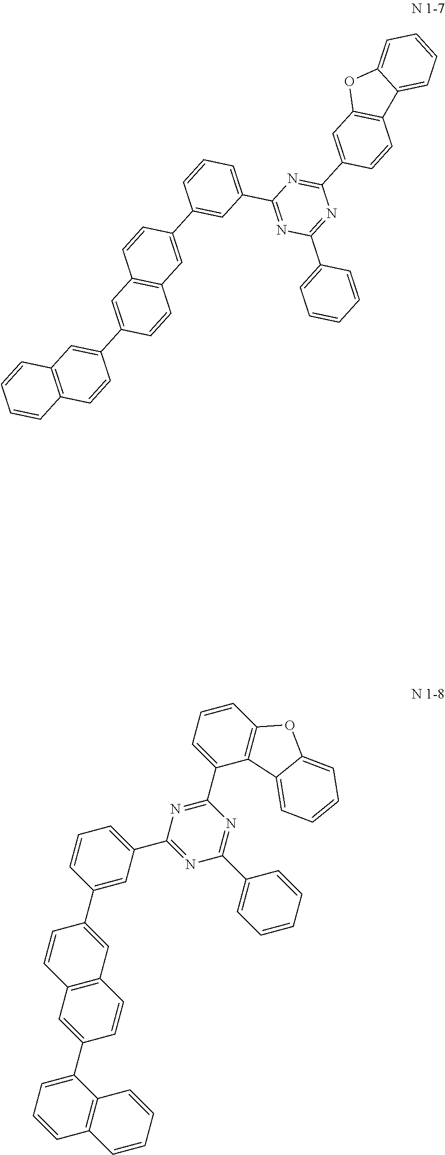 [SMILES: c1ccc(-c2nc(-c3cccc(-c4ccc5cc(-c6ccc7ccccc7c6)ccc5c4)c3)nc(-c3ccc4c(c3)oc3ccccc34)n2)cc1 ; c1ccc(-c2nc(-c3cccc(-c4ccc5cc(-c6cccc7ccccc67)ccc5c4)c3)nc(-c3cccc4oc5ccccc5c34)n2)cc1]

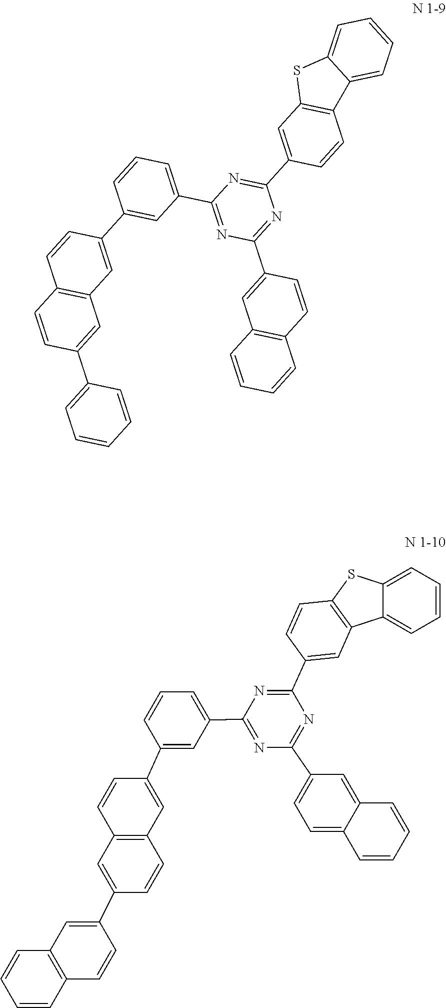 [SMILES: c1ccc(-c2ccc3ccc(-c4cccc(-c5nc(-c6ccc7ccccc7c6)nc(-c6ccc7c(c6)sc6ccccc67)n5)c4)cc3c2)cc1 ; c1cc(-c2ccc3cc(-c4ccc5ccccc5c4)ccc3c2)cc(-c2nc(-c3ccc4ccccc4c3)nc(-c3ccc4sc5ccccc5c4c3)n2)c1]

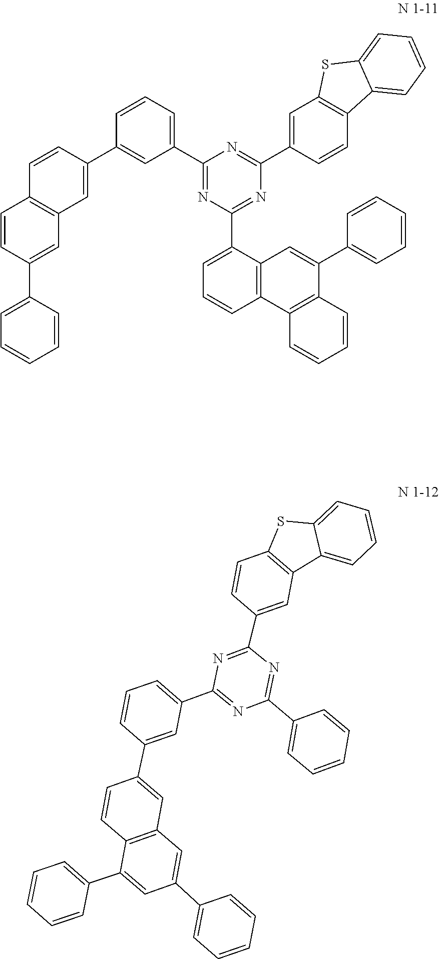 [SMILES: c1ccc(-c2ccc3ccc(-c4cccc(-c5nc(-c6ccc7c(c6)sc6ccccc67)nc(-c6cccc7c6cc(-c6ccccc6)c6ccccc67)n5)c4)cc3c2)cc1 ; c1ccc(-c2cc(-c3ccccc3)c3ccc(-c4cccc(-c5nc(-c6ccccc6)nc(-c6ccc7sc8ccccc8c7c6)n5)c4)cc3c2)cc1]

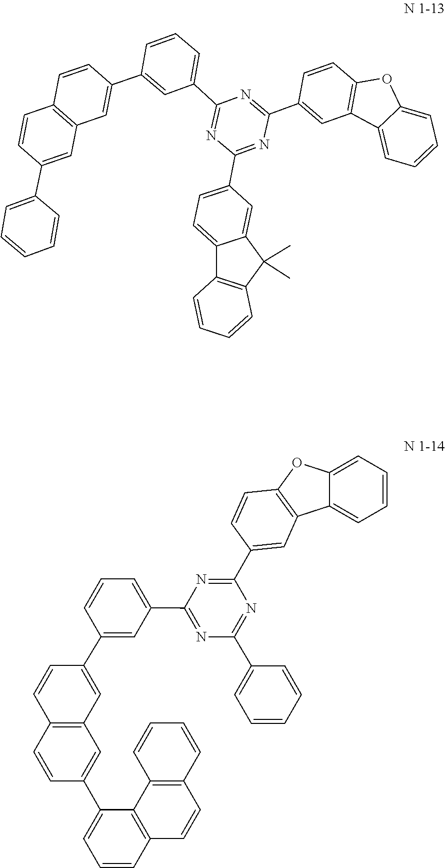 [SMILES: CC1(C)c2ccccc2-c2ccc(-c3nc(-c4cccc(-c5ccc6ccc(-c7ccccc7)cc6c5)c4)nc(-c4ccc5oc6ccccc6c5c4)n3)cc21 ; c1ccc(-c2nc(-c3cccc(-c4ccc5ccc(-c6cccc7ccc8ccccc8c67)cc5c4)c3)nc(-c3ccc4oc5ccccc5c4c3)n2)cc1]

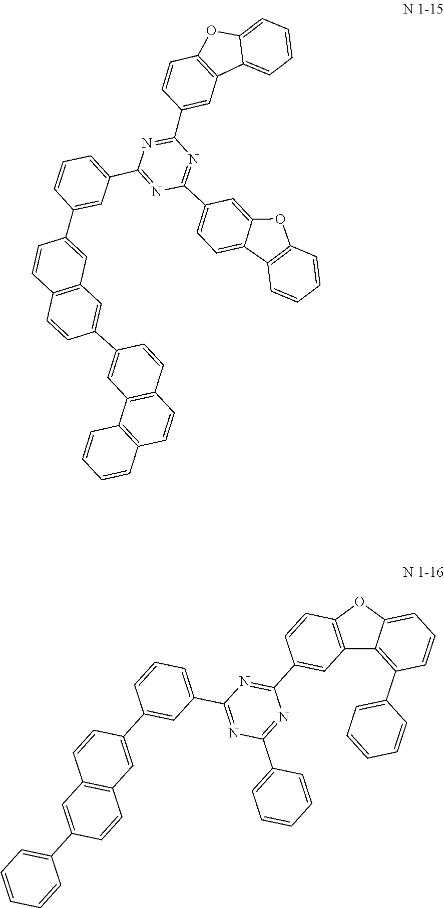 [SMILES: c1cc(-c2ccc3ccc(-c4ccc5ccc6ccccc6c5c4)cc3c2)cc(-c2nc(-c3ccc4c(c3)oc3ccccc34)nc(-c3ccc4oc5ccccc5c4c3)n2)c1 ; c1ccc(-c2ccc3cc(-c4cccc(-c5nc(-c6ccccc6)nc(-c6ccc7oc8cccc(-c9ccccc9)c8c7c6)n5)c4)ccc3c2)cc1]

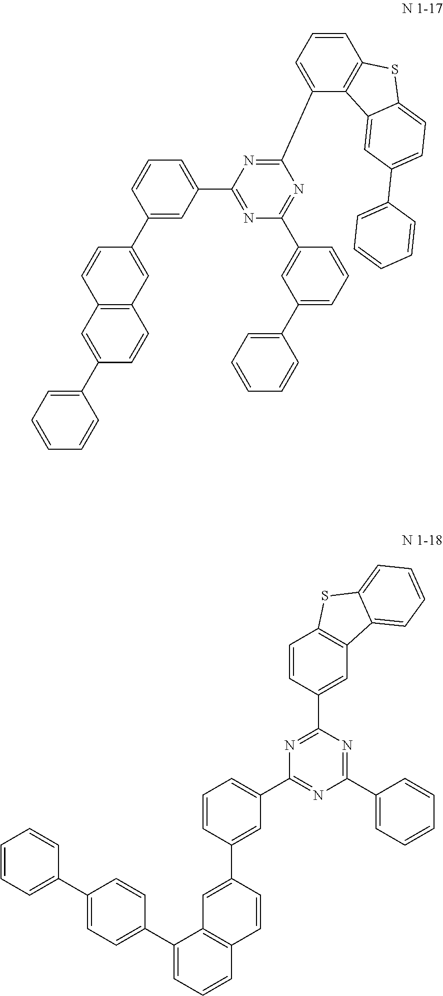 [SMILES: c1ccc(-c2cccc(-c3nc(-c4cccc(-c5ccc6cc(-c7ccccc7)ccc6c5)c4)nc(-c4cccc5sc6ccc(-c7ccccc7)cc6c45)n3)c2)cc1 ; c1ccc(-c2ccc(-c3cccc4ccc(-c5cccc(-c6nc(-c7ccccc7)nc(-c7ccc8sc9ccccc9c8c7)n6)c5)cc34)cc2)cc1]

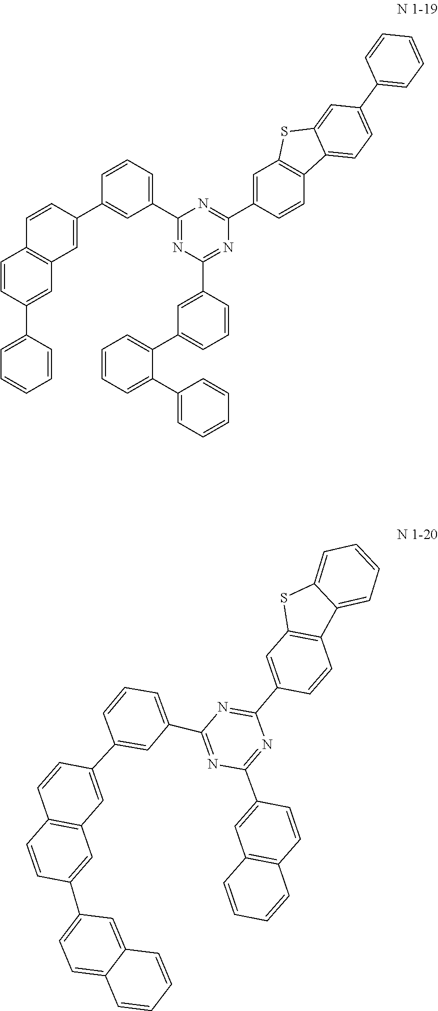 [SMILES: c1ccc(-c2ccc3ccc(-c4cccc(-c5nc(-c6cccc(-c7ccccc7-c7ccccc7)c6)nc(-c6ccc7c(c6)sc6cc(-c8ccccc8)ccc67)n5)c4)cc3c2)cc1 ; c1cc(-c2ccc3ccc(-c4ccc5ccccc5c4)cc3c2)cc(-c2nc(-c3ccc4ccccc4c3)nc(-c3ccc4c(c3)sc3ccccc34)n2)c1]

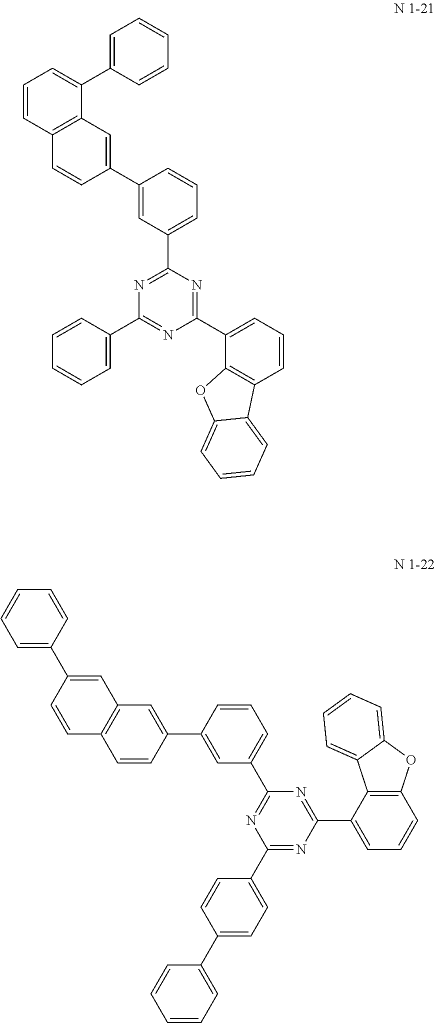 [SMILES: c1ccc(-c2nc(-c3cccc(-c4ccc5cccc(-c6ccccc6)c5c4)c3)nc(-c3cccc4c3oc3ccccc34)n2)cc1 ; c1ccc(-c2ccc(-c3nc(-c4cccc(-c5ccc6ccc(-c7ccccc7)cc6c5)c4)nc(-c4cccc5oc6ccccc6c45)n3)cc2)cc1]

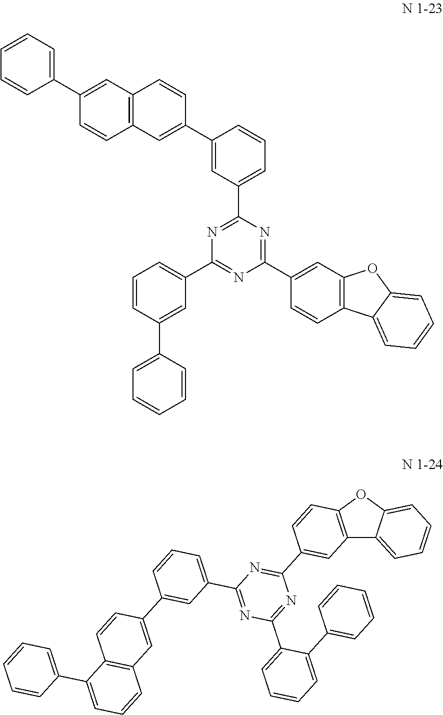 [SMILES: c1ccc(-c2cccc(-c3nc(-c4cccc(-c5ccc6cc(-c7ccccc7)ccc6c5)c4)nc(-c4ccc5c(c4)oc4ccccc45)n3)c2)cc1 ; c1ccc(-c2ccccc2-c2nc(-c3cccc(-c4ccc5c(-c6ccccc6)cccc5c4)c3)nc(-c3ccc4oc5ccccc5c4c3)n2)cc1]

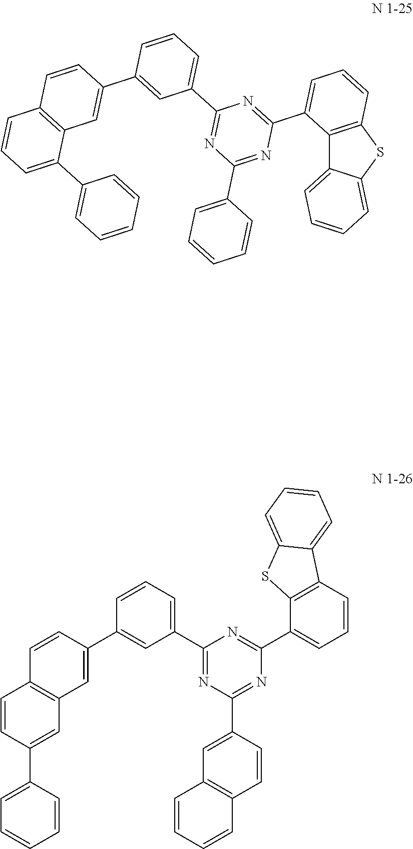 [SMILES: c1ccc(-c2nc(-c3cccc(-c4ccc5cccc(-c6ccccc6)c5c4)c3)nc(-c3cccc4sc5ccccc5c34)n2)cc1 ; c1ccc(-c2ccc3ccc(-c4cccc(-c5nc(-c6ccc7ccccc7c6)nc(-c6cccc7c6sc6ccccc67)n5)c4)cc3c2)cc1]

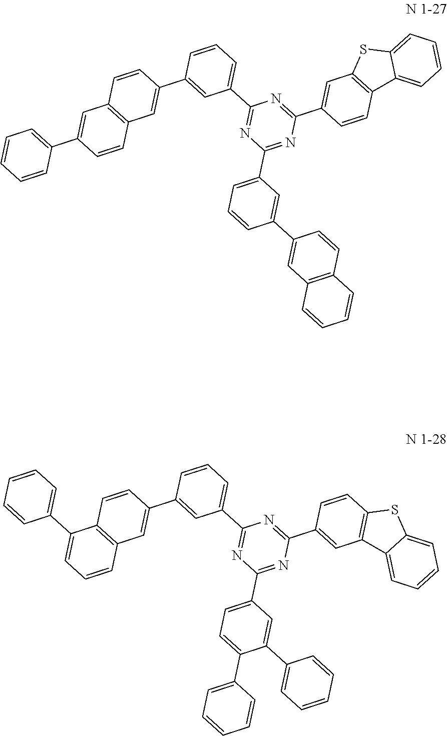 [SMILES: c1ccc(-c2ccc3cc(-c4cccc(-c5nc(-c6cccc(-c7ccc8ccccc8c7)c6)nc(-c6ccc7c(c6)sc6ccccc67)n5)c4)ccc3c2)cc1 ; c1ccc(-c2ccc(-c3nc(-c4cccc(-c5ccc6c(-c7ccccc7)cccc6c5)c4)nc(-c4ccc5sc6ccccc6c5c4)n3)cc2-c2ccccc2)cc1]

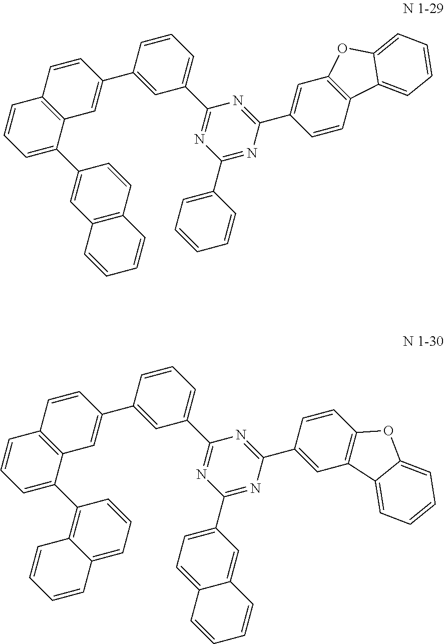 [SMILES: c1ccc(-c2nc(-c3cccc(-c4ccc5cccc(-c6ccc7ccccc7c6)c5c4)c3)nc(-c3ccc4c(c3)oc3ccccc34)n2)cc1 ; c1cc(-c2ccc3cccc(-c4cccc5ccccc45)c3c2)cc(-c2nc(-c3ccc4ccccc4c3)nc(-c3ccc4oc5ccccc5c4c3)n2)c1]

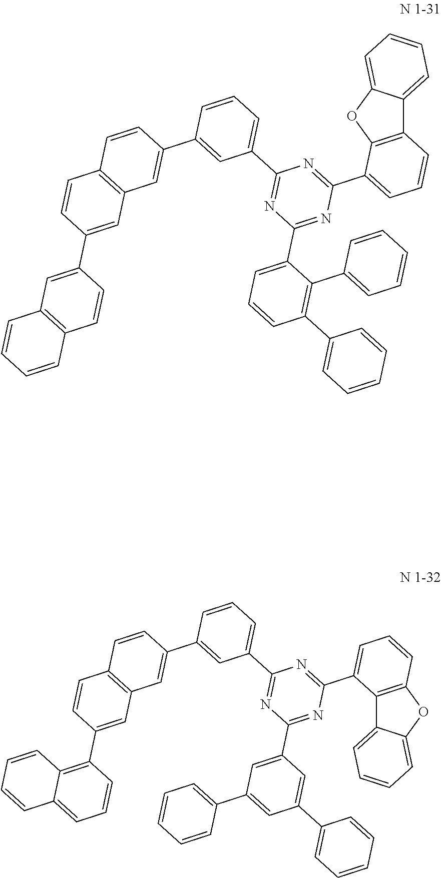 [SMILES: c1ccc(-c2cccc(-c3nc(-c4cccc(-c5ccc6ccc(-c7ccc8ccccc8c7)cc6c5)c4)nc(-c4cccc5c4oc4ccccc45)n3)c2-c2ccccc2)cc1 ; c1ccc(-c2cc(-c3ccccc3)cc(-c3nc(-c4cccc(-c5ccc6ccc(-c7cccc8ccccc78)cc6c5)c4)nc(-c4cccc5oc6ccccc6c45)n3)c2)cc1]

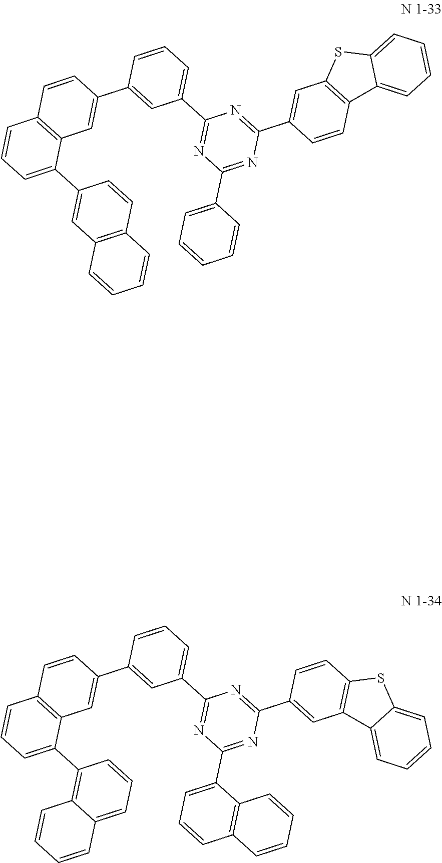 [SMILES: c1ccc(-c2nc(-c3cccc(-c4ccc5cccc(-c6ccc7ccccc7c6)c5c4)c3)nc(-c3ccc4c(c3)sc3ccccc34)n2)cc1 ; c1cc(-c2ccc3cccc(-c4cccc5ccccc45)c3c2)cc(-c2nc(-c3ccc4sc5ccccc5c4c3)nc(-c3cccc4ccccc34)n2)c1]

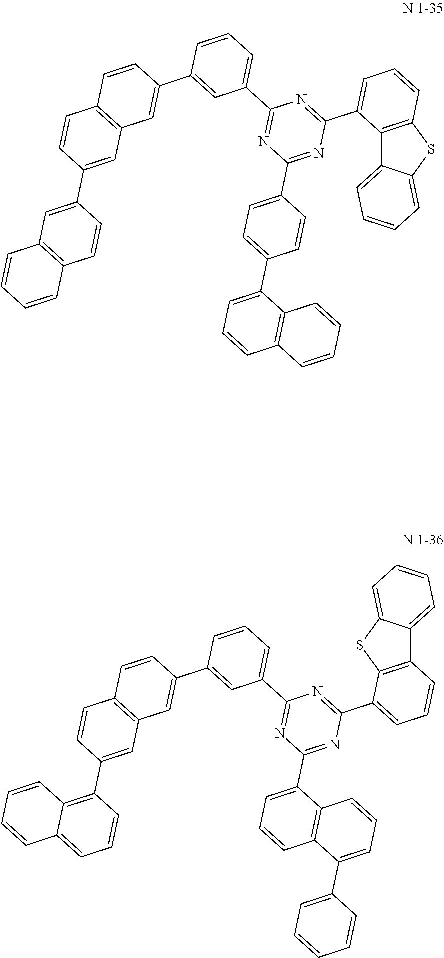 [SMILES: c1cc(-c2ccc3ccc(-c4ccc5ccccc5c4)cc3c2)cc(-c2nc(-c3ccc(-c4cccc5ccccc45)cc3)nc(-c3cccc4sc5ccccc5c34)n2)c1 ; c1ccc(-c2cccc3c(-c4nc(-c5cccc(-c6ccc7ccc(-c8cccc9ccccc89)cc7c6)c5)nc(-c5cccc6c5sc5ccccc56)n4)cccc23)cc1]

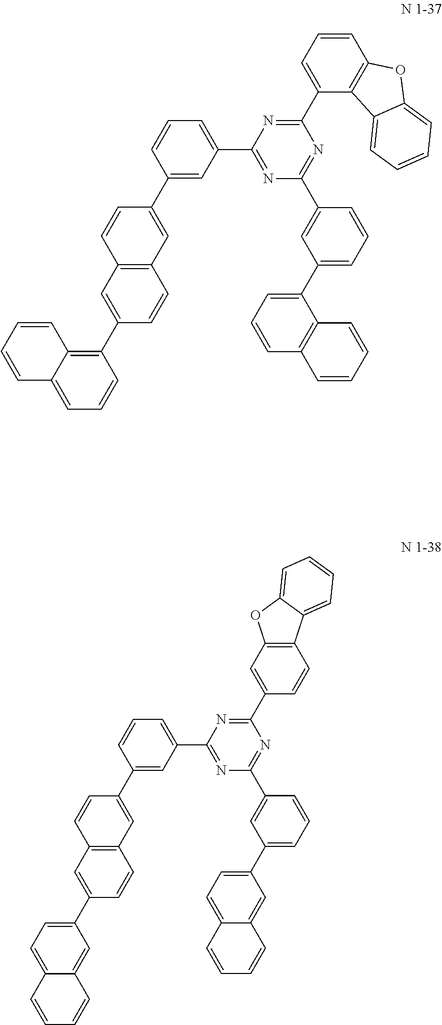 [SMILES: c1cc(-c2ccc3cc(-c4cccc5ccccc45)ccc3c2)cc(-c2nc(-c3cccc(-c4cccc5ccccc45)c3)nc(-c3cccc4oc5ccccc5c34)n2)c1 ; c1cc(-c2ccc3ccccc3c2)cc(-c2nc(-c3cccc(-c4ccc5cc(-c6ccc7ccccc7c6)ccc5c4)c3)nc(-c3ccc4c(c3)oc3ccccc34)n2)c1]

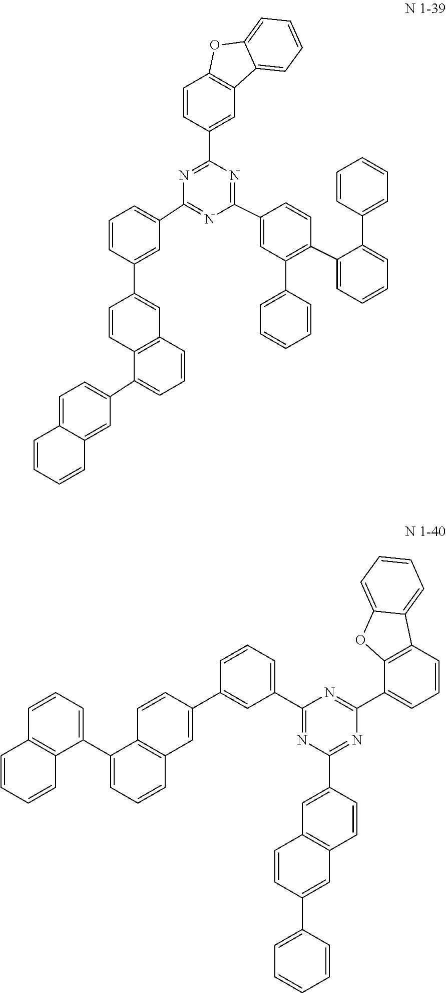 [SMILES: c1ccc(-c2ccccc2-c2ccc(-c3nc(-c4cccc(-c5ccc6c(-c7ccc8ccccc8c7)cccc6c5)c4)nc(-c4ccc5oc6ccccc6c5c4)n3)cc2-c2ccccc2)cc1 ; c1ccc(-c2ccc3cc(-c4nc(-c5cccc(-c6ccc7c(-c8cccc9ccccc89)cccc7c6)c5)nc(-c5cccc6c5oc5ccccc56)n4)ccc3c2)cc1]

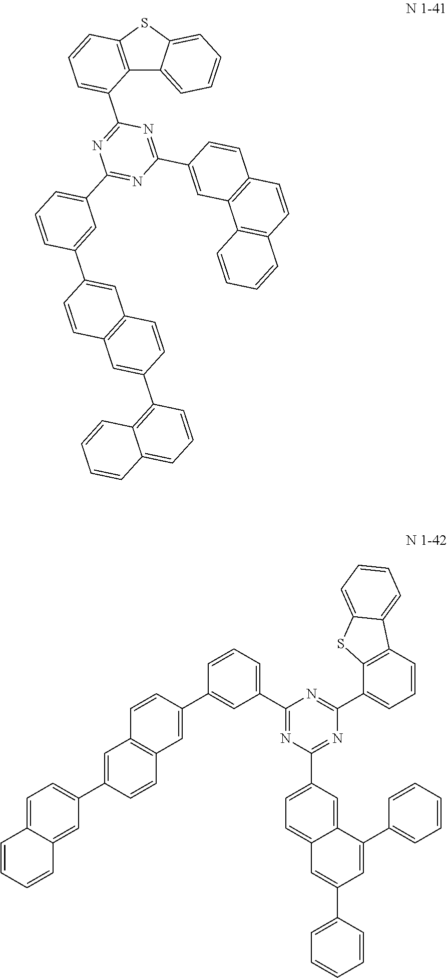 [SMILES: c1cc(-c2ccc3cc(-c4cccc5ccccc45)ccc3c2)cc(-c2nc(-c3ccc4ccc5ccccc5c4c3)nc(-c3cccc4sc5ccccc5c34)n2)c1 ; c1ccc(-c2cc(-c3ccccc3)c3cc(-c4nc(-c5cccc(-c6ccc7cc(-c8ccc9ccccc9c8)ccc7c6)c5)nc(-c5cccc6c5sc5ccccc56)n4)ccc3c2)cc1]

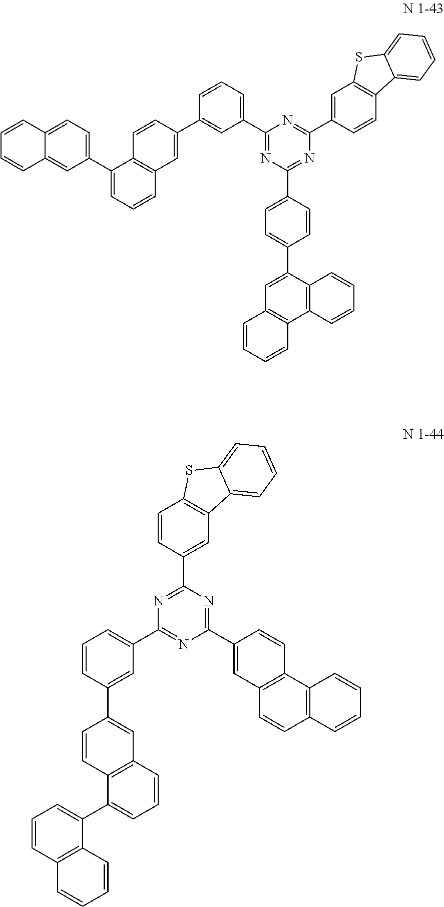 [SMILES: c1cc(-c2ccc3c(-c4ccc5ccccc5c4)cccc3c2)cc(-c2nc(-c3ccc(-c4cc5ccccc5c5ccccc45)cc3)nc(-c3ccc4c(c3)sc3ccccc34)n2)c1 ; c1cc(-c2ccc3c(-c4cccc5ccccc45)cccc3c2)cc(-c2nc(-c3ccc4c(ccc5ccccc54)c3)nc(-c3ccc4sc5ccccc5c4c3)n2)c1]

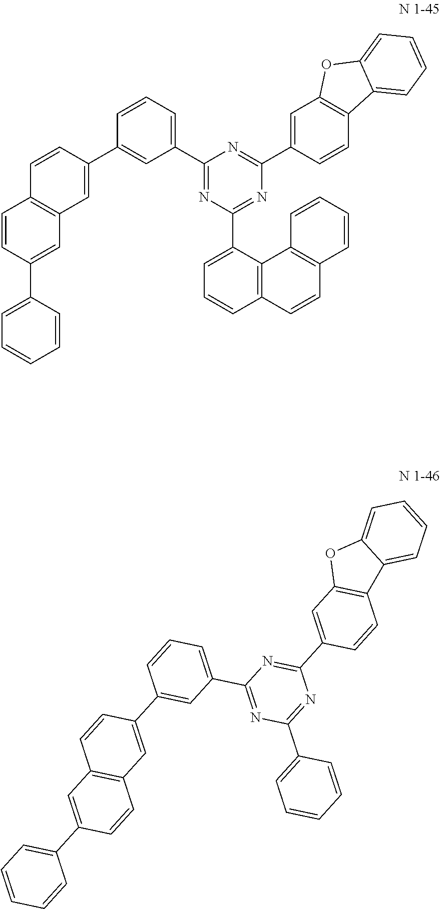 [SMILES: c1ccc(-c2ccc3ccc(-c4cccc(-c5nc(-c6ccc7c(c6)oc6ccccc67)nc(-c6cccc7ccc8ccccc8c67)n5)c4)cc3c2)cc1 ; c1ccc(-c2ccc3cc(-c4cccc(-c5nc(-c6ccccc6)nc(-c6ccc7c(c6)oc6ccccc67)n5)c4)ccc3c2)cc1]

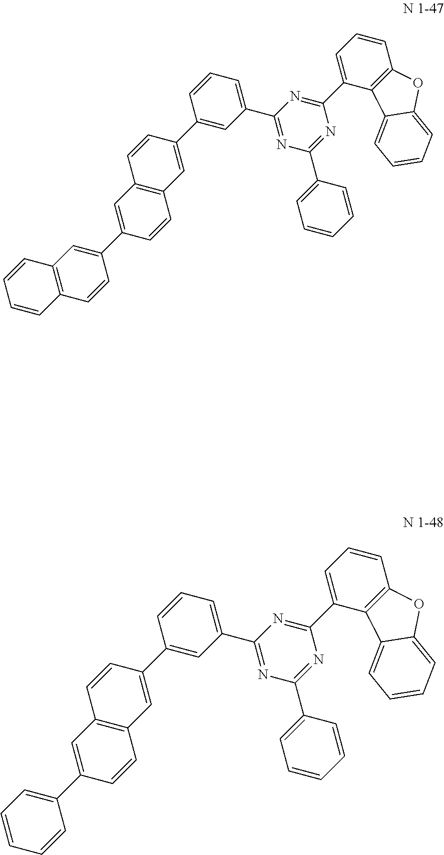 [SMILES: c1ccc(-c2nc(-c3cccc(-c4ccc5cc(-c6ccc7ccccc7c6)ccc5c4)c3)nc(-c3cccc4oc5ccccc5c34)n2)cc1 ; c1ccc(-c2ccc3cc(-c4cccc(-c5nc(-c6ccccc6)nc(-c6cccc7oc8ccccc8c67)n5)c4)ccc3c2)cc1]

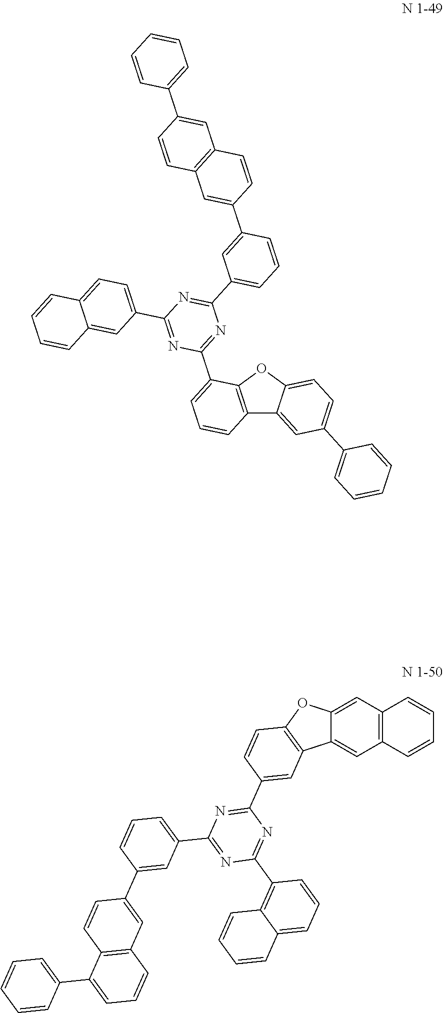 [SMILES: c1ccc(-c2ccc3cc(-c4cccc(-c5nc(-c6ccc7ccccc7c6)nc(-c6cccc7c6oc6ccc(-c8ccccc8)cc67)n5)c4)ccc3c2)cc1 ; c1ccc(-c2cccc3cc(-c4cccc(-c5nc(-c6ccc7oc8cc9ccccc9cc8c7c6)nc(-c6cccc7ccccc67)n5)c4)ccc23)cc1]

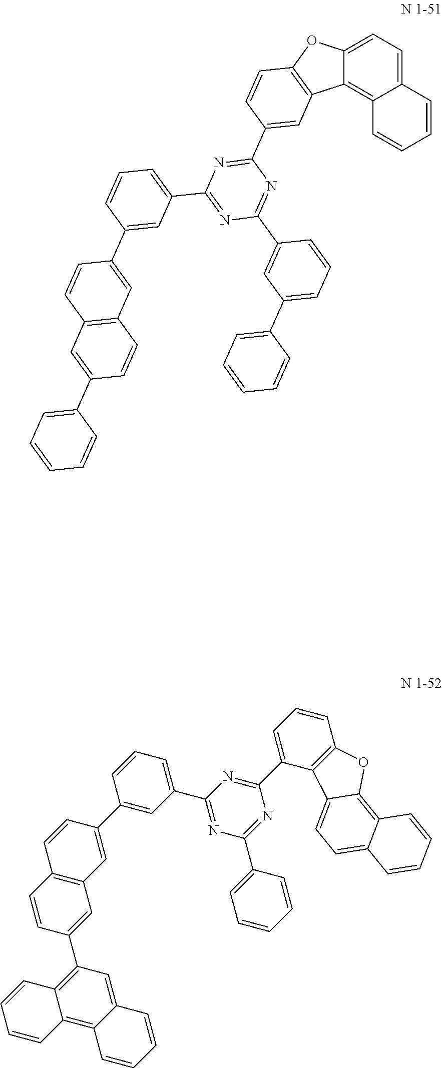 [SMILES: c1ccc(-c2cccc(-c3nc(-c4cccc(-c5ccc6cc(-c7ccccc7)ccc6c5)c4)nc(-c4ccc5oc6ccc7ccccc7c6c5c4)n3)c2)cc1 ; c1ccc(-c2nc(-c3cccc(-c4ccc5ccc(-c6cc7ccccc7c7ccccc67)cc5c4)c3)nc(-c3cccc4oc5c6ccccc6ccc5c34)n2)cc1]

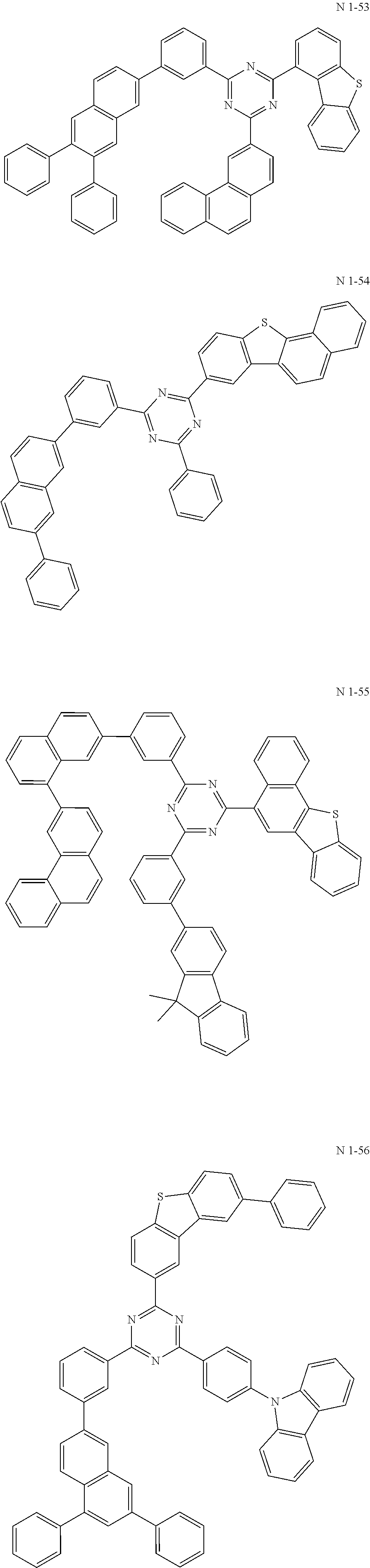 [SMILES: c1ccc(-c2cc3ccc(-c4cccc(-c5nc(-c6ccc7ccc8ccccc8c7c6)nc(-c6cccc7sc8ccccc8c67)n5)c4)cc3cc2-c2ccccc2)cc1 ; c1ccc(-c2ccc3ccc(-c4cccc(-c5nc(-c6ccccc6)nc(-c6ccc7sc8c9ccccc9ccc8c7c6)n5)c4)cc3c2)cc1 ; CC1(C)c2ccccc2-c2ccc(-c3cccc(-c4nc(-c5cccc(-c6ccc7cccc(-c8ccc9ccc%10ccccc%10c9c8)c7c6)c5)nc(-c5cc6c7ccccc7sc6c6ccccc56)n4)c3)cc21 ; c1ccc(-c2cc(-c3ccccc3)c3ccc(-c4cccc(-c5nc(-c6ccc(-n7c8ccccc8c8ccccc87)cc6)nc(-c6ccc7sc8ccc(-c9ccccc9)cc8c7c6)n5)c4)cc3c2)cc1]

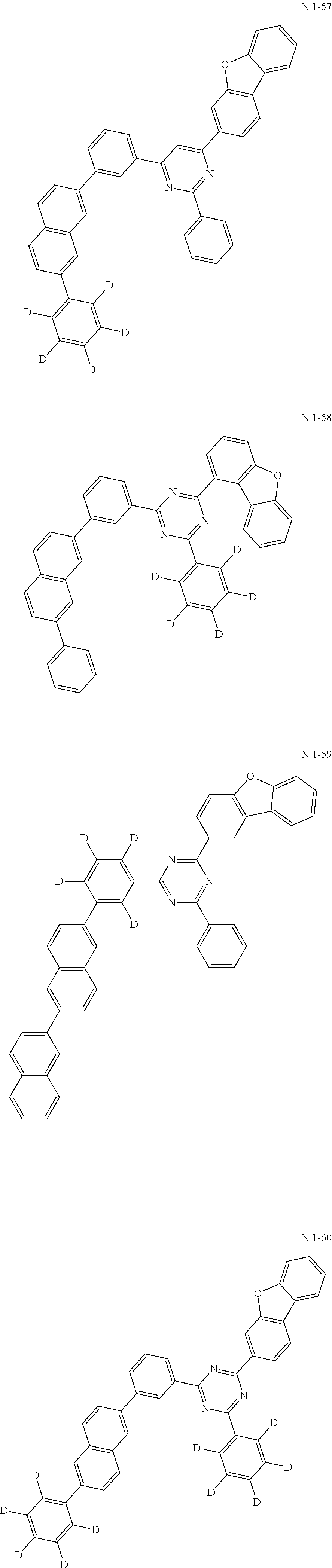 [SMILES: [2H]c1c([2H])c([2H])c(-c2ccc3ccc(-c4cccc(-c5cc(-c6ccc7c(c6)oc6ccccc67)nc(-c6ccccc6)n5)c4)cc3c2)c([2H])c1[2H] ; [2H]c1c([2H])c([2H])c(-c2nc(-c3cccc(-c4ccc5ccc(-c6ccccc6)cc5c4)c3)nc(-c3cccc4oc5ccccc5c34)n2)c([2H])c1[2H] ; [2H]c1c([2H])c(-c2ccc3cc(-c4ccc5ccccc5c4)ccc3c2)c([2H])c(-c2nc(-c3ccccc3)nc(-c3ccc4oc5ccccc5c4c3)n2)c1[2H] ; [2H]c1c([2H])c([2H])c(-c2ccc3cc(-c4cccc(-c5nc(-c6ccc7c(c6)oc6ccccc67)nc(-c6c([2H])c([2H])c([2H])c([2H])c6[2H])n5)c4)ccc3c2)c([2H])c1[2H]]

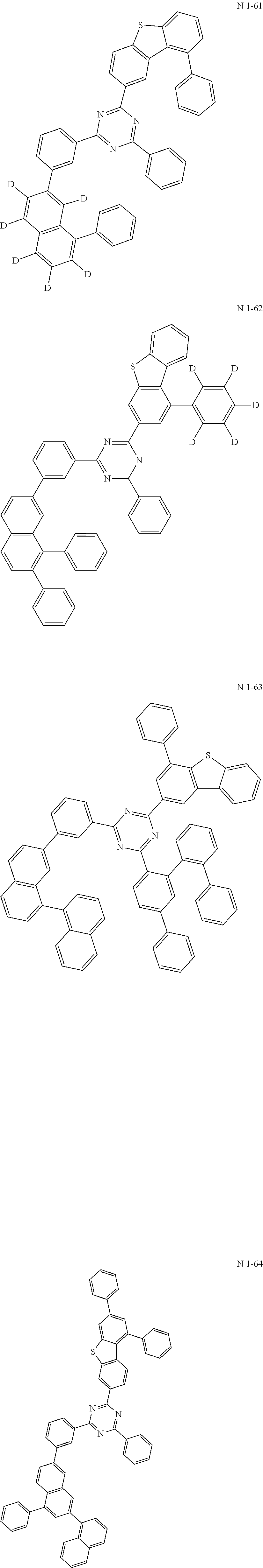

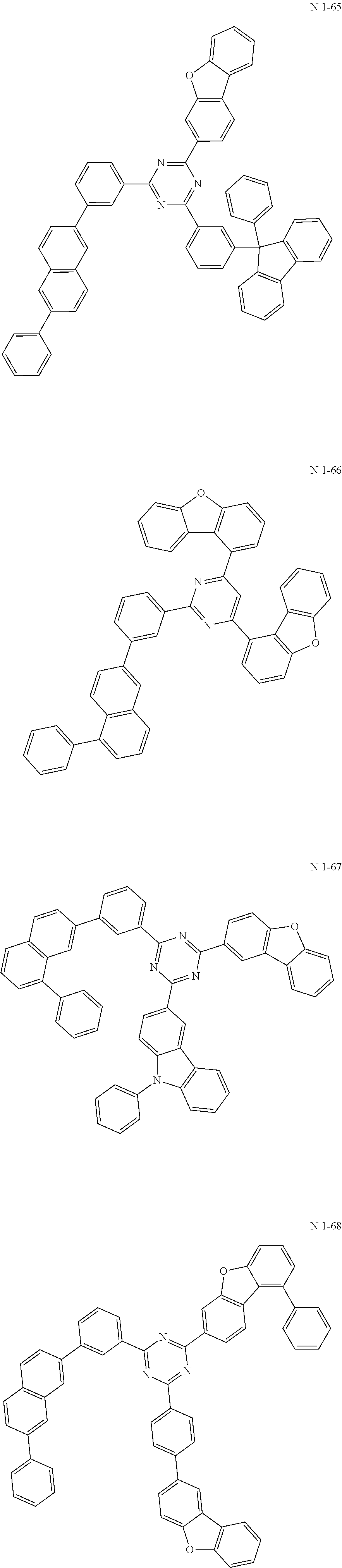 [SMILES: c1ccc(-c2ccc3cc(-c4cccc(-c5nc(-c6cccc(C7(c8ccccc8)c8ccccc8-c8ccccc87)c6)nc(-c6ccc7c(c6)oc6ccccc67)n5)c4)ccc3c2)cc1 ; c1ccc(-c2cccc3cc(-c4cccc(-c5nc(-c6cccc7oc8ccccc8c67)cc(-c6cccc7oc8ccccc8c67)n5)c4)ccc23)cc1 ; c1ccc(-c2cccc3ccc(-c4cccc(-c5nc(-c6ccc7oc8ccccc8c7c6)nc(-c6ccc7c(c6)c6ccccc6n7-c6ccccc6)n5)c4)cc23)cc1 ; c1ccc(-c2ccc3ccc(-c4cccc(-c5nc(-c6ccc(-c7ccc8oc9ccccc9c8c7)cc6)nc(-c6ccc7c(c6)oc6cccc(-c8ccccc8)c67)n5)c4)cc3c2)cc1]

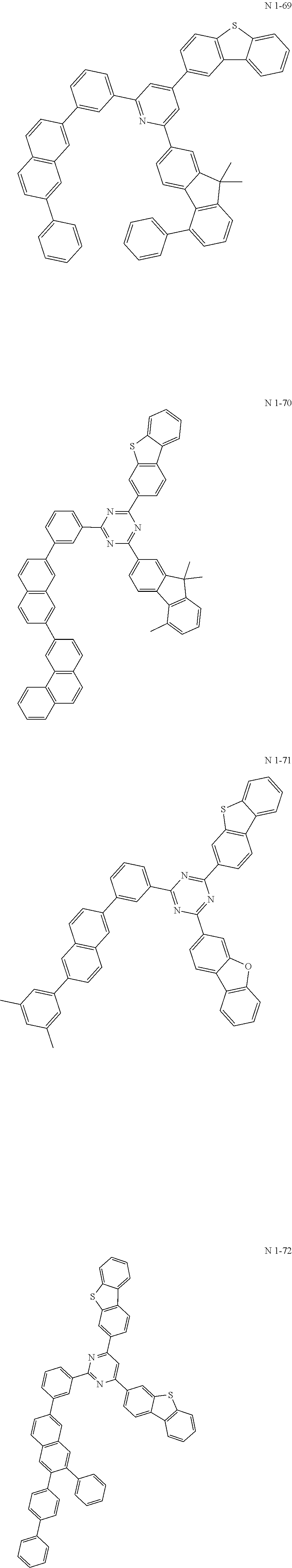

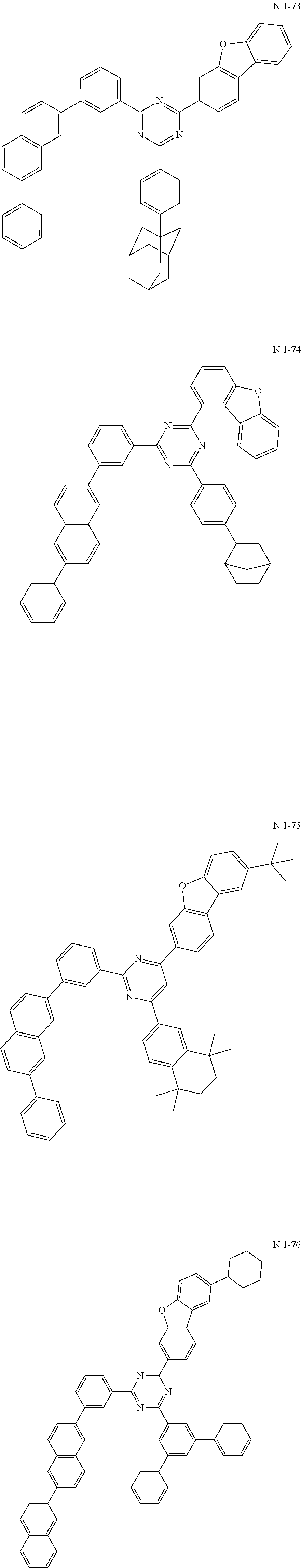 [SMILES: c1ccc(-c2ccc3ccc(-c4cccc(-c5nc(-c6ccc(C78CC9CC(CC(C9)C7)C8)cc6)nc(-c6ccc7c(c6)oc6ccccc67)n5)c4)cc3c2)cc1 ; c1ccc(-c2ccc3cc(-c4cccc(-c5nc(-c6ccc(C7CC8CCC7C8)cc6)nc(-c6cccc7oc8ccccc8c67)n5)c4)ccc3c2)cc1 ; CC(C)(C)c1ccc2oc3cc(-c4cc(-c5ccc6c(c5)C(C)(C)CCC6(C)C)nc(-c5cccc(-c6ccc7ccc(-c8ccccc8)cc7c6)c5)n4)ccc3c2c1 ; c1ccc(-c2cc(-c3ccccc3)cc(-c3nc(-c4cccc(-c5ccc6cc(-c7ccc8ccccc8c7)ccc6c5)c4)nc(-c4ccc5c(c4)oc4ccc(C6CCCCC6)cc45)n3)c2)cc1]

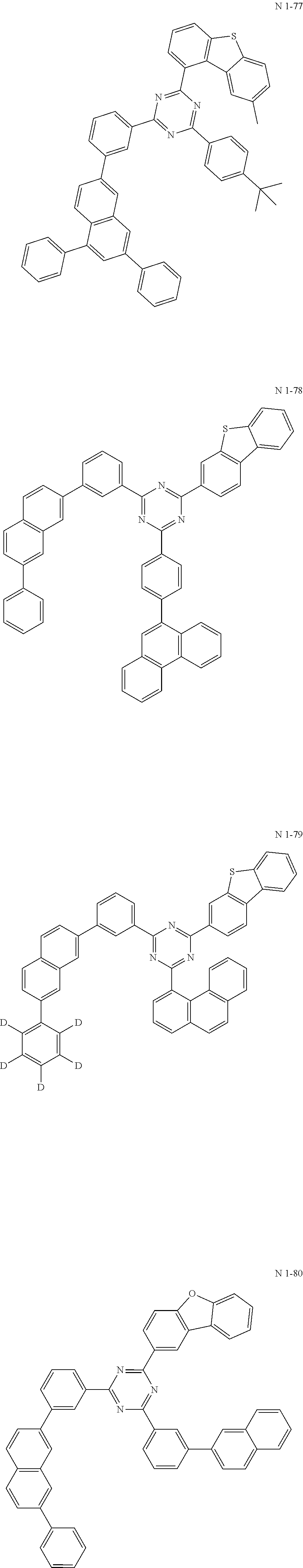 [SMILES: Cc1ccc2sc3cccc(-c4nc(-c5ccc(C(C)(C)C)cc5)nc(-c5cccc(-c6ccc7c(-c8ccccc8)cc(-c8ccccc8)cc7c6)c5)n4)c3c2c1 ; c1ccc(-c2ccc3ccc(-c4cccc(-c5nc(-c6ccc(-c7cc8ccccc8c8ccccc78)cc6)nc(-c6ccc7c(c6)sc6ccccc67)n5)c4)cc3c2)cc1 ; [2H]c1c([2H])c([2H])c(-c2ccc3ccc(-c4cccc(-c5nc(-c6ccc7c(c6)sc6ccccc67)nc(-c6cccc7ccc8ccccc8c67)n5)c4)cc3c2)c([2H])c1[2H] ; c1ccc(-c2ccc3ccc(-c4cccc(-c5nc(-c6cccc(-c7ccc8ccccc8c7)c6)nc(-c6ccc7oc8ccccc8c7c6)n5)c4)cc3c2)cc1]

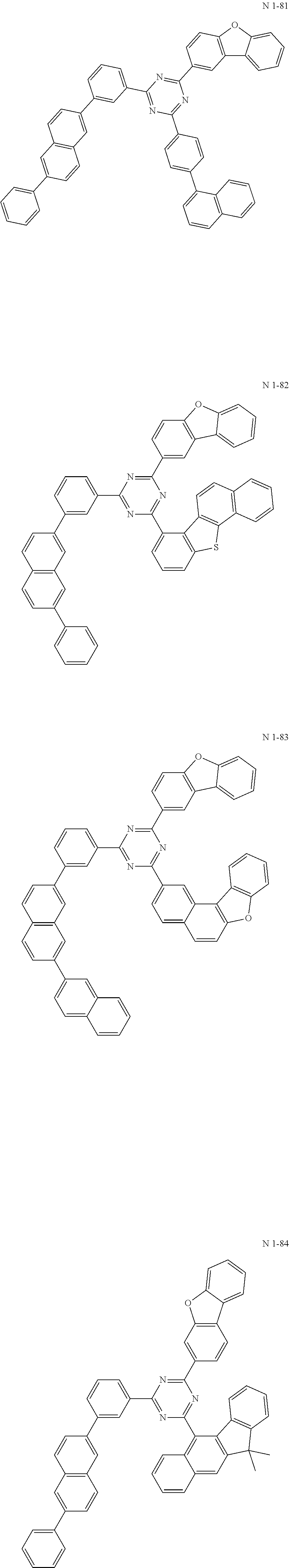 [SMILES: c1ccc(-c2ccc3cc(-c4cccc(-c5nc(-c6ccc(-c7cccc8ccccc78)cc6)nc(-c6ccc7oc8ccccc8c7c6)n5)c4)ccc3c2)cc1 ; c1ccc(-c2ccc3ccc(-c4cccc(-c5nc(-c6ccc7oc8ccccc8c7c6)nc(-c6cccc7sc8c9ccccc9ccc8c67)n5)c4)cc3c2)cc1 ; c1cc(-c2ccc3ccc(-c4ccc5ccccc5c4)cc3c2)cc(-c2nc(-c3ccc4oc5ccccc5c4c3)nc(-c3ccc4ccc5oc6ccccc6c5c4c3)n2)c1 ; CC1(C)c2ccccc2-c2c1cc1ccccc1c2-c1nc(-c2cccc(-c3ccc4cc(-c5ccccc5)ccc4c3)c2)nc(-c2ccc3c(c2)oc2ccccc23)n1]

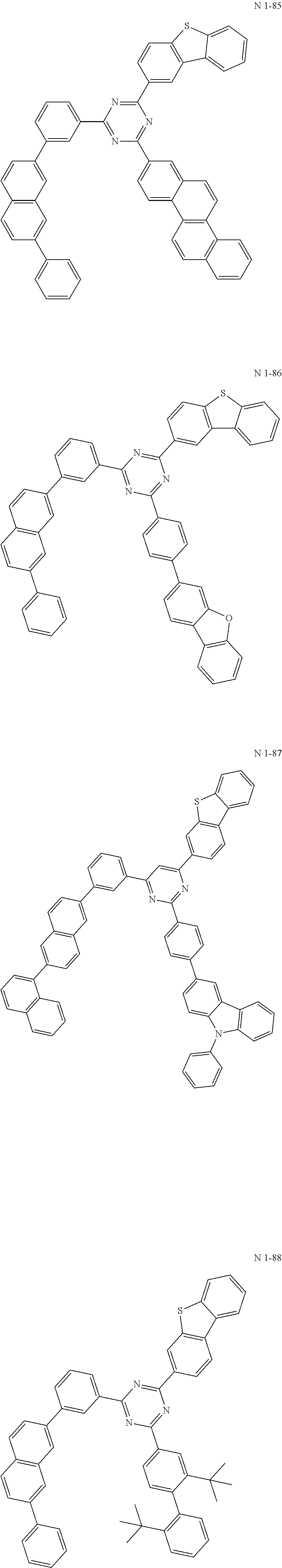 [SMILES: c1ccc(-c2ccc3ccc(-c4cccc(-c5nc(-c6ccc7c(ccc8c9ccccc9ccc78)c6)nc(-c6ccc7sc8ccccc8c7c6)n5)c4)cc3c2)cc1 ; c1ccc(-c2ccc3ccc(-c4cccc(-c5nc(-c6ccc(-c7ccc8c(c7)oc7ccccc78)cc6)nc(-c6ccc7sc8ccccc8c7c6)n5)c4)cc3c2)cc1 ; c1ccc(-n2c3ccccc3c3cc(-c4ccc(-c5nc(-c6cccc(-c7ccc8cc(-c9cccc%10ccccc9%10)ccc8c7)c6)cc(-c6ccc7c(c6)sc6ccccc67)n5)cc4)ccc32)cc1 ; CC(C)(C)c1ccccc1-c1ccc(-c2nc(-c3cccc(-c4ccc5ccc(-c6ccccc6)cc5c4)c3)nc(-c3ccc4c(c3)sc3ccccc34)n2)cc1C(C)(C)C]

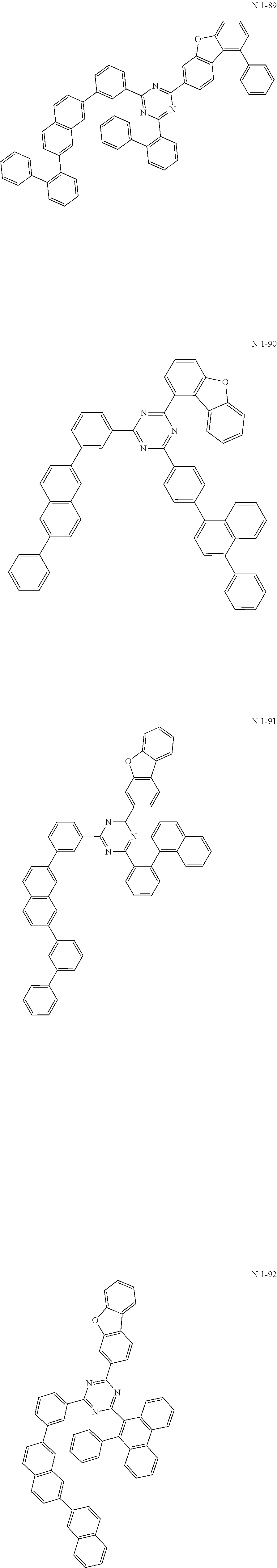 [SMILES: c1ccc(-c2ccccc2-c2ccc3ccc(-c4cccc(-c5nc(-c6ccc7c(c6)oc6cccc(-c8ccccc8)c67)nc(-c6ccccc6-c6ccccc6)n5)c4)cc3c2)cc1 ; c1ccc(-c2ccc3cc(-c4cccc(-c5nc(-c6ccc(-c7ccc(-c8ccccc8)c8ccccc78)cc6)nc(-c6cccc7oc8ccccc8c67)n5)c4)ccc3c2)cc1 ; c1ccc(-c2cccc(-c3ccc4ccc(-c5cccc(-c6nc(-c7ccc8c(c7)oc7ccccc78)nc(-c7ccccc7-c7cccc8ccccc78)n6)c5)cc4c3)c2)cc1 ; c1ccc(-c2c(-c3nc(-c4cccc(-c5ccc6ccc(-c7ccc8ccccc8c7)cc6c5)c4)nc(-c4ccc5c(c4)oc4ccccc45)n3)c3ccccc3c3ccccc23)cc1]

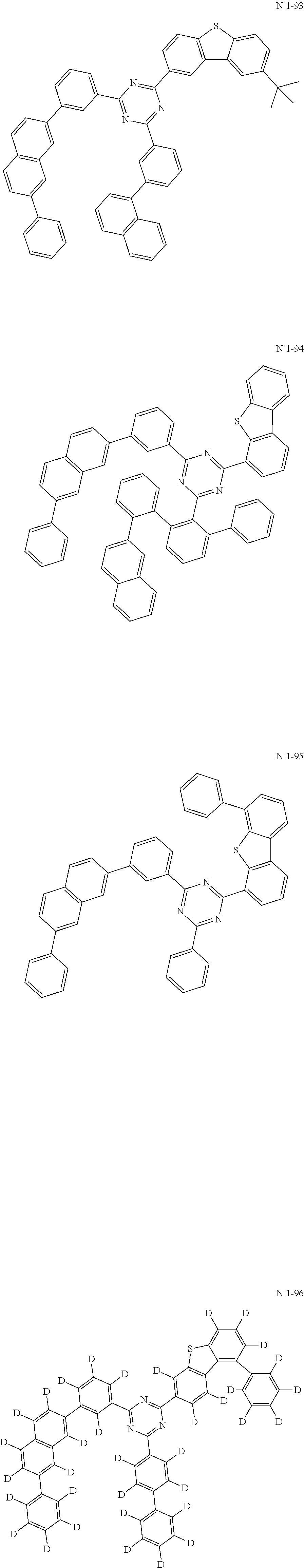

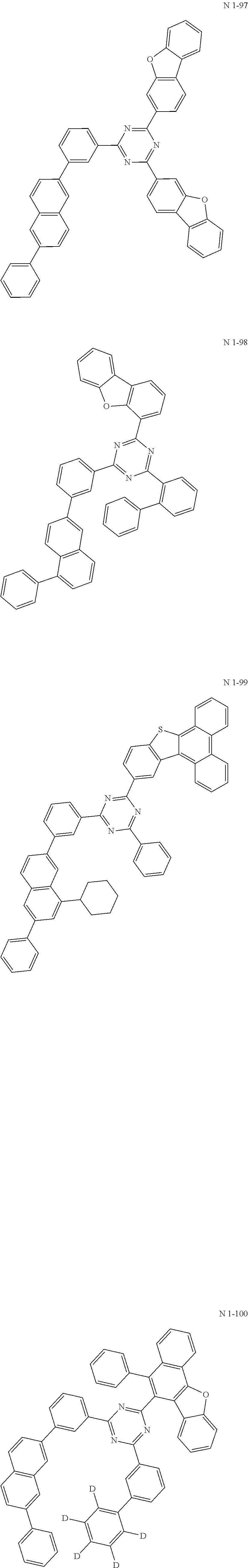 [SMILES: c1ccc(-c2ccc3cc(-c4cccc(-c5nc(-c6ccc7c(c6)oc6ccccc67)nc(-c6ccc7c(c6)oc6ccccc67)n5)c4)ccc3c2)cc1 ; c1ccc(-c2ccccc2-c2nc(-c3cccc(-c4ccc5c(-c6ccccc6)cccc5c4)c3)nc(-c3cccc4c3oc3ccccc34)n2)cc1 ; c1ccc(-c2cc(C3CCCCC3)c3cc(-c4cccc(-c5nc(-c6ccccc6)nc(-c6ccc7sc8c9ccccc9c9ccccc9c8c7c6)n5)c4)ccc3c2)cc1 ; [2H]c1c([2H])c([2H])c(-c2cccc(-c3nc(-c4cccc(-c5ccc6ccc(-c7ccccc7)cc6c5)c4)nc(-c4c(-c5ccccc5)c5ccccc5c5oc6ccccc6c45)n3)c2)c([2H])c1[2H]]

Reorganization energy refers to the energy lost due to changes in molecular structure arrangement during the movement of charges (electrons, holes). It depends on the molecular geometry, and has a characteristic that the value becomes smaller as the difference between the PES (Potential Energy Surface) of the neutral state and the PES of the charged state is small. RE value can be obtained by the following formula.

REₕₒₗₑ:λ⁺=(E_{NOCE}−E_{COCE})+(E_{CONE}−E_{NONE})

RE_{elec}:λ⁻=(E_{NOAE}−E_{AOAE})+(E_{AONE}−E_{NONE})

Each factor is described as NONE: Neutral geometry of Neutral molecules (=NO opt.), NOAE: Anion geometry of Neutral molecules, NOCE: Cation geometry of Neutral molecules, AONE: Neutral geometry of Anion molecules, AOAE: Anion geometry of Anion molecules (=AO opt.), CONE: Neutral geometry of Cation molecules, COCE: Cation geometry of Cation molecules (=CO opt.)

The value of Reorganization Energy is inversely proportional to mobility, and under the condition that they have the same r and T values, RE value of each material directly affects the mobility. The relation between RE value and mobility is expressed as follows.

$$μ = k \frac{{r}^{2}}{2 {k}_{B} T / e}$$

$$k = ( \frac{4 {π}^{2}}{h} ) \frac{{t}^{2}}{\sqrt{4 π λ {k}_{B} T}} exp [ - \frac{λ}{4 {k}_{B} T} ]$$

Each factor is described as λ: Reorganization energy/μ: mobility/r: dimer displacement/t: intermolecular charge transfer matrix element. From the above equation, it can be seen that the lower RE value, the faster the mobility.

Reorganization energy value requires a simulation tool that can calculate the potential energy according to the molecular structure, we used Gaussian09 (hereinafter G09) and Jaguar module of Schrodinger Materials Science (hereinafter JG). Both G09 and JG are tools to analyze the properties of molecules through quantum mechanical (QM) calculations, and have the function of optimizing the molecular structure or calculating the energy for a given molecular structure (single-point energy).

The process of performing QM calculations in molecular structures requires large computational resources, and our company uses 2 cluster servers for these calculations. Each cluster server consists of 4 node workstations and 1 master workstation, each node performed molecular QM calculations by Parallel computing through symmetric multi-processing (SMP) using a CPU with more than 36 cores.

Using G09, the optimized molecular structure and its potential energy (NONE/COCE) in the neutral/charged state required for rearrangement energy were calculated. The charge state potential energy (NOCE) of the structure optimized for the neutral state and the neutral state potential energy (CONE) of the structure optimized for the charge state were calculated by changing only the charges to the 2 optimized structures. After that, the rearrangement energy was calculated according to the following relation.

RE_{charge}:λ=(E_{NOCE}−E_{COCE})+(E_{CONE}−E_{NONE})

Because Schrödinger provides a function to automatically perform such a calculation process, the potential energy according to each state was sequentially calculated through the JG module by providing the molecular structure (NO) of the basic state, and the RE value was calculated.

According to an embodiment of the present invention, more electrons are attracted to an element having a greater electronegativity among two atoms in one covalent bond. Here, the relatively high electronegative atom has a δ− charge, the low electronegativity atoms have a δ+ charge. As described above, the difference in polarity of two atoms is called a dipole. At this time, Dipole moment can be calculated as a vector quantity multiplied by the intensity of the two poles and the distance between the two atomic nuclei. In other words, Dipole moment can be calculated by the following equation.

μ=δ*d

Each factor is described by μ: dipole moment/δ: magnitude of the partial charges δ⁺ and δ⁻/d: distance between δ⁺ and δ⁻.

Our company used G09 to optimize the molecular structure with B3LYP/6-31G(d). Based on the result, Mulliken Charge value of each atom was obtained, and Dipole moment was calculated by multiplying the vector in the axial direction. Dipole moment is the vector sum of each bond dipole moment. Dipole moment value means the magnitude of the vector dipole moment, and it can be expressed as the value of the vector length as follows.

|μ|=\sqrt{x²+y²+z²}

The RE value of Formula 3 calculated in this way may be preferably 0.100 to 0.290, more preferably 0.150 to 0.260.

Also, the present invention relates to an organic electronic element comprising a first electrode; a second electrode; and an organic material layer formed between the first electrode and the second electrode, wherein the organic material layer includes an emitting layer, wherein the emitting layer is a phosphorescent emitting layer, and comprises a first host compound represented by Formula 3 and a second host compound represented by Formula 4 or Formula 5.

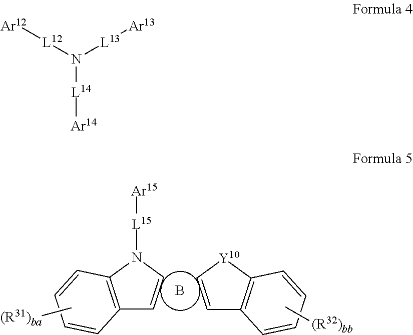

In Formula 4 and Formula 5, each symbol may be defined as follows.

Ar¹², Ar¹³ and Ar¹⁴ are each independently selected from the group consisting of a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring;

When Ar¹², Ar¹³ and Ar¹⁴ are an aryl group, it may be preferably a C₆-C₃₀ aryl group, and more preferably a C₆-C₂₅ aryl group, for example, it may be phenylene, biphenyl, naphthalene, terphenyl, phenanthrene, etc,

When Ar¹², Ar¹³ and Ar¹⁴ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

When Ar¹², Ar¹³ and Ar¹⁴ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Ar¹⁵ is each independently selected from the group consisting of a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and -L″-NR^{f}R^{g};

When Ar¹⁵ is an aryl group, it may be preferably a C₆-C₃₀ aryl group, and more preferably a C₆-C₂₅ aryl group, for example, it may be phenylene, biphenyl, naphthalene, terphenyl, phenanthrene, etc,

When Ar¹⁵ is a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

When Ar¹⁵ is a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

L¹², L¹³, L¹⁴, L¹⁵ and L″ are each independently selected from the group consisting of a single bond; a C₆-C₆₀ arylene group; fluorenylene group; a fused ring group of a C₃-C₆₀ aliphatic ring and a C₆-C₆₀ aromatic ring; a C₂-C₆₀ heterocyclic group including at least one heteroatom of O, N, S, Si or P; When L¹², L¹³, L¹⁴, L¹⁵ and L″ are an arylene group, it may be preferably a C₆-C₃₀ arylene group, more preferably a C₆-C₂₄ arylene group, for example, phenylene, biphenyl, naphthalene, terphenyl, etc.,

When L¹², L¹³, L¹⁴, L¹⁵ and L″ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring,

When L¹², L¹³, L¹⁴, L¹⁵ and L″ are a heterocyclic group, it may be preferably a C₂-C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

R^{f} and R^{g} are each independently selected from the group consisting of a C₆-C₆₀ aryl group; fluorenyl group; a C₂-C₆₀ heterocyclic group including at least one heteroatom of O, N, S, Si or P; a C₃-C₆₀ aliphatic ring;

When R^{f} and R^{g} are an aryl group, it may be preferably a C₆-C₃₀ aryl group, and more preferably a C₆-C₂₅ aryl group, for example, it may be phenylene, biphenyl, naphthalene, terphenyl, etc,

When R^{f} and R^{g} are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

When R^{f} and R^{g} are aliphatic groups, they may be preferably C₃-C₃₀ aliphatic groups, more preferably C₃-C₂₄ aliphatic groups.

Y¹⁰ is O, S, CR⁵¹R⁵² or NR⁵³,

B ring is a C₆˜C₂₀ aryl group,

R³¹ and R³² are the same or different, and each independently selected from the group consisting of a hydrogen; deuterium; halogen; cyano group; nitro group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; and a C₆-C₆₀ aryloxy group; or a plurality of adjacent R³¹s or a plurality of R³²s may be bonded to each other to form a ring,

When R³¹ and R³² are an aryl group, it may be preferably a C₆-C₃₀ aryl group, and more preferably a C₆-C₂₅ aryl group, for example, it may be phenyl, biphenyl, naphthalene, terphenyl, phenanthrene, etc,

When R³¹ and R³² are a heterocyclic group, it may be preferably a C₂˜C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine etc.,

When R³¹ and R³² are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

When R³¹ and R³² are an alkyl group, it may be preferably a C₁-C₃₀ alkyl group, and more preferably a C₁-C₂₄ alkyl group.

When R³¹ and R³² are an alkenyl group, it may be preferably an C₂-C₃₀ alkenyl group, and more preferably a C₂-C₂₄ alkenyl group.

When R³¹ and R³² are an alkynyl group, it may be preferably an C₂-C₃₀ alkynyl group, and more preferably a C₂-C₂₄ alkynyl group.

When R³¹ and R³² are alkoxyl groups, they may be preferably C₁˜C₃₀ alkoxyl groups, and more preferably C₁˜C₂₄ alkoxyl groups,

When R³¹ and R³² are an aryloxy group, it may be preferably a C₆˜C₃₀ aryloxy group, and more preferably a C₆˜C₂₄ aryloxy,

R⁵¹, R⁵² and R⁵³ are each independently selected from the group consisting of a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; a C₆-C₆₀ aryloxy group; or

R⁵¹ and R⁵² may be bonded to each other to form a spiro,

When R⁵¹, R⁵² and R⁵³ are an aryl group, it may be preferably a C₆-C₃₀ aryl group, and more preferably a C₆-C₂₅ aryl group, for example, it may be phenylene, biphenyl, naphthalene, terphenyl, phenanthrene, etc,

When R⁵¹, R⁵² and R⁵³ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

When R⁵¹, R⁵² and R⁵³ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

When R⁵¹, R⁵² and R⁵³ are an alkyl group, it may be preferably a C₁-C₃₀ alkyl group, and more preferably a C₁-C₂₄ alkyl group.

When R⁵¹, R⁵² and R⁵³ are an alkenyl group, it may be preferably an C₂-C₃₀ alkenyl group, and more preferably a C₂-C₂₄ alkenyl group.

When R⁵¹, R⁵² and R⁵³ are an alkynyl group, it may be preferably an C₂-C₃₀ alkynyl group, and more preferably a C₂-C₂₄ alkynyl group.

When R⁵¹, R⁵² and R⁵³ are alkoxyl groups, they may be preferably C₁˜C₃₀ alkoxyl groups, and more preferably C₁˜C₂₄ alkoxyl groups,

When R⁵¹, R⁵² and R⁵³ are an aryloxy group, it may be preferably a C₆˜C₃₀ aryloxy group, and more preferably a C₆˜C₂₄ aryloxy,

ba and bb are independently integers from 0 to 4;

wherein the aryl group, arylene group, heterocyclic group, fluorenyl group, fluorenylene group, aliphatic ring group, fused ring group, alkyl group, alkenyl group, alkynyl group, alkoxyl group, and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Formula 4 may be represented by any one of Formulas 4-1 to 4-3.

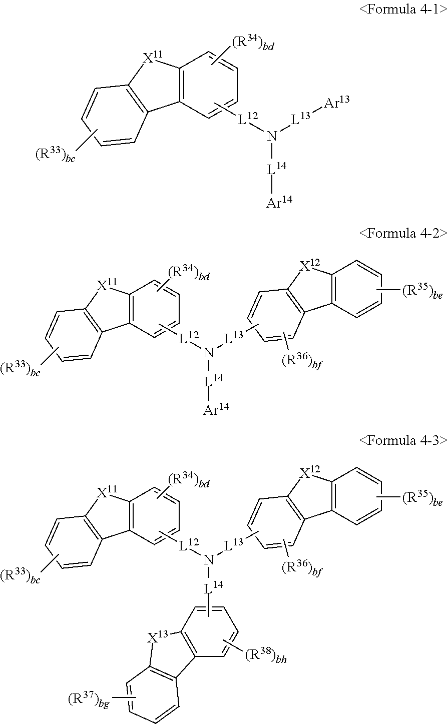

Wherein,
Ar¹³, Ar¹⁴, L¹², L¹³ and L¹⁴ are the same as defined in Formula 4,
X¹¹, X¹² and X¹³ are the same as the definition of Y¹⁰,
R³³, R³⁴, R³⁵, R³⁶, R³⁷ and R³⁸ are the same as the definition of R³¹, or an adjacent plurality of R³³s, a plurality of R³⁴s, a plurality of R³⁵s, a plurality of R³⁶s, a plurality of R³⁷s, or a plurality of R³⁸s may bond to each other to form a ring,
bc, be and bg are each independently integers from 0 to 4, and bd, bf and bh are each independently integers from 0 to 3,

Formula 5 may be represented by any one of Formulas 5-1 to 5-6.

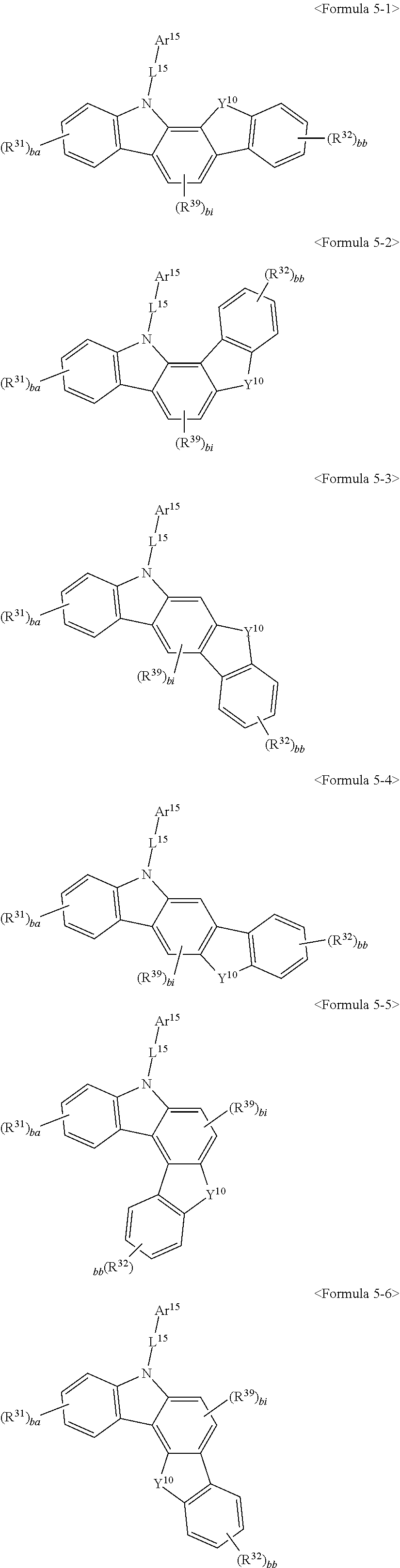

Wherein,
Y¹⁰, R³¹, R³², Ar¹⁵, L¹⁵, ba and bb are the same as defined in Formula 5,
R³⁹ is the e same as defined in Formula 5, same as the definition of R³¹,
bi is an integer of 0 to 2.

Formula 5 may be represented by any one of Formulas 5-7 to 5-9.

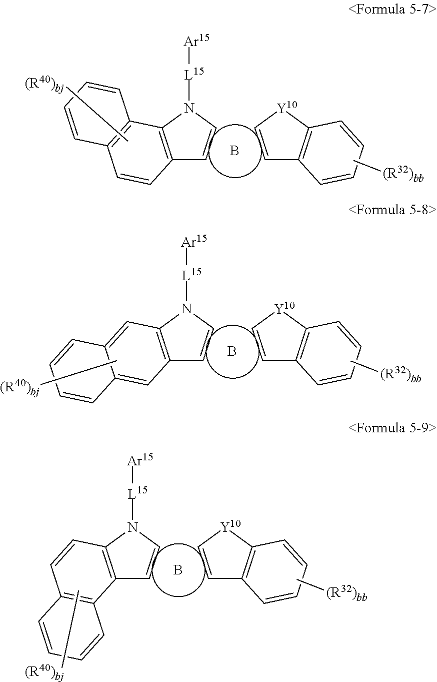

Wherein,
Y¹⁰, B ring, R³², bb, L¹⁵ and Ar¹⁵ are the same as defined in Formula 5,
R⁴⁰ is the e same as defined in Formula 5, same as the definition of R³¹,
bj is an integer of 0 to 6.

Formula 5 may be represented by any one of Formulas 5-10 to 5-12.

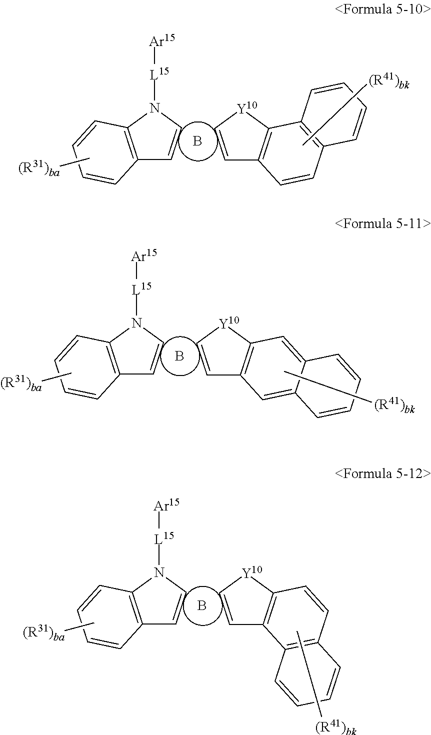

Wherein,
Y¹⁰, B ring, R³¹, ba, L¹⁵ and Ar¹⁵ are the same as defined in Formula 5,
R⁴¹ is the e same as defined in Formula 5, same as the definition of R³¹,
bk is an integer of 0 to 6.

Formula 5 may be represented by any one of Formulas 5-13 to 5-18.

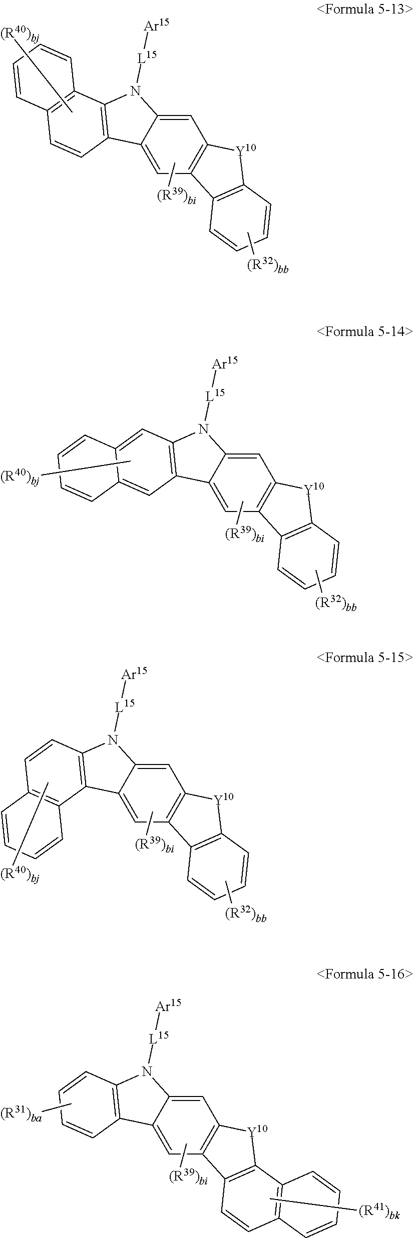

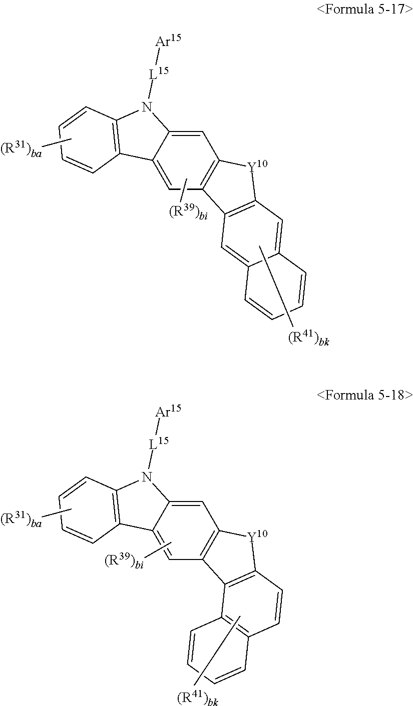

Wherein,
Y¹⁰, R³¹, R³², ba, bb, L¹⁵ and Ar¹⁵ are the same as defined in Formula 5,
R³⁹, R⁴⁰ and R⁴¹ are the e same as defined in Formula 5, same as the definition of R³¹,
bi is an integer of 0 to 2, bj and bk are each independently integers from 0 to 6.

Formula 5 may be represented by Formula 5-19.

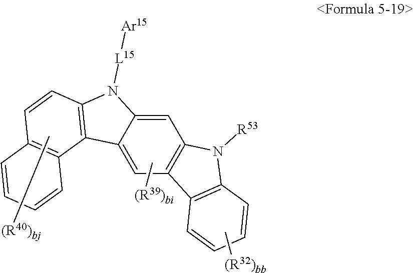

Wherein,
R³², R⁵³, bb, L¹⁵ and Ar¹⁵ are the same as defined in Formula 5,
R³⁹ and R⁴⁰ are the e same as defined in Formula 5, same as the definition of R³¹,
bi is an integer of 0 to 2, bj is an integer from 0 to 6.

Specifically, the compound represented by Formula 4 may be any one of the following compounds H-1 to H-100, but is not limited thereto.

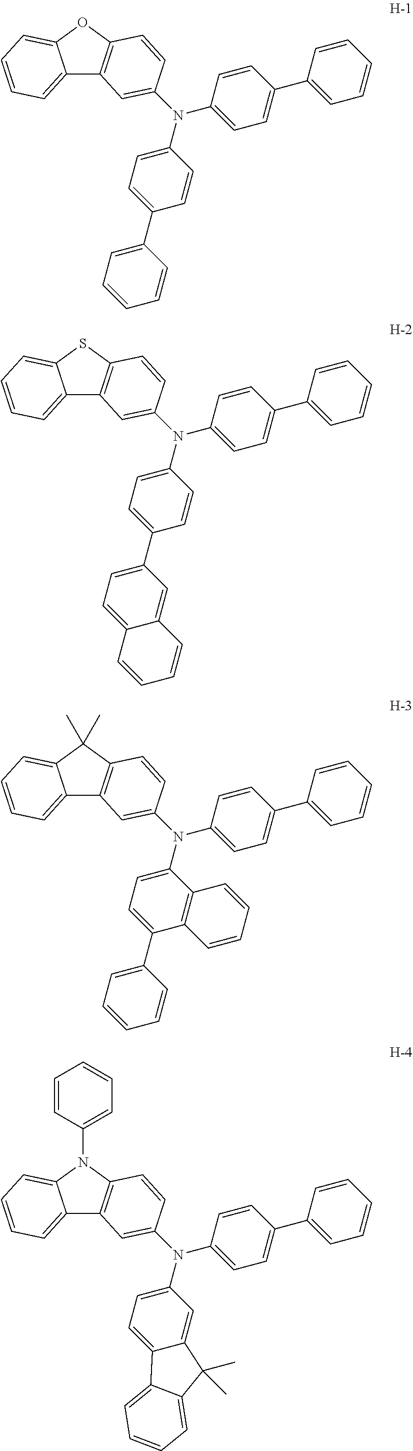 [SMILES: c1ccc(-c2ccc(N(c3ccc(-c4ccccc4)cc3)c3ccc4oc5ccccc5c4c3)cc2)cc1 ; c1ccc(-c2ccc(N(c3ccc(-c4ccc5ccccc5c4)cc3)c3ccc4sc5ccccc5c4c3)cc2)cc1 ; CC1(C)c2ccccc2-c2cc(N(c3ccc(-c4ccccc4)cc3)c3ccc(-c4ccccc4)c4ccccc34)ccc21 ; CC1(C)c2ccccc2-c2ccc(N(c3ccc(-c4ccccc4)cc3)c3ccc4c(c3)c3ccccc3n4-c3ccccc3)cc21]

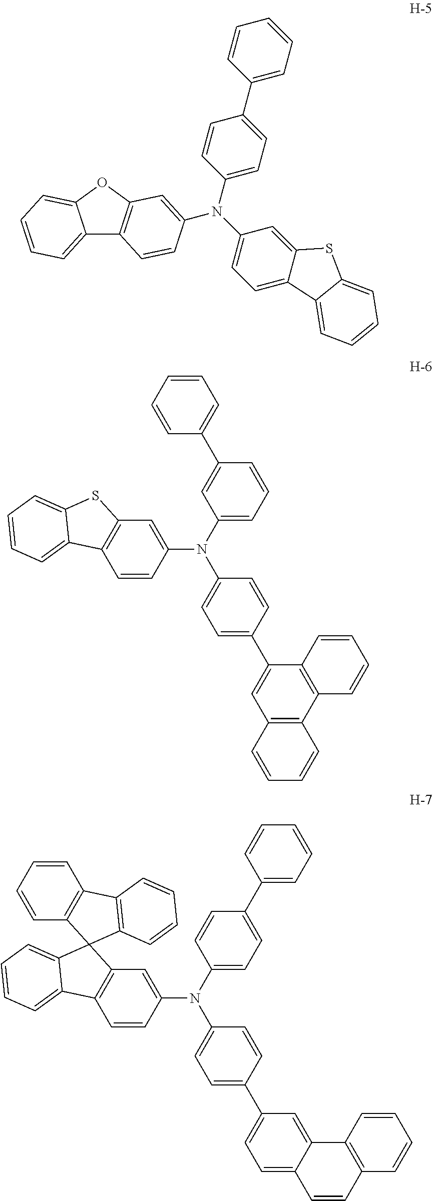

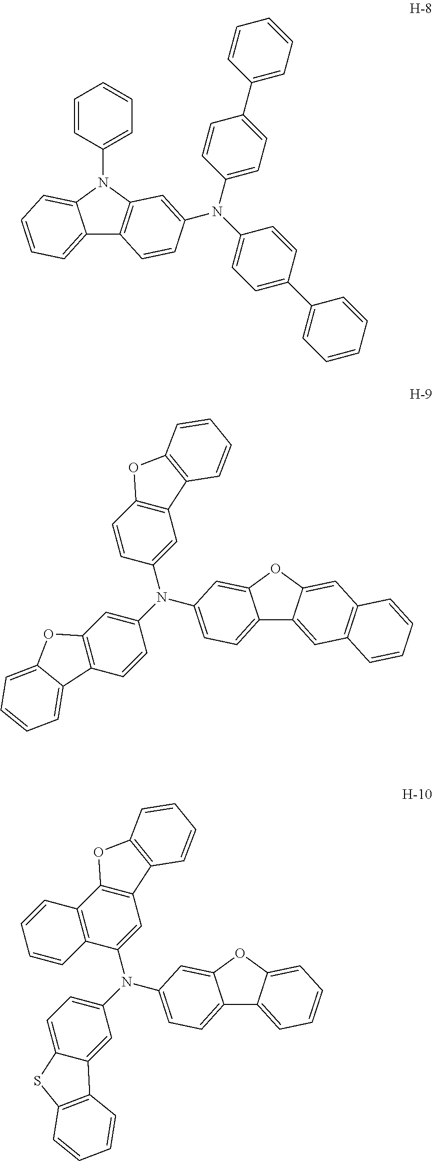 [SMILES: c1ccc(-c2ccc(N(c3ccc(-c4ccccc4)cc3)c3ccc4c5ccccc5n(-c5ccccc5)c4c3)cc2)cc1 ; c1ccc2cc3c(cc2c1)oc1cc(N(c2ccc4c(c2)oc2ccccc24)c2ccc4oc5ccccc5c4c2)ccc13 ; c1ccc2c(c1)oc1cc(N(c3ccc4sc5ccccc5c4c3)c3cc4c5ccccc5oc4c4ccccc34)ccc12]

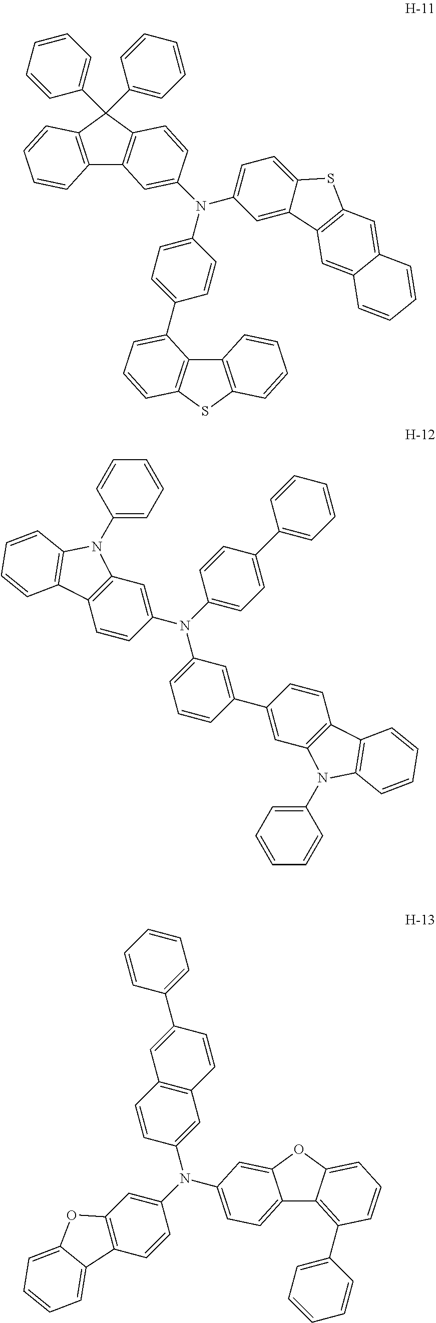 [SMILES: c1ccc(C2(c3ccccc3)c3ccccc3-c3cc(N(c4ccc(-c5cccc6sc7ccccc7c56)cc4)c4ccc5sc6cc7ccccc7cc6c5c4)ccc32)cc1 ; c1ccc(-c2ccc(N(c3cccc(-c4ccc5c6ccccc6n(-c6ccccc6)c5c4)c3)c3ccc4c5ccccc5n(-c5ccccc5)c4c3)cc2)cc1 ; c1ccc(-c2ccc3cc(N(c4ccc5c(c4)oc4ccccc45)c4ccc5c(c4)oc4cccc(-c6ccccc6)c45)ccc3c2)cc1]

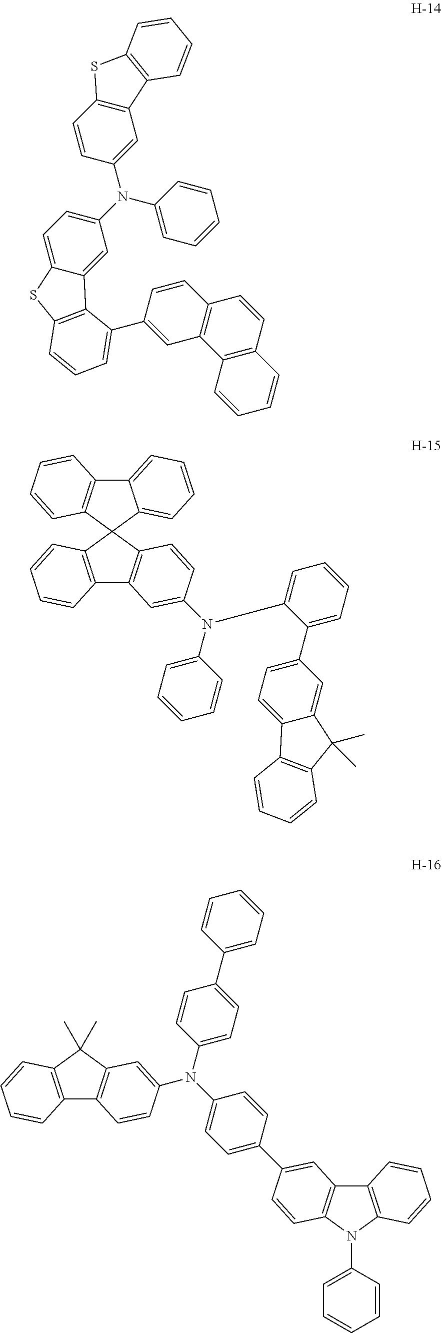 [SMILES: c1ccc(N(c2ccc3sc4ccccc4c3c2)c2ccc3sc4cccc(-c5ccc6ccc7ccccc7c6c5)c4c3c2)cc1 ; CC1(C)c2ccccc2-c2ccc(-c3ccccc3N(c3ccccc3)c3ccc4c(c3)-c3ccccc3C43c4ccccc4-c4ccccc43)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

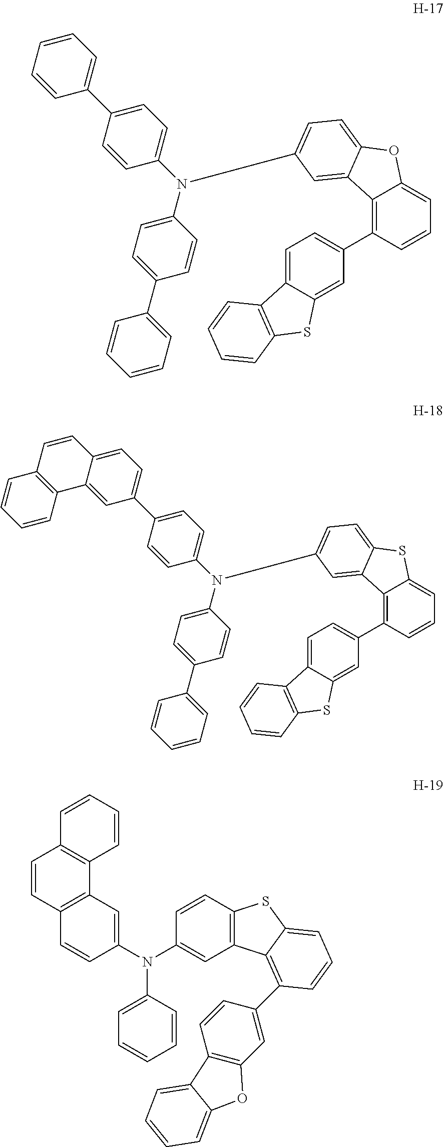 [SMILES: c1ccc(-c2ccc(N(c3ccc(-c4ccccc4)cc3)c3ccc4oc5cccc(-c6ccc7c(c6)sc6ccccc67)c5c4c3)cc2)cc1 ; c1ccc(-c2ccc(N(c3ccc(-c4ccc5ccc6ccccc6c5c4)cc3)c3ccc4sc5cccc(-c6ccc7c(c6)sc6ccccc67)c5c4c3)cc2)cc1 ; c1ccc(N(c2ccc3ccc4ccccc4c3c2)c2ccc3sc4cccc(-c5ccc6c(c5)oc5ccccc56)c4c3c2)cc1]

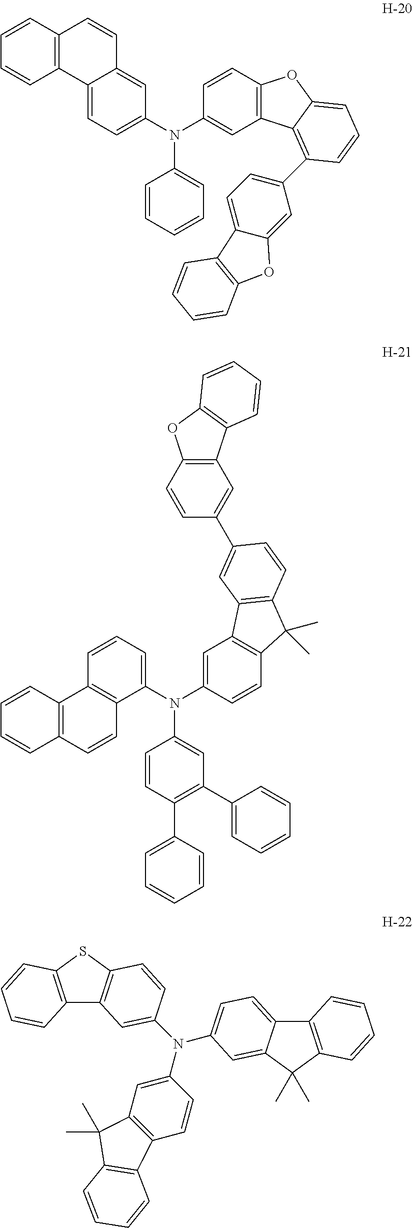 [SMILES: c1ccc(N(c2ccc3c(ccc4ccccc43)c2)c2ccc3oc4cccc(-c5ccc6c(c5)oc5ccccc56)c4c3c2)cc1 ; CC1(C)c2ccc(-c3ccc4oc5ccccc5c4c3)cc2-c2cc(N(c3ccc(-c4ccccc4)c(-c4ccccc4)c3)c3cccc4c3ccc3ccccc34)ccc21 ; CC1(C)c2ccccc2-c2ccc(N(c3ccc4c(c3)C(C)(C)c3ccccc3-4)c3ccc4sc5ccccc5c4c3)cc21]

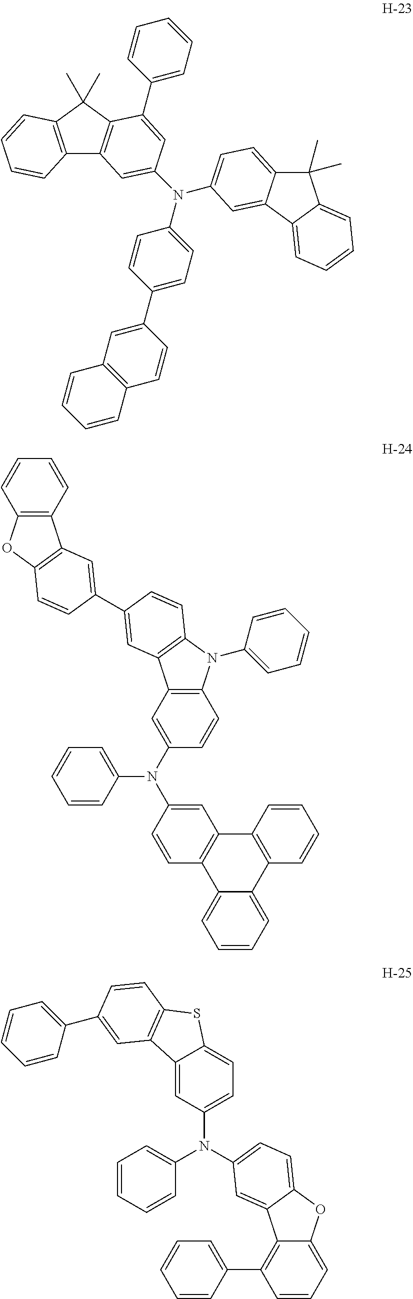

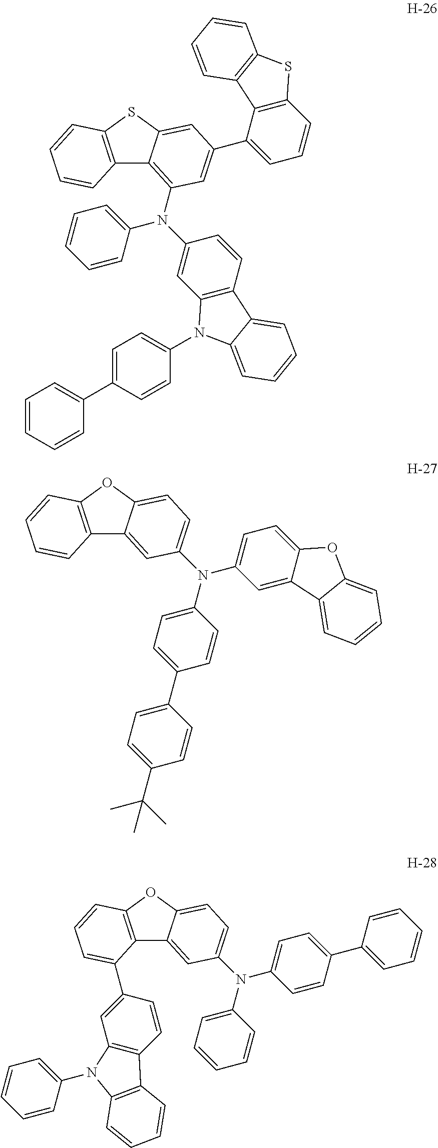

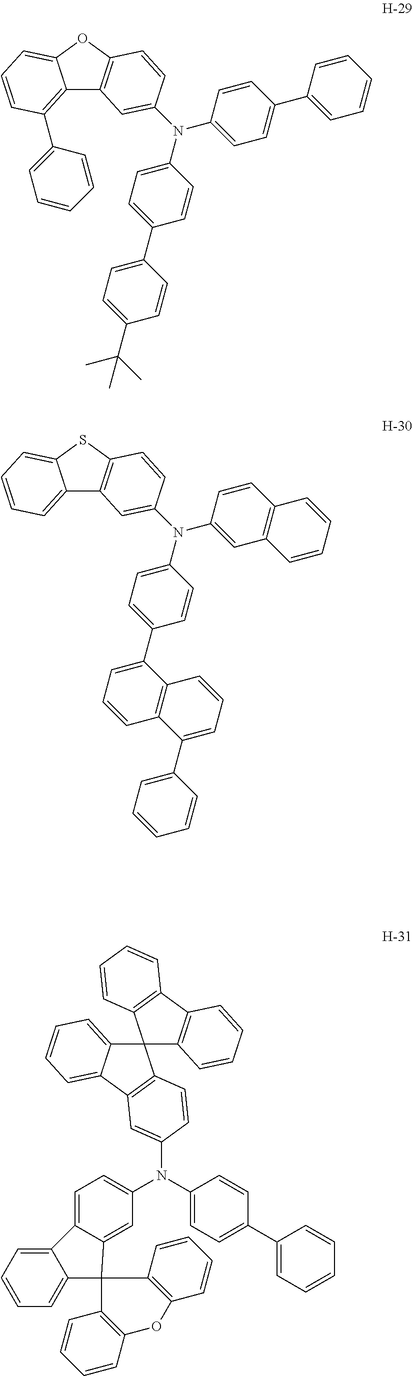 [SMILES: CC(C)(C)c1ccc(-c2ccc(N(c3ccc(-c4ccccc4)cc3)c3ccc4oc5cccc(-c6ccccc6)c5c4c3)cc2)cc1 ; c1ccc(-c2cccc3c(-c4ccc(N(c5ccc6ccccc6c5)c5ccc6sc7ccccc7c6c5)cc4)cccc23)cc1 ; c1ccc(-c2ccc(N(c3ccc4c(c3)-c3ccccc3C43c4ccccc4-c4ccccc43)c3ccc4c(c3)C3(c5ccccc5Oc5ccccc53)c3ccccc3-4)cc2)cc1]

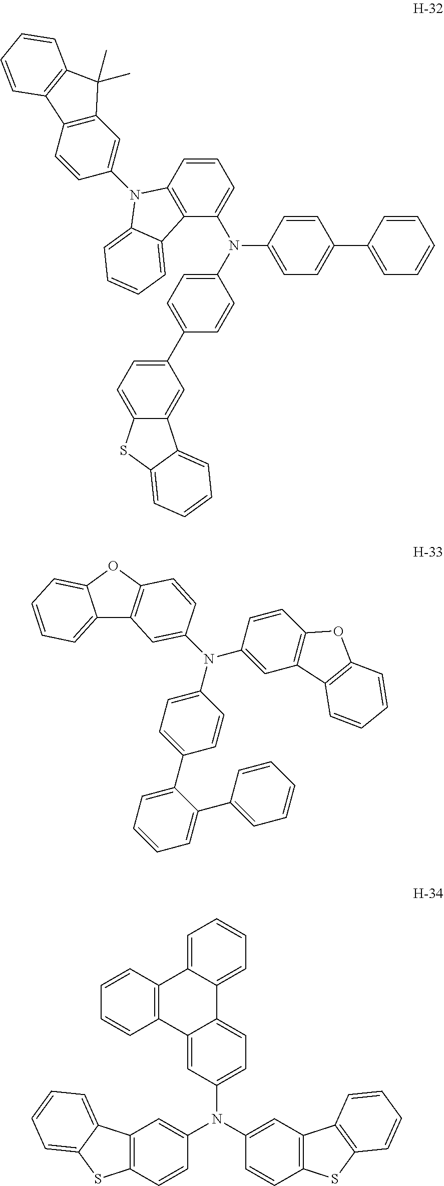 [SMILES: CC1(C)c2ccccc2-c2ccc(-n3c4ccccc4c4c(N(c5ccc(-c6ccccc6)cc5)c5ccc(-c6ccc7sc8ccccc8c7c6)cc5)cccc43)cc21 ; c1ccc(-c2ccccc2-c2ccc(N(c3ccc4oc5ccccc5c4c3)c3ccc4oc5ccccc5c4c3)cc2)cc1 ; c1ccc2c(c1)sc1ccc(N(c3ccc4sc5ccccc5c4c3)c3ccc4c5ccccc5c5ccccc5c4c3)cc12]

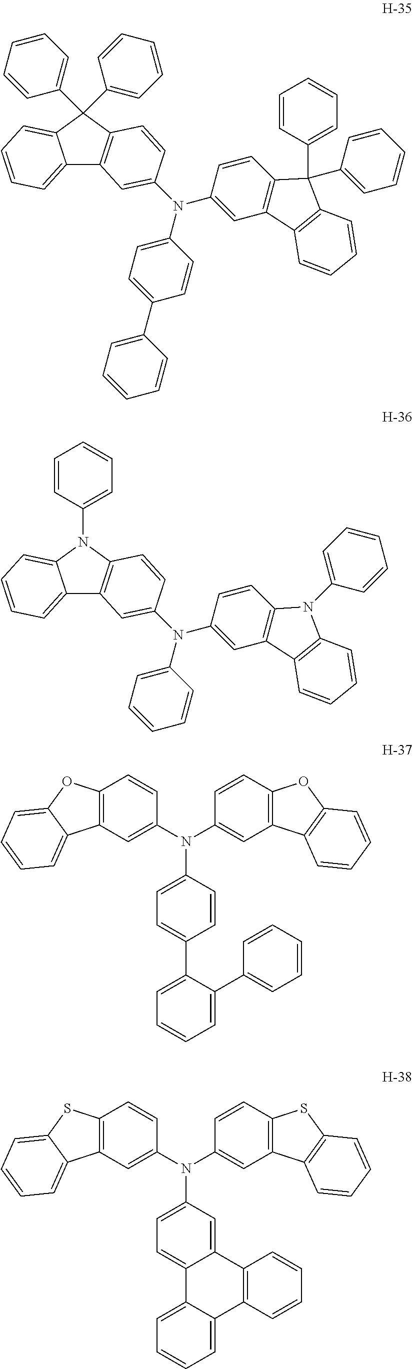 [SMILES: c1ccc(-c2ccc(N(c3ccc4c(c3)-c3ccccc3C4(c3ccccc3)c3ccccc3)c3ccc4c(c3)-c3ccccc3C4(c3ccccc3)c3ccccc3)cc2)cc1 ; c1ccc(N(c2ccc3c(c2)c2ccccc2n3-c2ccccc2)c2ccc3c(c2)c2ccccc2n3-c2ccccc2)cc1 ; c1ccc(-c2ccccc2-c2ccc(N(c3ccc4oc5ccccc5c4c3)c3ccc4oc5ccccc5c4c3)cc2)cc1 ; c1ccc2c(c1)sc1ccc(N(c3ccc4sc5ccccc5c4c3)c3ccc4c5ccccc5c5ccccc5c4c3)cc12]

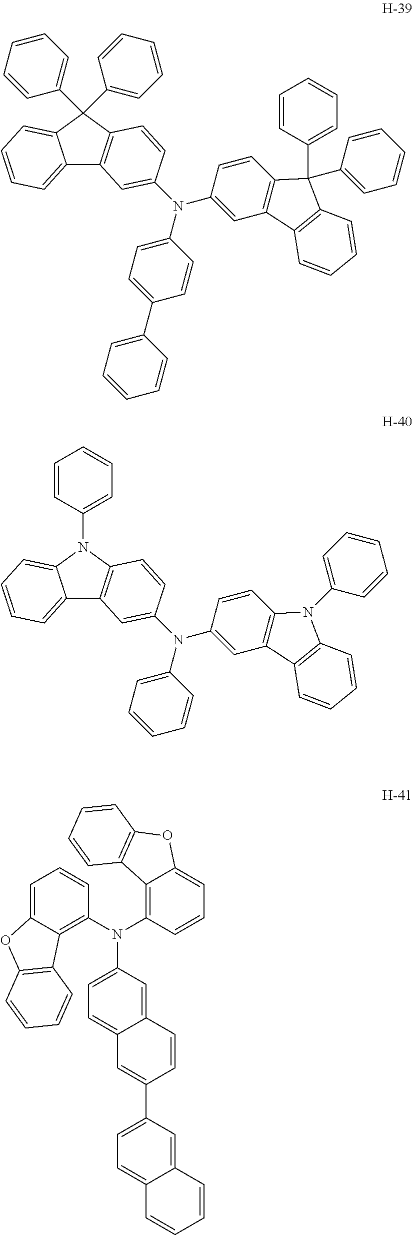 [SMILES: c1ccc(-c2ccc(N(c3ccc4c(c3)-c3ccccc3C4(c3ccccc3)c3ccccc3)c3ccc4c(c3)-c3ccccc3C4(c3ccccc3)c3ccccc3)cc2)cc1 ; c1ccc(N(c2ccc3c(c2)c2ccccc2n3-c2ccccc2)c2ccc3c(c2)c2ccccc2n3-c2ccccc2)cc1 ; c1ccc2cc(-c3ccc4cc(N(c5cccc6oc7ccccc7c56)c5cccc6oc7ccccc7c56)ccc4c3)ccc2c1]

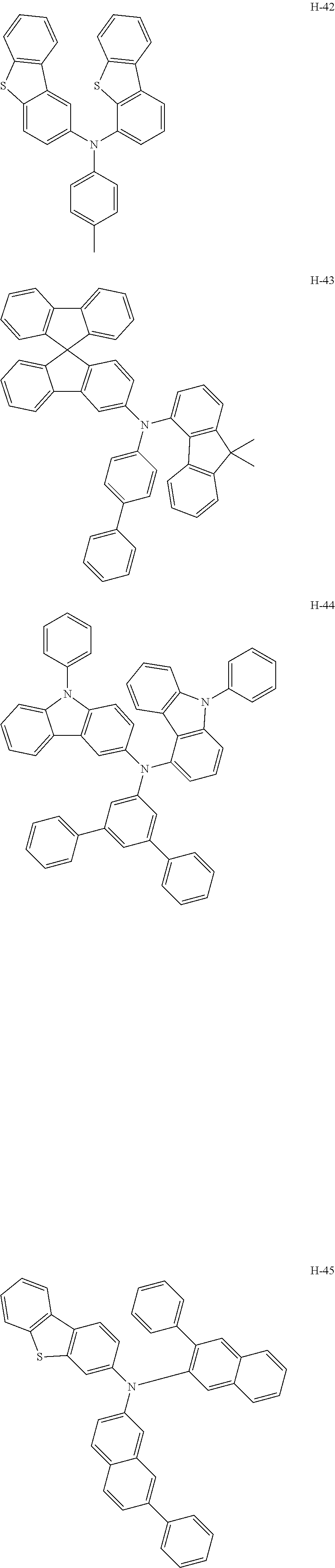

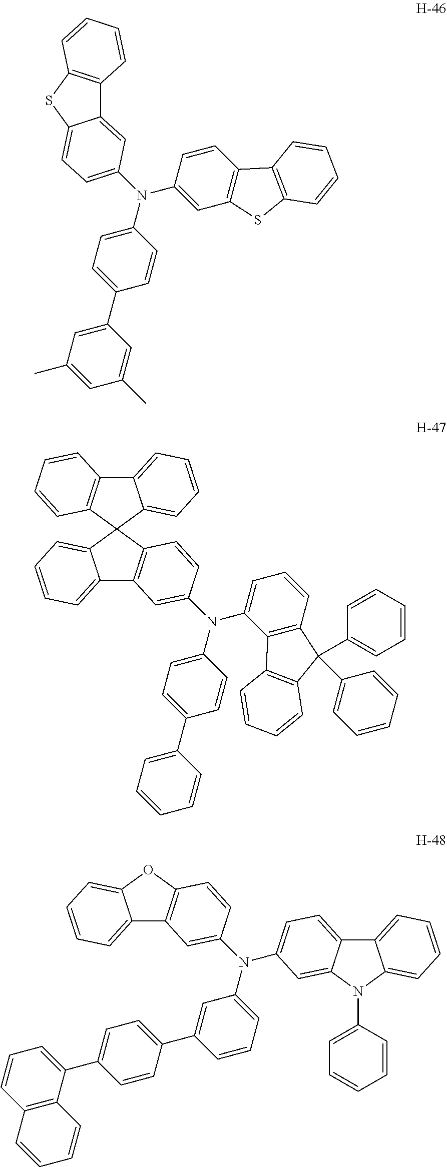 [SMILES: Cc1cc(C)cc(-c2ccc(N(c3ccc4c(c3)sc3ccccc34)c3ccc4sc5ccccc5c4c3)cc2)c1 ; c1ccc(-c2ccc(N(c3ccc4c(c3)-c3ccccc3C43c4ccccc4-c4ccccc43)c3cccc4c3-c3ccccc3C4(c3ccccc3)c3ccccc3)cc2)cc1 ; c1ccc(-n2c3ccccc3c3ccc(N(c4cccc(-c5ccc(-c6cccc7ccccc67)cc5)c4)c4ccc5oc6ccccc6c5c4)cc32)cc1]

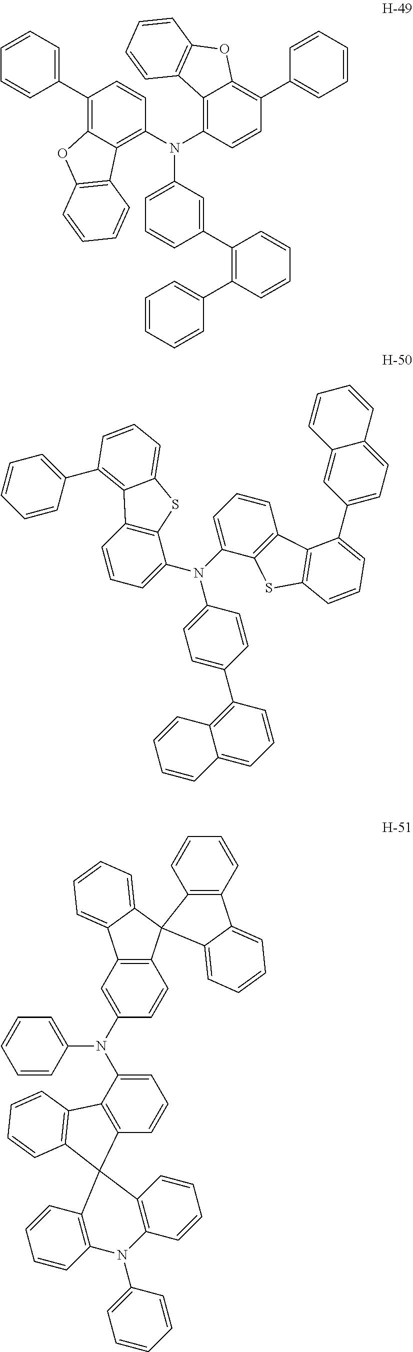 [SMILES: c1ccc(-c2ccccc2-c2cccc(N(c3ccc(-c4ccccc4)c4oc5ccccc5c34)c3ccc(-c4ccccc4)c4oc5ccccc5c34)c2)cc1 ; c1ccc(-c2cccc3sc4c(N(c5ccc(-c6cccc7ccccc67)cc5)c5cccc6c5sc5cccc(-c7ccc8ccccc8c7)c56)cccc4c23)cc1 ; c1ccc(N(c2ccc3c(c2)-c2ccccc2C32c3ccccc3-c3ccccc32)c2cccc3c2-c2ccccc2C32c3ccccc3N(c3ccccc3)c3ccccc32)cc1]

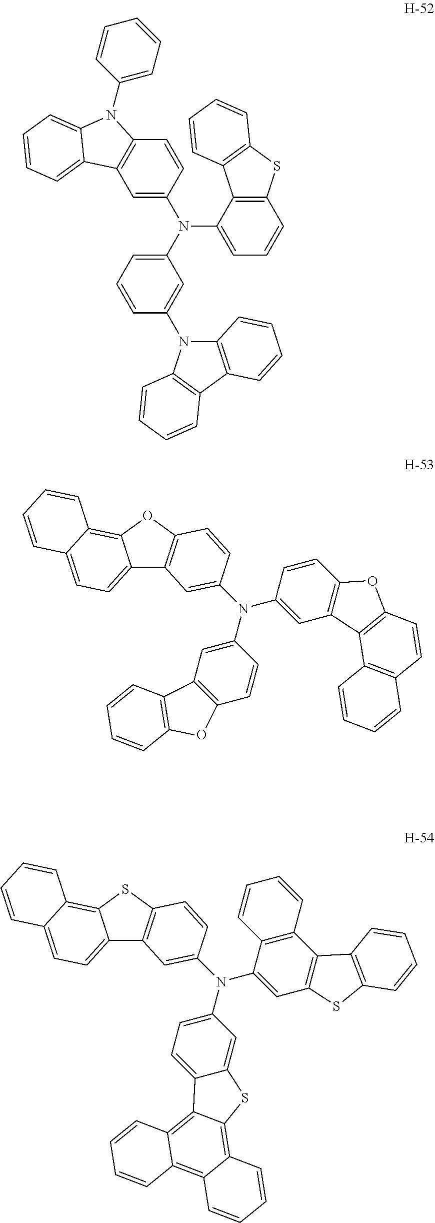 [SMILES: c1ccc(-n2c3ccccc3c3cc(N(c4cccc(-n5c6ccccc6c6ccccc65)c4)c4cccc5sc6ccccc6c45)ccc32)cc1 ; c1ccc2c(c1)ccc1c3cc(N(c4ccc5oc6ccccc6c5c4)c4ccc5oc6ccc7ccccc7c6c5c4)ccc3oc21 ; c1ccc2c(c1)ccc1c3cc(N(c4ccc5c(c4)sc4c6ccccc6c6ccccc6c54)c4cc5sc6ccccc6c5c5ccccc45)ccc3sc21]

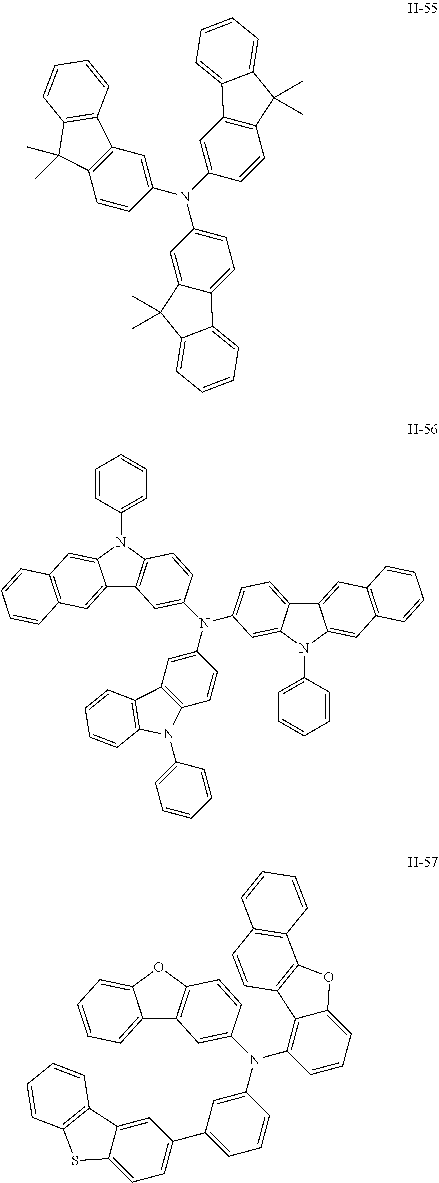

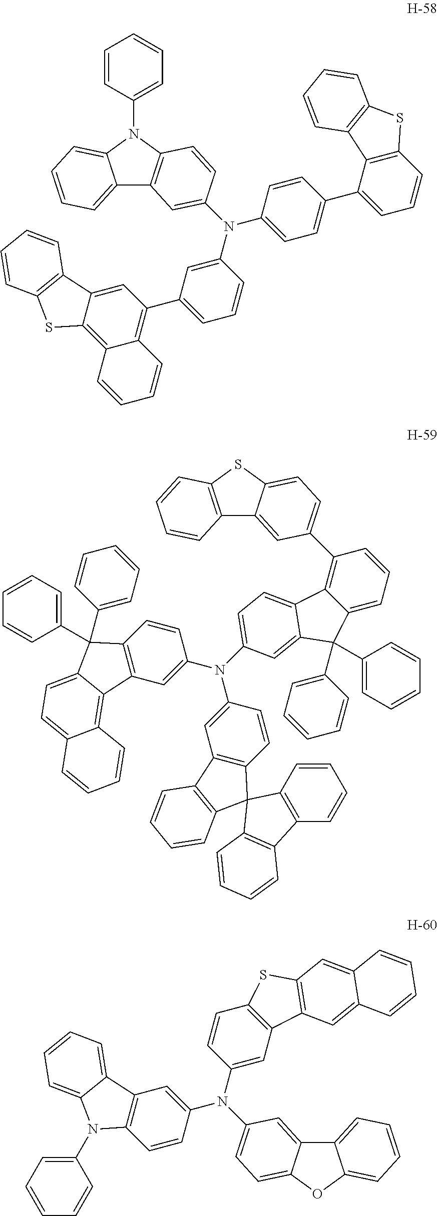 [SMILES: c1ccc(-n2c3ccccc3c3cc(N(c4ccc(-c5cccc6sc7ccccc7c56)cc4)c4cccc(-c5cc6c7ccccc7sc6c6ccccc56)c4)ccc32)cc1 ; c1ccc(C2(c3ccccc3)c3cc(N(c4ccc5c(c4)-c4c(ccc6ccccc46)C5(c4ccccc4)c4ccccc4)c4ccc5c(c4)-c4ccccc4C54c5ccccc5-c5ccccc54)ccc3-c3c(-c4ccc5sc6ccccc6c5c4)cccc32)cc1 ; c1ccc(-n2c3ccccc3c3cc(N(c4ccc5oc6ccccc6c5c4)c4ccc5sc6cc7ccccc7cc6c5c4)ccc32)cc1]

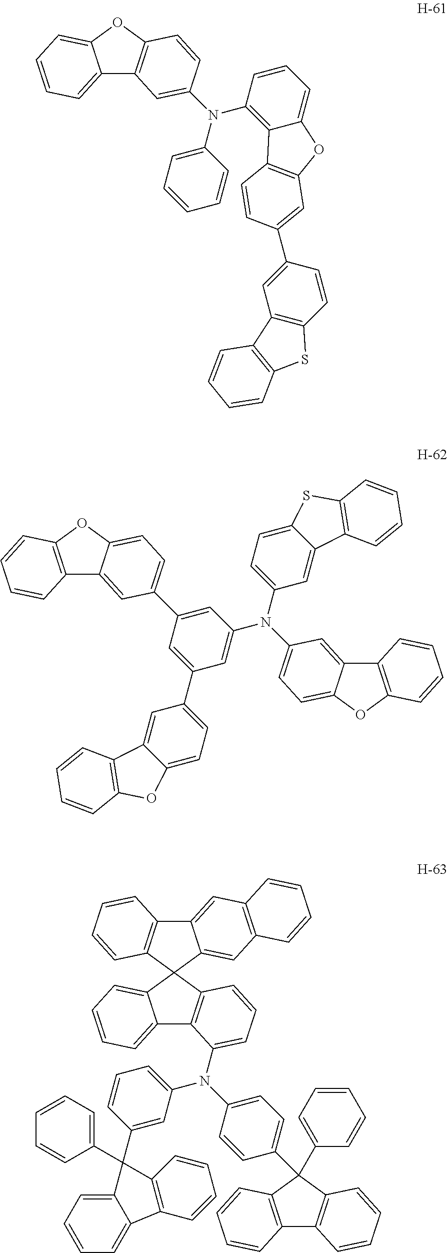 [SMILES: c1ccc(N(c2ccc3oc4ccccc4c3c2)c2cccc3oc4cc(-c5ccc6sc7ccccc7c6c5)ccc4c23)cc1 ; c1ccc2c(c1)oc1ccc(-c3cc(-c4ccc5oc6ccccc6c5c4)cc(N(c4ccc5oc6ccccc6c5c4)c4ccc5sc6ccccc6c5c4)c3)cc12 ; c1ccc(C2(c3ccc(N(c4cccc(C5(c6ccccc6)c6ccccc6-c6ccccc65)c4)c4cccc5c4-c4ccccc4C54c5ccccc5-c5cc6ccccc6cc54)cc3)c3ccccc3-c3ccccc32)cc1]

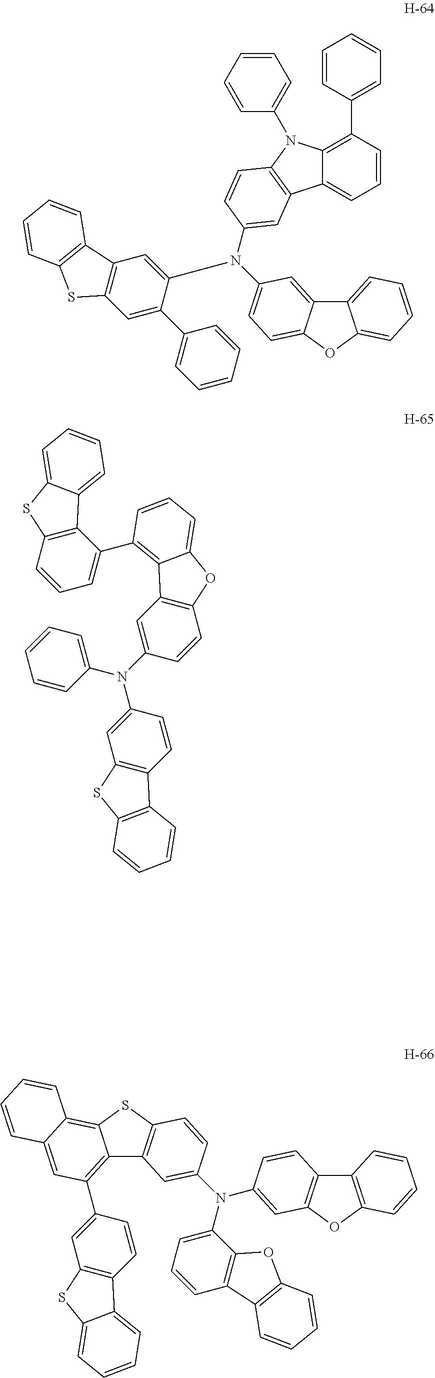 [SMILES: c1ccc(-c2cc3sc4ccccc4c3cc2N(c2ccc3oc4ccccc4c3c2)c2ccc3c(c2)c2cccc(-c4ccccc4)c2n3-c2ccccc2)cc1 ; c1ccc(N(c2ccc3c(c2)sc2ccccc23)c2ccc3oc4cccc(-c5cccc6sc7ccccc7c56)c4c3c2)cc1 ; c1ccc2c(c1)cc(-c1ccc3c(c1)sc1ccccc13)c1c3cc(N(c4ccc5c(c4)oc4ccccc45)c4cccc5c4oc4ccccc45)ccc3sc21]

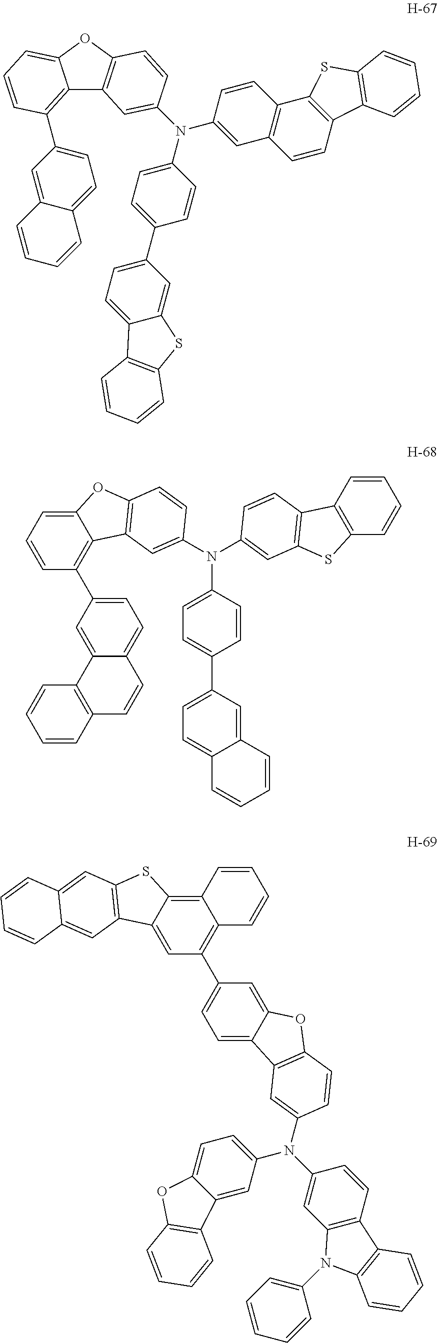 [SMILES: c1ccc2cc(-c3cccc4oc5ccc(N(c6ccc(-c7ccc8c(c7)sc7ccccc78)cc6)c6ccc7c(ccc8c9ccccc9sc78)c6)cc5c34)ccc2c1 ; c1ccc2cc(-c3ccc(N(c4ccc5c(c4)sc4ccccc45)c4ccc5oc6cccc(-c7ccc8ccc9ccccc9c8c7)c6c5c4)cc3)ccc2c1 ; c1ccc(-n2c3ccccc3c3ccc(N(c4ccc5oc6ccccc6c5c4)c4ccc5oc6cc(-c7cc8c9cc%10ccccc%10cc9sc8c8ccccc78)ccc6c5c4)cc32)cc1]

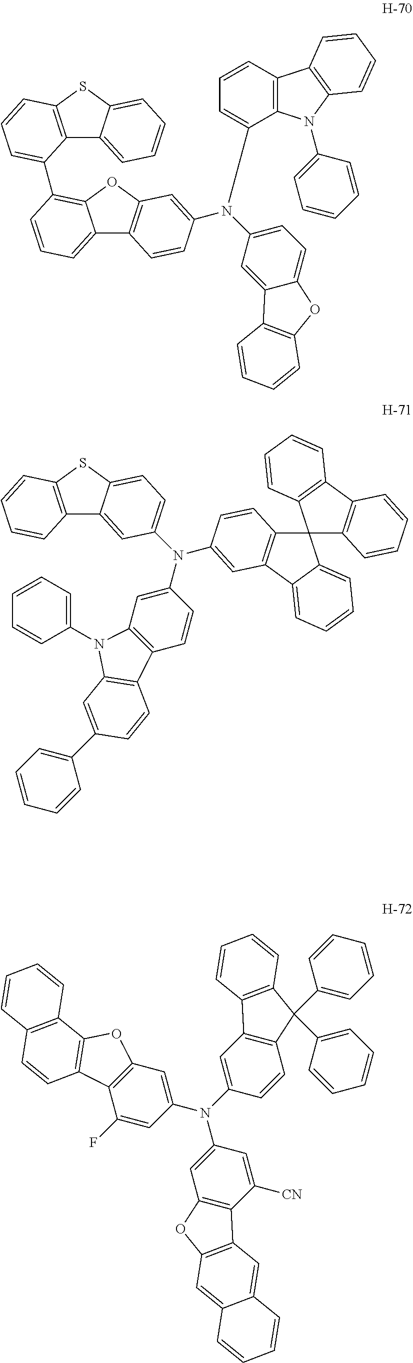 [SMILES: c1ccc(-n2c3ccccc3c3cccc(N(c4ccc5c(c4)oc4c(-c6cccc7sc8ccccc8c67)cccc45)c4ccc5oc6ccccc6c5c4)c32)cc1 ; c1ccc(-c2ccc3c4ccc(N(c5ccc6c(c5)-c5ccccc5C65c6ccccc6-c6ccccc65)c5ccc6sc7ccccc7c6c5)cc4n(-c4ccccc4)c3c2)cc1 ; N#Cc1cc(N(c2ccc3c(c2)-c2ccccc2C3(c2ccccc2)c2ccccc2)c2cc(F)c3c(c2)oc2c4ccccc4ccc23)cc2oc3cc4ccccc4cc3c12]

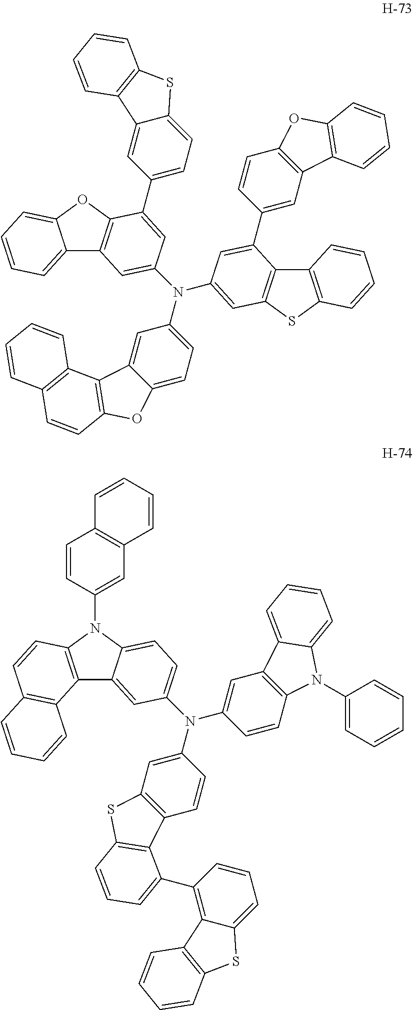 [SMILES: c1ccc2c(c1)ccc1oc3ccc(N(c4cc(-c5ccc6oc7ccccc7c6c5)c5c(c4)sc4ccccc45)c4cc(-c5ccc6sc7ccccc7c6c5)c5oc6ccccc6c5c4)cc3c12 ; c1ccc(-n2c3ccccc3c3cc(N(c4ccc5c(c4)sc4cccc(-c6cccc7sc8ccccc8c67)c45)c4ccc5c(c4)c4c6ccccc6ccc4n5-c4ccc5ccccc5c4)ccc32)cc1]

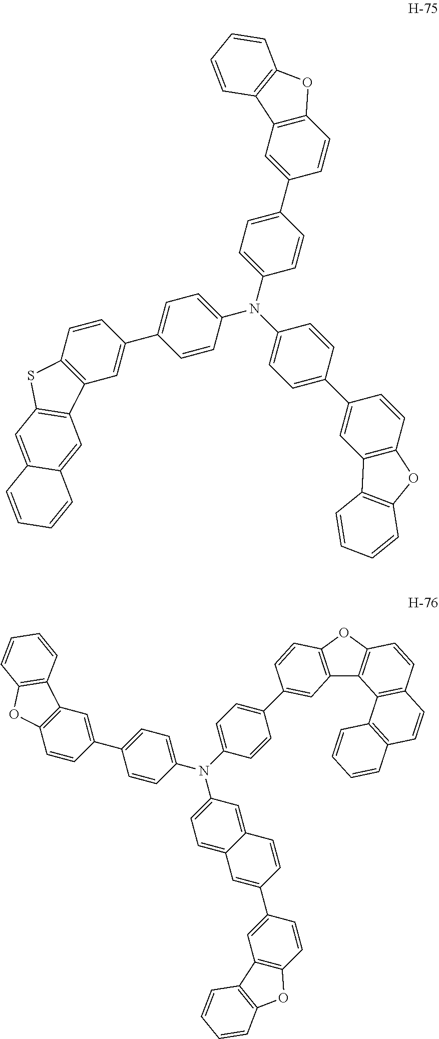 [SMILES: c1ccc2cc3c(cc2c1)sc1ccc(-c2ccc(N(c4ccc(-c5ccc6oc7ccccc7c6c5)cc4)c4ccc(-c5ccc6oc7ccccc7c6c5)cc4)cc2)cc13 ; c1ccc2c(c1)ccc1ccc3oc4ccc(-c5ccc(N(c6ccc(-c7ccc8oc9ccccc9c8c7)cc6)c6ccc7cc(-c8ccc9oc%10ccccc%10c9c8)ccc7c6)cc5)cc4c3c12]

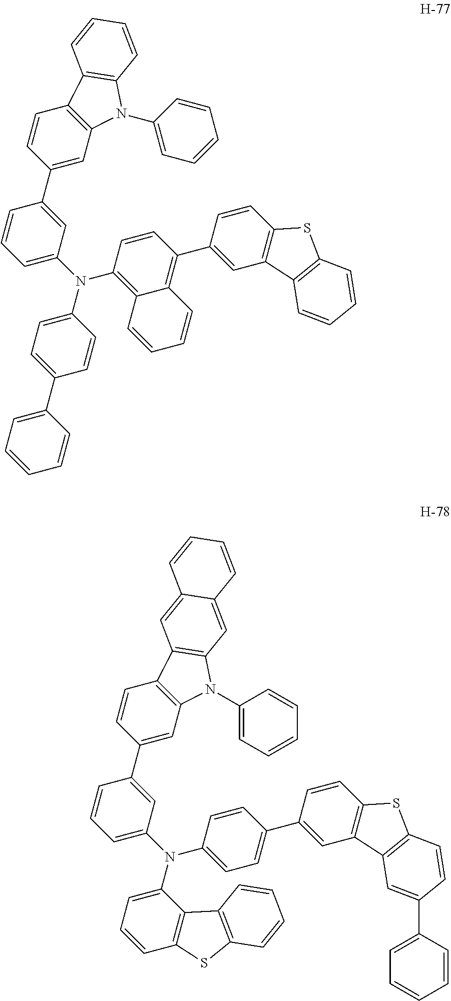 [SMILES: c1ccc(-c2ccc(N(c3cccc(-c4ccc5c6ccccc6n(-c6ccccc6)c5c4)c3)c3ccc(-c4ccc5sc6ccccc6c5c4)c4ccccc34)cc2)cc1 ; c1ccc(-c2ccc3sc4ccc(-c5ccc(N(c6cccc(-c7ccc8c9cc%10ccccc%10cc9n(-c9ccccc9)c8c7)c6)c6cccc7sc8ccccc8c67)cc5)cc4c3c2)cc1]

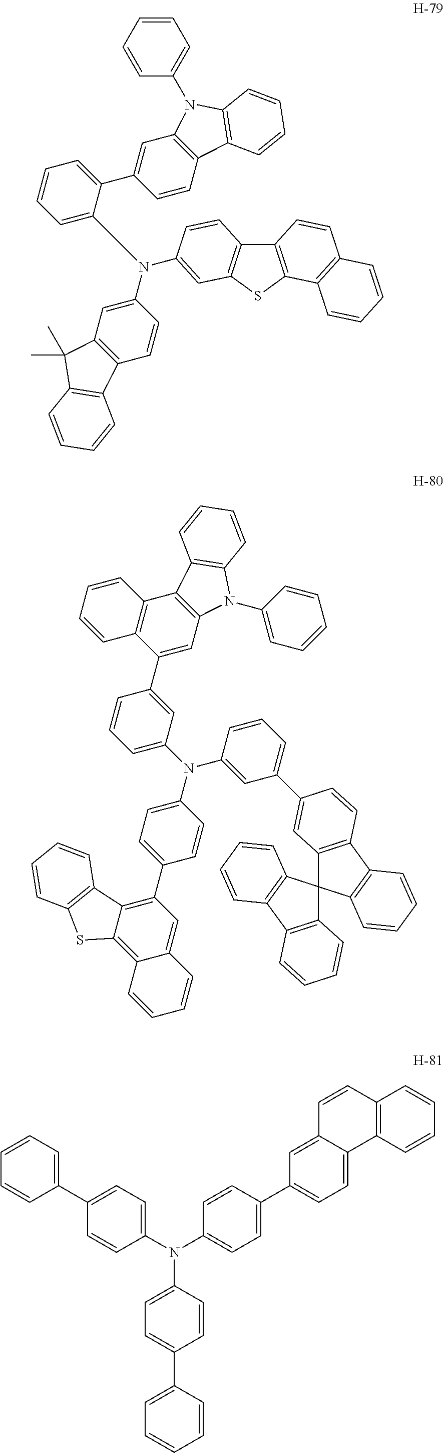 [SMILES: CC1(C)c2ccccc2-c2ccc(N(c3ccc4c(c3)sc3c5ccccc5ccc43)c3ccccc3-c3ccc4c5ccccc5n(-c5ccccc5)c4c3)cc21 ; c1ccc(-n2c3ccccc3c3c4ccccc4c(-c4cccc(N(c5ccc(-c6cc7ccccc7c7sc8ccccc8c67)cc5)c5cccc(-c6ccc7c(c6)C6(c8ccccc8-c8ccccc86)c6ccccc6-7)c5)c4)cc32)cc1 ; c1ccc(-c2ccc(N(c3ccc(-c4ccccc4)cc3)c3ccc(-c4ccc5c(ccc6ccccc65)c4)cc3)cc2)cc1]

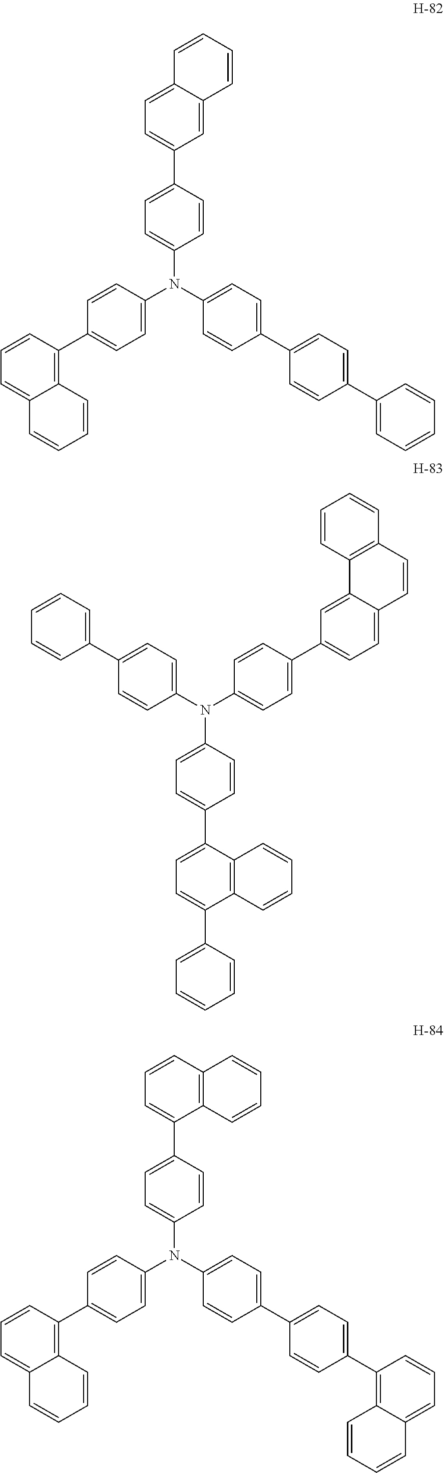 [SMILES: c1ccc(-c2ccc(-c3ccc(N(c4ccc(-c5ccc6ccccc6c5)cc4)c4ccc(-c5cccc6ccccc56)cc4)cc3)cc2)cc1 ; c1ccc(-c2ccc(N(c3ccc(-c4ccc5ccc6ccccc6c5c4)cc3)c3ccc(-c4ccc(-c5ccccc5)c5ccccc45)cc3)cc2)cc1 ; c1ccc2c(-c3ccc(-c4ccc(N(c5ccc(-c6cccc7ccccc67)cc5)c5ccc(-c6cccc7ccccc67)cc5)cc4)cc3)cccc2c1]

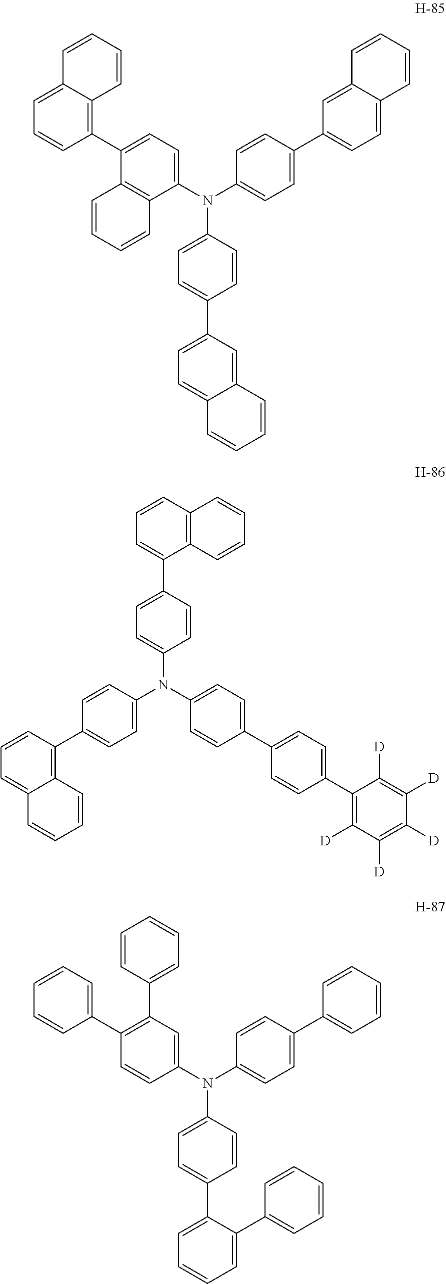 [SMILES: c1ccc2cc(-c3ccc(N(c4ccc(-c5ccc6ccccc6c5)cc4)c4ccc(-c5cccc6ccccc56)c5ccccc45)cc3)ccc2c1 ; [2H]c1c([2H])c([2H])c(-c2ccc(-c3ccc(N(c4ccc(-c5cccc6ccccc56)cc4)c4ccc(-c5cccc6ccccc56)cc4)cc3)cc2)c([2H])c1[2H] ; c1ccc(-c2ccc(N(c3ccc(-c4ccccc4-c4ccccc4)cc3)c3ccc(-c4ccccc4)c(-c4ccccc4)c3)cc2)cc1]

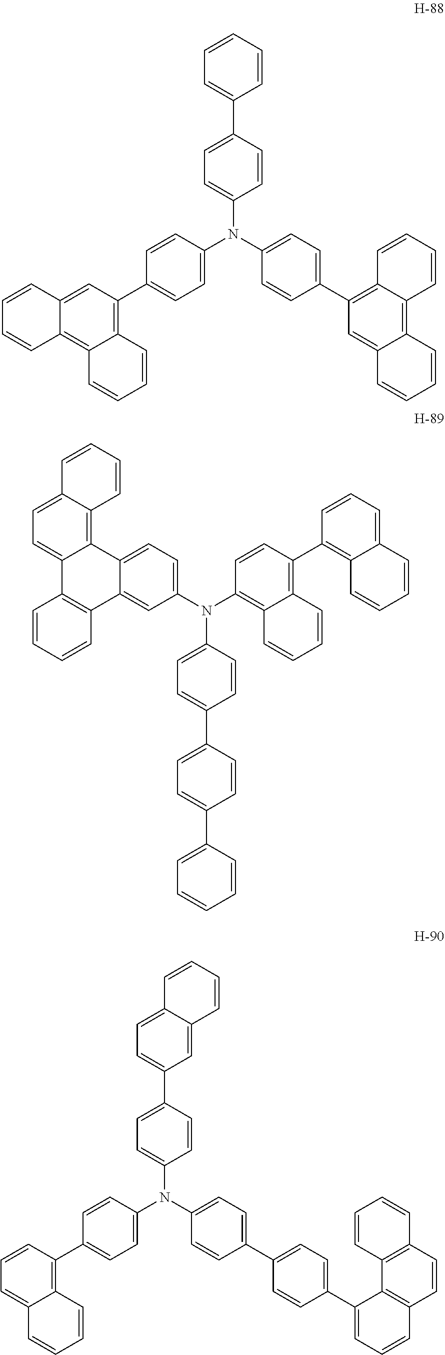 [SMILES: c1ccc(-c2ccc(N(c3ccc(-c4cc5ccccc5c5ccccc45)cc3)c3ccc(-c4cc5ccccc5c5ccccc45)cc3)cc2)cc1 ; c1ccc(-c2ccc(-c3ccc(N(c4ccc5c(c4)c4ccccc4c4ccc6ccccc6c45)c4ccc(-c5cccc6ccccc56)c5ccccc45)cc3)cc2)cc1 ; c1ccc2cc(-c3ccc(N(c4ccc(-c5ccc(-c6cccc7ccc8ccccc8c67)cc5)cc4)c4ccc(-c5cccc6ccccc56)cc4)cc3)ccc2c1]

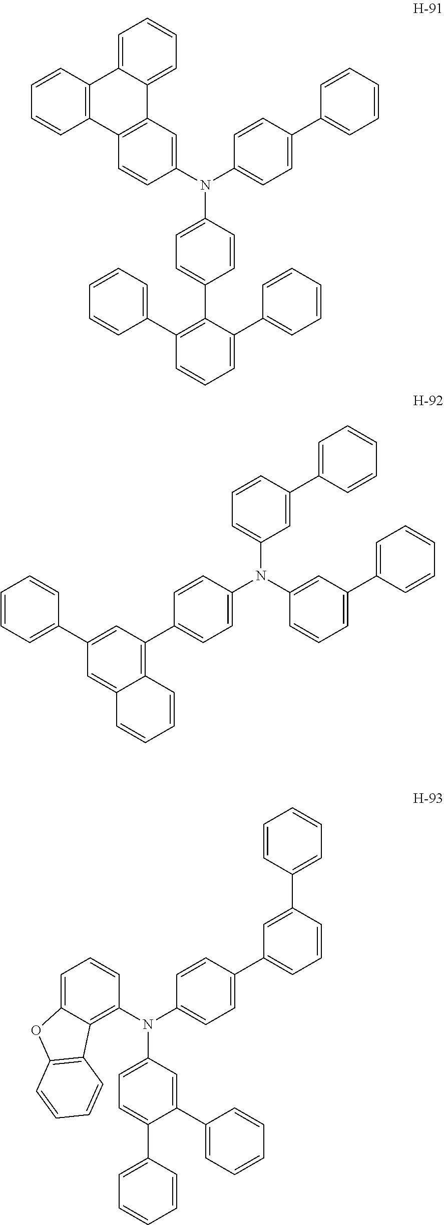 [SMILES: c1ccc(-c2ccc(N(c3ccc(-c4c(-c5ccccc5)cccc4-c4ccccc4)cc3)c3ccc4c5ccccc5c5ccccc5c4c3)cc2)cc1 ; c1ccc(-c2cccc(N(c3ccc(-c4cc(-c5ccccc5)cc5ccccc45)cc3)c3cccc(-c4ccccc4)c3)c2)cc1 ; c1ccc(-c2cccc(-c3ccc(N(c4ccc(-c5ccccc5)c(-c5ccccc5)c4)c4cccc5oc6ccccc6c45)cc3)c2)cc1]

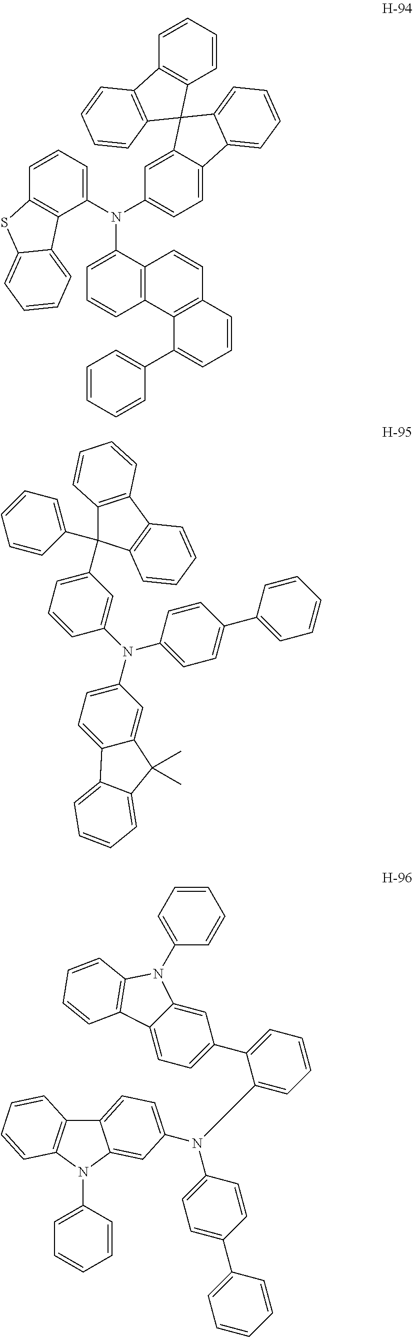

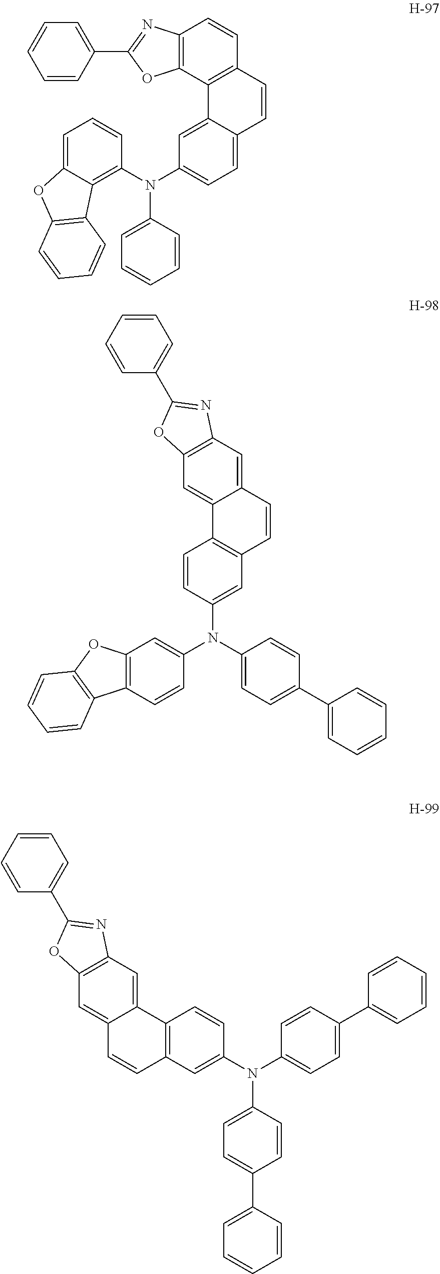

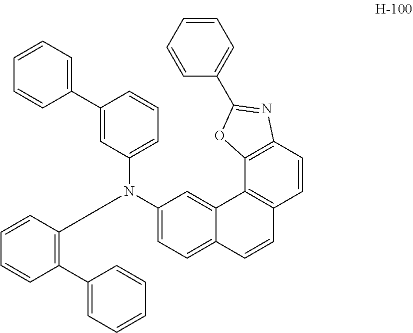 [SMILES: c1ccc(-c2cccc(N(c3ccc4ccc5ccc6nc(-c7ccccc7)oc6c5c4c3)c3ccccc3-c3ccccc3)c2)cc1]

Specifically, the compound represented by Formula 5 may be any one of the following compounds S-1 to S-108, but is not limited thereto.

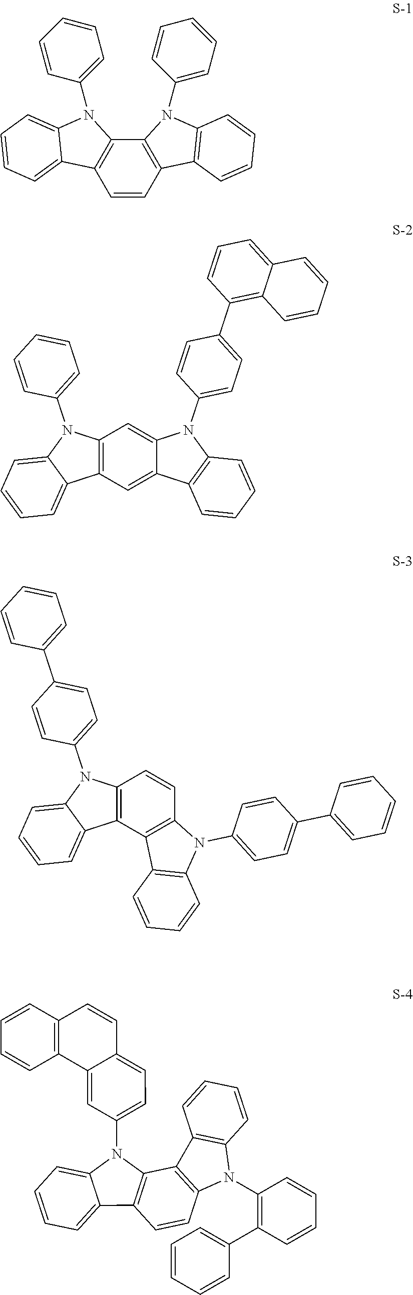 [SMILES: c1ccc(-n2c3ccccc3c3ccc4c5ccccc5n(-c5ccccc5)c4c32)cc1 ; c1ccc(-n2c3ccccc3c3cc4c5ccccc5n(-c5ccc(-c6cccc7ccccc67)cc5)c4cc32)cc1 ; c1ccc(-c2ccc(-n3c4ccccc4c4c5c6ccccc6n(-c6ccc(-c7ccccc7)cc6)c5ccc43)cc2)cc1 ; c1ccc(-c2ccccc2-n2c3ccccc3c3c2ccc2c4ccccc4n(-c4ccc5ccc6ccccc6c5c4)c23)cc1]

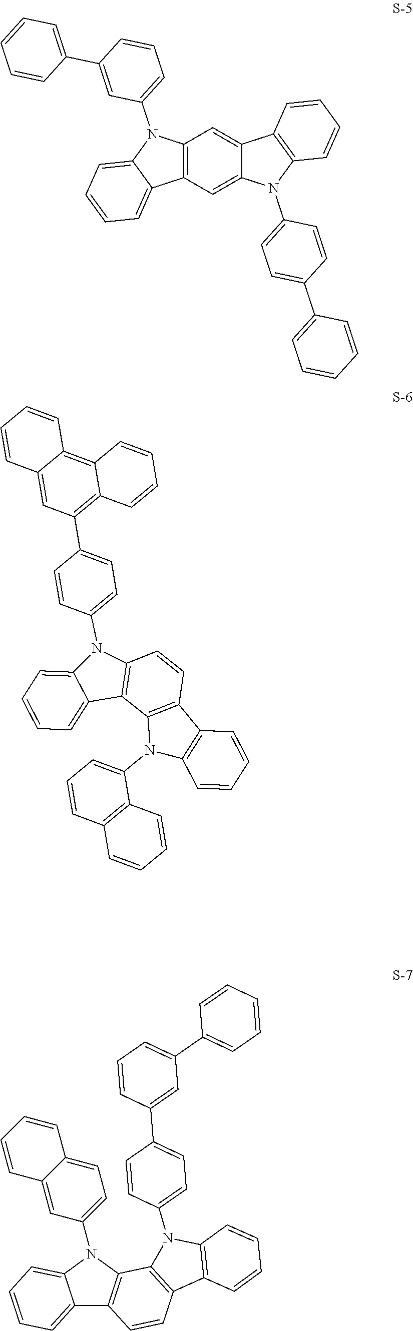 [SMILES: c1ccc(-c2ccc(-n3c4ccccc4c4cc5c(cc43)c3ccccc3n5-c3cccc(-c4ccccc4)c3)cc2)cc1 ; c1ccc2c(-n3c4ccccc4c4ccc5c(c6ccccc6n5-c5ccc(-c6cc7ccccc7c7ccccc67)cc5)c43)cccc2c1 ; c1ccc(-c2cccc(-c3ccc(-n4c5ccccc5c5ccc6c7ccccc7n(-c7ccc8ccccc8c7)c6c54)cc3)c2)cc1]

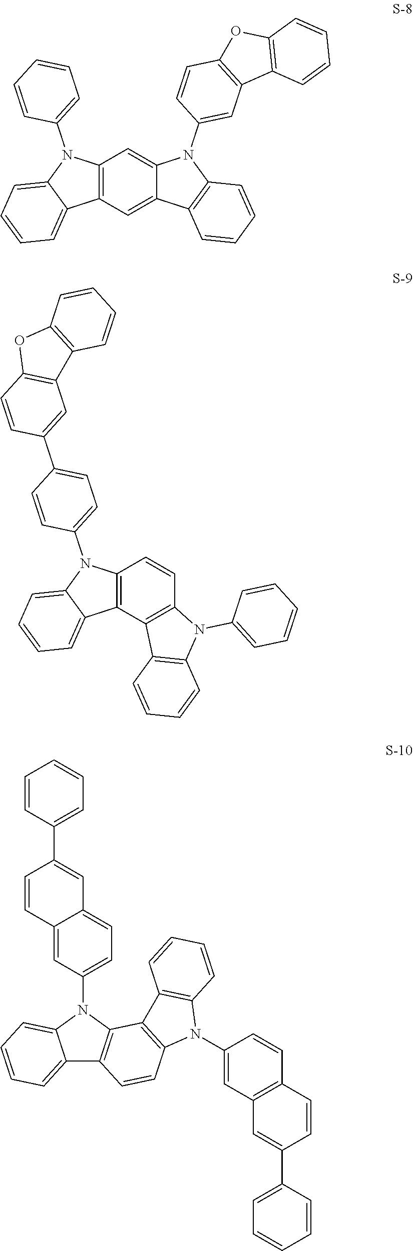 [SMILES: c1ccc(-n2c3ccccc3c3cc4c5ccccc5n(-c5ccc6oc7ccccc7c6c5)c4cc32)cc1 ; c1ccc(-n2c3ccccc3c3c4c5ccccc5n(-c5ccc(-c6ccc7oc8ccccc8c7c6)cc5)c4ccc32)cc1 ; c1ccc(-c2ccc3cc(-n4c5ccccc5c5ccc6c(c7ccccc7n6-c6ccc7ccc(-c8ccccc8)cc7c6)c54)ccc3c2)cc1]

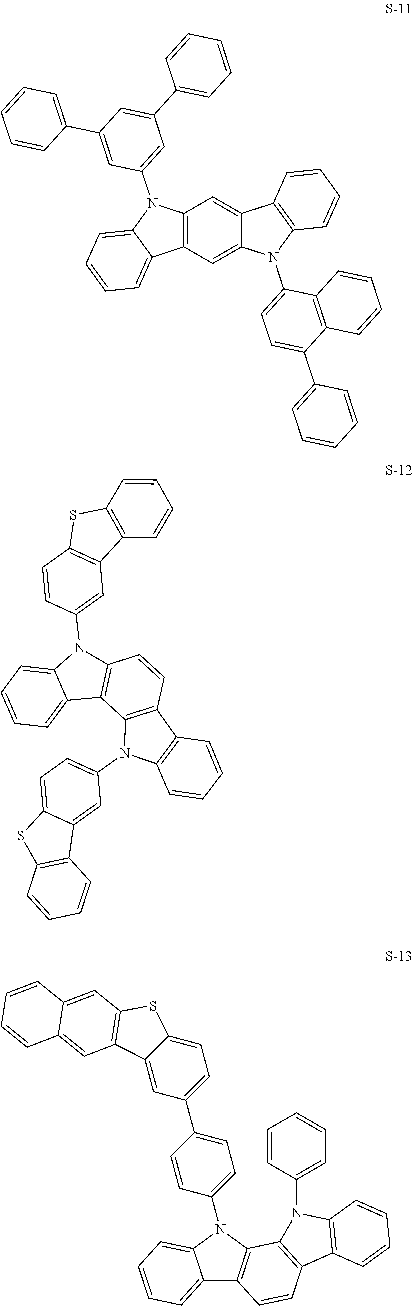 [SMILES: c1ccc(-c2cc(-c3ccccc3)cc(-n3c4ccccc4c4cc5c(cc43)c3ccccc3n5-c3ccc(-c4ccccc4)c4ccccc34)c2)cc1 ; c1ccc2c(c1)sc1ccc(-n3c4ccccc4c4c3ccc3c5ccccc5n(-c5ccc6sc7ccccc7c6c5)c34)cc12 ; c1ccc(-n2c3ccccc3c3ccc4c5ccccc5n(-c5ccc(-c6ccc7sc8cc9ccccc9cc8c7c6)cc5)c4c32)cc1]

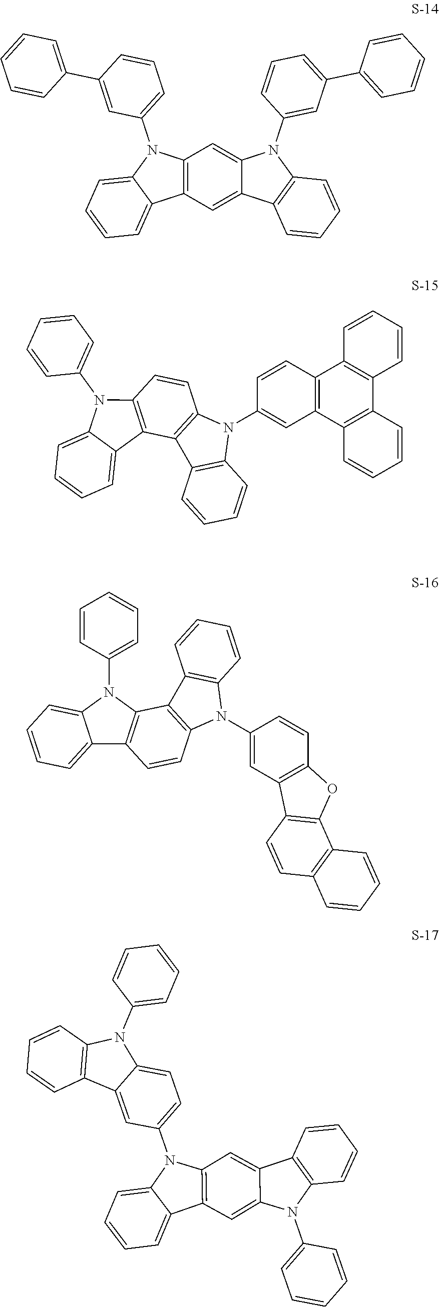 [SMILES: c1ccc(-c2cccc(-n3c4ccccc4c4cc5c6ccccc6n(-c6cccc(-c7ccccc7)c6)c5cc43)c2)cc1 ; c1ccc(-n2c3ccccc3c3c4c5ccccc5n(-c5ccc6c7ccccc7c7ccccc7c6c5)c4ccc32)cc1 ; c1ccc(-n2c3ccccc3c3ccc4c(c5ccccc5n4-c4ccc5oc6c7ccccc7ccc6c5c4)c32)cc1 ; c1ccc(-n2c3ccccc3c3cc(-n4c5ccccc5c5cc6c(cc54)c4ccccc4n6-c4ccccc4)ccc32)cc1]

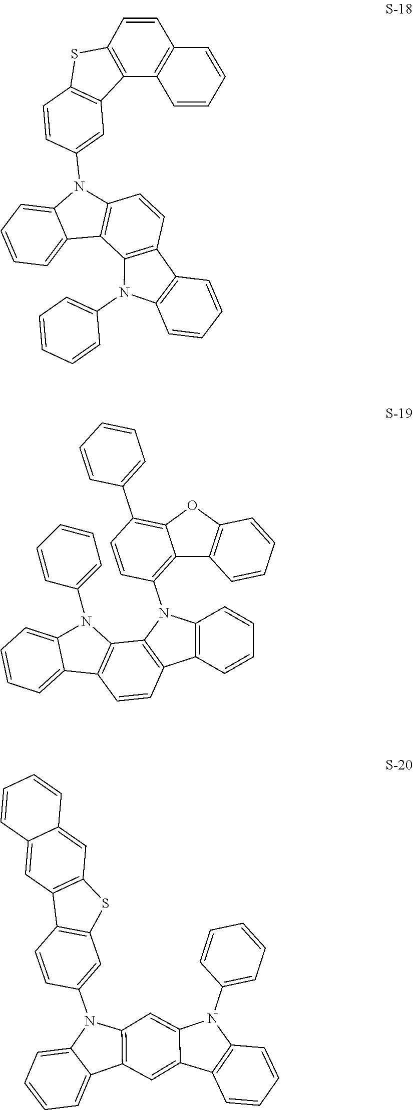 [SMILES: c1ccc(-n2c3ccccc3c3ccc4c(c5ccccc5n4-c4ccc5sc6ccc7ccccc7c6c5c4)c32)cc1 ; c1ccc(-c2ccc(-n3c4ccccc4c4ccc5c6ccccc6n(-c6ccccc6)c5c43)c3c2oc2ccccc23)cc1 ; c1ccc(-n2c3ccccc3c3cc4c5ccccc5n(-c5ccc6c(c5)sc5cc7ccccc7cc56)c4cc32)cc1]

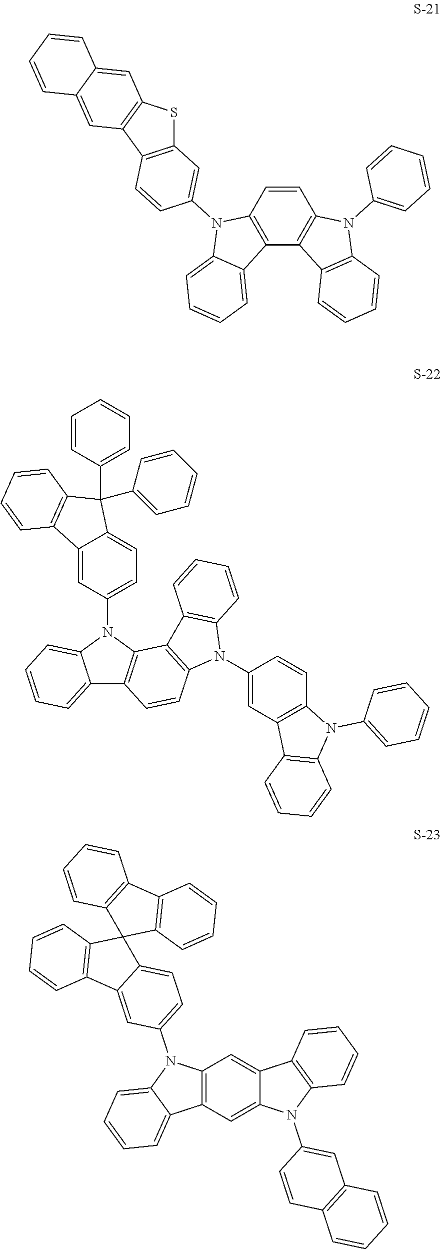 [SMILES: c1ccc(-n2c3ccccc3c3c4c5ccccc5n(-c5ccc6c(c5)sc5cc7ccccc7cc56)c4ccc32)cc1 ; c1ccc(-n2c3ccccc3c3cc(-n4c5ccccc5c5c4ccc4c6ccccc6n(-c6ccc7c(c6)-c6ccccc6C7(c6ccccc6)c6ccccc6)c45)ccc32)cc1 ; c1ccc2c(c1)-c1ccccc1C21c2ccccc2-c2cc(-n3c4ccccc4c4cc5c(cc43)c3ccccc3n5-c3ccc4ccccc4c3)ccc21]

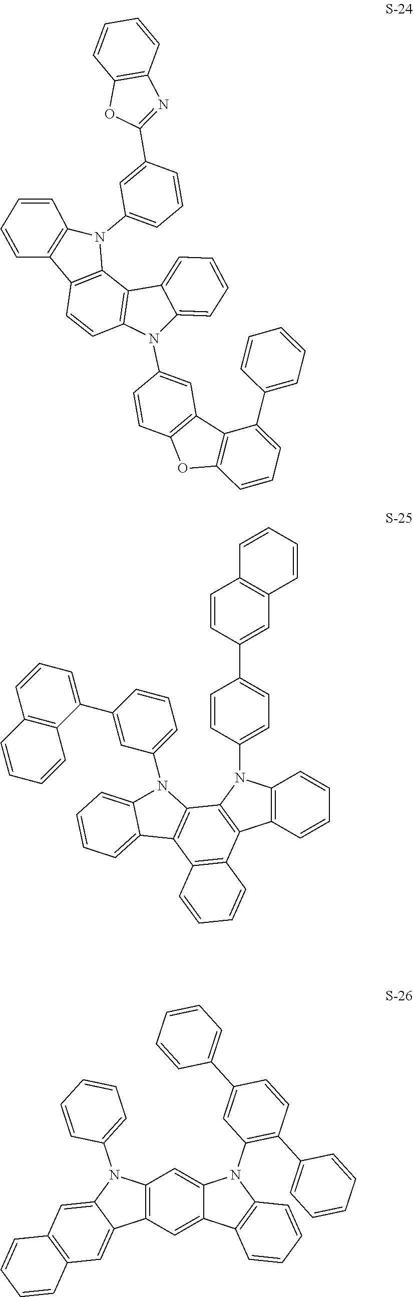 [SMILES: c1ccc(-c2cccc3oc4ccc(-n5c6ccccc6c6c5ccc5c7ccccc7n(-c7cccc(-c8nc9ccccc9o8)c7)c56)cc4c23)cc1 ; c1cc(-c2cccc3ccccc23)cc(-n2c3ccccc3c3c4ccccc4c4c5ccccc5n(-c5ccc(-c6ccc7ccccc7c6)cc5)c4c32)c1 ; c1ccc(-c2ccc(-c3ccccc3)c(-n3c4ccccc4c4cc5c6cc7ccccc7cc6n(-c6ccccc6)c5cc43)c2)cc1]

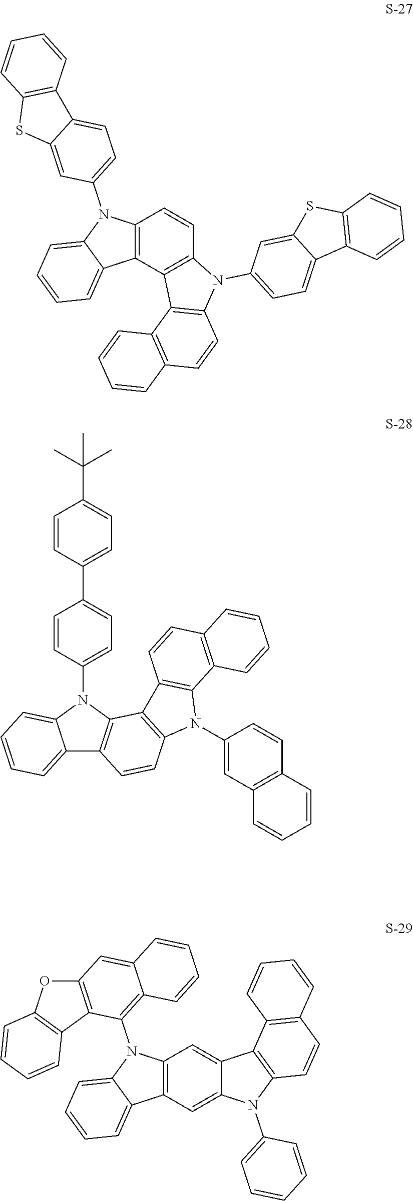 [SMILES: c1ccc2c(c1)ccc1c2c2c3c4ccccc4n(-c4ccc5c(c4)sc4ccccc45)c3ccc2n1-c1ccc2c(c1)sc1ccccc12 ; CC(C)(C)c1ccc(-c2ccc(-n3c4ccccc4c4ccc5c(c6ccc7ccccc7c6n5-c5ccc6ccccc6c5)c43)cc2)cc1 ; c1ccc(-n2c3cc4c5ccccc5n(-c5c6ccccc6cc6oc7ccccc7c56)c4cc3c3c4ccccc4ccc32)cc1]

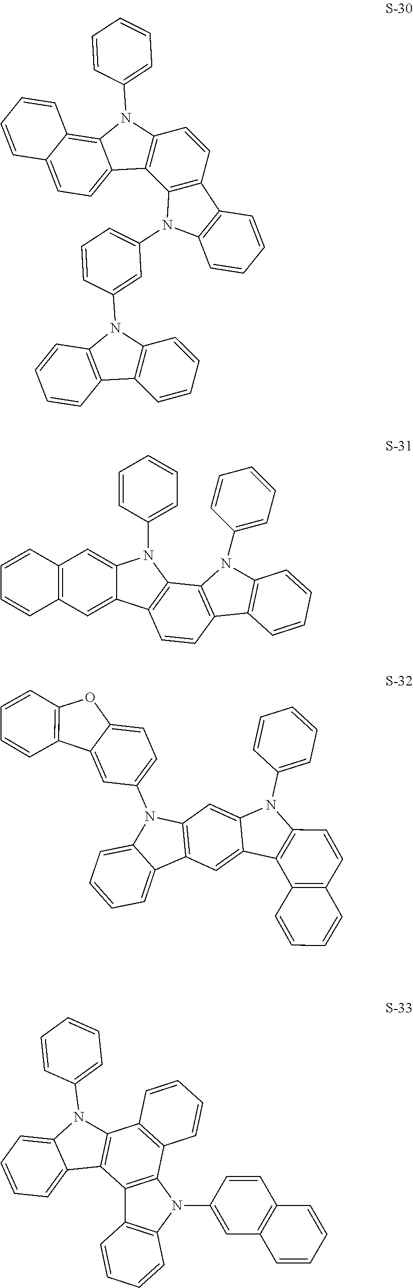 [SMILES: c1ccc(-n2c3ccc4c5ccccc5n(-c5cccc(-n6c7ccccc7c7ccccc76)c5)c4c3c3ccc4ccccc4c32)cc1 ; c1ccc(-n2c3ccccc3c3ccc4c5cc6ccccc6cc5n(-c5ccccc5)c4c32)cc1 ; c1ccc(-n2c3cc4c(cc3c3c5ccccc5ccc32)c2ccccc2n4-c2ccc3oc4ccccc4c3c2)cc1 ; c1ccc(-n2c3ccccc3c3c4c5ccccc5n(-c5ccc6ccccc6c5)c4c4ccccc4c32)cc1]

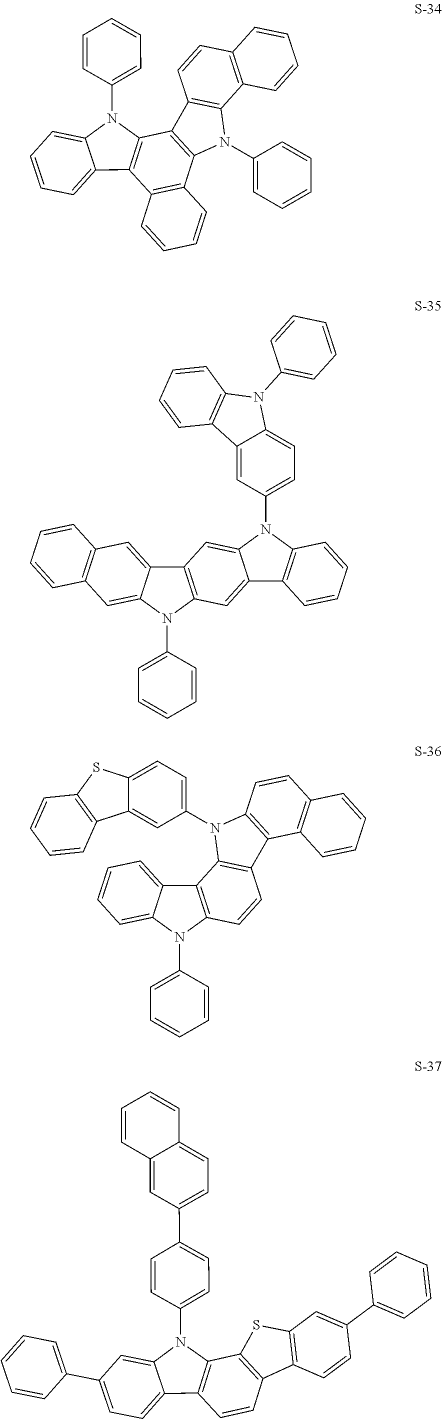

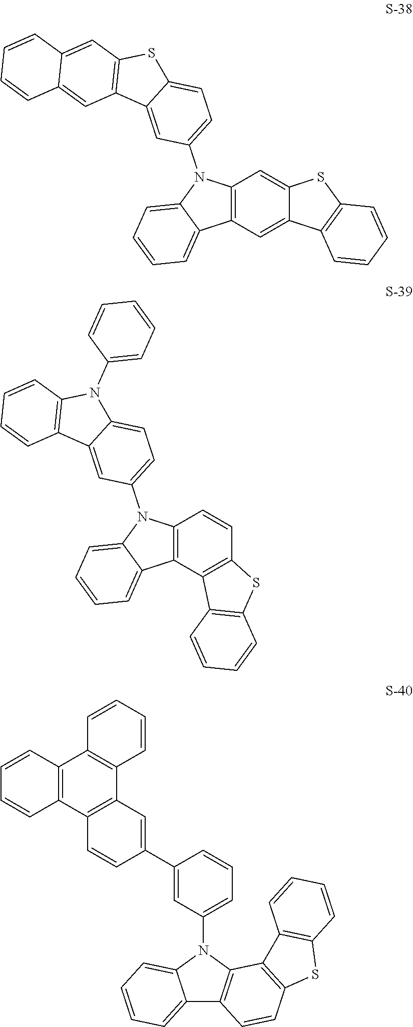

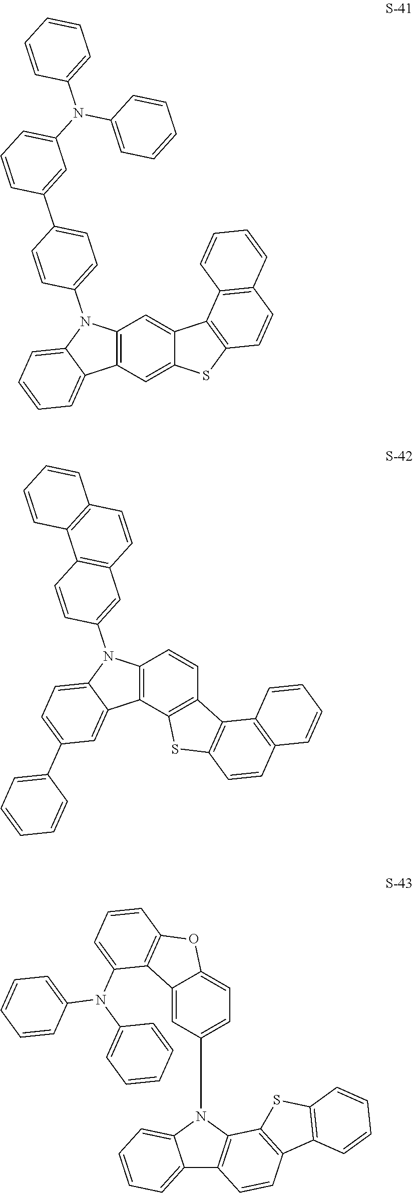 [SMILES: c1ccc(N(c2ccccc2)c2cccc(-c3ccc(-n4c5ccccc5c5cc6sc7ccc8ccccc8c7c6cc54)cc3)c2)cc1 ; c1ccc(-c2ccc3c(c2)c2c4sc5ccc6ccccc6c5c4ccc2n3-c2ccc3c(ccc4ccccc43)c2)cc1 ; c1ccc(N(c2ccccc2)c2cccc3oc4ccc(-n5c6ccccc6c6ccc7c8ccccc8sc7c65)cc4c23)cc1]

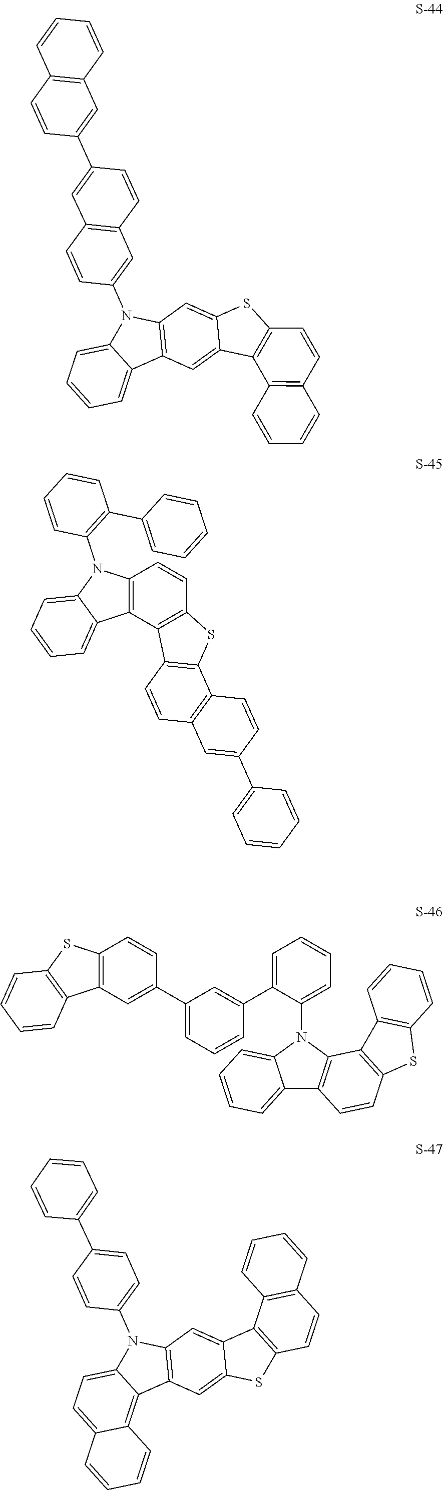 [SMILES: c1ccc2cc(-c3ccc4cc(-n5c6ccccc6c6cc7c(cc65)sc5ccc6ccccc6c57)ccc4c3)ccc2c1 ; c1ccc(-c2ccc3c(ccc4c3sc3ccc5c(c6ccccc6n5-c5ccccc5-c5ccccc5)c34)c2)cc1 ; c1cc(-c2ccc3sc4ccccc4c3c2)cc(-c2ccccc2-n2c3ccccc3c3ccc4sc5ccccc5c4c32)c1 ; c1ccc(-c2ccc(-n3c4cc5c(cc4c4c6ccccc6ccc43)sc3ccc4ccccc4c35)cc2)cc1]

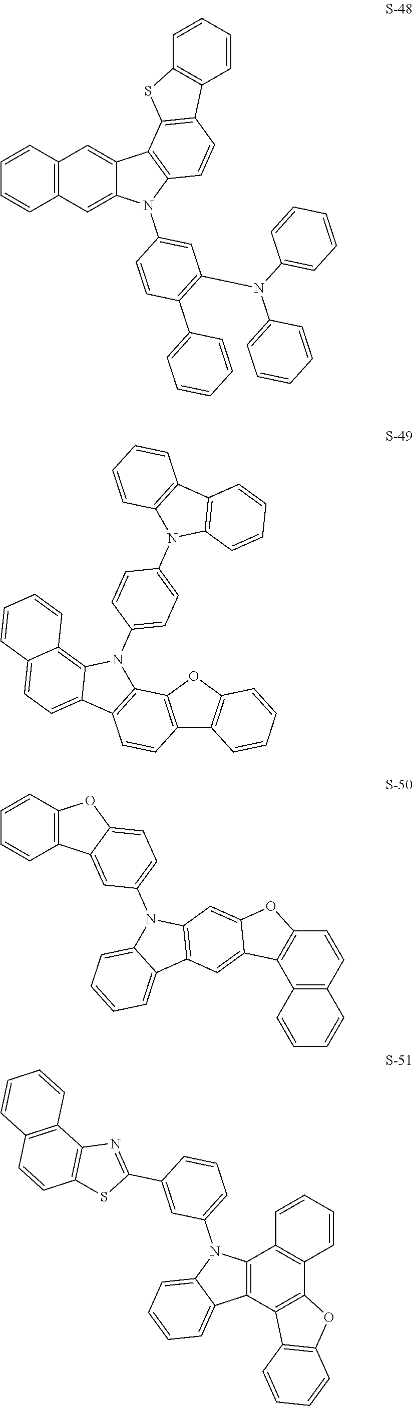 [SMILES: c1ccc(-c2ccc(-n3c4cc5ccccc5cc4c4c5sc6ccccc6c5ccc43)cc2N(c2ccccc2)c2ccccc2)cc1 ; c1ccc2c(c1)ccc1c3ccc4c5ccccc5oc4c3n(-c3ccc(-n4c5ccccc5c5ccccc54)cc3)c21 ; c1ccc2c(c1)ccc1oc3cc4c(cc3c12)c1ccccc1n4-c1ccc2oc3ccccc3c2c1 ; c1cc(-c2nc3c(ccc4ccccc43)s2)cc(-n2c3ccccc3c3c4c5ccccc5oc4c4ccccc4c32)c1]

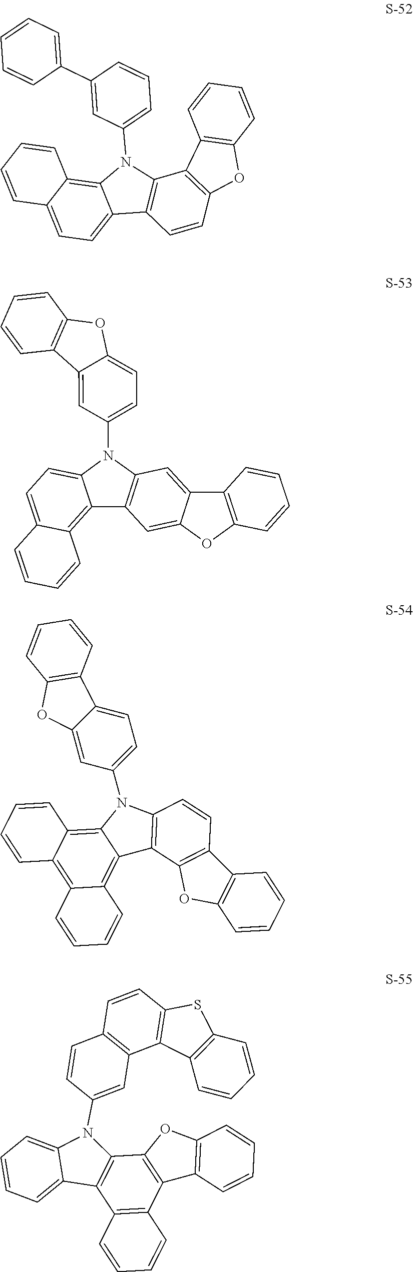

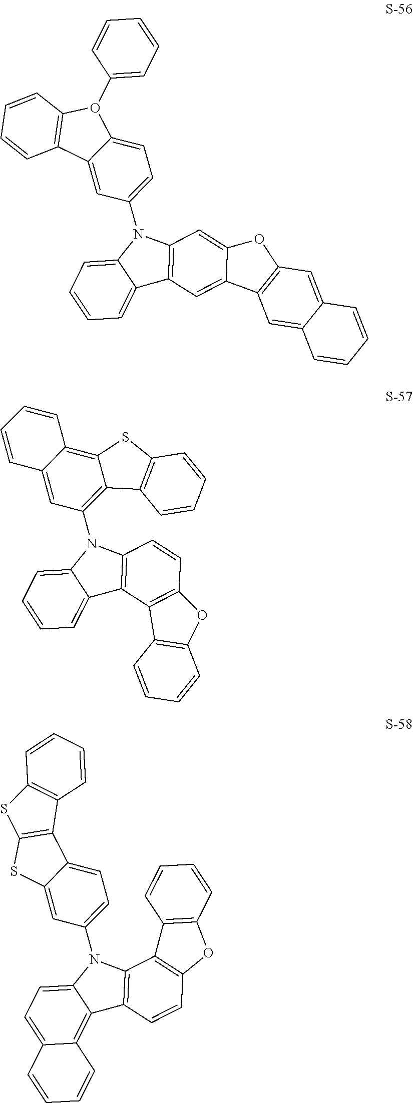

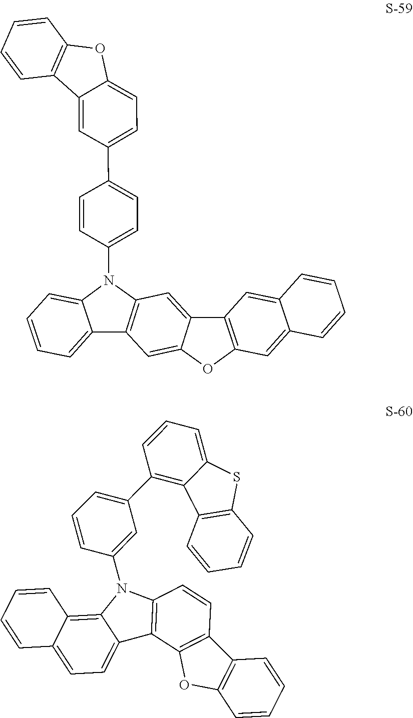 [SMILES: c1ccc2cc3c(cc2c1)oc1cc2c4ccccc4n(-c4ccc(-c5ccc6oc7ccccc7c6c5)cc4)c2cc13 ; c1cc(-c2cccc3sc4ccccc4c23)cc(-n2c3ccc4c5ccccc5oc4c3c3ccc4ccccc4c32)c1]

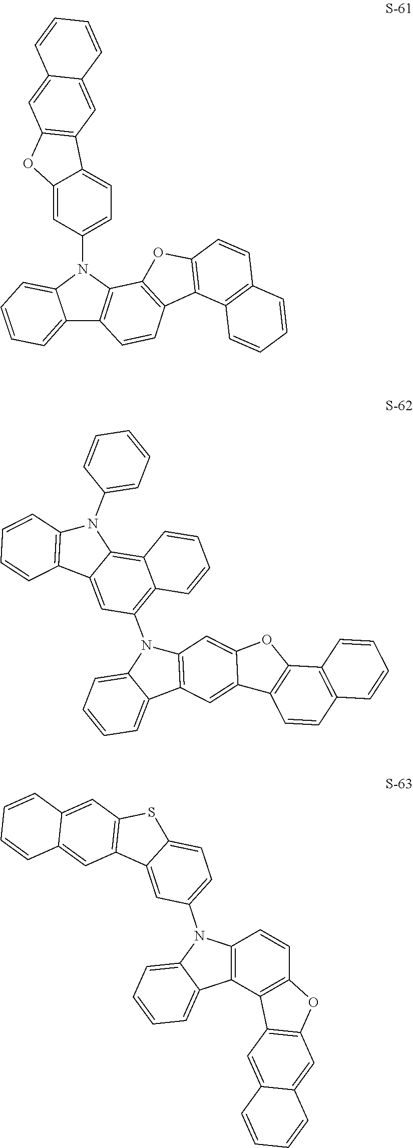 [SMILES: c1ccc2cc3c(cc2c1)oc1cc(-n2c4ccccc4c4ccc5c(oc6ccc7ccccc7c65)c42)ccc13 ; c1ccc(-n2c3ccccc3c3cc(-n4c5ccccc5c5cc6c(cc54)oc4c5ccccc5ccc64)c4ccccc4c32)cc1 ; c1ccc2cc3c(cc2c1)oc1ccc2c(c4ccccc4n2-c2ccc4sc5cc6ccccc6cc5c4c2)c13]

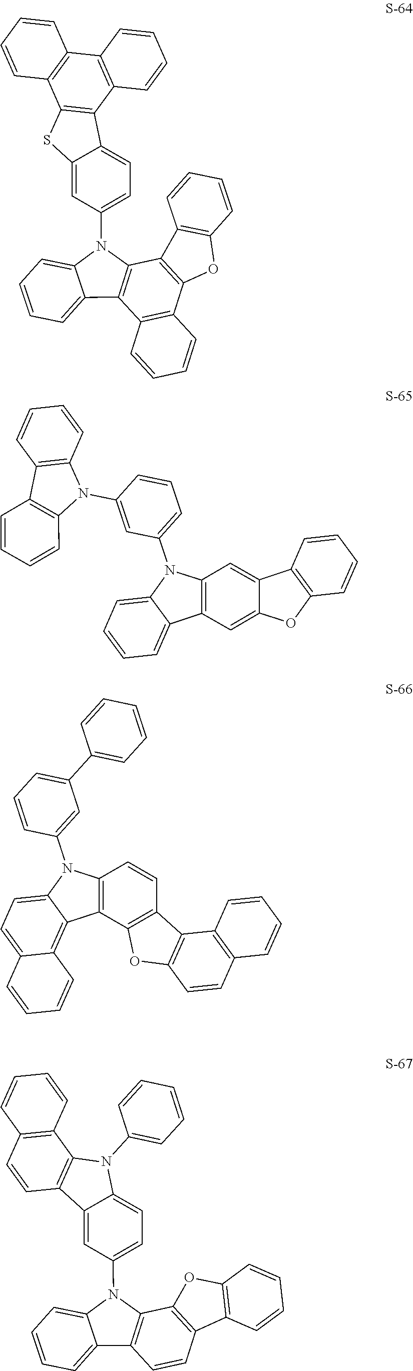

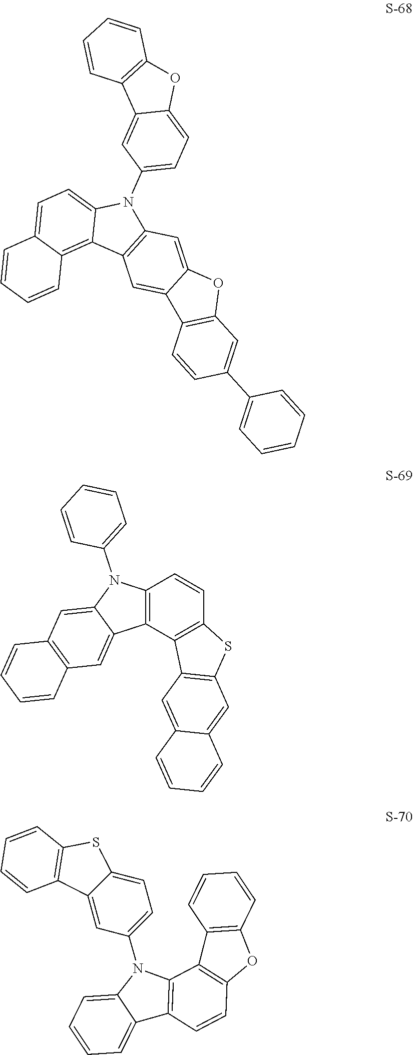 [SMILES: c1ccc(-c2ccc3c(c2)oc2cc4c(cc23)c2c3ccccc3ccc2n4-c2ccc3oc4ccccc4c3c2)cc1 ; c1ccc(-n2c3cc4ccccc4cc3c3c4c(ccc32)sc2cc3ccccc3cc24)cc1 ; c1ccc2c(c1)oc1ccc3c4ccccc4n(-c4ccc5sc6ccccc6c5c4)c3c12]

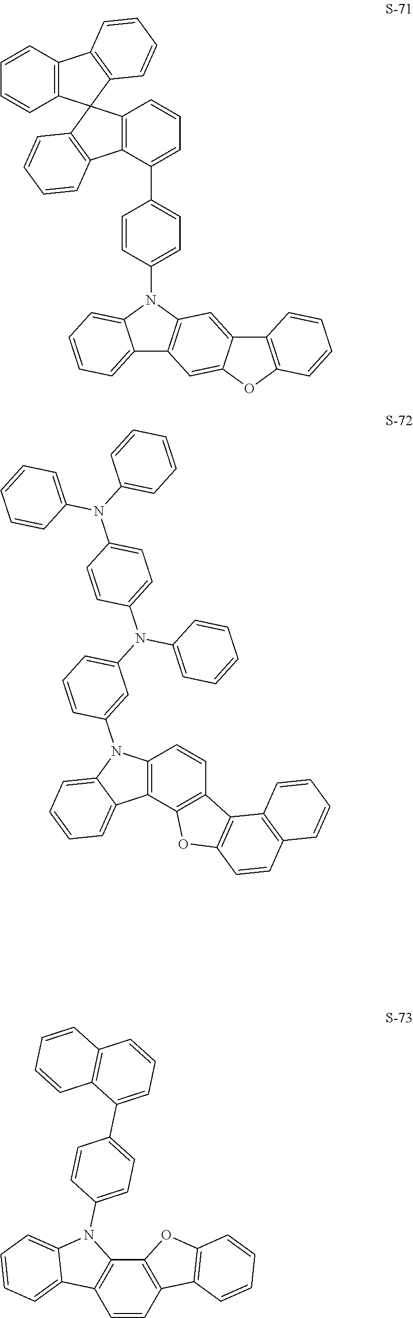 [SMILES: c1ccc2c(c1)-c1ccccc1C21c2ccccc2-c2c(-c3ccc(-n4c5ccccc5c5cc6oc7ccccc7c6cc54)cc3)cccc21 ; c1ccc(N(c2ccccc2)c2ccc(N(c3ccccc3)c3cccc(-n4c5ccccc5c5c6oc7ccc8ccccc8c7c6ccc54)c3)cc2)cc1 ; c1ccc2c(-c3ccc(-n4c5ccccc5c5ccc6c7ccccc7oc6c54)cc3)cccc2c1]

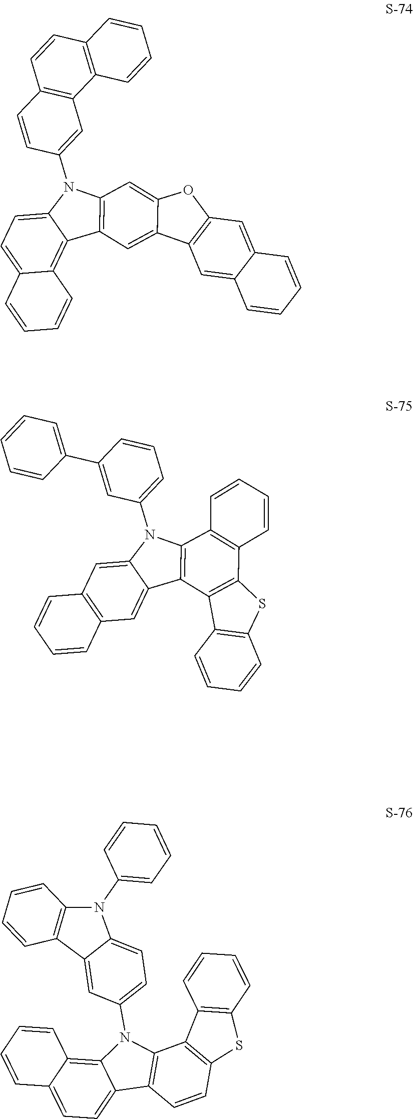 [SMILES: c1ccc2cc3c(cc2c1)oc1cc2c(cc13)c1c3ccccc3ccc1n2-c1ccc2ccc3ccccc3c2c1 ; c1ccc(-c2cccc(-n3c4cc5ccccc5cc4c4c5c6ccccc6sc5c5ccccc5c43)c2)cc1 ; c1ccc(-n2c3ccccc3c3cc(-n4c5c6ccccc6ccc5c5ccc6sc7ccccc7c6c54)ccc32)cc1]

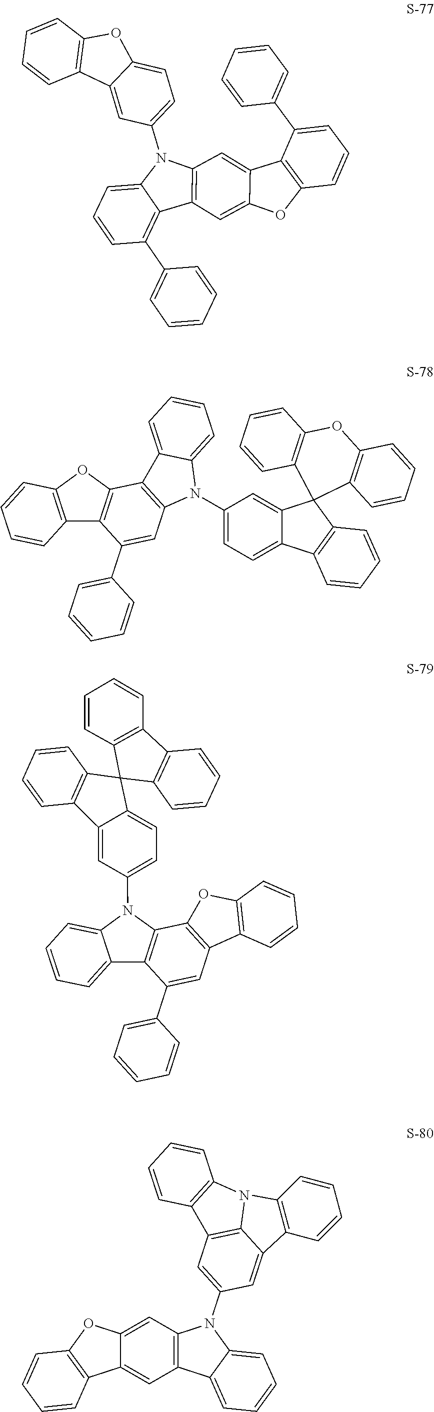

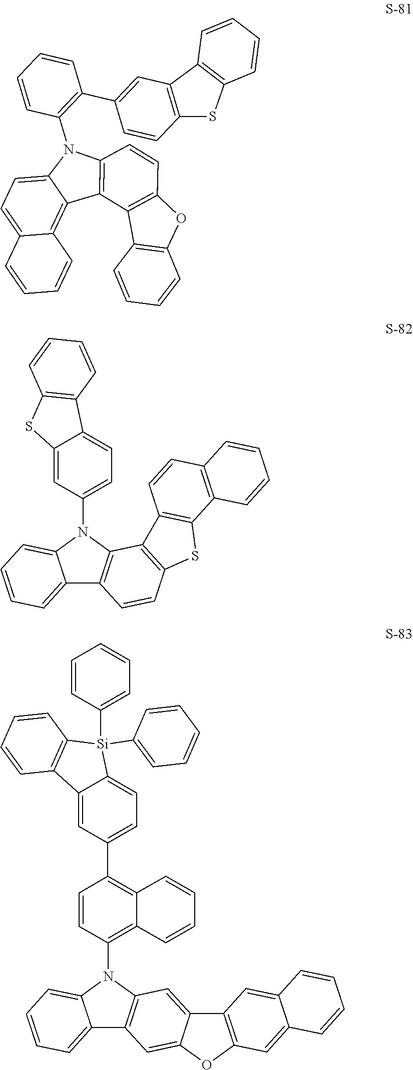

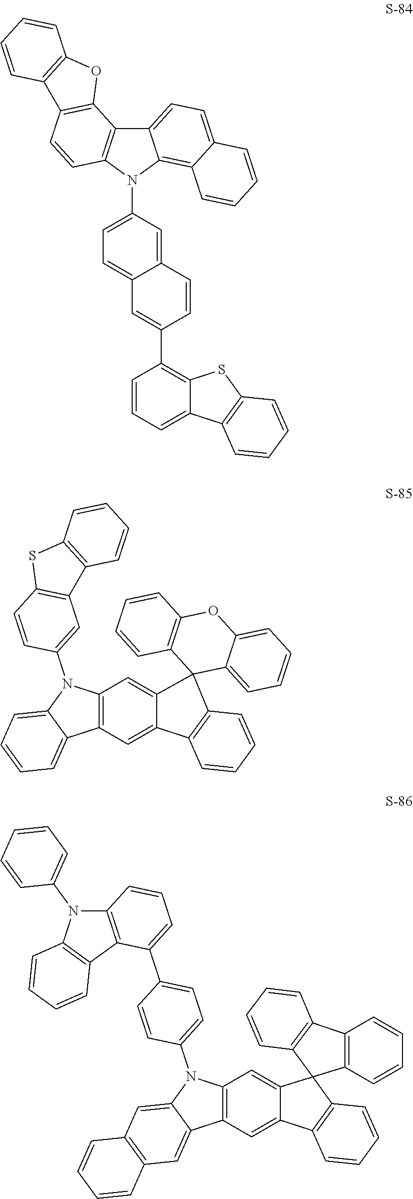 [SMILES: c1ccc2c(c1)ccc1c3c4oc5ccccc5c4ccc3n(-c3ccc4cc(-c5cccc6c5sc5ccccc56)ccc4c3)c21 ; c1ccc2c(c1)Oc1ccccc1C21c2ccccc2-c2cc3c4ccccc4n(-c4ccc5sc6ccccc6c5c4)c3cc21 ; c1ccc(-n2c3ccccc3c3c(-c4ccc(-n5c6cc7c(cc6c6cc8ccccc8cc65)-c5ccccc5C75c6ccccc6-c6ccccc65)cc4)cccc32)cc1]

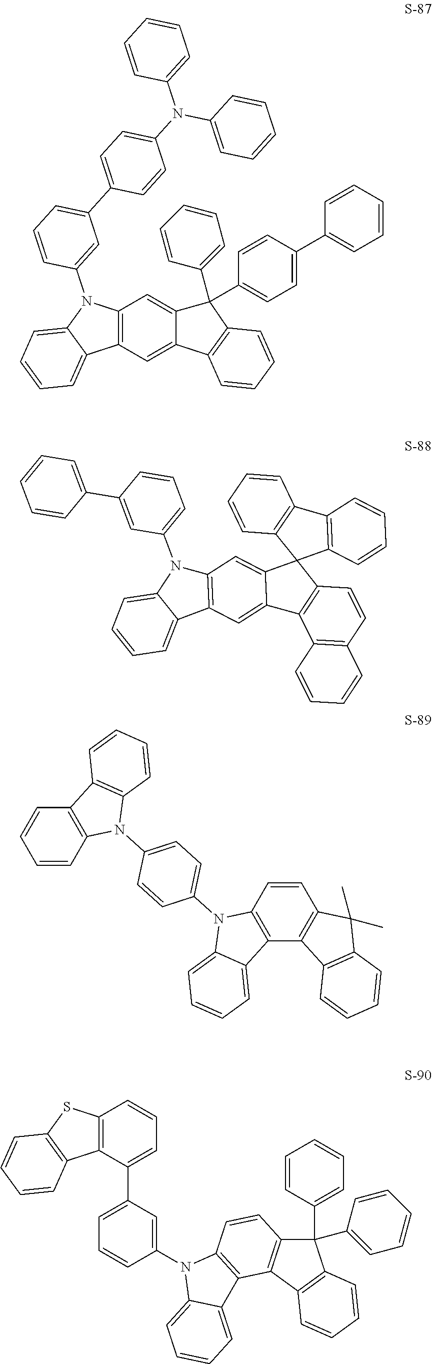 [SMILES: c1ccc(-c2ccc(C3(c4ccccc4)c4ccccc4-c4cc5c6ccccc6n(-c6cccc(-c7ccc(N(c8ccccc8)c8ccccc8)cc7)c6)c5cc43)cc2)cc1 ; c1ccc(-c2cccc(-n3c4ccccc4c4cc5c(cc43)C3(c4ccccc4-c4ccccc43)c3ccc4ccccc4c3-5)c2)cc1 ; CC1(C)c2ccccc2-c2c1ccc1c2c2ccccc2n1-c1ccc(-n2c3ccccc3c3ccccc32)cc1 ; c1ccc(C2(c3ccccc3)c3ccccc3-c3c2ccc2c3c3ccccc3n2-c2cccc(-c3cccc4sc5ccccc5c34)c2)cc1]

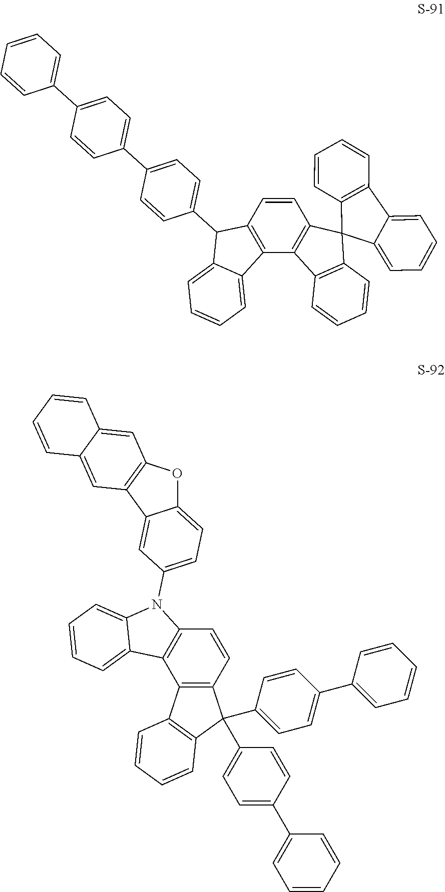

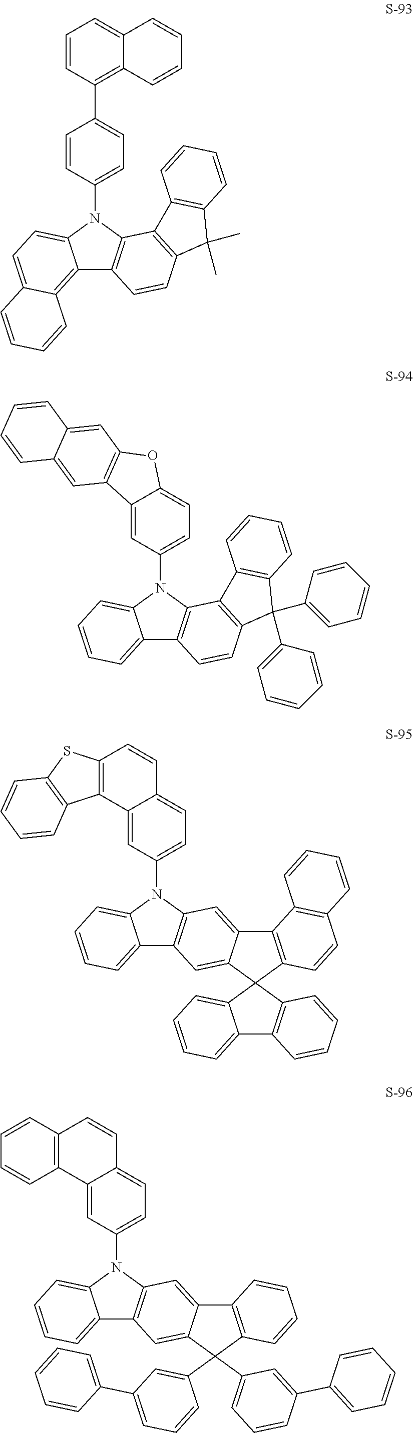 [SMILES: CC1(C)c2ccccc2-c2c1ccc1c3c4ccccc4ccc3n(-c3ccc(-c4cccc5ccccc45)cc3)c21 ; c1ccc(C2(c3ccccc3)c3ccccc3-c3c2ccc2c4ccccc4n(-c4ccc5oc6cc7ccccc7cc6c5c4)c32)cc1 ; c1ccc2c(c1)-c1ccccc1C21c2cc3c4ccccc4n(-c4ccc5ccc6sc7ccccc7c6c5c4)c3cc2-c2c1ccc1ccccc21 ; c1ccc(-c2cccc(C3(c4cccc(-c5ccccc5)c4)c4ccccc4-c4cc5c(cc43)c3ccccc3n5-c3ccc4ccc5ccccc5c4c3)c2)cc1]

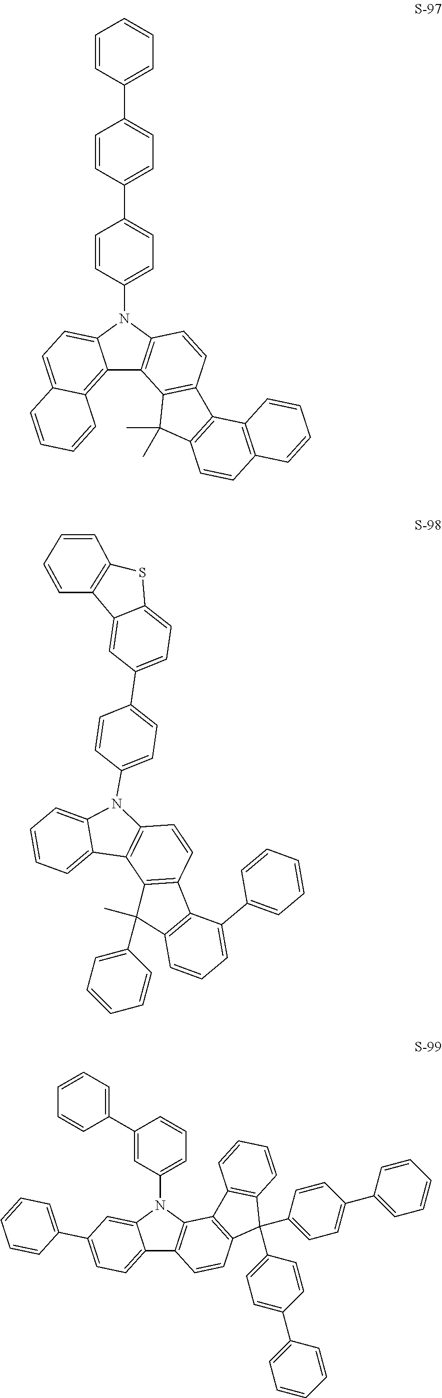 [SMILES: CC1(C)c2ccc3ccccc3c2-c2ccc3c(c21)c1c2ccccc2ccc1n3-c1ccc(-c2ccc(-c3ccccc3)cc2)cc1 ; CC1(c2ccccc2)c2cccc(-c3ccccc3)c2-c2ccc3c(c21)c1ccccc1n3-c1ccc(-c2ccc3sc4ccccc4c3c2)cc1 ; c1ccc(-c2ccc(C3(c4ccc(-c5ccccc5)cc4)c4ccccc4-c4c3ccc3c5ccc(-c6ccccc6)cc5n(-c5cccc(-c6ccccc6)c5)c43)cc2)cc1]

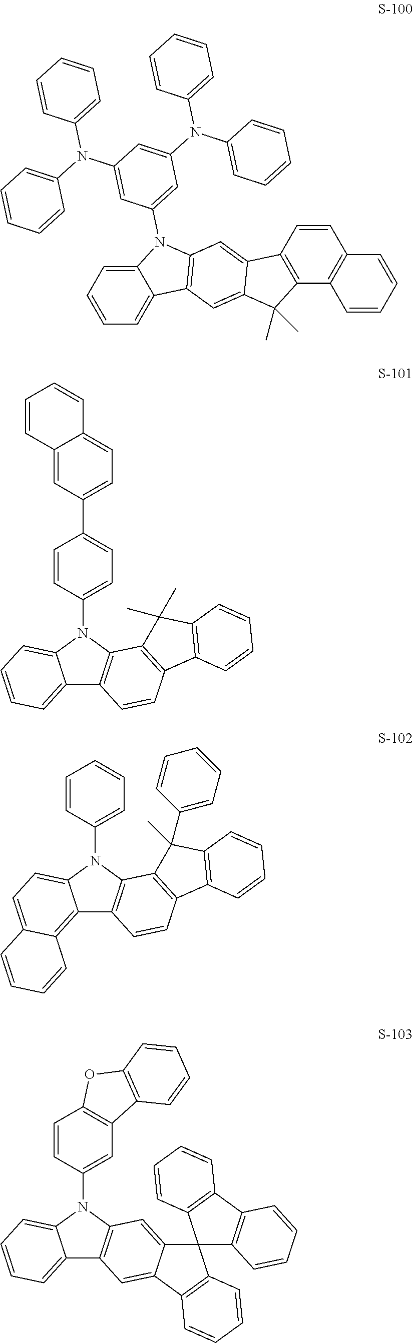 [SMILES: CC1(C)c2cc3c4ccccc4n(-c4cc(N(c5ccccc5)c5ccccc5)cc(N(c5ccccc5)c5ccccc5)c4)c3cc2-c2ccc3ccccc3c21 ; CC1(C)c2ccccc2-c2ccc3c4ccccc4n(-c4ccc(-c5ccc6ccccc6c5)cc4)c3c21 ; CC1(c2ccccc2)c2ccccc2-c2ccc3c4c5ccccc5ccc4n(-c4ccccc4)c3c21 ; c1ccc2c(c1)-c1ccccc1C21c2ccccc2-c2cc3c4ccccc4n(-c4ccc5oc6ccccc6c5c4)c3cc21]

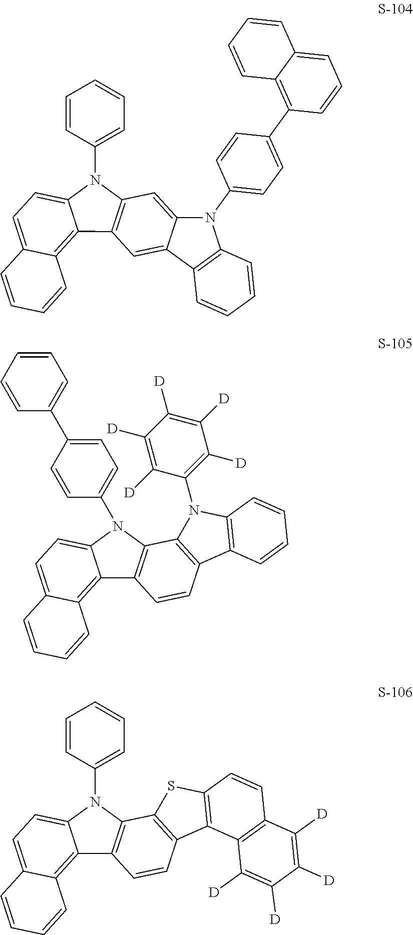

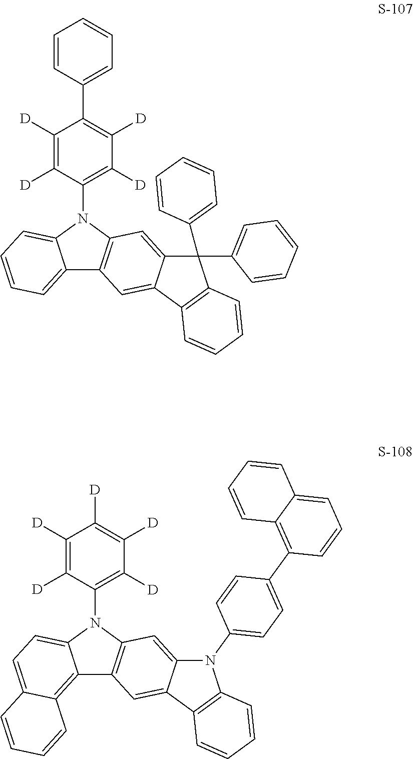 [SMILES: [2H]c1c([2H])c(-n2c3ccccc3c3cc4c(cc32)C(c2ccccc2)(c2ccccc2)c2ccccc2-4)c([2H])c([2H])c1-c1ccccc1 ; [2H]c1c([2H])c([2H])c(-n2c3cc4c(cc3c3c5ccccc5ccc32)c2ccccc2n4-c2ccc(-c3cccc4ccccc34)cc2)c([2H])c1[2H]]

In another aspect, the present invention provides a method for reusing the compound represented by Formula 3 comprising:
a step of depositing an organic emitting material including the compound represented by Formula 3 in a manufacturing process of an organic light emitting device;
a step of removing impurities from the crude organic light emitting material recovered from the deposition apparatus;
a step of recovering the removed impurities; and
a step of purifying the recovered impurities to a purity of 99.9% or higher.

The step of removing impurities from the crude organic light emitting material recovered from the deposition apparatus may preferably comprise performing a pre-purification process to obtain a purity of 98% or more by recrystallization in a recrystallization solvent.

The recrystallization solvent may be a polar solvent having a polarity index (PI) of 5.5 to 7.2.

The recrystallization solvent may preferably be used by mixing a polar solvent having a polarity value of 5.5 to 7.2 and a non-polar solvent having a polarity value of 2.0 to 4.7.

When a mixture of a polar solvent and a non-polar solvent is used, the recrystallization solvent may be used in an amount of 15% (v/v) or less of the non-polar solvent compared to the polar solvent.

The recrystallization solvent may preferably be used by mixing N-Methylpyrrolidone (NMP) single solvent; or a polar solvent mixed any one selected from the group consisting of 1,3-Dimethyl-2-imidazolidinone, 2-pyrrolidone, N, N-Dimethyl formamide, Dimethyl acetamide, and Dimethyl sulfoxide to the N-Methylpyrrolidone; or alone; or mixed non-polar solvents; selected from the group consisting of Toluene, Dichloromethane (DCM), Dichloroethane (DCE), Tetrahydrofuran (THF), Chloroform, Ethyl acetate and Butanone; or a polar solvent and a non-polar solvent.

The pre-purification process may comprise a step of precipitating crystals of by cooling to 0° C. to 5° C. after dissolving the crude organic light emitting material recovered from the deposition apparatus in a polar solvent at 90° C. to 120° C.

The pre-purification process may comprise a step of precipitating crystals by cooling to 35° C. to 40° C., adding a non-polar solvent, and then cooling to 0° C. to 5° C. after dissolving the crude organic light emitting material recovered from the deposition apparatus in a polar solvent at 90° C. to 120° C.

The pre-purification process may comprise a step of precipitating crystals while concentrating the solvent and removing the non-polar solvent, after dissolving the crude organic light emitting material recovered from the deposition apparatus in a non-polar solvent.

The pre-purification process may comprise a step of recrystallizing again with a non-polar solvent after recrystallizing first with a polar solvent.

The step of purifying the recovered impurities to a purity of 99.9% or higher may comprise performing an adsorption separation process to adsorb and remove impurities by adsorbing on the adsorbent.

The adsorbent may be activated carbon, silica gel, alumina, or a material for known adsorption purposes.

The step of purifying the recovered impurities to a purity of 99.9% or higher may comprise performing sublimation purification.

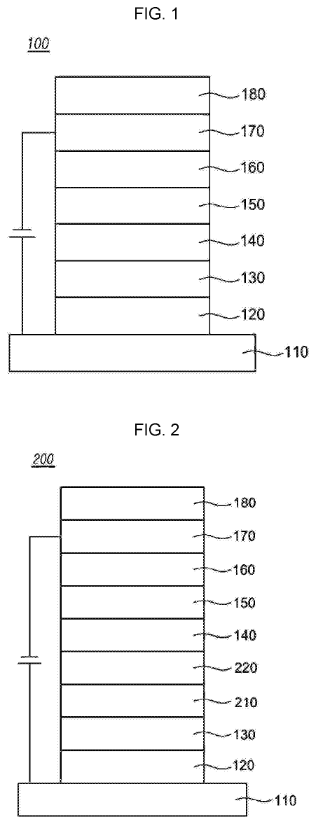

Referring to FIG. 1, the organic electronic element (100) according to the present invention includes a first electrode (110), a second electrode (170), an organic material layer comprising single compound represented by Formula 3 or 2 or more compounds between the first electrode (110) and the second electrode (170), Here, the first electrode (110) may be an anode or a positive electrode, and the second electrode (170) may be a cathode or a negative electrode. In the case of an inverted organic electronic element, the first electrode may be a cathode, and the second electrode may be an anode.

The organic material layer may sequentially comprise a hole injection layer (120), a hole transport layer (130), an emitting layer (140), an electron transport layer (150), and an electron injection layer (160) formed in sequence on the first electrode (110). Here, the remaining layers except the emitting layer (140) may not be formed. The organic material layer may further comprise a hole blocking layer, an electron blocking layer, an emitting-auxiliary layer (220), a buffer layer (210), etc., and the electron transport layer (150) and the like may serve as a hole blocking layer (see FIG. 2).

Also, the organic electronic element according to an embodiment of the present invention may further include a protective layer or a light efficiency enhancing layer (180). The light efficiency enhancing layer may be formed on a surface not in contact with the organic material layer among both surfaces of the first electrode or on a surface not in contact with the organic material layer among both surfaces of the second electrode. The compound according to an embodiment of the present invention applied to the organic material layer may be used as a material for a hole injection layer (120), a hole transport layer (130), an emitting-auxiliary layer (220), an electron transport auxiliary layer, an electron transport layer (150), an electron injection layer (160), a host or dopant of an emitting layer (140), or the light efficiency enhancing layer. Preferably, for example, the compound according to Formula 3 of the present invention may be used as a host material for the emitting layer.

The organic material layer may include 2 or more stacks comprising a hole transport layer, an emitting layer, and an electron transport layer sequentially formed on the anode, and may further comprise a charge generation layer formed between the 2 or more stacks (see FIG. 3).

Otherwise, even if the same core is used, the band gap, the electrical characteristics, the interface characteristics, and the like may vary depending on which substituent is bonded at which position, therefore the choice of core and the combination of sub-substituents associated therewith is also very important, and in particular, when the optimal combination of energy levels and T1 values and unique properties of materials (mobility, interfacial characteristics, etc.) of each organic material layer is achieved, a long life span and high efficiency can be achieved at the same time.

The organic electroluminescent device according to an embodiment of the present invention may be manufactured using a PVD (physical vapor deposition) method. For example, a metal or a metal oxide having conductivity or an alloy thereof is deposited on a substrate to form a cathode, and the organic material layer including the hole injection layer (120), the hole transport layer (130), the emitting layer (140), the electron transport layer (150), and the electron injection layer (160) is formed thereon, and then depositing a material usable as a cathode thereon can manufacture an organic electroluminescent device according to an embodiment of the present invention.

Also, the present invention provides the organic electronic element wherein the organic material layer is formed by one of a spin coating process, a nozzle printing process, an inkjet printing process, a slot coating process, a dip coating process or a roll-to-roll process, and the organic material layer provides an organic electronic element comprising the compound as an electron transport material.

As another specific example, the present invention provides an organic electronic element that is used by mixing the same or different compounds of the compound represented by Formula 3 to the organic material layer.

Also, the present invention provides an emitting layer composition comprising a compound represented by Formula 3, and provides an organic electronic element comprising the emitting layer.

Also, the present invention also provides an electronic device comprising a display device including the organic electronic element; and a control unit for driving the display device.

According to another aspect, the present invention provides an display device wherein the organic electronic element is at least one of an OLED, an organic solar cell, an organic photo conductor, an organic transistor (organic TFT) and an element for monochromic or white illumination. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, Synthesis examples of the compound represented by Formulas 2 to 5 and preparation examples of the organic electronic element of the present invention will be described in detail by way of example, but are not limited to the following examples.

### [Synthesis Example 1] Compound Represented by Formula 2

The compound (final product 2) represented by Formula 2 according to the present invention may be prepared by reacting as in Reaction Scheme 4, but is not limited thereto.

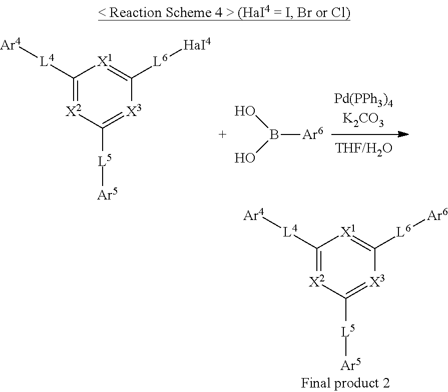

I. Synthesis of Final Product 2

1. Synthesis Example of N-1

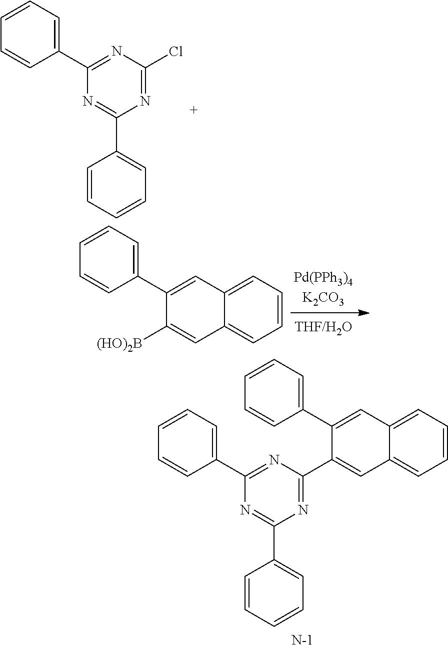

2-chloro-4,6-diphenyl-1,3,5-triazine (8 g, 30 mmol) and (3-phenylnaphthalen-2-yl)boronic acid (8.2 g, 33 mmol), K₂CO₃ (12.4 g, 90 mmol), Pd(PPh₃)₄ (1.7 g, 1.5 mmol) were placed in a round flask, after dissolving in THF and water, the mixture was refluxed at 80° C. for 12 hours. When the reaction was completed, the reactant was cooled to room temperature, extracted with CH₂Cl₂, and washed with water. After drying the organic layer with MgSO₄ and concentrating, the resulting organic material was separated using a silica gel column to obtain the desired product (9.54 g, 73%).

2. Synthesis Example of N-19

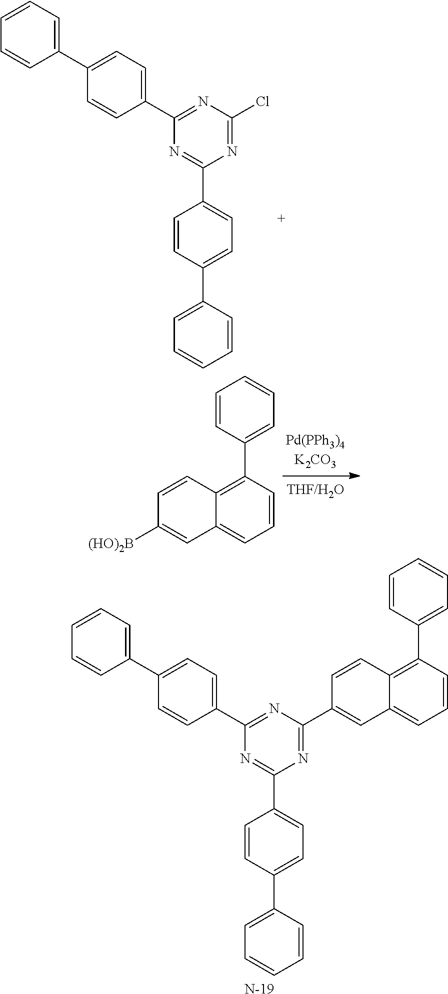

2,4-di([1,1′-biphenyl]-4-yl)-6-chloro-1,3,5-triazine (12.6 g, 30 mmol) and (5-phenylnaphthalen-2-yl)boronic acid (8.2 g, 33 mmol) were used for the synthesis method of N-1 to obtain 15.5 g (yield: 88%) of a product.

3. Synthesis Example of N-33

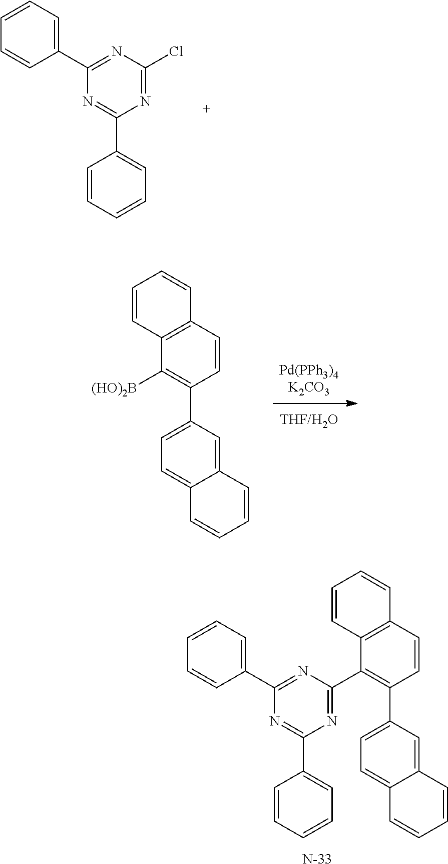

2-chloro-4,6-diphenyl-1,3,5-triazine (8 g, 30 mmol) and [2,2′-binaphthalen]-1-ylboronic acid (9.8 g, 33 mmol) were used for the synthesis method of N-1 to obtain 9.8 g (yield: 67%) of a product.

4. Synthesis Example of N-53

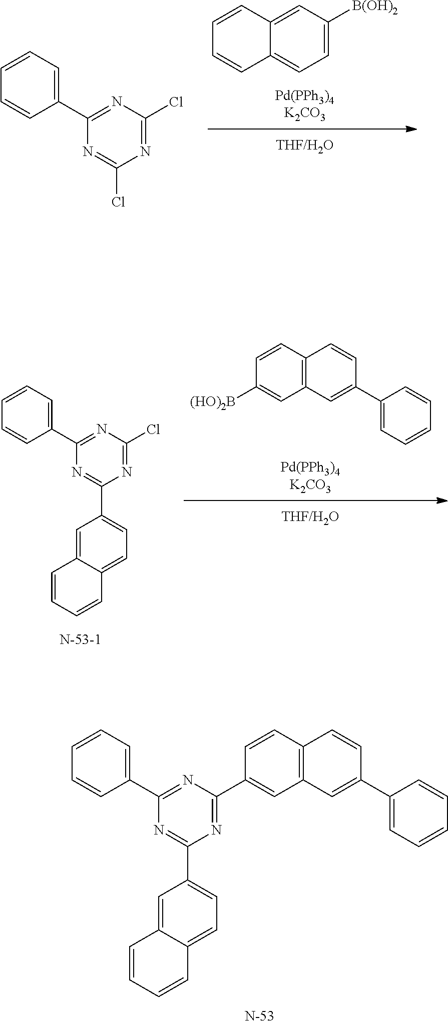

(1) Synthesis of N-53-1

2,4-dichloro-6-phenyl-1,3,5-triazine (6.8 g, 30 mmol) and naphthalen-2-ylboronic acid (5.1 g, 30 mmol) were used for the synthesis method of N-1 to obtain 6.7 g (yield: 70%) of a product.

(2) Synthesis of N-53

2-chloro-4-(naphthalen-2-yl)-6-phenyl-1,3,5-triazine (9.5 g, 30 mmol) and (7-phenylnaphthalen-2-yl)boronic acid (8.2 g, 33 mmol) were used for the synthesis method of N-1 to obtain 12.4 g (yield: 85%) of a product.

5. Synthesis Example of N-87

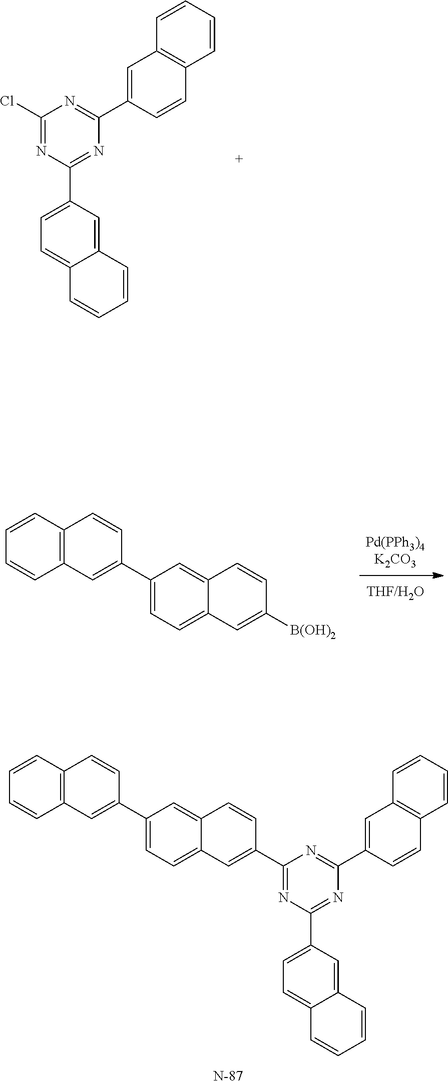

2-chloro-4,6-di(naphthalen-2-yl)-1,3,5-triazine (11 g, 30 mmol) and [2,2′-binaphthalen]-6-ylboronic acid (9.8 g, 33 mmol) were used for the synthesis method of N-1 to obtain 14.8 g (yield: 84%) of a product.

6. Synthesis Example of N-113

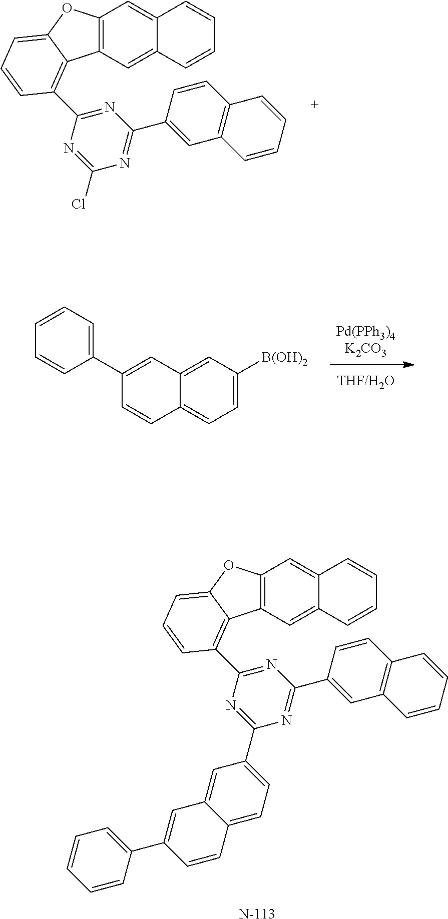 [SMILES: Clc1nc(-c2ccc3ccccc3c2)nc(-c2cccc3oc4cc5ccccc5cc4c23)n1 ; OB(O)c1ccc2ccc(-c3ccccc3)cc2c1 ; c1ccc(-c2ccc3ccc(-c4nc(-c5ccc6ccccc6c5)nc(-c5cccc6oc7cc8ccccc8cc7c56)n4)cc3c2)cc1 ; "N-113"]

2-chloro-4-(naphthalen-2-yl)-6-(naphtho[2,3-b]benzofuran-1-yl)-1,3,5-triazine (13.7 g, 30 mmol) and (7-phenylnaphthalen-2-yl)boronic acid (8.2 g, 33 mmol) were used for the synthesis method of N-1 to obtain 14.5 g (yield: 77%) of a product.

7. Synthesis Example of N-115

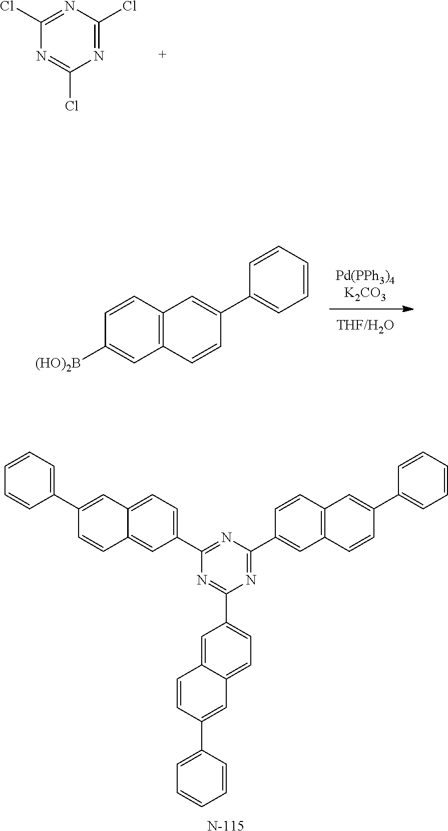

2,4,6-trichloro-1,3,5-triazine (5.5 g, 30 mmol) and (6-phenylnaphthalen-2-yl)boronic acid (23 g, 93 mmol) were used for the synthesis method of N-1 to obtain 15 g (yield: 73%) of a product.

8. Synthesis Example of N-165

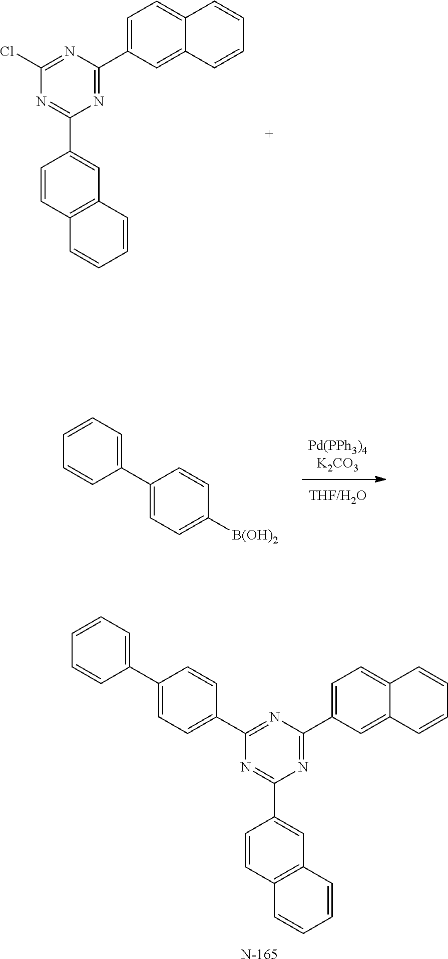

2-chloro-4,6-di(naphthalen-2-yl)-1,3,5-triazine (11 g, 30 mmol) and [1,1′-biphenyl]-4-ylboronic acid (5.9 g, 30 mmol) were used for the synthesis method of N-1 to obtain 11.9 g (yield: 82%) of a product.

9. Synthesis Example of N-117

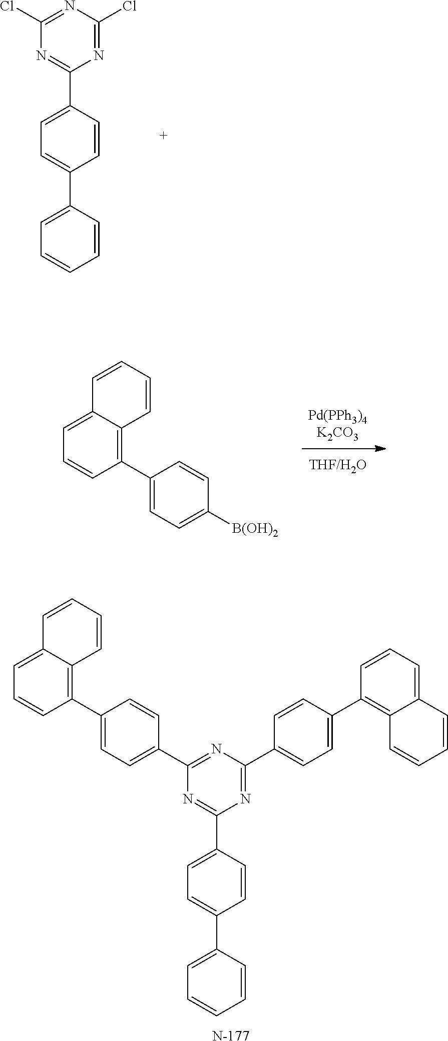

2-([1,1′-biphenyl]-4-yl)-4,6-dichloro-1,3,5-triazine (9 g, 30 mmol) and (4-(naphthalen-1-yl)phenyl)boronic acid (15.4 g, 62 mmol) were used for the synthesis method of N-1 to obtain 12.8 g (yield: 67%) of a product.

Further, the FD-MS values of the compounds N-1 to N-184 of the present invention prepared according to the Synthesis Example as described above are shown in Table 4.

TABLE 4

| Compound | FD-MS | Compound | FD-MS |
|---|---|---|---|
| N-1 | m/z = 435.17(C₃₁H₂₁N₃ = 435.53) | N-2 | m/z = 435.17(C₃₁H₂₁N₃ = 435.53) |
| N-3 | m/z = 435.17(C₃₁H₂₁N₃ = 435.53) | N-4 | m/z = 435.17(C₃₁H₂₁N₃ = 435.53) |
| N-5 | m/z = 435.17(C₃₁H₂₁N₃ = 435.53) | N-6 | m/z = 435.17(C₃₁H₂₁N₃ = 435.53) |
| N-7 | m/z = 435.17(C₃₁H₂₁N₃ = 435.53) | N-8 | m/z = 434.18(C₃₂H₂₂N₂ = 434.54) |
| N-9 | m/z = 511.2(C₃₇H₂₅N₃ = 511.63) | N-10 | m/z = 611.24(C₄₅H₂₉N₃ = 611.75) |
| N-11 | m/z = 485.19(C₃₅H₂₃N₃ = 485.59) | N-12 | m/z = 511.2(C₃₇H₂₅N₃ = 511.63) |
| N-13 | m/z = 511.2(C₃₇H₂₅N₃ = 511.63) | N-14 | m/z = 485.19(C₃₅H₂₃N₃ = 485.59) |
| N-15 | m/z = 434.18(C₃₂H₂₂N₂ = 434.54) | N-16 | m/z = 434.18(C₃₂H₂₂N₂ = 434.54) |
| N-17 | m/z = 511.2(C₃₇H₂₅N₃ = 511.63) | N-18 | m/z = 561.22(C₄₁H₂₇N₃ = 561.69) |
| N-19 | m/z = 587.24(C₄₃H₂₉N₃ = 587.73) | N-20 | m/z = 511.2(C₃₇H₂₅N₃ = 511.63) |
| N-21 | m/z = 511.2(C₃₇H₂₅N₃ = 511.63) | N-22 | m/z = 511.2(C₃₇H₂₅N₃ = 511.63) |
| N-23 | m/z = 511.2(C₃₇H₂₅N₃ = 511.63) | N-24 | m/z = 587.24(C₄₃H₂₉N₃ = 587.73) |
| N-25 | m/z = 435.17(C₃₁H₂₁N₃ = 435.53) | N-26 | m/z = 435.17(C₃₁H₂₁N₃ = 435.53) |
| N-27 | m/z = 435.17(C₃₁H₂₁N₃ = 435.53) | N-28 | m/z = 435.17(C₃₁H₂₁N₃ = 435.53) |
| N-29 | m/z = 435.17(C₃₁H₂₁N₃ = 435.53) | N-30 | m/z = 435.17(C₃₁H₂₁N₃ = 435.53) |
| N-31 | m/z = 435.17(C₃₁H₂₁N₃ = 435.53) | N-32 | m/z = 434.18(C₃₂H₂₂N₂ = 434.54) |
| N-33 | m/z = 485.19(C₃₅H₂₃N₃ = 485.59) | N-34 | m/z = 511.2(C₃₇H₂₅N₃ = 511.63) |
| N-35 | m/z = 511.2(C₃₇H₂₅N₃ = 511.63) | N-36 | m/z = 511.2(C₃₇H₂₅N₃ = 511.63) |
| N-37 | m/z = 485.19(C₃₅H₂₃N₃ = 485.59) | N-38 | m/z = 511.2(C₃₇H₂₅N₃ = 511.63) |
| N-39 | m/z = 485.19(C₃₅H₂₃N₃ = 485.59) | N-40 | m/z = 611.24(C₄₅H₂₉N₃ = 611.75) |
| N-41 | m/z = 511.2(C₃₇H₂₅N₃ = 511.63) | N-42 | m/z = 511.2(C₃₇H₂₅N₃ = 511.63) |
| N-43 | m/z = 587.24(C₄₃H₂₉N₃ = 587.73) | N-44 | m/z = 587.24(C₄₃H₂₉N₃ = 587.73) |
| N-45 | m/z = 587.24(C₄₃H₂₉N₃ = 587.73) | N-46 | m/z = 561.22(C₄₁H₂₇N₃ = 561.69) |
| N-47 | m/z = 511.2(C₃₇H₂₅N₃ = 511.63) | N-48 | m/z = 587.24(C₄₃H₂₉N₃ = 587.73) |
| N-49 | m/z = 485.19(C₃₅H₂₃N₃ = 485.59) | N-50 | m/z = 485.19(C₃₅H₂₃N₃ = 485.59) |
| N-51 | m/z = 485.19(C₃₅H₂₃N₃ = 485.59) | N-52 | m/z = 485.19(C₃₅H₂₃N₃ = 485.59) |
| N-53 | m/z = 485.19(C₃₅H₂₃N₃ = 485.59) | N-54 | m/z = 535.2(C₃₉H₂₅N₃ = 535.65) |
| N-55 | m/z = 485.19(C₃₅H₂₃N₃ = 485.59) | N-56 | m/z = 485.19(C₃₅H₂₃N₃ = 485.59) |
| N-57 | m/z = 561.22(C₄₁H₂₇N₃ = 561.69) | N-58 | m/z = 561.22(C₄₁H₂₇N₃ = 561.69) |
| N-59 | m/z = 561.22(C₄₁H₂₇N₃ = 561.69) | N-60 | m/z = 637.25(C₄₇H₃₁N₃ = 637.79) |
| N-61 | m/z = 561.22(C₄₁H₂₇N₃ = 561.69) | N-62 | m/z = 561.22(C₄₁H₂₇N₃ = 561.69) |
| N-63 | m/z = 637.25(C₄₇H₃₁N₃ = 637.79) | N-64 | m/z = 637.25(C₄₇H₃₁N₃ = 637.79) |
| N-65 | m/z = 637.25(C₄₇H₃₁N₃ = 637.79) | N-66 | m/z = 485.19(C₃₅H₂₃N₃ = 485.59) |
| N-67 | m/z = 485.19(C₃₅H₂₃N₃ = 485.59) | N-68 | m/z = 611.24(C₄₅H₂₉N₃ = 611.75) |
| N-69 | m/z = 485.19(C₃₅H₂₃N₃ = 485.59) | N-70 | m/z = 485.19(C₃₅H₂₃N₃ = 485.59) |
| N-71 | m/z = 611.24(C₄₅H₂₉N₃ = 611.75) | N-72 | m/z = 485.19(C₃₅H₂₃N₃ = 485.59) |
| N-73 | m/z = 485.19(C₃₅H₂₃N₃ = 485.59) | N-74 | m/z = 561.22(C₄₁H₂₇N₃ = 561.69) |
| N-75 | m/z = 485.19(C₃₅H₂₃N₃ = 485.59) | N-76 | m/z = 485.19(C₃₅H₂₃N₃ = 485.59) |
| N-77 | m/z = 611.24(C₄₅H₂₉N₃ = 611.75) | N-78 | m/z = 611.24(C₄₅H₂₉N₃ = 611.75) |
| N-79 | m/z = 485.19(C₃₅H₂₃N₃ = 485.59) | N-80 | m/z = 485.19(C₃₅H₂₃N₃ = 485.59) |
| N-81 | m/z = 485.19(C₃₅H₂₃N₃ = 485.59) | N-82 | m/z = 485.19(C₃₅H₂₃N₃ = 485.59) |
| N-83 | m/z = 535.2(C₃₉H₂₅N₃ = 535.65) | N-84 | m/z = 535.2(C₃₉H₂₅N₃ = 535.65) |
| N-85 | m/z = 585.22(C₄₃H₂₇N₃ = 585.71) | N-86 | m/z = 535.2(C₃₉H₂₅N₃ = 535.65) |
| N-87 | m/z = 585.22(C₄₃H₂₇N₃ = 585.71) | N-88 | m/z = 585.22(C₄₃H₂₇N₃ = 585.71) |
| N-89 | m/z = 611.24(C₄₅H₂₉N₃ = 611.75) | N-90 | m/z = 611.24(C₄₅H₂₉N₃ = 611.75) |
| N-91 | m/z = 585.22(C₄₃H₂₇N₃ = 585.71) | N-92 | m/z = 611.24(C₄₅H₂₉N₃ = 611.75) |
| N-93 | m/z = 687.27(C₅₁H₃₃N₃ = 687.85) | N-94 | m/z = 611.24(C₄₅H₂₉N₃ = 611.75) |
| N-95 | m/z = 511.2(C₃₇H₂₅N₃ = 511.63) | N-96 | m/z = 611.24(C₄₅H₂₉N₃ = 611.75) |
| N-97 | m/z = 561.22(C₄₁H₂₇N₃ = 561.69) | N-98 | m/z = 587.24(C₄₃H₂₉N₃ = 587.73) |
| N-99 | m/z = 663.27(C₄₉H₃₃N₃ = 663.82) | N-100 | m/z = 713.28(C₅₃H₃₅N₃ = 713.88) |
| N-101 | m/z = 575.2(C₄₁H₂₅N₃O =575.67) | N-102 | m/z = 601.22(C₄₃H₂₇N₃O = 601.71) |
| N-103 | m/z = 700.26(C₅₁H₃₂N₄ = 700.85) | N-104 | m/z = 701.25(C₅₁H₃₁N₃O = 701.83) |
| N-105 | m/z = 667.21(C₄₇H₂₉N₃S =667.83) | N-106 | m/z = 541.16(C₃₇H₂₃N₃S = 541.67) |
| N-107 | m/z = 612.23(C₄₄H₂₈N₄ = 612.74) | N-108 | m/z = 562.22(C₄₀H₂₆N₄ = 562.68) |
| N-109 | m/z = 689.26(C₄₉H₃₁N₅ = 689.82) | N-110 | m/z = 639.24(C₄₅H₂₉N₅ = 639.76) |
| N-111 | m/z = 701.25(C₅₁H₃₁N₃O = 701.83) | N-112 | m/z = 631.17(C₄₃H₂₅N₃S = 631.75) |
| N-113 | m/z = 625.22(C₄₅H₂₇N₃O = 625.73) | N-114 | m/z = 591.18(C₄₁H₂₅N₃S = 591.73) |
| N-115 | m/z = 687.27(C₅₁H₃₃N₃ = 687.85) | N-116 | m/z = 701.25(C₅₁H₃₁N₃O = 701.83) |
| N-117 | m/z = 619.3(C₄₅H₃₇N₃ = 619.81) | N-118 | m/z = 601.25(C₄₄H₃₁N₃ = 601.75) |
| N-119 | m/z = 667.23(C₄₇H₂₉N₃O₂ = 667.77) | N-120 | m/z = 540.24(C₃₉H₂₀D₅N₃ = 540.68) |
| N-121 | m/z = 521.17(C₃₅H₂₁F₂N₃ = 521.57) | N-122 | m/z = 510.18(C₃₆H₂₂N₄ = 510.6) |
| N-123 | m/z = 652.23(C₄₆H₂₈N₄O = 652.76) | N-124 | m/z = 527.24(C₃₈H₂₉N₃ = 527.67) |
| N-125 | m/z = 535.2(C₃₉H₂₅N₃ = 535.65) | N-126 | m/z = 535.2(C₃₉H₂₅N₃ = 535.65) |
| N-127 | m/z = 535.2(C₃₉H₂₅N₃ = 535.65) | N-128 | m/z = 535.2(C₃₉H₂₅N₃ = 535.65) |
| N-129 | m/z = 587.24(C₄₃H₂₉N₃ = 587.73) | N-130 | m/z = 612.23(C₄₄H₂₈N₄ = 612.74) |
| N-131 | m/z = 561.22(C₄₁H₂₇N₃ = 561.69) | N-132 | m/z = 687.27(C₅₁H₃₃N₃ = 687.85) |
| N-133 | m/z = 663.27(C₄₉H₃₃N₃ = 663.82) | N-134 | m/z = 601.22(C₄₃H₂₇N₃O = 601.71) |
| N-135 | m/z = 617.19(C₄₃H₂₇N₃S = 617.77) | N-136 | m/z = 752.29(C₅₅H₃₆N₄ = 752.92) |
| N-137 | m/z = 651.23(C₄₇H₂₉N₃O = 651.77) | N-138 | m/z = 677.25(C₄₉H₃₁N₃O = 677.81) |
| N-139 | m/z = 541.16(C₃₇H₂₃N₃S = 541.67) | N-140 | m/z = 750.28(C₅₅H₃₄N₄ = 750.91) |
| N-141 | m/z = 707.24(C₅₀H₃₃N₃S = 707.9) | N-142 | m/z = 651.23(C₄₇H₂₉N₃O = 651.77) |
| N-143 | m/z = 617.19(C₄₃H₂₇N₃S = 617.77) | N-144 | m/z = 667.21(C₄₇H₂₉N₃S = 667.83) |
| N-145 | m/z = 631.17(C₄₃H₂₅N₃OS = 631.75) | N-146 | m/z = 767.26(C₅₅H₃N₃O₂ = 767.89) |
| N-147 | m/z = 647.15(C₄₃H₂₅N₃S₂ = 647.81) | N-148 | m/z = 690.24(C₄₉H₃₀N₄O = 690.81) |
| N-149 | m/z = 511.2(C₃₇H₂₅N₃ = 511.63) | N-150 | m/z = 587.24(C₄₃H₂₉N₃ = 587.73) |
| N-151 | m/z = 511.2(C₃₇H₂₅N₃ = 511.63) | N-152 | m/z = 511.2(C₃₇H₂₅N₃ = 511.63) |
| N-153 | m/z = 511.2(C₃₇H₂₅N₃ = 511.63) | N-154 | m/z = 511.2(C₃₇H₂₅N₃ = 511.63) |
| N-155 | m/z = 511.2(C₃₇H₂₅N₃ = 511.63) | N-156 | m/z = 561.22(C₄₁H₂₇N₃ = 561.69) |
| N-157 | m/z = 485.19(C₃₅H₂₃N₃ = 485.59) | N-158 | m/z = 561.22(C₄₁H₂₇N₃ = 561.69) |
| N-159 | m/z = 611.24(C₄₅H₂₉N₃ = 611.75) | N-160 | m/z = 561.22(C₄₁H₂₇N₃ = 561.69) |
| N-161 | m/z = 535.2(C₃₉H₂₅N₃ = 535.65) | N-162 | m/z = 561.22(C₄₁H₂₇N₃ = 561.69) |
| N-163 | m/z = 611.24(C₄₅H₂₉N₃ = 611.75) | N-164 | m/z = 561.22(C₄₁H₂₇N₃ = 561.69) |
| N-165 | m/z = 485.19(C₃₅H₂₃N₃ = 485.59) | N-166 | m/z = 561.22(C₄₁H₂₇N₃ = 561.69) |
| N-167 | m/z = 611.24(C₄₅H₂₉N₃ = 611.75) | N-168 | m/z = 561.22(C₄₁H₂₇N₃ = 561.69) |
| N-169 | m/z = 485.19(C₃₅H₂₃N₃ = 485.59) | N-170 | m/z = 561.22(C₄₁H₂₇N₃ = 561.69) |
| N-171 | m/z = 535.2(C₃₉H₂₅N₃ = 535.65) | N-172 | m/z = 535.2(C₃₉H₂₅N₃ = 535.65) |
| N-173 | m/z = 561.22(C₄₁H₂₇N₃ = 561.69) | N-174 | m/z = 637.25(C₄₇H₃₁N₃ = 637.79) |
| N-175 | m/z = 587.24(C₄₃H₂₉N₃ = 587.73) | N-176 | m/z = 687.27(C₅₁H₃₃N₃ = 687.85) |
| N-177 | m/z = 637.25(C₄₇H₃₁N₃ = 637.79) | N-178 | m/z = 561.22(C₄₁H₂₇N₃ = 561.69) |
| N-179 | m/z = 499.17(C₃₅H₂₁N₃O = 499.57) | N-180 | m/z = 541.16(C₃₇H₂₃N₃S = 541.67) |
| N-181 | m/z = 611.24(C₄₅H₂₉N₃ = 611.75) | N-182 | m/z = 687.27(C₅₁H₃₃N₃ = 687.85) |
| N-183 | m/z = 676.26(C₄₉H₃₂N₄ = 676.82) | N-184 | m/z = 525.22(C₃₈H₂₇N₃ = 525.66) |

### [Synthesis Example 2] Compound Represented by Formula 3

The compound (Final Product) represented by Formula 3 according to the present invention is synthesized as shown in Reaction Scheme 5, but is not limited thereto.

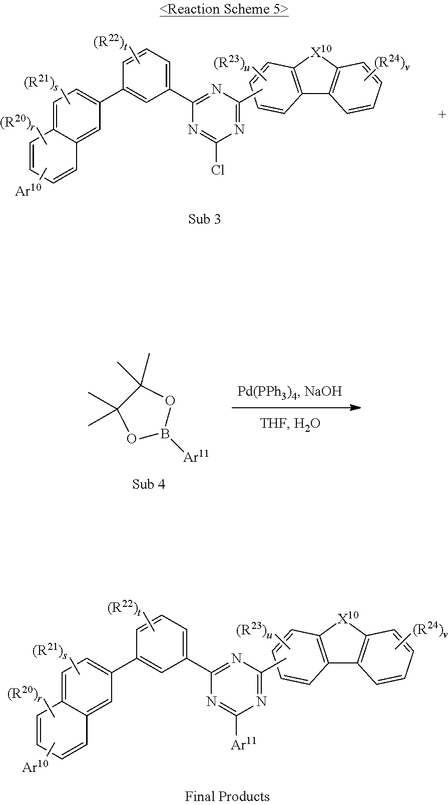

Wherein, R²⁰, R²¹, R²², R²³, R²⁴, Ar¹⁰, Ar¹¹, X¹⁰, r, s, t, u and v are the same as defined in Formula 3.

I. Synthesis of Sub 3

1. Synthesis Example of Sub 3-1

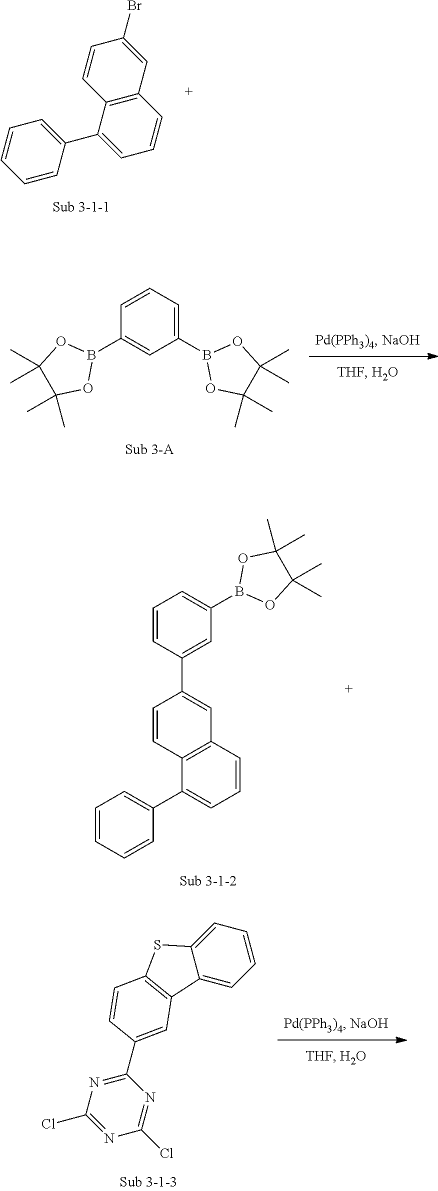

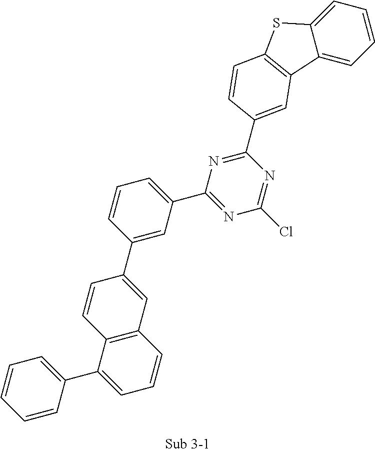 [SMILES: Clc1nc(-c2cccc(-c3ccc4c(-c5ccccc5)cccc4c3)c2)nc(-c2ccc3sc4ccccc4c3c2)n1]

1) Synthesis of Sub 3-1-2

After putting Sub 3-1-1 (85.8 g, 303.0 mmol) in a round bottom flask and dissolving in THF (1515 ml), Sub 3-A (100.0 g, 303.0 mmol), Pd(PPh₃)₄ (21.0 g, 18.2 mmol), NaOH (36.4 g, 909.0 mmol), Water (757 ml) are added and the reaction proceeds at 80° C. When the reaction is complete, the mixture was extracted with CH₂Cl₂ and water, the organic layer was dried over MgSO₄, concentrated, and the resulting compound was recrystallized by silicagel column to obtain 102.7 g (yield: 83.4%) of the product.

2) Synthesis of Sub 3-1

After putting Sub 3-1-2 (102.7 g, 252.7 mmol) in a round bottom flask and dissolving in THF (1263 ml), Sub 3-1-3 (83.9 g, 252.7 mmol), Pd(PPh₃)₄ (17.5 g, 15.2 mmol), NaOH (30.3 g, 758.1 mmol), Water (632 ml) were added and tested in the same manner as in Sub 3-1-2 to obtain 118.8 g of product. (Yield: 81.6%)

2. Synthesis Example of Sub 3-6

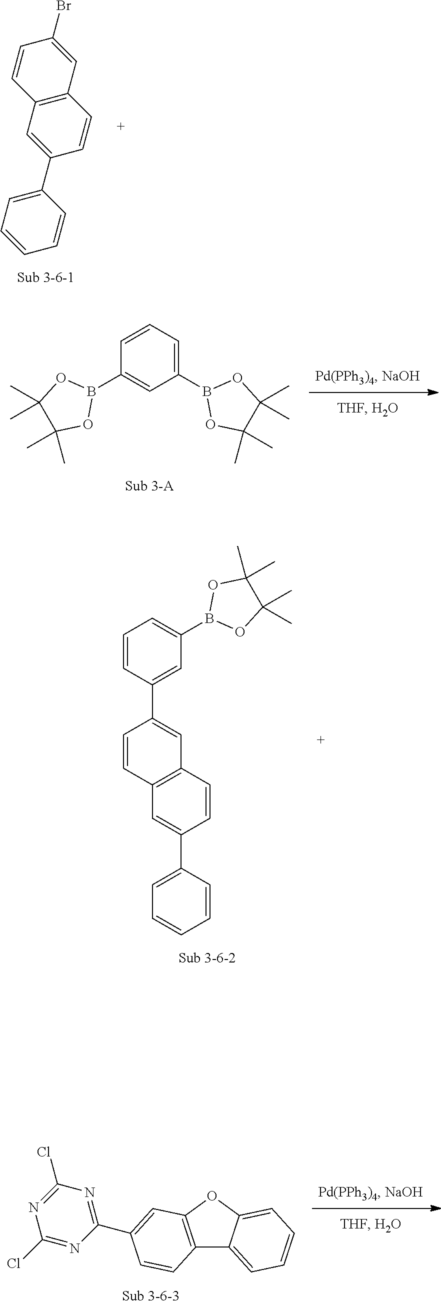

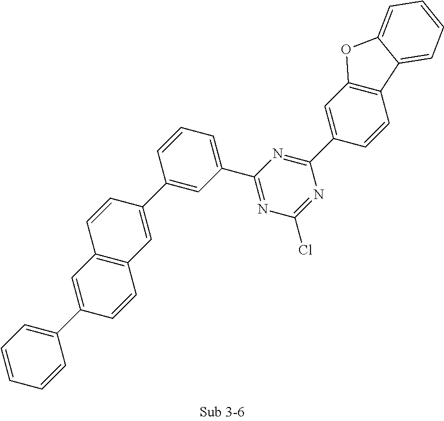 [SMILES: Clc1nc(-c2cccc(-c3ccc4cc(-c5ccccc5)ccc4c3)c2)nc(-c2ccc3c(c2)oc2ccccc23)n1]

1) Synthesis of Sub 3-6-2

After putting Sub 3-6-1 (85.8 g, 303.0 mmol) in a round bottom flask and dissolving in THF (1515 ml), Sub 3-A (100.0 g, 303.0 mmol), Pd(PPh₃)₄ (21.0 g, 18.2 mmol), NaOH (36.4 g, 909.0 mmol), Water (757 ml) were added and tested in the same manner as in Sub 3-1-2 to obtain 103.5 g of product. (Yield: 81.4%)

2) Synthesis of Sub 3-6

After putting Sub 3-6-2 (103.5 g, 254.8 mmol) in a round bottom flask and dissolving in THF (1274 ml), Sub 3-6-3 (80.6 g, 254.8 mmol), Pd(PPh₃)₄ (17.7 g, 15.3 mmol), NaOH (30.6 g, 764.5 mmol), Water (637 ml) were added and tested in the same manner as in Sub 3-1-2 to obtain 117.3 g of product. (Yield: 82.2%)

3. Synthesis of Sub 3-13

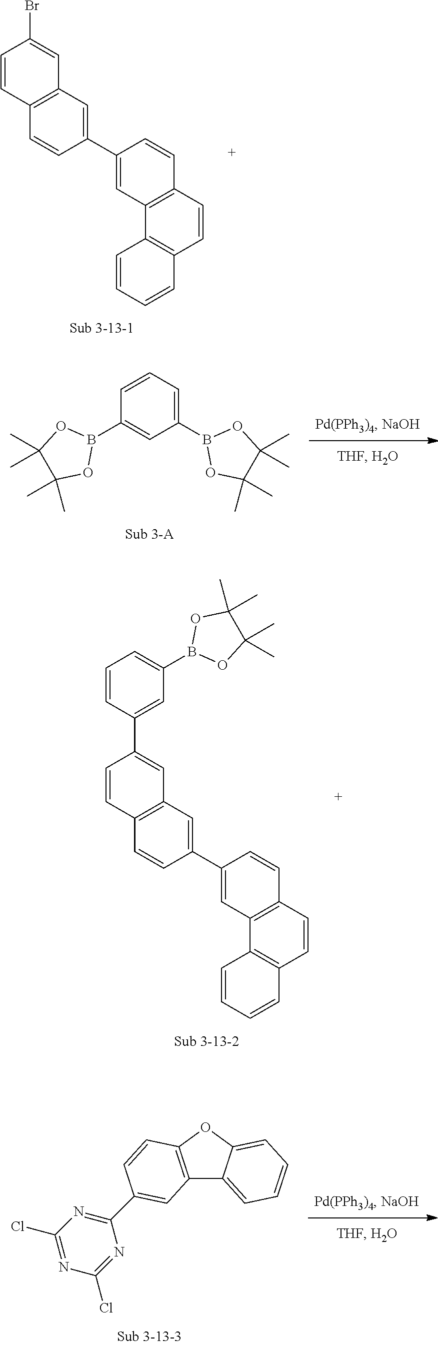

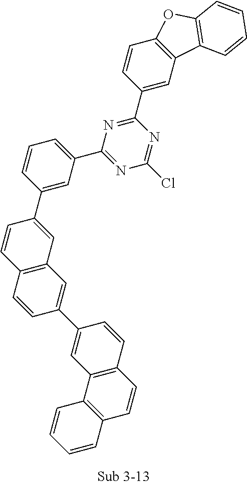 [SMILES: Clc1nc(-c2cccc(-c3ccc4ccc(-c5ccc6ccc7ccccc7c6c5)cc4c3)c2)nc(-c2ccc3oc4ccccc4c3c2)n1]

1) Synthesis of Sub 3-13-2

After putting Sub 3-13-1 (116.1 g, 303.0 mmol) in a round bottom flask and dissolving in THF (1515 ml), Sub 3-A (100.0 g, 303.0 mmol), Pd(PPh₃)₄ (21.0 g, 18.2 mmol), NaOH (36.4 g, 909.0 mmol), Water (757 ml) were added and tested in the same manner as in Sub 3-1-2 to obtain 125.4 g of product. (Yield: 81.7%)

2) Synthesis of Sub 3-13

After putting Sub 3-13-2 (125.4 g, 247.5 mmol) in a round bottom flask and dissolving in THF (1238 ml), Sub 3-13-3 (78.3 g, 247.5 mmol), Pd(PPh₃)₄ (17.2 g, 14.9 mmol), NaOH (29.7 g, 742.6 mmol), Water (619 ml) were added and tested in the same manner as in Sub 3-1-2 to obtain 131.6 g of product. (Yield: 80.5%)

4. Synthesis of Sub 3-41

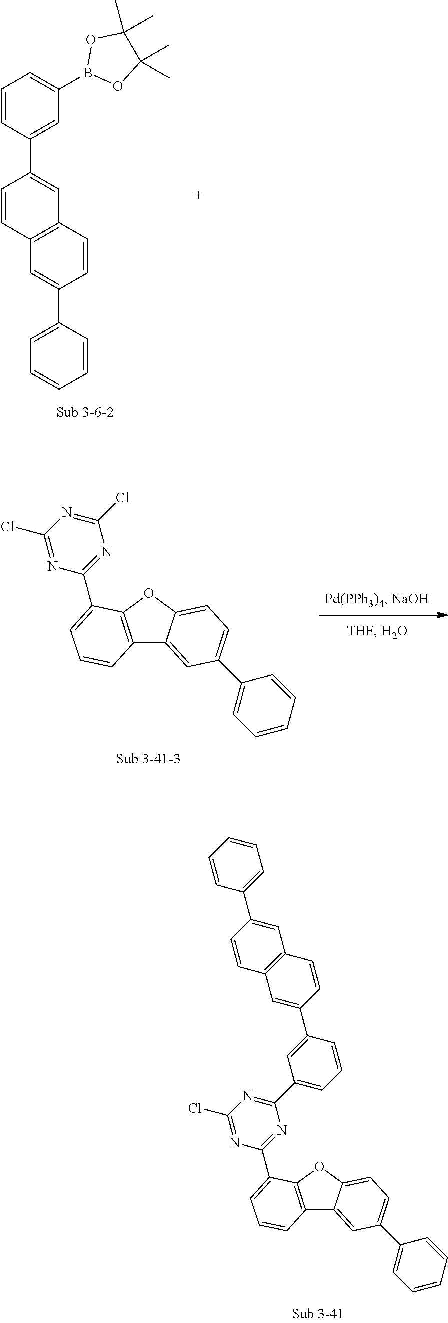 [SMILES: CC1(C)OB(c2cccc(-c3ccc4cc(-c5ccccc5)ccc4c3)c2)OC1(C)C ; Clc1nc(Cl)nc(-c2cccc3c2oc2ccc(-c4ccccc4)cc23)n1 ; Clc1nc(-c2cccc(-c3ccc4cc(-c5ccccc5)ccc4c3)c2)nc(-c2cccc3c2oc2ccc(-c4ccccc4)cc23)n1]

After putting Sub 3-6-2 (100.0 g, 246.1 mmol) in a round bottom flask and dissolving in THF (1231 ml), Sub 3-41-3 (96.5 g, 246.1 mmol), Pd(PPh₃)₄ (17.1 g, 14.8 mmol), NaOH (29.5 g, 738.3 mmol), Water (615 ml) were added and tested in the same manner as in Sub 3-1-2 to obtain 128.7 g of product. (Yield: 82.2%)

5. Synthesis of Sub 3-42

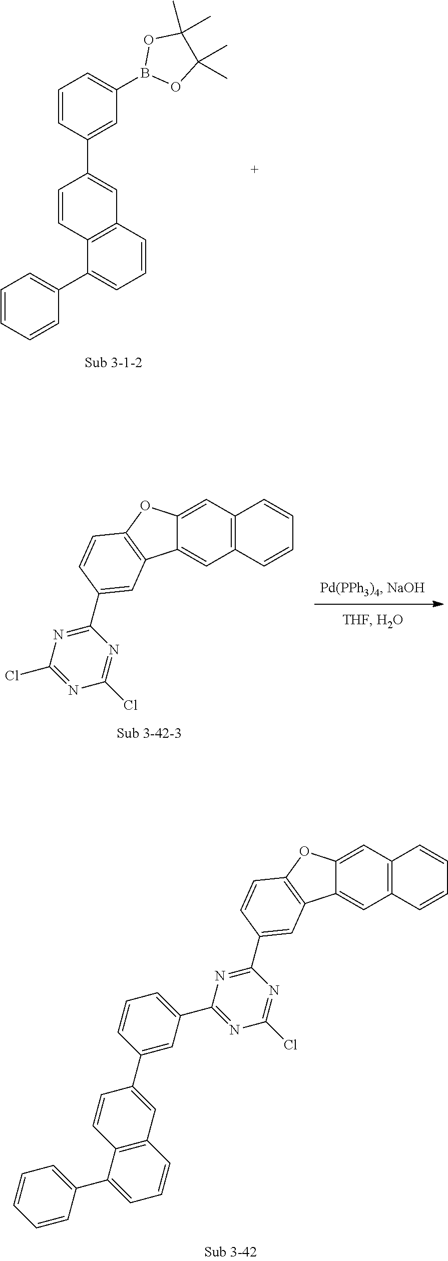 [SMILES: CC1(C)OB(c2cccc(-c3ccc4c(-c5ccccc5)cccc4c3)c2)OC1(C)C ; Clc1nc(Cl)nc(-c2ccc3oc4cc5ccccc5cc4c3c2)n1 ; Clc1nc(-c2cccc(-c3ccc4c(-c5ccccc5)cccc4c3)c2)nc(-c2ccc3oc4cc5ccccc5cc4c3c2)n1]

After putting Sub 3-1-2 (100.0 g, 246.1 mmol) in a round bottom flask and dissolving in THF (1231 ml), Sub 3-42-3 (90.1 g, 246.1 mmol), Pd(PPh₃)₄ (17.1 g, 14.8 mmol), NaOH (29.5 g, 738.3 mmol), Water (615 ml) were added and tested in the same manner as in Sub 3-1-2 to obtain 122.1 g of product. (Yield: 81.3%)

6. Synthesis of Sub 3-48

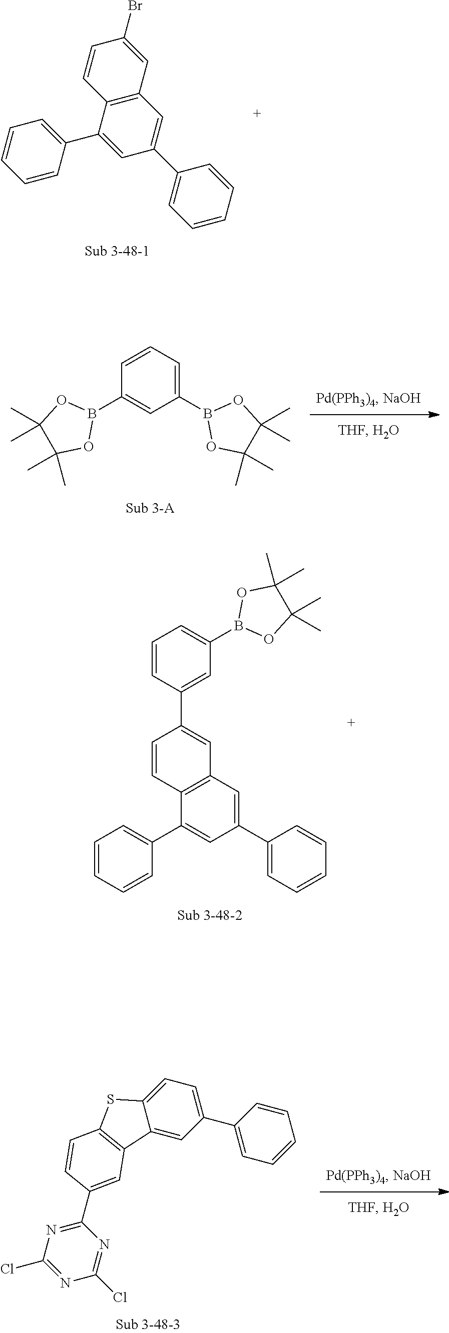

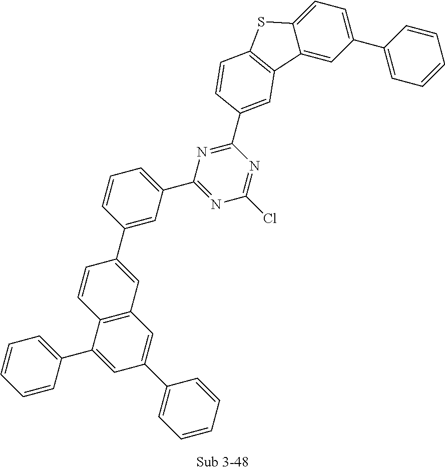 [SMILES: Clc1nc(-c2cccc(-c3ccc4c(-c5ccccc5)cc(-c5ccccc5)cc4c3)c2)nc(-c2ccc3sc4ccc(-c5ccccc5)cc4c3c2)n1]

1) Synthesis of Sub 3-48-2

After putting Sub 3-48-1 (108.9 g, 303.0 mmol) in a round bottom flask and dissolving in THF (1515 ml), Sub 3-A (100.0 g, 303.0 mmol), Pd(PPh₃)₄ (21.0 g, 18.2 mmol), NaOH (36.4 g, 909.0 mmol), Water (757 ml) were added and tested in the same manner as in Sub 3-1-2 to obtain 118.3 g of product. (Yield: 80.9%)

2) Synthesis of Sub 3-48

After putting Sub 3-48-2 (118.3 g, 245.1 mmol) in a round bottom flask and dissolving in THF (1226 ml), Sub 3-48-3 (100.1 g, 245.1 mmol), Pd(PPh₃)₄ (17.0 g, 14.7 mmol), NaOH (29.4 g, 735.4 mmol), Water (613 ml) were added and tested in the same manner as in Sub 3-1-2 to obtain 143.0 g of product. (Yield: 80.1%)

7. Synthesis of Sub 3-64

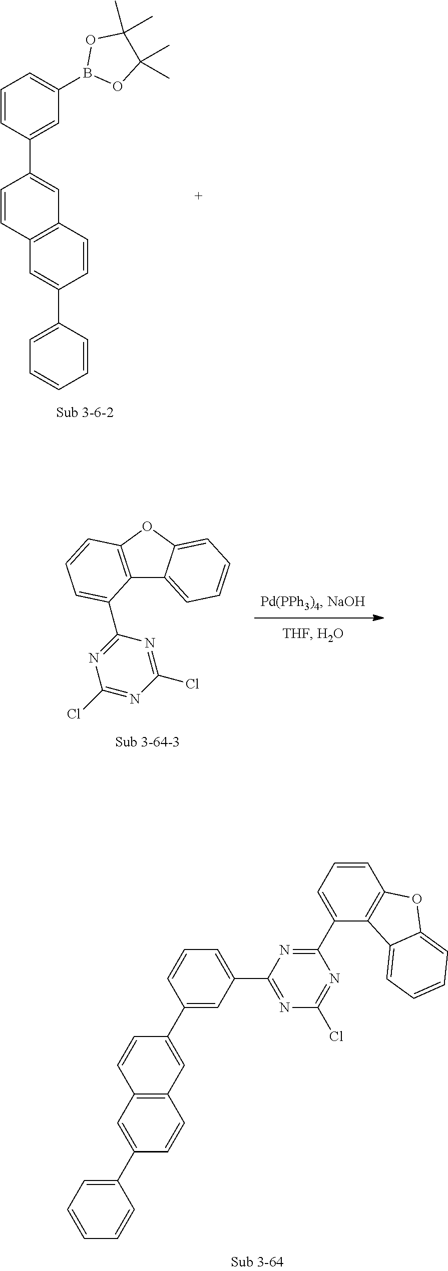 [SMILES: CC1(C)OB(c2cccc(-c3ccc4cc(-c5ccccc5)ccc4c3)c2)OC1(C)C ; Clc1nc(Cl)nc(-c2cccc3oc4ccccc4c23)n1 ; Clc1nc(-c2cccc(-c3ccc4cc(-c5ccccc5)ccc4c3)c2)nc(-c2cccc3oc4ccccc4c23)n1]

After putting Sub 3-6-2 (100.0 g, 246.1 mmol) in a round bottom flask and dissolving in THF (1231 ml), Sub 3-64-3 (77.8 g, 246.1 mmol), Pd(PPh₃)₄ (17.1 g, 14.8 mmol), NaOH (29.5 g, 738.3 mmol), Water (615 ml) were added and tested in the same manner as in Sub 3-1-2 to obtain 114.4 g of product. (Yield: 83.0%)

8. Synthesis of Sub 3-9

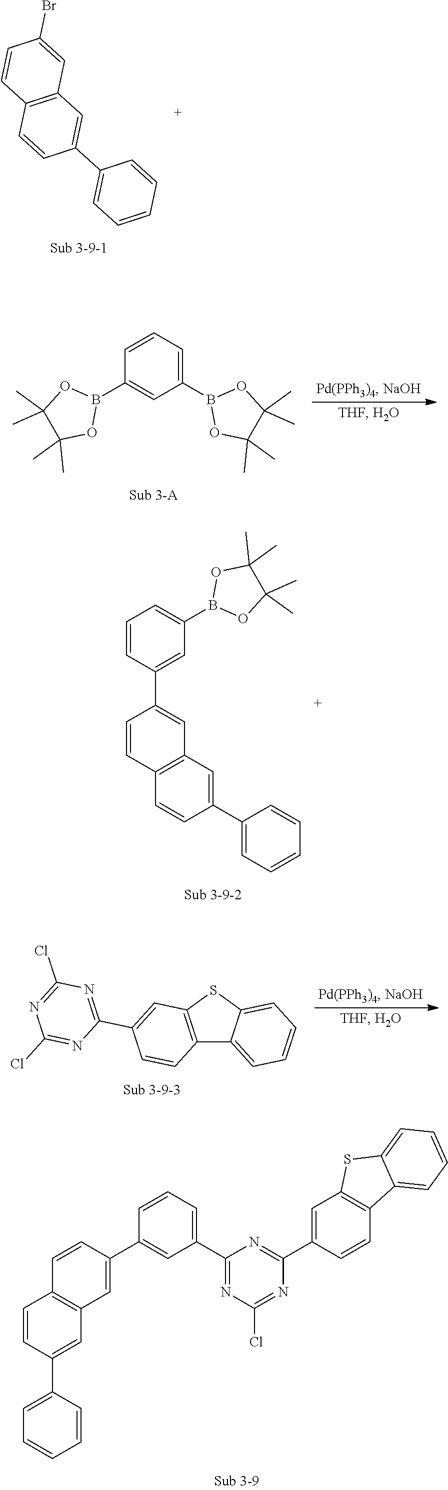

1) Synthesis of Sub 3-9-2

After putting Sub 3-9-1 (85.8 g, 303.0 mmol) in a round bottom flask and dissolving in THF (1515 ml), Sub 3-A (100.0 g, 303.0 mmol), Pd(PPh₃)₄ (21.0 g, 18.2 mmol), NaOH (36.4 g, 909.0 mmol), Water (757 ml) were added and tested in the same manner as in Sub 3-1-2 to obtain 100.8 g of product. (Yield: 81.9%)

2) Synthesis of Sub 3-9

After putting Sub 3-9-2 (100.8 g, 248.2 mmol) in a round bottom flask and dissolving in THF (1223 ml), Sub 3-9-3 (82.4 g, 248.2 mmol), Pd(PPh₃)₄ (17.2 g, 14.9 mmol), NaOH (29.8 g, 744.5 mmol), Water (620 ml) were added and tested in the same manner as in Sub 3-1-2 to obtain 117.4 g of product. (Yield: 82.1%)

The compound belonging to Sub 3 may be the following compounds, but is not limited thereto, and Table 5 shows the FD-MS (Field Desorption-Mass Spectrometry) values of the compounds belonging to Sub 3.

[chemical structure]

[chemical structure]

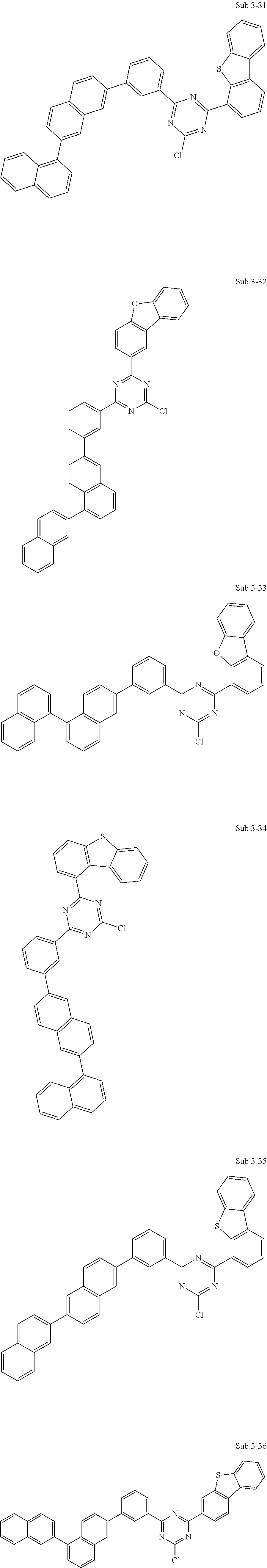 [SMILES: Clc1nc(-c2cccc(-c3ccc4ccc(-c5cccc6ccccc56)cc4c3)c2)nc(-c2cccc3c2sc2ccccc23)n1 ; Clc1nc(-c2cccc(-c3ccc4c(-c5ccc6ccccc6c5)cccc4c3)c2)nc(-c2ccc3oc4ccccc4c3c2)n1 ; Clc1nc(-c2cccc(-c3ccc4c(-c5cccc6ccccc56)cccc4c3)c2)nc(-c2cccc3c2oc2ccccc23)n1 ; Clc1nc(-c2cccc(-c3ccc4cc(-c5cccc6ccccc56)ccc4c3)c2)nc(-c2cccc3sc4ccccc4c23)n1 ; Clc1nc(-c2cccc(-c3ccc4cc(-c5ccc6ccccc6c5)ccc4c3)c2)nc(-c2cccc3c2sc2ccccc23)n1 ; Clc1nc(-c2cccc(-c3ccc4c(-c5ccc6ccccc6c5)cccc4c3)c2)nc(-c2ccc3c(c2)sc2ccccc23)n1]

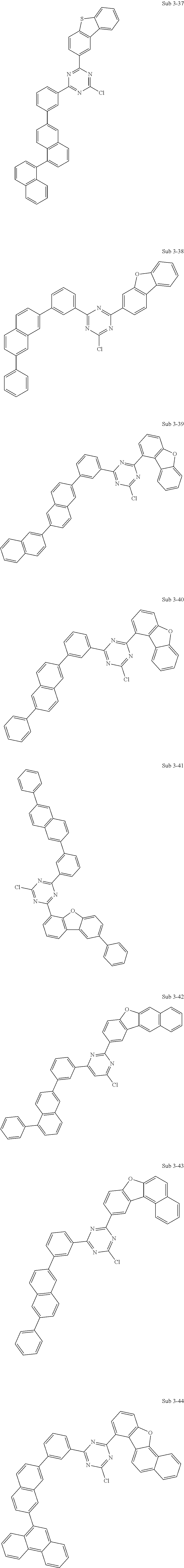

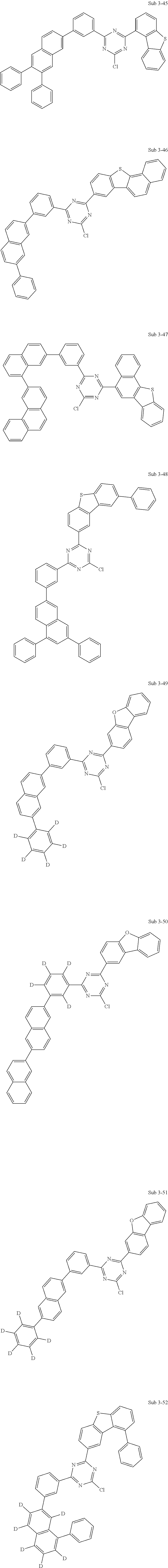

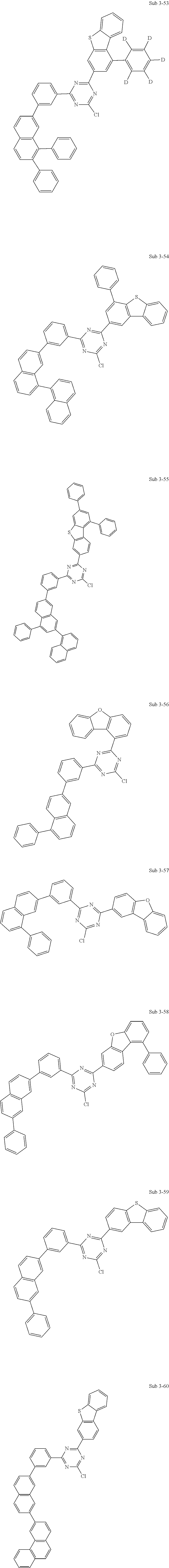

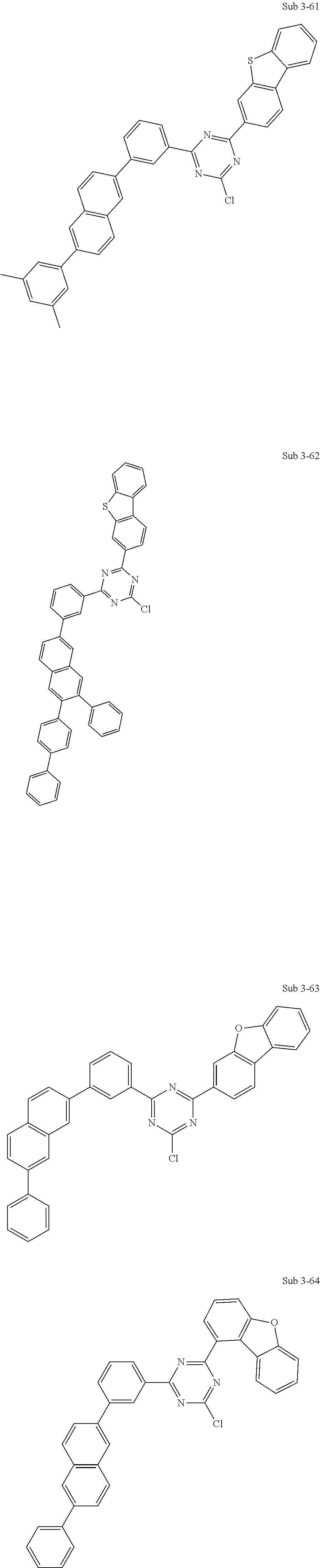 [SMILES: Cc1cc(C)cc(-c2ccc3cc(-c4cccc(-c5nc(Cl)nc(-c6ccc7c(c6)sc6ccccc67)n5)c4)ccc3c2)c1 ; Clc1nc(-c2cccc(-c3ccc4cc(-c5ccc(-c6ccccc6)cc5)c(-c5ccccc5)cc4c3)c2)nc(-c2ccc3c(c2)sc2ccccc23)n1 ; Clc1nc(-c2cccc(-c3ccc4ccc(-c5ccccc5)cc4c3)c2)nc(-c2ccc3c(c2)oc2ccccc23)n1 ; Clc1nc(-c2cccc(-c3ccc4cc(-c5ccccc5)ccc4c3)c2)nc(-c2cccc3oc4ccccc4c23)n1]

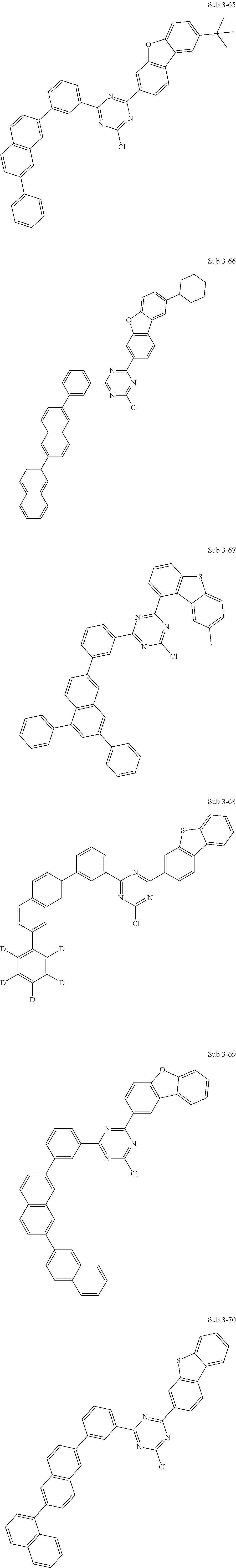

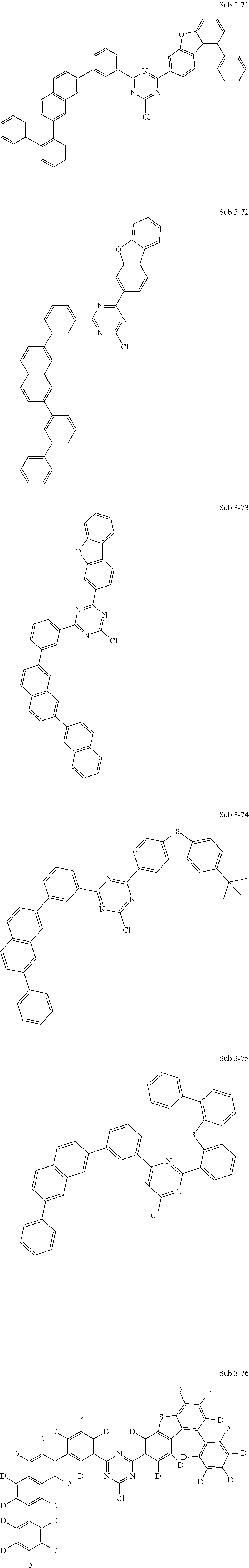

TABLE 5

| Compound | FD-MS | Compound | FD-MS |
|---|---|---|---|
| Sub 3-1 | m/z = 575.12(C₃₇H₂₂ClN₃S = 576.11) | Sub 3-2 | m/z = 575.12(C₃₇H₂₂ClN₃ = 576.11) |
| Sub 3-3 | m/z = 651.15(C₄₃H₂₆ClN₃S = 652.21) | Sub 3-4 | m/z = 575.12(C₃₇H₂₂ClN₃S = 576.11) |
| Sub 3-5 | m/z = 559.15(C₃₇H₂₂ClN₃O = 560.05) | Sub 3-6 | m/z = 559.15(C₃₇H₂₂ClN₃O = 560.05) |
| Sub 3-7 | m/z = 609.16(C₄₁H₂₄ClN₃O = 610.11) | Sub 3-8 | m/z = 609.16(C₄₁H₂₄ClN₃O = 610.11) |
| Sub 3-9 | m/z = 575.12(C₃₇H₂₂ClN₃S = 576.11) | Sub 3-10 | m/z = 625.14(C₄₁H₂₄ClN₃S = 626.17) |
| Sub 3-11 | m/z = 559.15(C₃₇H₂₂ClN₃O = 560.05) | Sub 3-12 | m/z = 659.18(C₄₅H₂₆ClN₃O = 660.17) |
| Sub 3-13 | m/z = 659.18(C₄₅H₂₆ClN₃O = 660.17) | Sub 3-14 | m/z = 635.18(C₄₃H₂₆ClN₃O = 636.15) |
| Sub 3-15 | m/z = 651.15(C₄₃H₂₆ClN₃S = 652.21) | Sub 3-16 | m/z = 651.15(C₄₃H₂₆ClN₃S = 652.21) |
| Sub 3-17 | m/z = 651.15(C₄₃H₂₆ClN₃S = 652.21) | Sub 3-18 | m/z = 625.14(C₄₁H₂₄ClN₃S = 626.17) |
| Sub 3-19 | m/z = 559.15(C₃₇H₂₂ClN₃O = 560.05) | Sub 3-20 | m/z = 559.15(C₃₇H₂₂ClN₃O = 560.05) |
| Sub 3-21 | m/z = 559.15(C₃₇H₂₂ClN₃O = 560.05) | Sub 3-22 | m/z = 575.12(C₃₇H₂₂ClN₃S = 576.11) |
| Sub 3-23 | m/z = 575.12(C₃₇H₂₂ClN₃S = 576.11) | Sub 3-24 | m/z = 609.16(C₄₁H₂₄ClN₃O = 610.11) |
| Sub 3-25 | m/z = 609.16(C₄₁H₂₄ClN₃O = 610.11) | Sub 3-26 | m/z = 609.16(C₄₁H₂₄ClN₃O = 610.11) |
| Sub 3-27 | m/z = 609.16(C₄₁H₂₄ClN₃O = 610.11) | Sub 3-28 | m/z = 625.14(C₄₁H₂₄ClN₃S = 626.17) |
| Sub 3-29 | m/z = 625.14(C₄₁H₂₄ClN₃S = 626.17) | Sub 3-30 | m/z = 625.14(C₄₁H₂₄ClN₃S = 626.17) |
| Sub 3-31 | m/z = 625.14(C₄₁H₂₄ClN₃S = 626.17) | Sub 3-32 | m/z = 609.16(C₄₁H₂₄ClN₃O = 610.11) |
| Sub 3-33 | m/z = 609.16(C₄₁H₂₄ClN₃O = 610.11) | Sub 3-34 | m/z = 625.14(C₄₁H₂₄ClN₃S = 626.17) |
| Sub 3-35 | m/z = 625.14(C₄₁H₂₄ClN₃S = 626.17) | Sub 3-36 | m/z = 625.14(C₄₁H₂₄ClN₃S = 626.17) |
| Sub 3-37 | m/z = 625.14(C₄₁H₂₄ClN₃S = 626.17) | Sub 3-38 | m/z = 559.15(C₃₇H₂₂ClN₃O = 560.05) |
| Sub 3-39 | m/z = 609.16(C₄₁H₂₄ClN₃O = 610.11) | Sub 3-40 | m/z = 559.15(C₃₇H₂₂ClN₃O = 560.05) |
| Sub 3-41 | m/z = 635.18(C₄₃H₂₆ClN₃O = 636.15₎ | Sub 3-42 | m/z = 609.16(C₄₁H₂₄ClN₃O = 610.11) |
| Sub 3-43 | m/z = 609.16(C₄₁H₂₄ClN₃O = 610.11) | Sub 3-44 | m/z = 709.19(C₄₉H₂₈ClN₃O = 710.23) |
| Sub 3-45 | m/z = 651.15(C₄₃H₂₆ClN₃S = 652.21) | Sub 3-46 | m/z = 625.14(C₄₁H₂₄ClN₃S = 626.17) |
| Sub 3-47 | m/z = 725.17(C₄₉H₂₈ClN₃S = 726.29) | Sub 3-48 | m/z = 727.18(C₄₉H₃₀ClN₃S = 728.31) |
| Sub 3-49 | m/z = 564.18(C₃₇H₁₇D₅ClN₃O = 565.08) | Sub 3-50 | m/z = 613.19(C₄₁H₂₀D₄ClN₃O = 614.14) |
| Sub 3-51 | m/z = 564.18(C₃₇H₁₇D₅ClN₃O = 565.08) | Sub 3-52 | m/z = 657.19(C₄₃H₂₀D₆ClN₃S = 658.25) |
| Sub 3-53 | m/z = 732.22(C₄₉H₂₅D₅ClN₃S = 733.34) | Sub 3-54 | m/z = 701.17(C₄₇H₂₈ClN₃S = 702.27) |
| Sub 3-55 | m/z = 853.23(C₅₉H₃₆ClN₃S = 854.47) | Sub 3-56 | m/z = 559.15(C₃₇H₂₂ClN₃O = 560.05) |
| Sub 3-57 | m/z = 559.15(C₃₇H₂₂ClN₃O = 560.05) | Sub 3-58 | m/z = 635.18(C₄₃H₂₆ClN₃O = 636.15) |
| Sub 3-59 | m/z = 575.12(C₃₇H₂₂ClN₃S = 576.11) | Sub 3-60 | m/z = 675.15(C₄₅H₂₆ClN₃S = 676.23) |
| Sub 3-61 | m/z = 603.15(C₃₉H₂₆ClN₃S = 604.17) | Sub 3-62 | m/z = 727.18(C₄₉H₃₀ClN₃S = 728.31) |
| Sub 3-63 | m/z = 559.15(C₃₇H₂₂ClN₃O = 560.05) | Sub 3-64 | m/z = 559.15(C₃₇H₂₂ClN₃O = 560.05) |
| Sub 3-65 | m/z = 615.21(C₄₁H₃₀ClN₃O = 616.16) | Sub 3-66 | m/z = 691.24(C₄₇H₃₄ClN₃O = 692.26) |
| Sub 3-67 | m/z = 665.17(C₄₄H₂₈ClN₃S = 666.24) | Sub 3-68 | m/z = 580.15(C₃₇H₁₇D₅ClN₃S = 581.14) |
| Sub 3-69 | m/z = 609.16(C₄₁H₂₄ClN₃O = 610.11) | Sub 3-70 | m/z = 625.14(C₄₁H₂₄ClN₃S = 626.17) |
| Sub 3-71 | m/z = 711.21(C₄₉H₃₀ClN₃O = 712.25) | Sub 3-72 | m/z = 635.18(C₄₃H₂₆ClN₃O = 636.15) |
| Sub 3-73 | m/z = 609.16(C₄₁H₂₄ClN₃O = 610.11) | Sub 3-74 | m/z = 631.18(C₄₁H₃₀ClN₃S = 632.22) |
| Sub 3-75 | m/z = 651.15(C₄₃H₂₆ClN₃S = 652.21) | Sub 3-76 | m/z = 677.32(C₄₃D₂₆ClN₃S = 678.37) |

Compounds belonging to Sub 4 may be the following compounds, but are not limited thereto, and Table 6 shows ED-MS (Field Desorption-Mass Spectrometry) values of compounds belonging to Sub 6.

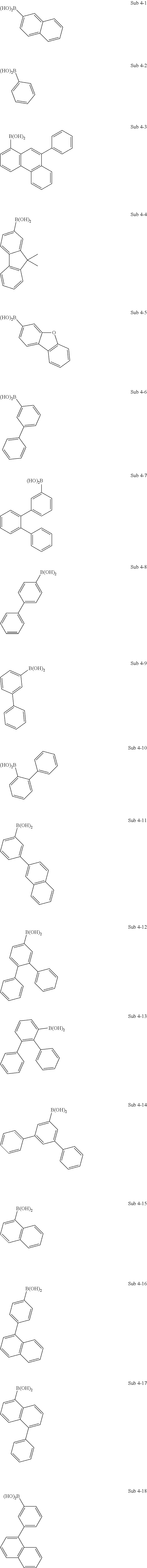

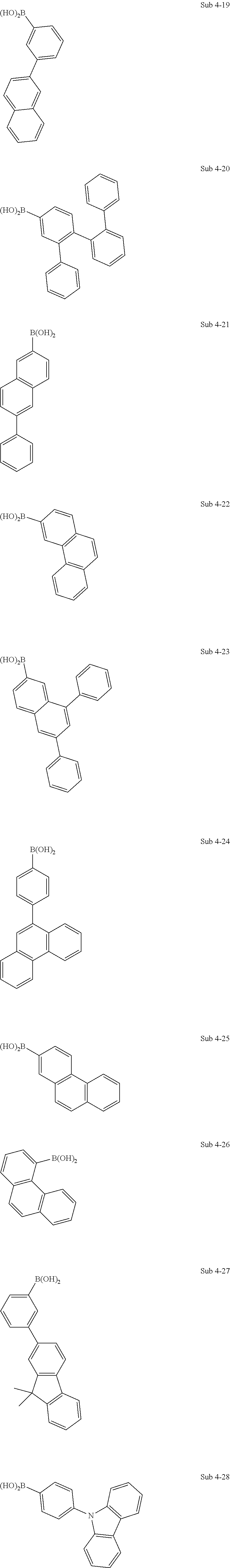

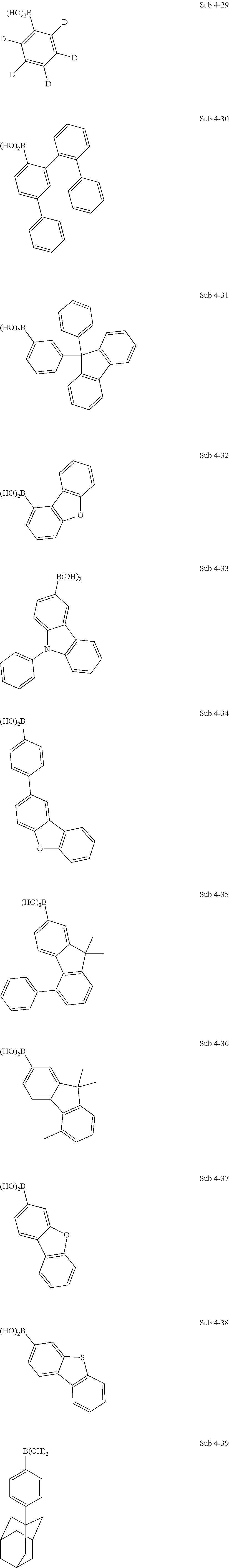

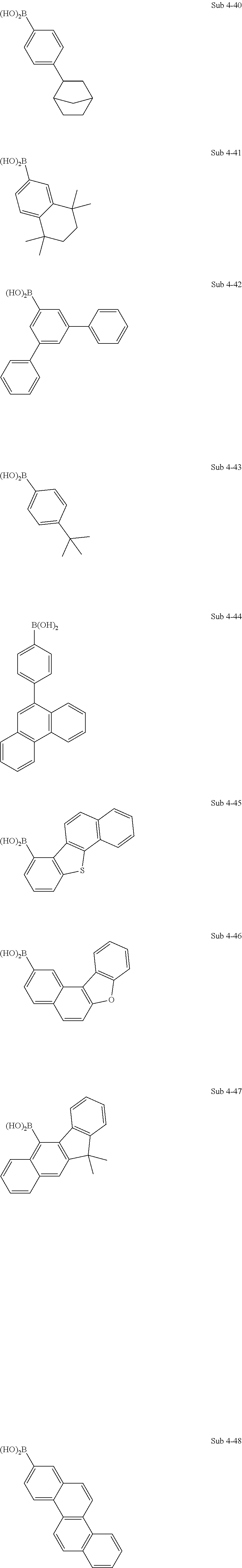

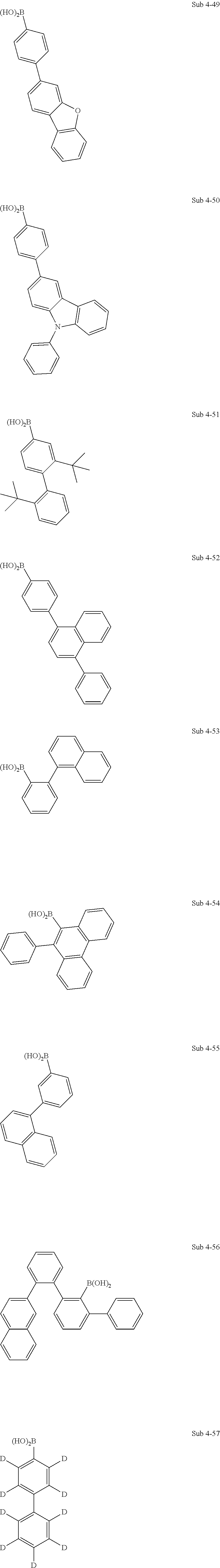

TABLE 6

| Compound | FD-MS | Compound | FD-MS |
|---|---|---|---|
| Sub 4-1 | m/z = 172.07(C₁₀H₉BO₂ = 171.99) | Sub 4-2 | m/z = 122.05(C₆H₇BO₂ = 121.93) |
| Sub 4-3 | m/z = 298.12(C₂₀H₁₅BO₂ = 298.15) | Sub 4-4 | m/z = 238.12(C₁₅H₁₅BO₂ = 238.09) |
| Sub 4-5 | m/z = 212.06(C₁₂H₉BO₃ = 212.01) | Sub 4-6 | m/z = 198.09(C₁₂H₁₁BO₂ = 198.03) |
| Sub 4-7 | m/z = 274.12(C₁₈H₁₅BO₂ = 274.13) | Sub 4-8 | m/z = 198.09(C₁₂H₁₁BO₂ = 198.03) |
| Sub 4-9 | m/z = 198.09(C₁₂H₁₁BO₂ = 198.03) | Sub 4-10 | m/z = 198.09(C₁₂H₁₁BO₂ = 198.03) |
| Sub 4-11 | m/z = 248.10(C₁₆H₁₃BO₂ = 248.09) | Sub 4-12 | m/z = 274.12(C₁₈H₁₅BO₂ = 274.13) |
| Sub 4-13 | m/z = 274.12(C₁₈H₁₅BO₂ = 274.13) | Sub 4-14 | m/z = 274.12(C₁₈H₁₅BO₂ = 274.13) |
| Sub 4-15 | m/z = 172.07(C₁₀H₉BO₂ = 171.99) | Sub 4-16 | m/z = 248.10(C₁₆H₁₃BO₂ = 248.09) |
| Sub 4-17 | m/z = 248.10(C₁₆H₁₃BO₂ = 248.09) | Sub 4-18 | m/z = 248.10(C₁₆H₁₃BO₂ = 248.09) |
| Sub 4-19 | m/z = 248.10(C₁₆H₁₃BO₂ = 248.09) | Sub 4-20 | m/z = 350.15(C₂₄H₁₉BO₂ = 350.22) |
| Sub 4-21 | m/z = 248.10(C₁₆H₁₃BO₂ = 248.09) | Sub 4-22 | m/z = 222.09(C₁₄H₁₁BO₂ = 222.05) |
| Sub 4-23 | m/z = 324.13(C₂₂H₁₇BO₂ = 324.19) | Sub 4-24 | m/z = 298.12(C₂₀H₁₅BO₂ = 298.15) |
| Sub 4-25 | m/z = 222.09(C₁₄H₁₁BO₂ = 222.05) | Sub 4-26 | m/z = 222.09(C₁₄H₁₁BO₂ = 222.05) |
| Sub 4-27 | m/z = 314.15(C₂₁H₁₉BO₂ = 314.19) | Sub 4-28 | m/z = 287.11(C₁₈H₁₄BNO₂ = 287.12) |
| Sub 4-29 | m/z = 127.09(C₆H₂D₅BO₂ = 126.96) | Sub 4-30 | m/z = 350.15(C₂₄H₁₉BO₂ = 350.22) |
| Sub 4-31 | m/z = 362.15(C₂₅H₁₉BO₂ = 362.23) | Sub 4-32 | m/z = 212.06(C₁₂H₉BO₃ = 212.01) |
| Sub 4-33 | m/z = 287.11(C₁₈H₁₄BNO₂ = 287.12) | Sub 4-34 | m/z = 288.10(C₁₈H₁₃BO₃ = 288.11) |
| Sub 4-35 | m/z = 314.15(C₂₁H₁₉BO₂ = 314.19) | Sub 4-36 | m/z = 252.13(C₁₆H₁₇BO₂ = 252.12) |
| Sub 4-37 | m/z = 212.06(C₁₂H₉BO₃ = 212.01) | Sub 4-38 | m/z = 228.04(C₁₂H₉BO₂S = 228.07) |
| Sub 4-39 | m/z = 256.16(C₁₆H₂₁BO₂ = 256.15) | Sub 4-40 | m/z = 216.13(C₁₃H₁₇BO₂ = 216.09) |
| Sub 4-41 | m/z = 232.16(C₁₄H₂₁BO₂ = 232.13) | Sub 4-42 | m/z = 274.12(C₁₈H₁₅BO₂ = 274.13) |
| Sub 4-43 | m/z = 178.12(C₁₀H₁₅BO₂ = 178.04) | Sub 4-44 | m/z = 298.12(C₂₀H₁₅BO₂ = 298.15) |
| Sub 4-45 | m/z = 278.06(C₁₆H₁₁BO₂S = 278.13) | Sub 4-46 | m/z = 262.08(C₁₆H₁₁BO₃ = 262.07) |
| Sub 4-47 | m/z = 288.13(C₁₉H₁₇BO₂ = 288.15) | Sub 4-48 | m/z = 272.10(C₁₈H₁₃BO₂ = 272.11) |
| Sub 4-49 | m/z = 288.10(C₁₈H₁₃BO₃ = 288.11) | Sub 4-50 | m/z = 363.14(C₂₄H₁₈BNO₂ = 363.22) |
| Sub 4-51 | m/z = 310.21(C₂₀H₂₇BO₂ = 310.24) | Sub 4-52 | m/z = 324.13(C₂₂H₁₇BO₂ = 324.19) |
| Sub 4-53 | m/z = 248.10(C₁₆H₁₃BO₂ = 248.09) | Sub 4-54 | m/z = 298.12(C₂₀H₁₅BO₂ = 298.15) |
| Sub 4-55 | m/z = 248.10(C₁₆H₁₃BO₂ = 248.09) | Sub 4-56 | m/z = 400.16(C₂₈H₂₁BO₂ = 400.28) |
| Sub 4-57 | m/z = 207.14(C₁₂H₂D₉BO₂ = 207.08) | | |

II. Synthesis of Final Product

1. Synthesis Example of N 1-1

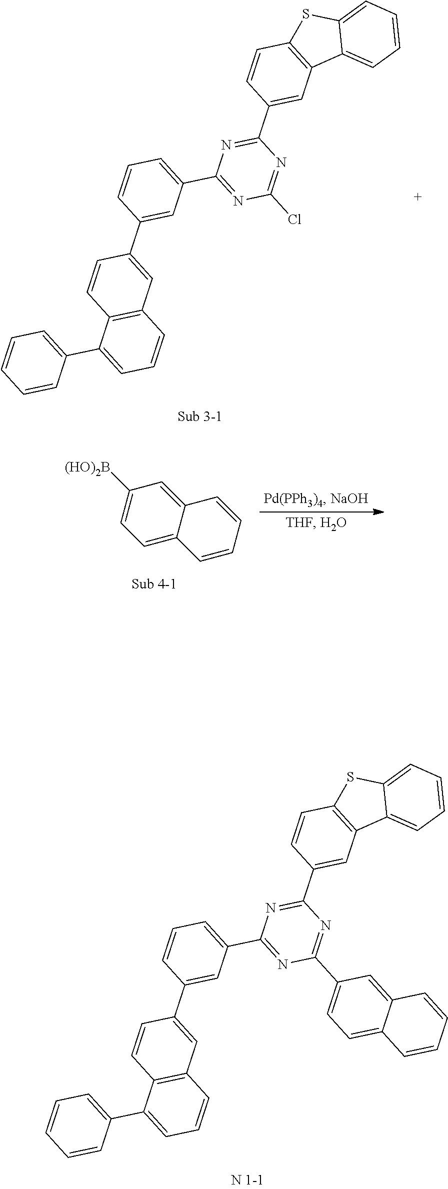

After putting Sub 3-1 (50.0 g, 86.8 mmol) in a round bottom flask and dissolving in THF (434 ml), Sub 4-1 (14.9 g, 86.8 mmol), Pd(PPh₃)₄ (6.0 g, 5.2 mmol), NaOH (10.4 g, 260.4 mmol), Water (217 ml) were added and tested in the same manner as in Sub 3-1-2 to obtain 47.2 g of product. (Yield: 81.4%)

2. Synthesis Example of N 1-6

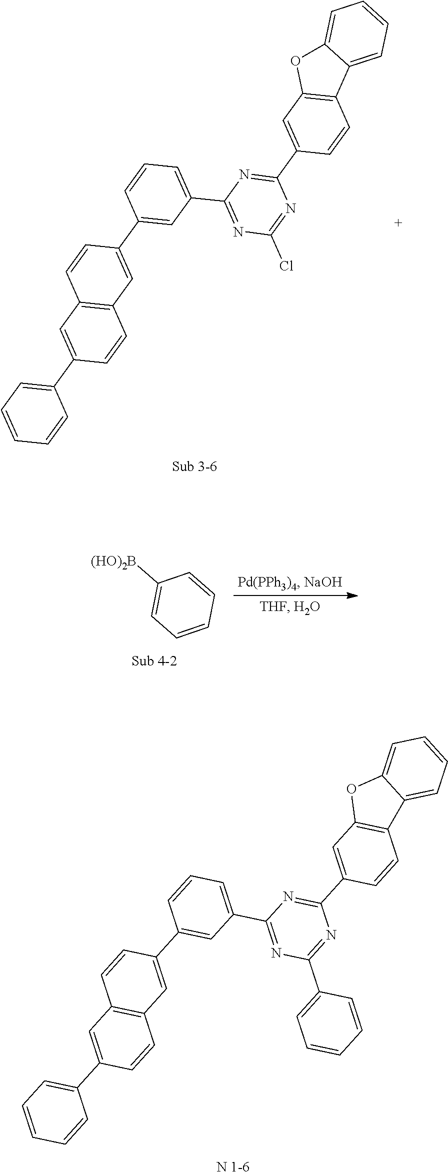

After putting Sub 3-6 (50.0 g, 89.3 mmol) in a round bottom flask and dissolving in THF (446 ml), Sub 4-2 (10.9 g, 89.3 mmol), Pd(PPh₃)₄ (6.2 g, 5.4 mmol), NaOH (10.7 g, 267.8 mmol), Water (223 ml) were added and tested in the same manner as in Sub 3-1-2 to obtain 44.5 g of product. (Yield: 82.8%)

3. Synthesis Example of N 1-15

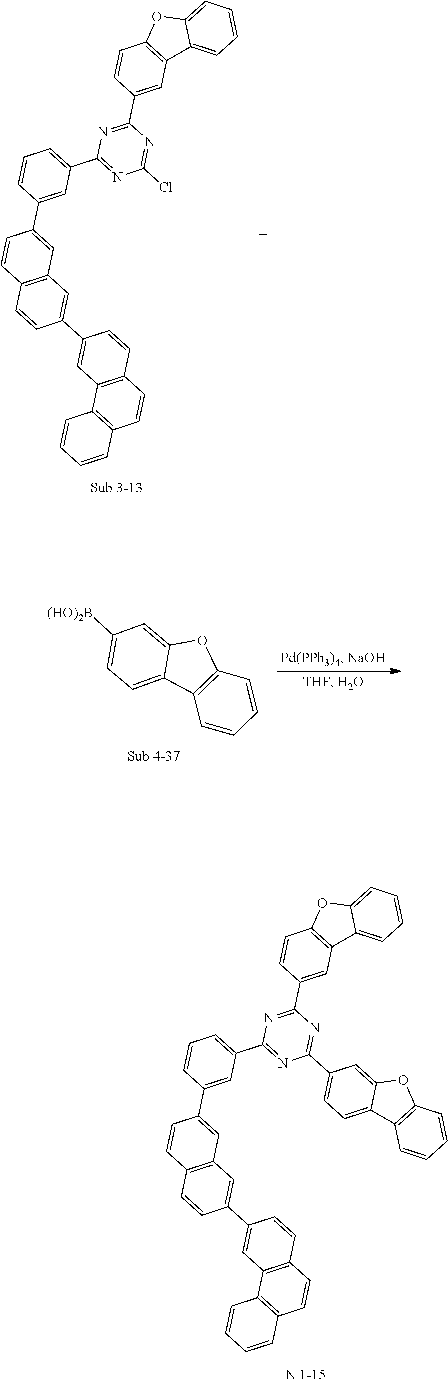

After putting Sub 3-13 (50.0 g, 75.7 mmol) in a round bottom flask and dissolving in THF (379 ml), Sub 4-37 (16.1 g, 75.7 mmol), Pd(PPh₃)₄ (5.3 g, 4.5 mmol), NaOH (9.1 g, 227.2 mmol), Water (189 ml) were added and tested in the same manner as in Sub 3-1-2 to obtain 48.4 g of product. (Yield: 80.7%)

4. Synthesis Example of N 1-49

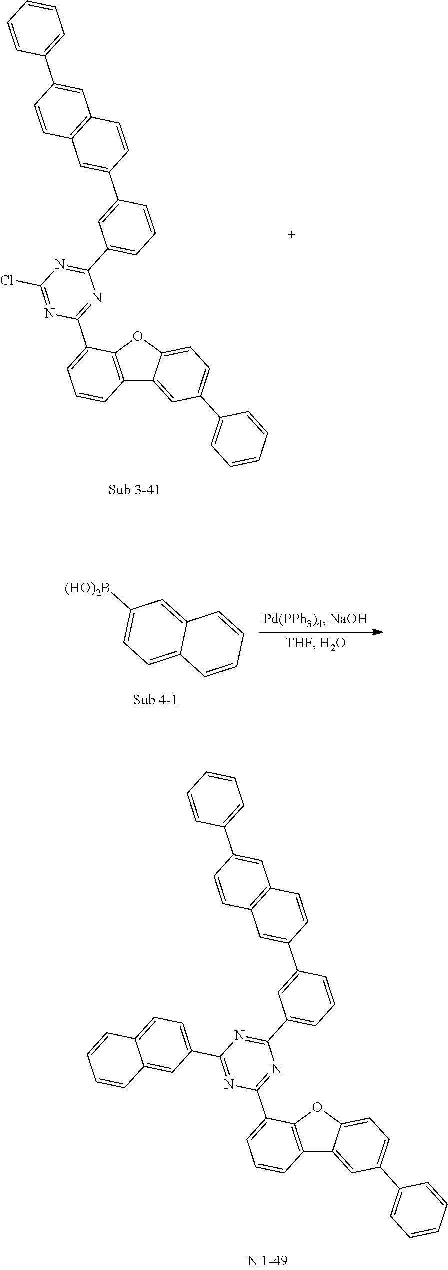

After putting Sub 3-41 (50.0 g, 78.6 mmol) in a round bottom flask and dissolving in THF (393 ml), Sub 4-1 (13.5 g, 78.6 mmol), Pd(PPh₃)₄ (5.5 g, 4.7 mmol), NaOH (9.4 g, 235.8 mmol), Water (196 ml) were added and tested in the same manner as in Sub 3-1-2 to obtain 46.9 g of product. (Yield: 81.9%)

5. Synthesis Example of N 1-50

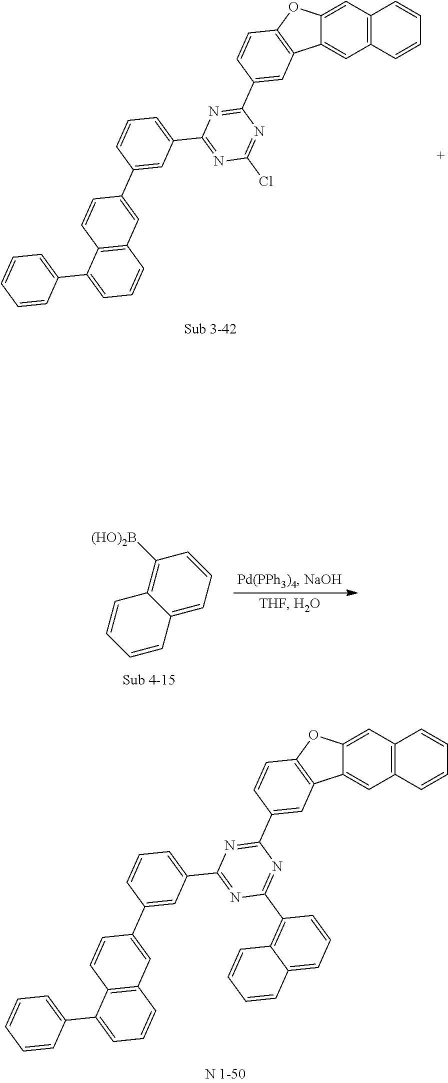

After putting Sub 3-42 (50.0 g, 82.0 mmol) in a round bottom flask and dissolving in THF (410 ml), Sub 4-15 (14.1 g, 82.0 mmol), Pd(PPh₃)₄ (5.7 g, 4.9 mmol), NaOH (9.8 g, 245.9 mmol), Water (205 ml) were added and tested in the same manner as in Sub 3-1-2 to obtain 46.4 g of product. (Yield: 80.7%)

6. Synthesis Example of N 1-56

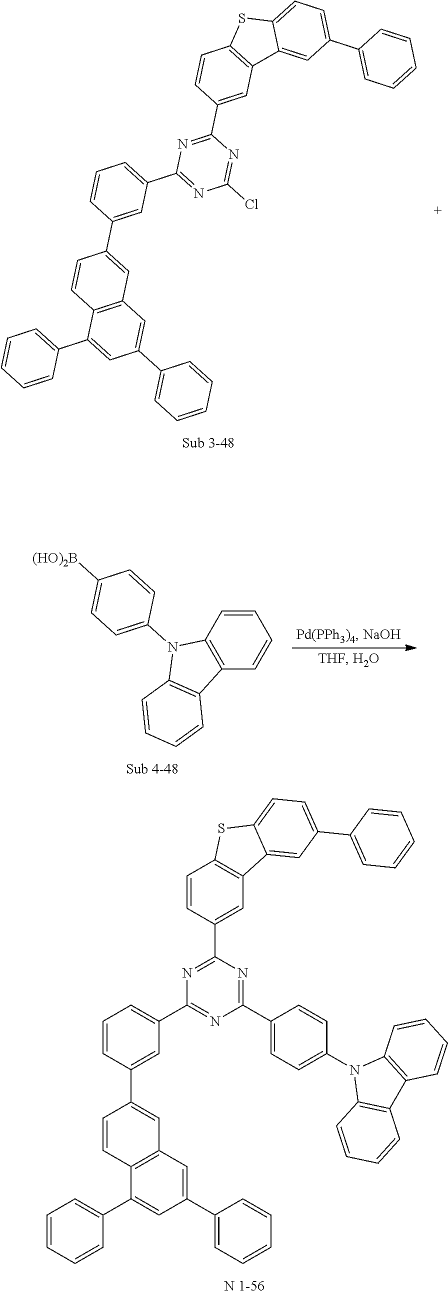

After putting Sub 3-48 (50.0 g, 68.7 mmol) in a round bottom flask and dissolving in THF (343 ml), Sub 4-28 (19.7 g, 68.7 mmol), Pd(PPh₃)₄ (4.8 g, 4.1 mmol), NaOH (8.2 g, 206.0 mmol), Water (172 ml) were added and tested in the same manner as in Sub 3-1-2 to obtain 51.4 g of product. (Yield: 80.0%)

7. Synthesis Example of N 1-60

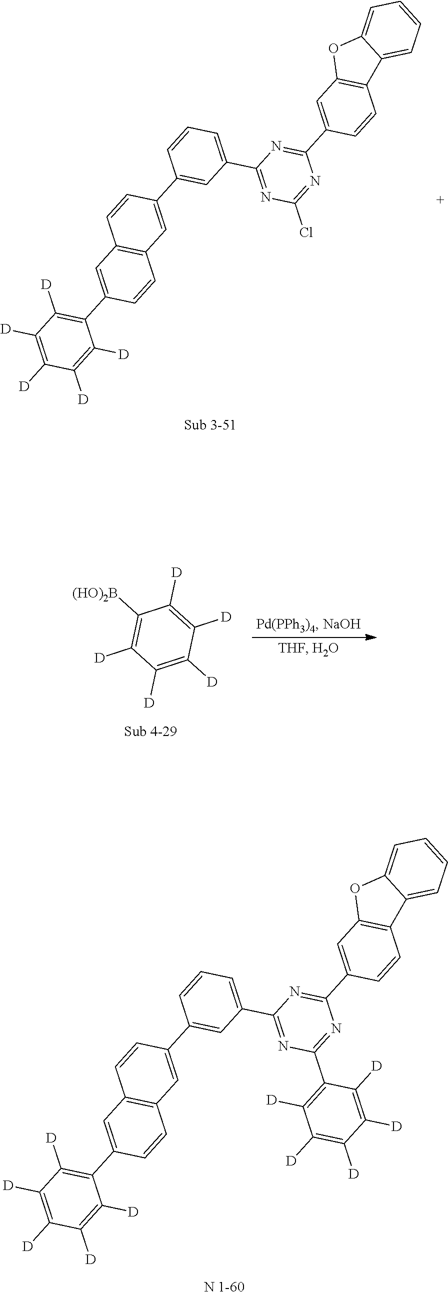

After putting Sub 3-51 (50.0 g, 88.5 mmol) in a round bottom flask and dissolving in THF (442 ml), Sub 4-29 (11.2 g, 88.5 mmol), Pd(PPh₃)₄ (6.1 g, 5.3 mmol), NaOH (10.6 g, 265.4 mmol), Water (221 ml) were added and tested in the same manner as in Sub 3-1-2 to obtain 45.5 g of product. (Yield: 84.1%)

8. Synthesis Example of N 1-74

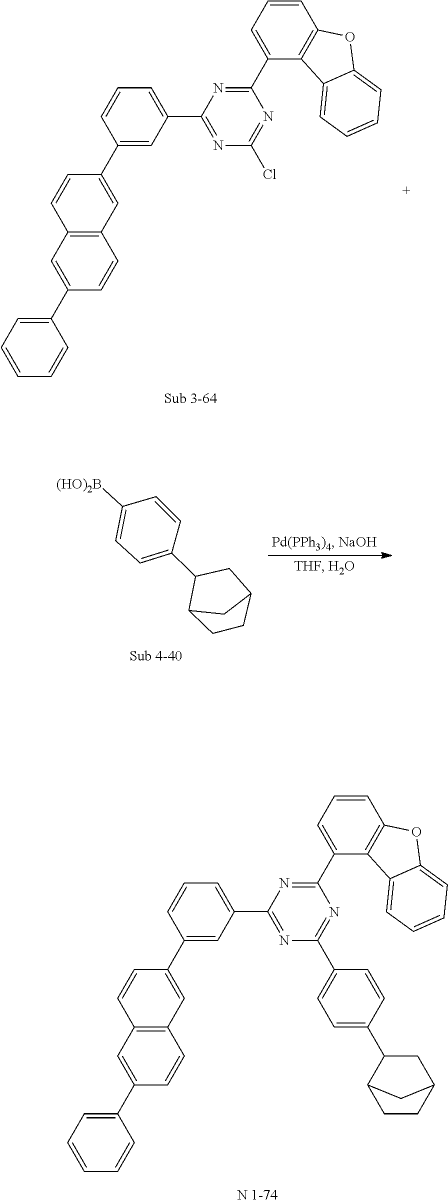

After putting Sub 3-64 (50.0 g, 89.3 mmol) in a round bottom flask and dissolving in THF (446 ml), Sub 4-40 (19.3 g, 89.3 mmol), Pd(PPh₃)₄ (6.2 g, 5.4 mmol), NaOH (10.7 g, 267.8 mmol), Water (223 ml) were added and tested in the same manner as in Sub 3-1-2 to obtain 51.8 g of product. (Yield: 83.4%)

9. Synthesis Example of N 1-78

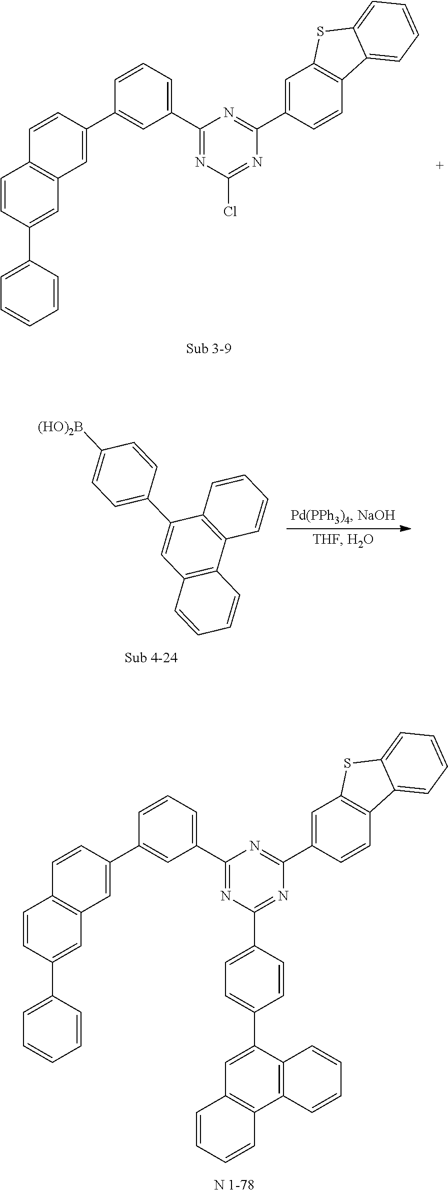

After putting Sub 3-9 (50.0 g, 86.8 mmol) in a round bottom flask and dissolving in THF (434 ml), Sub 4-24 (25.9 g, 86.8 mmol), Pd(PPh₃)₄ (6.0 g, 5.2 mmol), NaOH (10.4 g, 260.4 mmol), Water (217 ml) were added and tested in the same manner as in Sub 3-1-2 to obtain 55.5 g of product. (Yield: 80.5%)

Table 7 shows the FD-MS (Field Desorption-Mass Spectrometry) values of the compounds N 1-1 to N 1-100 of the present invention prepared according to the Synthesis Example as described.

TABLE 7

| Compound | FD-MS | Compound | FD-MS |
|---|---|---|---|
| N 1-1 | m/z = 667.21(C₄₇H₂₉N₃S = 667.83) | N 1-2 | m/z = 667.21(C₄₇H₂₉N₃S = 667.83) |
| N 1-3 | m/z = 693.22(C₄₉H₃₁N₃S = 693.87) | N 1-4 | m/z = 617.19(C₄₃H₂₇N₃S = 617.77) |
| N 1-5 | m/z = 601.22(C₄₃H₂₇N₃O = 601.71) | N 1-6 | m/z = 601.22(C₄₃H₂₇N₃O = 601.71) |
| N 1-7 | m/z = 651.23(C₄₇H₂₉N₃O = 651.77) | N 1-8 | m/z = 651.23(C₄₇H₂₉N₃O = 651.77) |
| N 1-9 | m/z = 667.21(C₄₇H₂₉N₃S = 667.83) | N 1-10 | m/z = 717.22(C₅₁H₃₁N₃S = 717.89) |
| N 1-11 | m/z = 793.26(C₅₇H₃₅N₃S = 793.99) | N 1-12 | m/z = 693.22(C₄₉H₃₁N₃S = 693.87) |
| N 1-13 | m/z = 717.28(C₅₂H₃₅N₃O = 717.87) | N 1-14 | m/z = 701.25(C₅₁H₃₁N₃O = 701.83) |
| N 1-15 | m/z = 791.26(C₅₇H₃₃N₃O₂ = 791.91) | N 1-16 | m/z = 677.25(C₄₉H₃₁N₃O = 677.81) |
| N 1-17 | m/z = 769.26(C₅₅H₃₅N₃S = 769.97) | N 1-18 | m/z = 693.22(C₄₉H₃₁N₃S = 693.87) |
| N 1-19 | m/z = 845.29(C₆₁H₃₉N₃S = 846.06) | N 1-20 | m/z = 717.22(C₅₁H₃₁N₃S = 717.89) |
| N 1-21 | m/z = 601.22(C₄₃H₂₇N₃O = 601.71) | N 1-22 | m/z = 677.25(C₄₉H₃₁N₃O = 677.81) |
| N 1-23 | m/z = 677.25(C₄₉H₃₁N₃O = 677.81) | N 1-24 | m/z = 677.25(C₄₉H₃₁N₃O = 677.81) |
| N 1-25 | m/z = 617.19(C₄₃H₂₇N₃S = 617.77) | N 1-26 | m/z = 667.21(C₄₇H₂₉N₃S = 667.83) |
| N 1-27 | m/z = 743.24(C₅₃H₃₃N₃S = 743.93) | N 1-28 | m/z = 769.26(C₅₅H₃₅N₃S = 769.97) |
| N 1-29 | m/z = 651.23(C₄₇H₂₉N₃O = 651.77) | N 1-30 | m/z = 701.25(C₅₁H₃₁N₃O = 701.83) |
| N 1-31 | m/z = 803.29(C₅₉H₃₇N₃O = 803.97) | N 1-32 | m/z = 803.29(C₅₉H₃₇N₃O = 803.97) |
| N 1-33 | m/z = 667.21(C₄₇H₂₉N₃S = 667.83) | N 1-34 | m/z = 717.22(C₅₁H₃₁N₃S = 717.89) |
| N 1-35 | m/z = 793.26(C₅₇H₃₅N₃S = 793.99) | N 1-36 | m/z = 793.26(C₅₇H₃₅N₃S = 793.99) |
| N 1-37 | m/z = 777.28(C₅₇H₃₅N₃O = 777.93) | N 1-38 | m/z = 777.28(C5₇H₃₅N₃O = 777.93) |
| N 1-39 | m/z = 879.32(C₆₅H₄₁N₃O = 880.06) | N 1-40 | m/z = 777.28(C₅₇H₃₅N₃O = 777.93) |
| N 1-41 | m/z = 767.24(C₅₅H₃₃N₃S = 767.95) | N 1-42 | m/z = 869.29(C₆₃H₃₉N₃S = 870.09) |
| N 1-43 | m/z = 843.27(C₆₁H₃₇N₃S = 844.05) | N 1-44 | m/z = 767.24(C₅₅H₃₃N₃S = 767.95) |
| N 1-45 | m/z = 701.25(C₅₁H₃₁N₃O = 701.83) | N 1-46 | m/z = 601.22(C₄₃H₂₇N₃O = 601.71) |
| N 1-47 | m/z = 651.23(C₄₇H₂₉N₃O = 651.77) | N 1-48 | m/z = 601.22(C₄₃H₂₇N₃O = 601.71) |
| N 1-49 | m/z = 727.26(C₅₃H₃N₃O = 727.87) | N 1-50 | m/z = 701.25(C₅₁H₃₁N₃O = 701.83) |
| N 1-51 | m/z = 727.26(C₅₃H₃₃N₃O = 727.87) | N 1-52 | m/z = 751.26(C₅₅H₃₃N₃O = 751.89) |
| N 1-53 | m/z = 793.26(C₅₇H₃₅N₃S = 793.99) | N 1-54 | m/z = 667.21(C₄₇H₂₉N₃S = 667.83) |
| N 1-55 | m/z = 959.33(C₇₀H₄₅N₃S = 960.21) | N 1-56 | m/z = 934.31(C₆₇H₄₂N₄S = 935.16) |
| N 1-57 | m/z = 606.25(C₄₃H₂₂D₅N₃O = 606.74) | N 1-58 | m/z = 606.25(C₄₃H₂₂D₅N₃O = 606.74) |
| N 1-59 | m/z = 655.26(C₄₇H₂₅D₄N₃O = 655.79) | N 1-60 | m/z = 611.28(C₄₃H₁₇D₁₀N₃O = 611.77) |
| N 1-61 | m/z = 699.26(C₄₉H₂₅D₆N₃S = 699.90) | N 1-62 | m/z = 774.29(C₅₅H₃₀D₅N₃S = 775.00) |
| N 1-63 | m/z = 971.33(C₇₁H₄₅N₃S = 972.22) | N 1-64 | m/z = 895.30(C₆₅H₄₁N₃S = 896.12) |
| N 1-65 | m/z = 841.31(C₆₂H₃₉N₃O = 842.01) | N 1-66 | m/z = 691.23(C₄₉H₂₉N₃O₂ = 691.79) |
| N 1-67 | m/z = 766.27(C₅₅H₃₄N₄O = 766.90) | N 1-68 | m/z = 843.29(C₆₁H₃₇N₃O₂ = 843.99) |
| N 1-69 | m/z = 809.29(C₅₈H₃₉N₃S = 810.03) | N 1-70 | m/z = 847.30(C₆₁H₄₁N₃S = 848.08) |
| N 1-71 | m/z = 735.23(C₅₁H₃₃N₃OS = 735.90) | N 1-72 | m/z = 875.24(C₆₁H₃₇N₃S₂ = 876.11) |
| N 1-73 | m/z = 735.32(C₅₃H₄₁N₃O = 735.93) | N 1-74 | m/z = 695.29(C₅₀H₃₇N₃O = 695.87) |
| N 1-75 | m/z = 767.39(C₅₅H₄₉N₃O = 768.02) | N 1-76 | m/z = 885.37(C₆₅H₄₇N₃O = 886.11) |
| N 1-77 | m/z = 763.30(C₅₄H₄₁N₃S = 764.00) | N 1-78 | m/z = 793.26(C₅₇H₃₅N₃S = 793.99) |
| N 1-79 | m/z = 722.26(C₅₁H₂₆D₅N₃S = 722.92) | N 1-80 | m/z = 743.24(C₅₃H₃₃N₃S = 743.93) |
| N 1-81 | m/z = 727.26(C₅₃H₃₃N₃O = 727.87) | N 1-82 | m/z = 757.22(C₅₃H₃₁N₃OS = 757.91) |
| N 1-83 | m/z = 791.26(C₅₇H₃₃N₃O₂ = 791.91) | N 1-84 | m/z = 767.29(C₅₆H₃₇N₃O = 767.93) |
| N 1-85 | m/z = 767.24(C₅₅H₃₃N₃S = 767.95) | N 1-86 | m/z = 783.23(C₅₅H₃₃N₃OS = 783.95) |
| N 1-87 | m/z = 908.30(C₆₅H₄₀N₄S = 909.12) | N 1-88 | m/z = 805.35(C₅₇H₄₇N₃S = 806.08) |
| N 1-89 | m/z = 829.31(C₆₁H₃₉N₃O = 830.00) | N 1-90 | m/z = 803.29(C₅₉H₃₇N₃O = 803.97) |
| N 1-91 | m/z = 803.29(C₅₉H₃₇N₃O = 803.97) | N 1-92 | m/z = 827.29(C₆₁H₃₇N₃O = 827.99) |
| N 1-93 | m/z = 799.30(C₅₇H₄₁N₃S = 800.04) | N 1-94 | m/z = 895.30(C₆₅H₄₁N₃S = 896.12) |
| N 1-95 | m/z = 693.22(C₄₉H₃₁N₃S = 693.87) | N 1-96 | m/z = 804.47(C₅₅D₃₅N₃S = 805.18) |
| N 1-97 | m/z = 691.23(C₄₉H₂₉N₃O₂ = 691.79) | N 1-98 | m/z = 677.25(C₄₉H₃₁N₃O = 677.81) |
| N 1-99 | m/z = 799.30(C₅₇H₄₁N₃S = 800.04) | N 1-100 | m/z = 808.33(C₅₉D₃₂N₃O = 809.00) |

### [Synthesis Example 3] A Compound Represented by Formula 4 or Formula 5

1. Synthesis Example of H-12

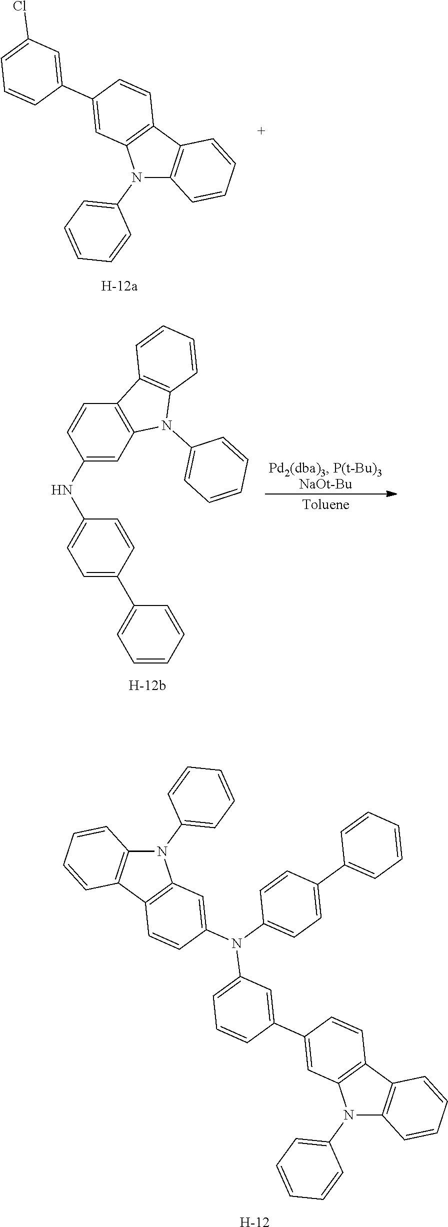

After dissolving H-12a (30 g, 0.08 mol) in Toluene (170 mL) in a round bottom flask, H-12b (34.8 g, 0.08 mol), Pd₂(dba)₃ (2.3 g, 0.003 mol), NaOt-Bu (24.5 g, 0.25 mol), P(t-Bu)₃ (2.1 g, 0.005 mol) were added and stirred at 135° C. for 6 hours. When the reaction was completed, the reactant was extracted with CH₂Cl₂ and water, and the organic layer was dried over MgSO₄, concentrated, and the resulting compound was recrystallized using a silicagel column to obtain 53 g of the product. (Yield: 85.8%)

2. Synthesis Example of H-19

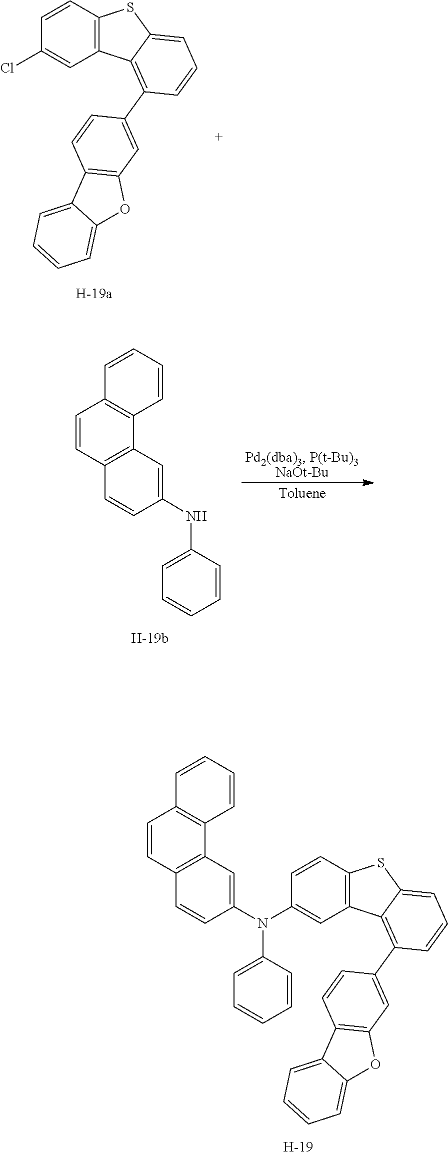

H-19a (50 g, 0.13 mol), H-19b (35 g, 0.13 mol), Pd₂(dba)₃ (3.6 g, 0.004 mol), NaOt-Bu (37.6 g, 0.40 mol), P(t-Bu)₃ (3.2 g, 0.008 mol), Toluene (260 mL) were added to a round bottom flask in the same manner as in H-12 to obtain 67 g of product. (Yield: 83.4%)

3. Synthesis Example of S-32

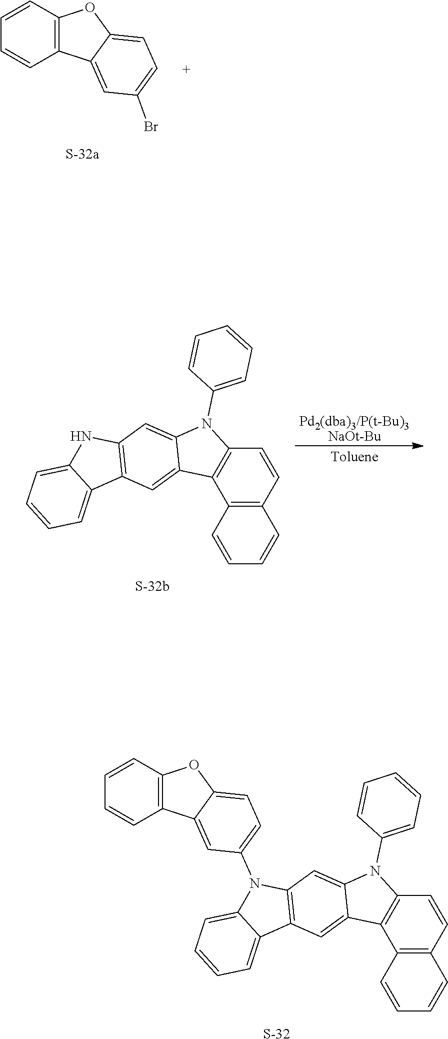

S-32a (10 g, 0.04 mol), S-32b (15.6 g, 0.04 mol), Pd₂(dba)₃ (1.1 g, 0.001 mol), NaOt-Bu (11.7 g, 0.12 mol), P(t-Bu)₃ (1.0 g, 0.002 mol), Toluene (80 mL) were added to a round bottom flask in the same manner as in H-12 to obtain 18 g of product. (Yield: 80.8%)

4. Synthesis Example of S-74

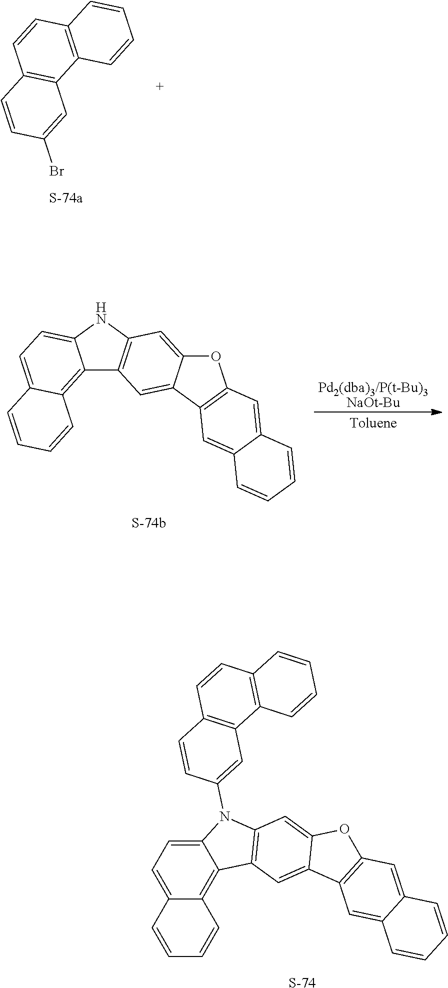

S-74a (15 g, 0.06 mol), S-74b (20.9 g, 0.06 mol), Pd₂(dba)₃ (1.6 g, 0.002 mol), NaOt-Bu (16.9 g, 0.18 mol), P(t-Bu)₃ (1.4 g, 0.004 mol), Toluene (120 mL) were tested in the same manner as in H-12 to obtain 27 g of product. (Yield: 86.4%)

5. Synthesis Example of S-104

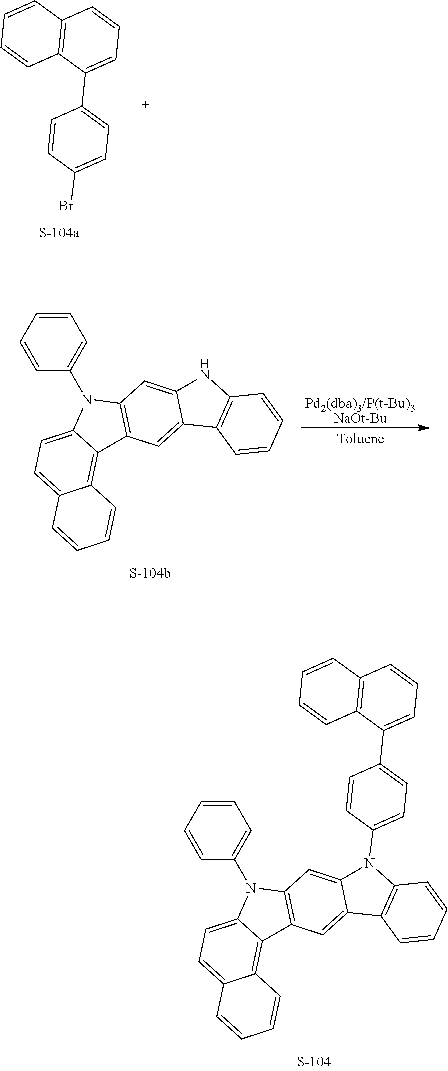

S-104a (30 g, 0.13 mol), S-104b (48.2.9 g, 0.13 mol), Pd₂(dba)₃ (3.5 g, 0.004 mol), NaOt-Bu (36.4 g, 0.38 mol), P(t-Bu)₃ (3.1 g, 0.008 mol), Toluene (120 mL) were tested in the same manner as in H-12 to obtain 60 g of product. (Yield: 81.5%)

Otherwise, the FD-MS values of the compounds H-1 to H-100 and S-1 to S-108 of the present invention prepared according to the synthesis examples as described above are shown in Tables 8 and 9.

TABLE 8

| Compound | FD-MS | Compound | FD-MS |
|---|---|---|---|
| H-1 | m/z = 487.19(C₃₆H₂₅NO = 487.6) | H-2 | m/z = 553.19(C₄₀H₂₇NS = 553.72) |
| H-3 | m/z = 563.26(C₄₃H₃₃N = 563.74) | H-4 | m/z = 602.27(C₄₅H₃4N₂ = 602.78) |
| H-5 | m/z = 517.15(C₃₆H₂3NOS = 517.65) | H-6 | m/z = 603.2(C₄₄H₂₉NS = 603.78) |
| H-7 | m/z = 735.29(C₅₇H₃₇N = 735.93) | H-8 | m/z = 562.24(C₄₂H₃₀N₂ = 562.72) |
| H-9 | m/z = 565.17(C₄₀H₂₃NO₃ = 565.63) | H-10 | m/z = 581.14(C₄₀H₂₃NO₂S = 581.69) |
| H-11 | m/z = 823.24(C₅₉H₃₇NS₂ = 824.07) | H-12 | m/z = 727.3(C₅₄H₃₇N₃ = 727.91) |
| H-13 | m/z = 627.22(C₄₆H₂₉NO₂ = 627.74) | H-14 | m/z = 633.16(C₄₄H₂₇NS₂ = 633.83) |
| H-15 | m/z = 675.29(C₅₂H₃₇N = 675.88) | H-16 | m/z = 678.3(C₅₁H₃₈N₂ = 678.88) |
| H-17 | m/z = 669.21(C₄₈H₃₁NOS = 669.84) | H-18 | m/z = 785.22(C₅₆H₃₅NS₂ = 786.02) |
| H-19 | m/z = 617.18(C₄₄H₂₇NOS = 617.77) | H-20 | m/z = 601.2(C₄₄H₂₇NO₂ = 601.71) |
| H-21 | m/z = 779.32(C₅₉H₄₁NO = 779.98) | H-22 | m/z = 583.23(C₄₂H₃₃NS = 583.79) |
| H-23 | m/z = 679.32(C₅₂H₄₁N = 679.91) | H-24 | m/z = 726.27(C₅₄H₃₄N₂O = 726.88) |
| H-25 | m/z = 593.18(C₄₂H₂₇NOS = 593.74) | H-26 | m/z = 774.22(C₅₄H₃₄N₂S₂ = 775) |
| H-27 | m/z = 557.24(C₄₀H₃₁NO₂ = 557.69) | H-28 | m/z = 652.25(C₄₈H₃₂N₂O = 652.8) |
| H-29 | m/z = 619.29(C₄₆H₃₇NO = 619.81) | H-30 | m/z = 603.2(C₄₄H₂₉NS = 603.78) |
| H-31 | m/z = 813.3(C₆₂H₃₉NO = 814) | H-32 | m/z = 784.29(C₅₇H₄₀N₂S = 785.02) |
| H-33 | m/z = 577.2(C₄₂H₂₇NO₂ = 577.68) | H-34 | m/z = 607.14(C₄₂H₂₅NS₂ = 607.79) |
| H-35 | m/z = 801.34(C₆₂H₄₃N = 802.03) | H-36 | m/z = 575.24(C₄₂H₂₉N₃ = 575.72) |
| H-37 | m/z = 577.2(C₄₂H₂₇NO₂ = 577.68) | H-38 | m/z = 607.14(C₄₂H₂₅NS₂ = 607.79) |
| H-39 | m/z = 801.34(C₆₂H₄₃N = 802.03) | H-40 | m/z = 575.24(C₄₂H₂₉N₃ = 575.72) |
| H-41 | m/z = 601.2(C₄₄H₂₇NO₂ = 601.71) | H-42 | m/z = 471.11(C₃₁H₂₁NS₂ = 471.64) |
| H-43 | m/z = 675.29(C₅₂H₃₇N = 675.88) | H-44 | m/z = 727.3(C₅₄H₃₇N₃ = 727.91) |
| H-45 | m/z = 603.2(C₄₄H₂₉ NS = 603.78) | H-46 | m/z = 561.16(C₃₈H₂₇NS₂ = 561.76) |
| H-47 | m/z = 799.32(C₆₂H₄₁N = 800.02) | H-48 | m/z = 702.27(C₅₂H₃₄N₂O = 702.86) |
| H-49 | m/z = 729.27(C₅₄H₃₅NO₂ = 729.88) | H-50 | m/z = 785.22(C₅₆H₃₅NS₂ = 786.02) |
| H-51 | m/z = 812.32(C₆₂H₄₀N₂ = 813.02) | H-52 | m/z = 681.22(C₄₈H₃₁N₃S = 681.86) |
| H-53 | m/z = 615.18(C₄₄H₂₅NO₃ = 615.69) | H-54 | m/z = 763.15(C₅₂H₂₉NS₃ = 763.99) |
| H-55 | m/z = 593.31(C₄₅H₃₉N = 593.81) | H-56 | m/z = 840.33(C₆₂H₄₀N₄ = 841.03) |
| H-57 | m/z = 657.18(C₄₆H₂₇NO₂S = 657.79) | H-58 | m/z = 824.23(C₅₈H₃₆N₂S₂ = 825.06) |
| H-59 | m/z = 1195.42(C₉₁ H₅₇NS = 1196.52) | H-60 | m/z = 656.19(C₄₆H₂₈N₂OS = 656.8) |
| H-61 | m/z = 607.16(C₄₂H₂₅NO₂S = 607.73) | H-62 | m/z = 773.2(C₅₄H₃₁NO₃S = 773.91) |
| H-63 | m/z = 1013.4(C₇₉H₅₁N = 1014.28) | H-64 | m/z = 758.24(C₅₄H₃₄N₂OS = 758.94) |
| H-65 | m/z = 623.14(C₄₂H₂₅NOS₂ = 623.79) | H-66 | m/z = 763.16(C₅₂H₂₉NO₂S₂ = 763.93) |
| H-67 | m/z = 799.2(C₅₆H₃₃NOS₂ = 800.01) | H-68 | m/z = 743.23(C₅₄H₃₃NOS = 743.92) |
| H-69 | m/z = 872.25(C₆₂H₃₆N₂O₂S = 873.04) | H-70 | m/z = 772.22(C₅₄H₃₂N₂O₂S = 772.92) |
| H-71 | m/z = 830.28(C₆₁H₃₈N₂S = 831.05) | H-72 | m/z = 808.25(C₅₈H₃₃FN₂O₂ = 808.91) |
| H-73 | m/z = 929.21(C₆₄H₃₅NO₃S₂ = 930.11) | H-74 | m/z = 963.27(C₆₈H₄₁N₃S₂ = 964.22) |
| H-75 | m/z = 809.24(C₅₈H₃₅NO₂S = 809.98) | H-76 | m/z = 893.29(C₆₆H₃₉NO₃ = 894.04) |
| H-77 | m/z = 794.28(C₅₈H₃₈N₂S = 795.02) | H-78 | m/z = 900.26(C₆₄H₄₀N₂S₂ = 901.16) |
| H-79 | m/z = 758.28(C₅₅H₃₈N₂S = 758.98) | H-80 | m/z = 1082.37(C₈₁H₅₀N₂S = 1083.37) |
| H-81 | m/z = 573.25(C₄₄H₃₁N = 573.74) | H-82 | m/z = 649.28(C₅₀H₃₅N = 649.84) |
| H-83 | m/z = 699.29(C₅₄H₃₇N = 699.9) | H-84 | m/z = 699.29(C₅₄H₃₇N = 699.9) |
| H-85 | m/z = 673.28(C₅₂H₃₅N = 673.86) | H-86 | m/z = 649.28(C₅₀H₃₅N = 649.84) |
| H-87 | m/z = 625.28(C₄₈H₃₅N = 625.82) | H-88 | m/z = 673.28(C₅₂H₃₅N= 673.86) |
| H-89 | m/z = 773.31(C₆₀H₃₉N = 773.98) | H-90 | m/z = 749.31(C₅₈H₃₉N = 749.96) |
| H-91 | m/z = 699.29(C₅₄H₃₇N = 699.9) | H-92 | m/z = 599.26(C₄₆H₃₃N = 599.78) |
| H-93 | m/z = 639.26(C₄₈H₃₃NO = 639.8) | H-94 | m/z = 765.25(C₅₇H₃₅NS = 765.97) |
| H-95 | m/z = 677.31(C₅₂H₃₉N = 677.89) | H-96 | m/z = 727.3(C₅₄H₃₇N₃ = 727.91) |
| H-97 | m/z = 552.18(C₃₉H₂₄N₂O₂ = 552.63) | H-98 | m/z = 628.22(C₄₅H₂₈N₂O₂ = 628.73) |
| H-99 | m/z = 614.24(C₄₅H₃₀N₂O = 614.75) | H-100 | m/z = 614.24(C₄₅H₃₀N₂O = 614.75) |

TABLE 9

| Compound | FD-MS | Compound | FD-MS |
|---|---|---|---|
| S-1 | m/z = 408.16(C₃₀H₂₀N₂ = 408.5) | S-2 | m/z = 534.21 (C₄₀H₂₆N₂ = 534.66) |
| S-3 | m/z = 560.23(C₄₂H₂₈N₂ = 560.7) | S-4 | m/z = 584.23(C₄₄H₂₈N₂ = 584.72) |
| S-5 | m/z = 560.23(C₄₂H₂₈N₂ = 560.7) | S-6 | m/z = 634.24(C₄₈H₃₀N₂ = 634.78) |
| S-7 | m/z = 610.24(C₄₆H₃₀N₂ = 610.76) | S-8 | m/z = 498.17(C₃₆H₂₂N₂O = 498.59) |
| S-9 | m/z = 574.2(C₄₂H₂₆N₂O = 574.68) | S-10 | m/z = 660.26(C₅₀H₃₂N₂ = 660.82) |
| S-11 | m/z = 686.27(C₅₂H₃₄N₂ = 686.86) | S-12 | m/z = 620.14(C₄₂H₂₄N₂S₂ = 620.79) |
| S-13 | m/z = 640.2(C₄₆H₂₈N₂S = 640.8) | S-14 | m/z = 560.23(C₄₂H₂₈N₂ = 560.7) |
| S-15 | m/z = 558.21(C₄₂H₂₆N₂ = 558.68) | S-16 | m/z = 548.19(C₄₀H₂₄N₂O = 548.65) |
| S-17 | m/z = 573.22(C₄₂H₂₇N₃ = 573.7) | S-18 | m/z = 564.17(C₄₀H₂₄N₂S = 564.71) |
| S-19 | m/z = 574.2(C₄₂H₂₆N₂O = 574.68) | S-20 | m/z = 564.17(C₄₀H₂₄N₂S = 564.71) |
| S-21 | m/z = 564.17(C₄₀H₂₄N₂S = 564.71) | S-22 | m/z = 813.31(C₆₁H₃₉N₃ = 814) |
| S-23 | m/z = 696.26(C₅₃H₃₂N₂ = 696.85) | S-24 | m/z = 691.23(C₄₉H₂₉N₃O₂ = 691.79) |
| S-25 | m/z = 710.27(C₅₄H₃₄N₂ = 710.88) | S-26 | m/z = 610.24(C₄₆H₃₀N₂ = 610.76) |
| S-27 | m/z = 670.15(C₄₆H₂₆N₂S₂ = 670.85) | S-28 | m/z = 640.29(C₄₈H₃₆N₂ = 640.83) |
| S-29 | m/z = 598.2(C₄₄H₂₆N₂O = 598.71) | S-30 | m/z = 623.24(C₄₆H₂₉N₃ = 623.76) |
| S-31 | m/z = 458.18(C₃₄H₂₂N₂ = 458.56) | S-32 | m/z = 548.19(C₄₀H₂₄N₂O = 548.65) |
| S-33 | m/z = 508.19(C₃₈H₂₄N₂ = 508.62) | S-34 | m/z = 508.19(C₃₈H₂₄N₂ = 508.62) |
| S-35 | m/z = 623.24(C₄₆H₂₉N₃ = 623.76) | S-36 | m/z = 564.17(C₄₀H₂₄N₂S = 564.71) |
| S-37 | m/z = 627.2(C₄₆H₂₉NS = 627.81) | S-38 | m/z = 505.1(C₃₄H₁₉NS₂ = 505.65) |
| S-39 | m/z = 514.15(C₃₆H₂₂N₂S = 514.65) | S-40 | m/z = 575.17(C₄₂H₂₅NS = 575.73) |
| S-41 | m/z = 642.21(C₄₆H₃₀N₂S = 642.82) | S-42 | m/z = 575.17(C₄₂H₂₅NS = 575.73) |
| S-43 | m/z = 606.18(C₄₂H₂₆N₂OS = 606.74) | S-44 | m/z = 575.17(C₄₂H₂₅NS = 575.73) |
| S-45 | m/z = 551.17(C₄₀H₂₅NS = 551.71) | S-46 | m/z = 607.14(C₄₂H₂₅NS₂ = 607.79) |
| S-47 | m/z = 525.16(C₃₈H₂₃NS = 525.67) | S-48 | m/z = 642.21(C₄₆H₃₀N₂S = 642.82) |
| S-49 | m/z = 548.19(C₄₀H₂₄N₂O = 548.65) | S-50 | m/z = 473.14(C₃₄H₁₉NO₂ = 473.53) |
| S-51 | m/z = 566.15(C₃₉H₂₂N₂OS = 566.68) | S-52 | m/z = 459.16(C₃₄H₂₁NO = 459.55) |
| S-53 | m/z = 473.14(C₃₄H₁₉NO₂ = 473.53) | S-54 | m/z = 523.16(C₃₈H₂₁NO₂ = 523.59) |
| S-55 | m/z = 539.13(C₃₈H₂₁NOS = 539.65) | S-56 | m/z = 548.19(C₄₀H₂₄N₂O = 548.65) |
| S-57 | m/z = 489.12(C₃₄H₁₉NOS = 489.59) | S-58 | m/z = 545.09(C₃₆H₁₉NOS₂ = 545.67) |
| S-59 | m/z = 549.17(C₄₀H₂₃NO₂ = 549.63) | S-60 | m/z = 565.15(C₄₀H₂₃NOS = 565.69) |
| S-61 | m/z = 523.16(C₃₈H₂₁NO₂ = 523.59) | S-62 | m/z = 598.2(C₄₄H₂₆N₂O = 598.71) |
| S-63 | m/z = 539.13(C₃₈H₂₁NOS = 539.65) | S-64 | m/z = 589.15(C₄₂H₂₃NOS = 589.71) |
| S-65 | m/z = 498.17(C₃₆H₂₂N₂O = 498.59) | S-66 | m/z = 509.18(C₃₈H₂₃NO = 509.61) |
| S-67 | m/z = 548.19(C₄₀H₂₄N₂O = 548.65) | S-68 | m/z = 549.17(C₄₀H₂₃NO₂ = 549.63) |
| S-69 | m/z = 449.12(C₃₂H₁₉NS = 449.57) | S-70 | m/z = 439.1(C₃₀H₁₇NOS = 439.53) |
| S-71 | m/z = 647.22(C₄₉H₂₉NO = 647.78) | S-72 | m/z = 717.28(C₅₂H₃₅N₃O = 717.87) |
| S-73 | m/z = 459.16(C₃₄H₂₁NO = 459.55) | S-74 | m/z = 533.18(C₄₀H₂₃NO = 533.63) |
| S-75 | m/z = 525.16(C₃₈H₂₃NS = 525.67) | S-76 | m/z = 564.17(C₄₀H₂₄N₂S = 564.71) |
| S-77 | m/z = 575.19(C₄₂H₂₅NO₂ = 575.67) | S-78 | m/z = 663.22(C₄₉H₂₉NO₂ = 663.78) |
| S-79 | m/z = 647.22(C₄₉H₂₉NO = 647.78) | S-80 | m/z = 496.16(C₃₆H₂₀N₂O = 496.57) |
| S-81 | m/z = 565.15(C₄₀H₂₃NOS = 565.69) | S-82 | m/z = 505.1(C₃₄H₁₉NS₂ = 505.65) |
| S-83 | m/z = 765.25(C₅₆H₃₅NOSi = 765.99) | S-84 | m/z = 615.17(C₄₄H₂₅NOS = 615.75) |
| S-85 | m/z = 603.17(C₄₃H₂₅NOS = 603.74) | S-86 | m/z = 772.29(C₅₉H₃₆N₂ = 772.95) |
| S-87 | m/z = 802.33(C₆₁H₄₂N₂ = 803.02) | S-88 | m/z = 607.23(C₄₇H₂₉N = 607.76) |
| S-89 | m/z = 524.23(C₃₉H₂₈N₂ = 524.67) | S-90 | m/z = 665.22(C₄₉H₃₁NS = 665.85) |
| S-91 | m/z = 633.25(C₄₉H₃₁N = 633.79) | S-92 | m/z = 775.29(C₅₉H₃₇NO = 775.95) |
| S-93 | m/z = 535.23(C₄₁H₂₉N = 535.69) | S-94 | m/z = 623.22(C₄₇H₂₉NO = 623.76) |
| S-95 | m/z = 687.2(C₅₁H₂₉NS = 687.86) | S-96 | m/z = 735.29(C₅₇H₃₇N = 735.93) |
| S-97 | m/z = 611.26(C₄₇H₃₃N = 611.79) | S-98 | m/z = 679.23(C₅₀H₃₃NS = 679.88) |
| S-99 | m/z = 787.32(C₆₁H₄₁N = 788.01) | S-100 | m/z = 743.33(C₅₅H₄₁N₃ = 743.95) |
| S-101 | m/z = 485.21(C₃₇H₂₇N = 485.63) | S-102 | m/z = 471.2(C₃₆H₂₅N = 471.6) |
| S-103 | m/z = 571.19(C₄₃H₂₅NO = 571.68) | S-104 | m/z = 584.23(C₄₄H₂₈N₂ = 584.72) |
| S-105 | m/z = 539.24(C₄₀H₂₁D₅N₂ = 539.69) | S-106 | m/z = 453.15(C₃₂H₁₅NS = 471.6) |
| S-107 | m/z = 563.26(C₄₃H₂₆D₄NO = 563.74) | S-108 | m/z = 589.26(C₄₄H₂₃D₅N₂ = 584.72) |

Otherwise, the synthesis examples of the present invention represented by the Formulas 2 to 5 have been described, but these are all based on the Buchwald-Hartwig cross coupling reaction, Miyaura boration reaction, Suzuki cross-coupling reaction, Intramolecular acid-induced cyclization reaction (J. mater. Chem. 1999, 9, 2095), Pd(II)-catalyzed oxidative cyclization reaction (Org. Lett. 2011, 13, 5504), and PPh₃-mediated reductive cyclization reaction (J. Org. Chem. 2005, 70, 5014), and It will be easily understood by those skilled in the art that the reaction proceeds even when other substituents defined in Formulas 2 to 5 are bonded in addition to the substituents specified in the specific synthesis examples.

### Manufacturing Evaluation of Organic Electronic Elements

### [Example 31] Red Organic Light Emitting Device (Phosphorescent Host)

After vacuum depositing N¹-(naphthalen-2-yl)-N⁴,N⁴-bis(4-(naphthalen-2-yl(phenyl)amino)phenyl)-N¹-phenylbenzene-1,4-diamine (hereinafter abbreviated as 2-TNATA) on the ITO layer (anode) formed on the glass substrate to form a hole injection layer with a thickness of 60 nm, a hole transport layer was formed by vacuum depositing N,N′-bis(1-naphthalenyl)-N,N′-bis-phenyl-(1,1′-biphenyl)-4,4′-diamine (hereinafter abbreviated as NPB) to a thickness of 60 nm on the hole injection layer.

Subsequently, tris(4-(9H-carbazol-9-yl)phenyl)amine (hereinafter abbreviated as TCTA) was vacuum-deposited to a thickness of 10 nm on the hole transport layer to form an emitting-auxiliary layer. Then, the host of the emitting layer uses N 1-1, the compound of the present invention as a first host and H-17, the compound of the present invention as a second host, but a mixture obtained by mixing the first host and the second host in a weight ratio of 5:5 is used, and bis-(1-phenylisoquinolyl)iridium(III)acetylacetonate (hereinafter abbreviated as ‘(piq)₂Ir(acac)’) was used as a dopant, but the dopant was doped so that the weight ratio of the host and the dopant was 95:5 to form an emitting layer having a thickness of 30 nm.

Next, (1,1′-biphenyl-4-olato)bis(2-methyl-8-quinolinolato)aluminum (hereinafter abbreviated as BAlq) was vacuum deposited on the emitting layer to form a hole blocking layer having a thickness of 10 nm, Tris(8-hydroxyquinolinato)aluminium (hereinafter abbreviated as Alq₃) was vacuum deposited on the hole blocking layer to a thickness of 40 nm to form an electron transport layer. Thereafter, 8-quinolinolato lithium (hereinafter abbreviated as Liq) was deposited on the electron transport layer to form an electron injection layer having a thickness of 0.2 nm, and then Al was deposited to form a cathode having a thickness of 150 nm.

### [Example 32] to [Example 70]

An organic light emitting device was manufactured in the same manner as in Example 31, except that the compound of the present invention described in Table 10 was used as the host material of the emitting layer.

### [Comparative Example 7] and [Comparative Example 8]

An organic light emitting device was manufactured in the same manner as in Example 31, except that Comparative Compound A or Comparative Compound B was used as the first host as the host material of the emitting layer.

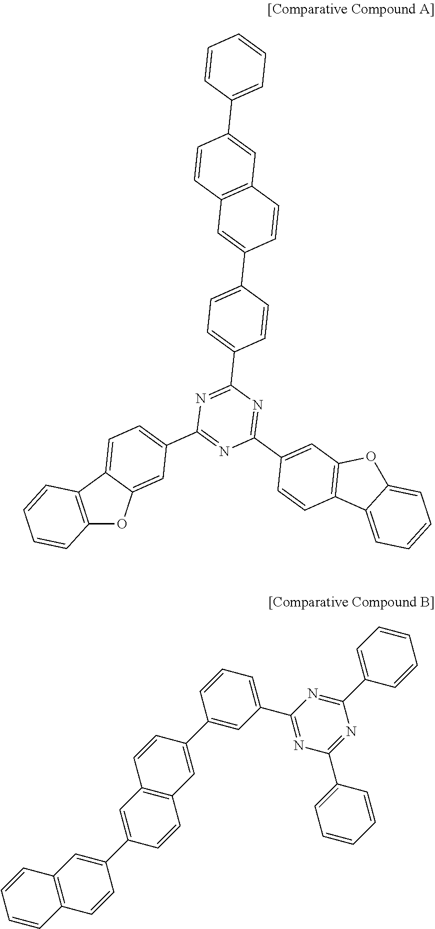 [SMILES: c1ccc(-c2ccc3cc(-c4ccc(-c5nc(-c6ccc7c(c6)oc6ccccc67)nc(-c6ccc7c(c6)oc6ccccc67)n5)cc4)ccc3c2)cc1 ; c1ccc(-c2nc(-c3ccccc3)nc(-c3cccc(-c4ccc5cc(-c6ccc7ccccc7c6)ccc5c4)c3)n2)cc1]

To the organic electroluminescent device manufactured by Examples 31 to 70, Comparative Examples 7 and 8 of the present invention, Electroluminescence (EL) characteristics were measured with a PR-650 of Photoresearch Co., by applying a forward bias DC voltage. As a result of the measurement, T95 life was measured at a standard luminance of 2,500 cd/m² through life measuring apparatus manufactured by McScience. Table 10 shows the results of device fabrication and evaluation.

This measuring device is independent form possible day-to-day variations of deposition rates, vacuum quality or other tool performance parameters, and allows assessing performance of new material in comparison with comparative compound under the same conditions.

At the time of assessment, each field contained 4 identically prepared OLEDs including a comparative compound, and since the performance of each of a total of 12 OLEDs in 3 fields is evaluated, the statistical evaluation of the obtained experimental results unequivocally showed the statistical significance.

TABLE 10

| | | | | Current | | | |
|---|---|---|---|---|---|---|---|
| | | Second | | Density | Brightness | Efficiency | |
| | Frist host | host | Voltage | (mA/cm²⁾ | (cd/m²) | (cd/A) | T(95) |
| Comparative | Comparative | H-17 | 5.5 | 10.1 | 2500.0 | 24.7 | 107.7 |
| example7 | compound A | | | | | | |
| Comparative | Comparative | H-17 | 5.6 | 10.9 | 2500.0 | 23.0 | 104.5 |
| example8 | compound B | | | | | | |
| Example 31 | N 1-1 | H-17 | 5.0 | 8.3 | 2500.0 | 30.3 | 125.1 |
| Example 32 | N 1-6 | H-17 | 4.7 | 7.4 | 2500.0 | 33.7 | 128.7 |
| Example 33 | N 1-13 | H-17 | 4.9 | 8.9 | 2500.0 | 28.1 | 123.0 |
| Example 34 | N 1-22 | H-17 | 4.9 | 7.9 | 2500.0 | 31.8 | 126.8 |
| Example 35 | N 1-36 | H-17 | 5.1 | 8.8 | 2500.0 | 28.5 | 123.4 |
| Example 36 | N 1-47 | H-17 | 4.8 | 7.8 | 2500.0 | 32.2 | 120.3 |
| Example 37 | N 1-52 | H-17 | 5.0 | 8.2 | 2500.0 | 30.6 | 120.6 |
| Example 38 | N 1-53 | H-17 | 5.1 | 7.8 | 2500.0 | 31.9 | 125.3 |
| Example 39 | N 1-60 | H-17 | 4.8 | 7.4 | 2500.0 | 34.0 | 129.4 |
| Example 40 | N 1-63 | H-17 | 5.0 | 8.2 | 2500.0 | 30.6 | 120.7 |
| Example 41 | N 1-67 | H-17 | 4.8 | 8.8 | 2500.0 | 28.3 | 121.7 |
| Example 42 | N 1-73 | H-17 | 4.8 | 7.9 | 2500.0 | 31.7 | 124.6 |
| Example 43 | N 1-75 | H-17 | 4.9 | 8.4 | 2500.0 | 29.7 | 121.6 |
| Example 44 | N 1-97 | H-17 | 4.8 | 8.1 | 2500.0 | 30.9 | 123.1 |
| Example 45 | N 1-98 | H-17 | 5.1 | 8.4 | 2500.0 | 29.7 | 123.4 |
| Example 46 | N 1-1 | H-84 | 5.1 | 8.2 | 2500.0 | 30.4 | 120.5 |
| Example 47 | N 1-22 | H-84 | 5.0 | 8.1 | 2500.0 | 30.9 | 125.5 |
| Example 48 | N 1-52 | H-84 | 5.1 | 8.7 | 2500.0 | 28.6 | 120.7 |
| Example 49 | N 1-67 | H-84 | 4.9 | 8.3 | 2500.0 | 30.0 | 120.4 |
| Example 50 | N 1-75 | H-84 | 5.0 | 8.4 | 2500.0 | 29.6 | 121.6 |
| Example 51 | N 1-98 | H-84 | 5.2 | 7.8 | 2500.0 | 32.0 | 125.4 |
| Example 52 | N 1-6 | H-98 | 4.7 | 7.3 | 2500.0 | 34.3 | 130.7 |
| Example 53 | N 1-47 | H-98 | 4.8 | 8.9 | 2500.0 | 28.1 | 128.4 |
| Example 54 | N 1-53 | H-98 | 5.0 | 8.3 | 2500.0 | 30.1 | 127.6 |
| Example 55 | N 1-60 | H-98 | 4.9 | 7.2 | 2500.0 | 34.6 | 131.1 |
| Example 56 | N 1-67 | H-98 | 4.8 | 8.0 | 2500.0 | 31.2 | 126.1 |
| Example 57 | N 1-97 | H-98 | 4.9 | 8.6 | 2500.0 | 29.0 | 129.7 |
| Example 58 | N 1-13 | S-16 | 5.1 | 8.5 | 2500.0 | 29.4 | 135.8 |
| Example 59 | N 1-36 | S-16 | 5.3 | 8.0 | 2500.0 | 31.4 | 135.1 |
| Example 60 | N 1-60 | S-16 | 5.0 | 7.9 | 2500.0 | 31.8 | 136.5 |
| Example 61 | N 1-63 | S-16 | 5.2 | 8.1 | 2500.0 | 30.7 | 134.9 |
| Example 62 | N 1-75 | S-16 | 5.1 | 8.2 | 2500.0 | 30.5 | 138.2 |
| Example 63 | N 1-98 | S-16 | 5.1 | 8.6 | 2500.0 | 29.0 | 135.7 |
| Example 64 | N 1-6 | S-108 | 4.8 | 7.0 | 2500.0 | 35.7 | 140.9 |
| Example 65 | N 1-13 | S-108 | 5.0 | 8.8 | 2500.0 | 28.3 | 138.8 |
| Example 66 | N 1-47 | S-108 | 4.9 | 8.3 | 2500.0 | 30.2 | 140.3 |
| Example 67 | N 1-53 | S-108 | 5.2 | 8.6 | 2500.0 | 29.2 | 137.6 |
| Example 68 | N 1-60 | S-108 | 4.9 | 7.1 | 2500.0 | 35.1 | 141.2 |
| Example 69 | N 1-73 | S-108 | 4.9 | 8.0 | 2500.0 | 31.2 | 139.7 |
| Example 70 | N 1-97 | S-108 | 4.9 | 8.1 | 2500.0 | 30.9 | 138.5 |

As can be seen from the results of Table 10, when a red organic light emitting device is manufactured using the material for an organic light emitting device of the present invention as a host material of an emitting layer, it is possible to improve the driving voltage, luminous efficiency and lifespan of the organic light emitting device, compared to the comparative example using Comparative Compound A or Comparative Compound B having a similar basic skeleton to the compound of the present invention.

Comparative Compound A and Comparative Compound B are similar to the compounds of the present invention in that they are triazine compounds in which the group represented by the phenyl-naphthyl-aryl group in the molecule is substituted, but in the case of Comparative Compound A, it is different from the compound of the present invention in that the phenyl group between triazine and naphthyl forms a para bond, and in the case of Comparative compound B, it is different from the compound of the present invention in that all triazine substituents are aryl groups.

In order to confirm the Reorganization Energy (hereinafter abbreviated as RE) of a compound that changes due to such a structural difference, the data measured using the DFT method (B3LYP/6-31g(D)) of the Gaussian program of the compound N 1-97 of the present invention having high similarity to the comparative compound Aare shown in Table 11.

TABLE 11

| compound | Reorganization Energy (RE) |
|---|---|
| N 1-97 | 0.187 |
| Comparative compound A | 0.232 |

RE in Table 11 is a RE_{elec} value.

As can be seen from the results of Table 11, it can be seen that the RE value of the compound N 1-97 of the present invention having high structural similarity with Comparative compound A is significantly different.

Due to this difference, the compound of the present invention having a low RE value has higher mobility and faster EOD than Comparative Compound A, so electron transfer and electron injection are remarkably improved, and as a result, the electron injection of the dopant is increased as the driving voltage is reduced and the emitting layer is enriched with electrons, so that the efficiency and lifespan are also significantly improved.

Table 12 shows the data measured using the DFT method (B3LYP/6-31g(D)) of the Gaussian program in order to confirm the energy level of the compound N 1-47 of the present invention, which has a high similarity to the Comparative Compound B.

TABLE 12

| compound | N 1-47 | Comparative compound B |
|---|---|---|
| LUMO(eV) | −1.784 | −1.671 |

From the results of Table 12, it can be seen that the energy levels of Comparative Compound B and Compound N 1-47 of the present invention are significantly different. More specifically, it is judged that as the LUMO level of the compound of the present invention is deeper than that of Comparative Compound B, injection of electrons from the electron transport region is performed more smoothly, and the performance of the element is also remarkably improved as the charge balance of the element is formed by increasing the formation of Exition in the emitting layer.

That is, as can be seen from the results of Tables 10 to 12, it can be confirmed that the compound of the present invention, which satisfies a specific configuration, exhibits a more significant effect in organic electronic elements than Comparative Compound A or Comparative Compound B, and that the compound of the present invention, which satisfies a specific structure, exhibits a significant effect compared to other comparative compounds not described herein.

These results suggests that even for compounds with similar molecular components, the properties of compounds such as hole properties, light efficiency properties, energy level, hole injection and mobility properties of molecules, Charge balance between holes and electrons, volume density and distance between molecules, etc. can vary significantly to the extent that it is difficult to predict, depending on the type and position of the substituent to be substituted, and also the performance of the device may vary due to complex factors, rather than one configuration affecting the overall result of the device.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment.

The scope of the present invention shall be construed on the basis of the accompanying claims, and it shall be construed that all of the technical ideas included within the scope equivalent to the claims belong to the present invention.

What is claimed is:

1. A compound represented by Formula 3-2 or Formula 3-3: 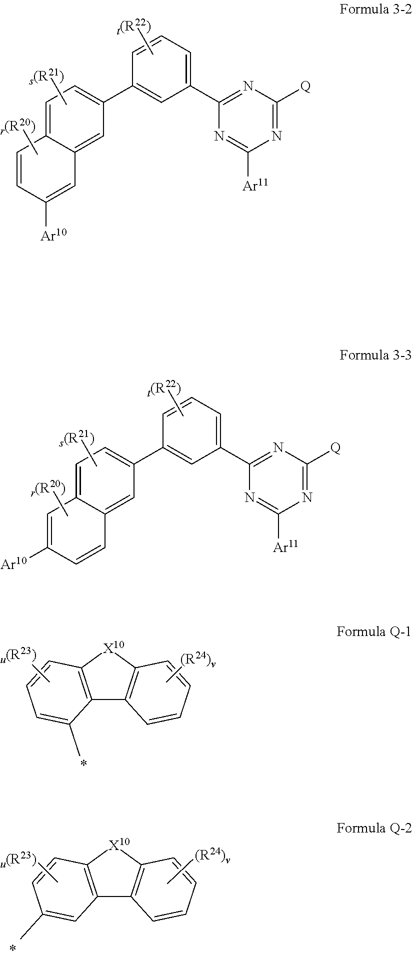
wherein:
1) Q is a substituent represented by Formula Q-1 or Formula Q-2,
2) Ar¹⁰ is a C₆-C₆₀ aryl group; or a C₆-C₆₀ aryl group substituted by deuterium,
3) Ar¹¹ is a C₆-C₆₀ aryl group; or a C₂-C₆₀ heterocyclic group including at least one heteroatom of O, N, S, Si or P,
4) R²⁰, R²¹ and R²² are the same or different, and each independently hydrogen; or deuterium,
5) R²³ and R²⁴ are the same or different, and each independently selected from the group consisting of hydrogen; deuterium; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₃-C₆₀ aliphatic ring; a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group; or an adjacent plurality of R²³s or a plurality of R²⁴s may be bonded to each other to form a ring,
6) X¹⁰ is O or S,
7) r, s and u are each independently an integer of 0 to 3, t and v are each independently an integer of 0 to 4,
8) * means a position for binding to Formula 3-2 or Formula 3-3,
wherein the aryl group, heterocyclic group, fluorenyl group, aliphatic ring group, fused ring group, alkyl group, alkenyl group, alkynyl group, alkoxy group, and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and also the substituents may be bonded to each other to form a saturated or unsaturated ring, wherein the term ‘ring’ means a C₃-C₆₀ aliphatic ring and a C₆-C₆₀ aromatic ring, or a C₂-C₆₀ heterocyclic group or a fused ring formed by the combination thereof.

2. The compound of claim 1, wherein Ar¹⁰ or Ar¹¹ are represented by one of Formulas Ar-1 to Ar-7: 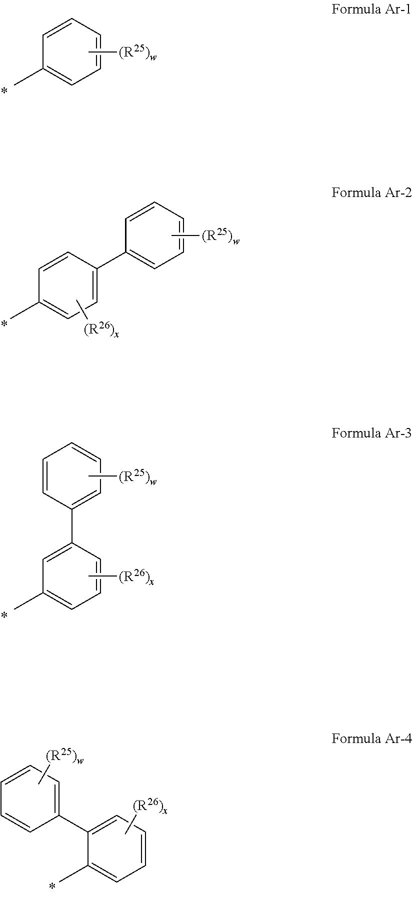 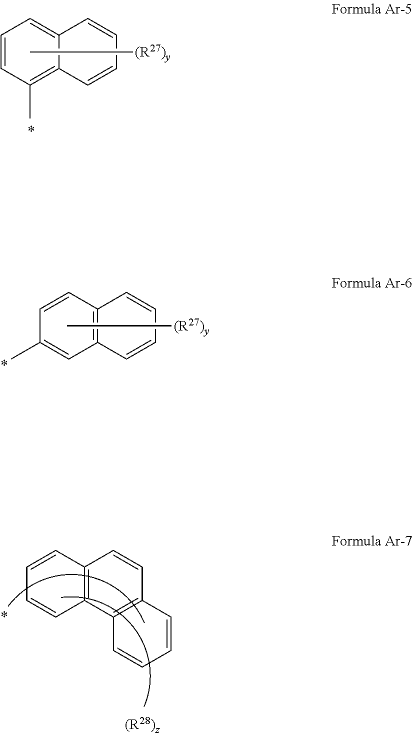
wherein:
1) R²⁵, R²⁶, R²⁷ and R²⁸ are the same as the definition of R²³ in claim 1, or an adjacent plurality of R²⁵s, a plurality of R²⁶s, a plurality of R²⁷s, or a plurality of R²⁸s may be bonded to each other to form a ring,
2) w is an integer from 0 to 5, x is an integer from 0 to 4, y is an integer from 0 to 7, z is an integer from 0 to 9,
3) * means a moiety bonded to Formula 3-2 or Formula 3-3.

3. The compound of claim 1, wherein Ar¹¹ is represented by Formula Ar-8: 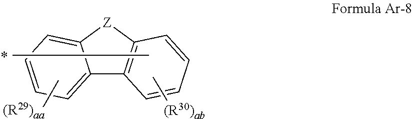
wherein:
1) Z is O, S, C(R³¹)(R³²), NR³³ or N, wherein Z is N in the case where Z is combined with Formula 3-2 or Formula 3-3,
2) R²⁹, R³⁰, R³¹, R³² and R³³ are the same as definition of R²³ in claim 1, or an adjacent plurality of R²⁹s, a plurality of R³⁰s, a plurality of R³¹s, a plurality of R³²s may be bonded to each other to form a ring,
3) aa and ab are independently an integer of 0 to 4;
4) * means a moiety bonded to Formula 3-2 or Formula 3-3.

4. The compound of claim 1, wherein Formula 3-2 or Formula 3-3 is represented by any one of the following compounds.

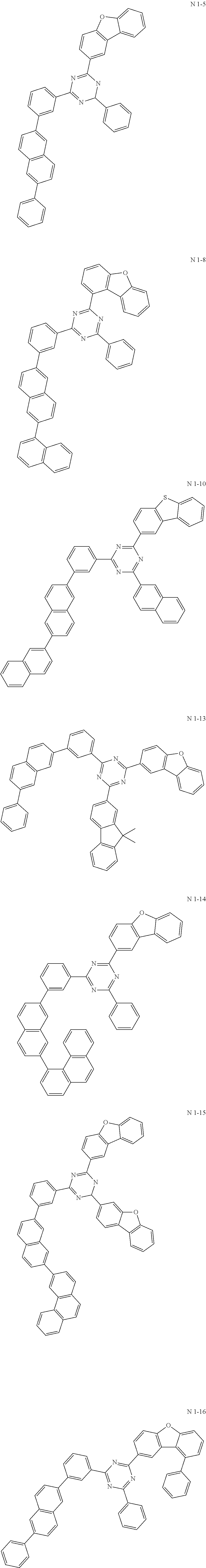

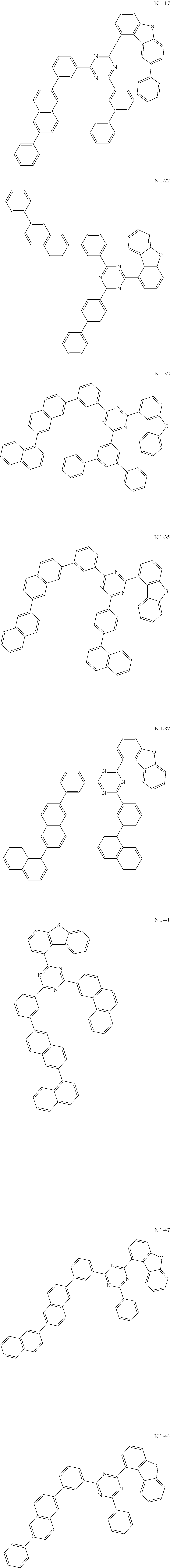

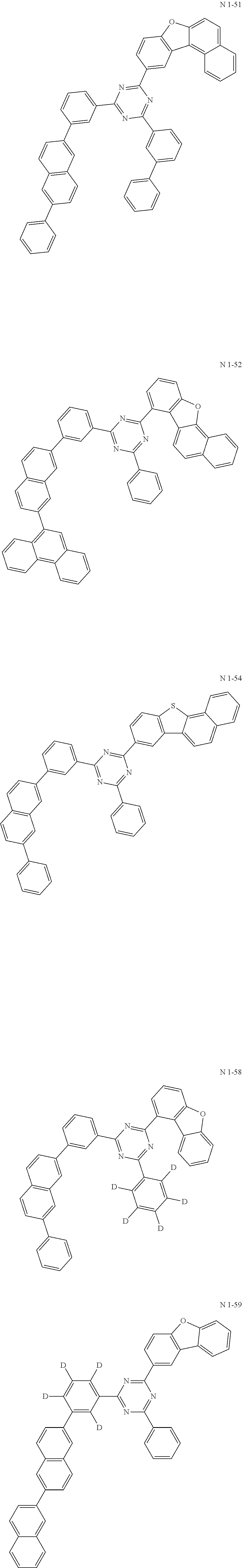

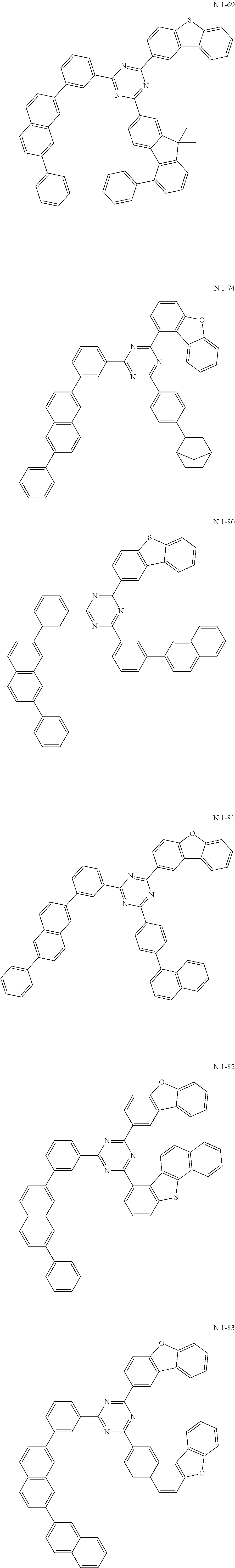

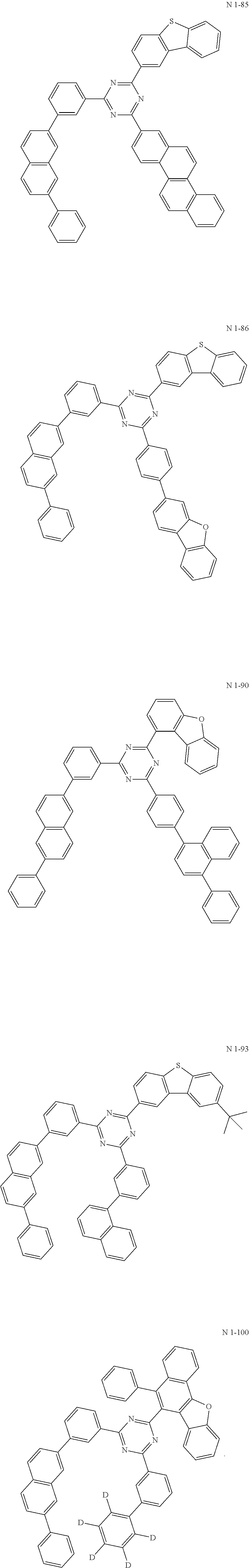

5. An organic electronic element comprising a first electrode; a second electrode; and an organic material layer formed between the first electrode and the second electrode, wherein the organic material layer includes an emitting layer, and the emitting layer is a phosphorescent emitting layer and comprises a first host compound represented by Formula 3-2 or Formula 3-3 in claim 1 and a second host compound represented by Formula 4 or Formula 5. 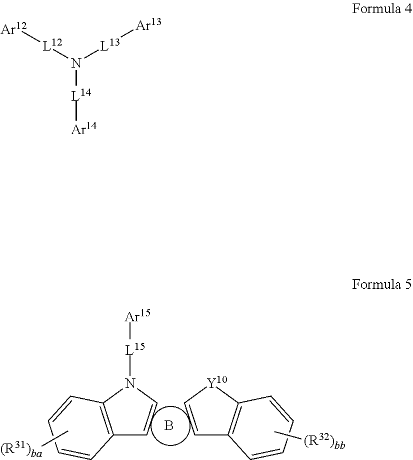
wherein:
Ar¹², Ar¹³ and Ar¹⁴ are each independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring;
Ar¹⁵ is each independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and -L″-NR^{f}R^{g};
L¹², L¹³ L¹⁴, L¹⁵ and L″ are each independently selected from the group consisting of single bond; a C₆-C₆₀ arylene group; a fluorenylene group; a fused ring group of a C₃-C₆₀ aliphatic ring and a C₆-C₆₀ aromatic ring; a C₂-C₆₀ heterocyclic group including at least one heteroatom of O, N, S, Si or P;
R^{f} and R^{g} are each independently selected from the group consisting of a C₆-C₆₀ aryl group; a fluorenyl group; a C₂-C₆₀ heterocyclic group including at least one heteroatom of O, N, S, Si or P; a C₃-C₆₀ aliphatic ring;
Y¹⁰ is O, S, CR⁵¹R⁵² or NR⁵³,
B ring is a C₆˜C₂₀ aryl group,
R³¹ and R³² are the same or different, and each independently selected from the group consisting of a hydrogen; deuterium; halogen; cyano group; nitro group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₆₀ alkenyl group; a C₂-C₆₀ alkynyl group; a C₁-C₅₀ alkoxy group; a C₆-C₆₀ aryloxy group; or a plurality of adjacent R³¹s or a plurality of R³²s may be bonded to each other to form a ring,
R⁵¹, R⁵² and R⁵³ are each independently selected from the group consisting of a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; a C₆-C₆₀ aryloxy group; or R⁵¹ and R⁵² may be bonded to each other to form a spiro,
ba and bb are independently an integer of 0 to 4;
wherein the aryl group, arylene group, heterocyclic group, fluorenyl group, fluorenylene group, aliphatic ring group, fused ring group, alkyl group, alkenyl group, alkynyl group, alkoxyl group, and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

6. The compound according to claim 5, wherein the compound represented by Formula 4 is any one of the following compounds H-1 to H-100:

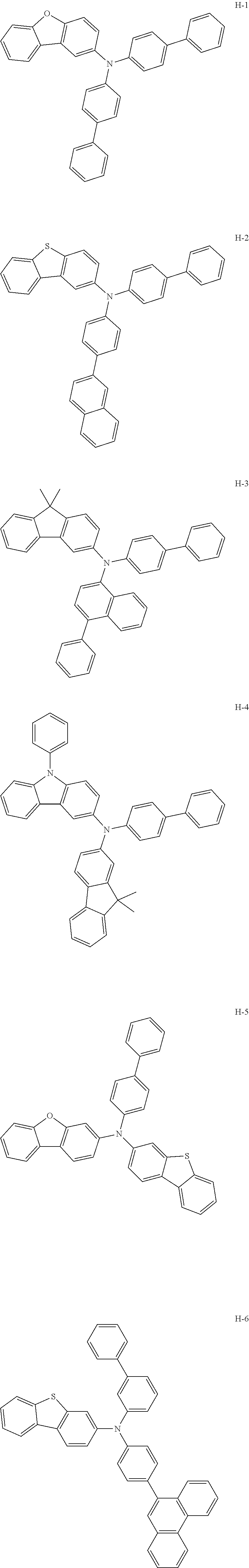 [SMILES: c1ccc(-c2ccc(N(c3ccc(-c4ccccc4)cc3)c3ccc4oc5ccccc5c4c3)cc2)cc1 ; c1ccc(-c2ccc(N(c3ccc(-c4ccc5ccccc5c4)cc3)c3ccc4sc5ccccc5c4c3)cc2)cc1 ; CC1(C)c2ccccc2-c2cc(N(c3ccc(-c4ccccc4)cc3)c3ccc(-c4ccccc4)c4ccccc34)ccc21 ; CC1(C)c2ccccc2-c2ccc(N(c3ccc(-c4ccccc4)cc3)c3ccc4c(c3)c3ccccc3n4-c3ccccc3)cc21 ; c1ccc(-c2ccc(N(c3ccc4c(c3)oc3ccccc34)c3ccc4c(c3)sc3ccccc34)cc2)cc1 ; c1ccc(-c2cccc(N(c3ccc(-c4cc5ccccc5c5ccccc45)cc3)c3ccc4c(c3)sc3ccccc34)c2)cc1]

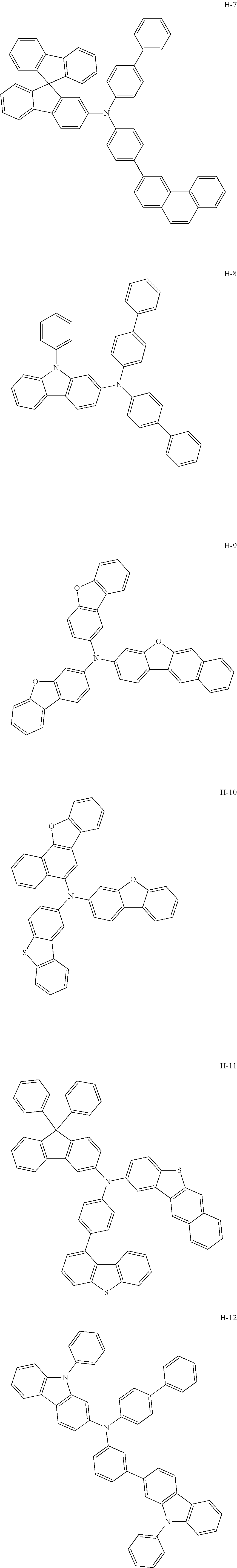

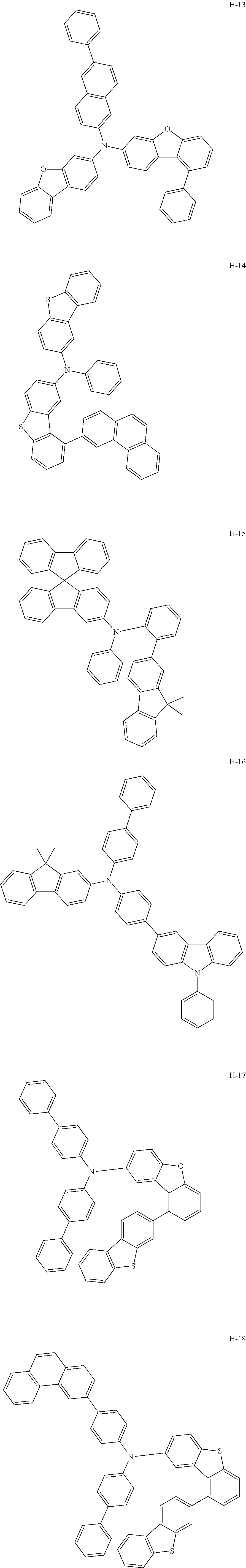

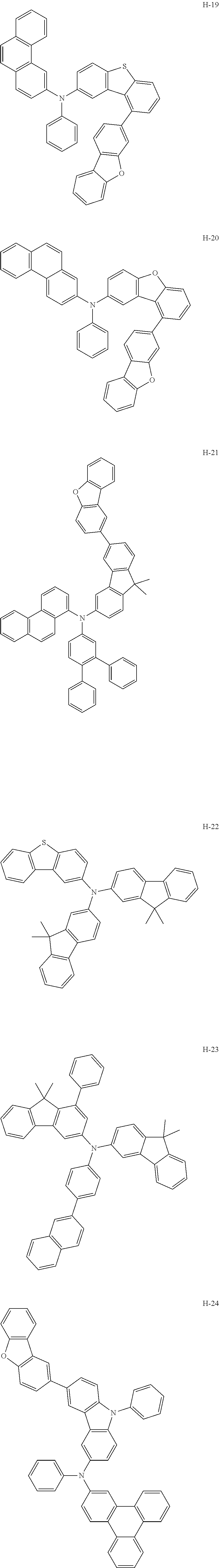

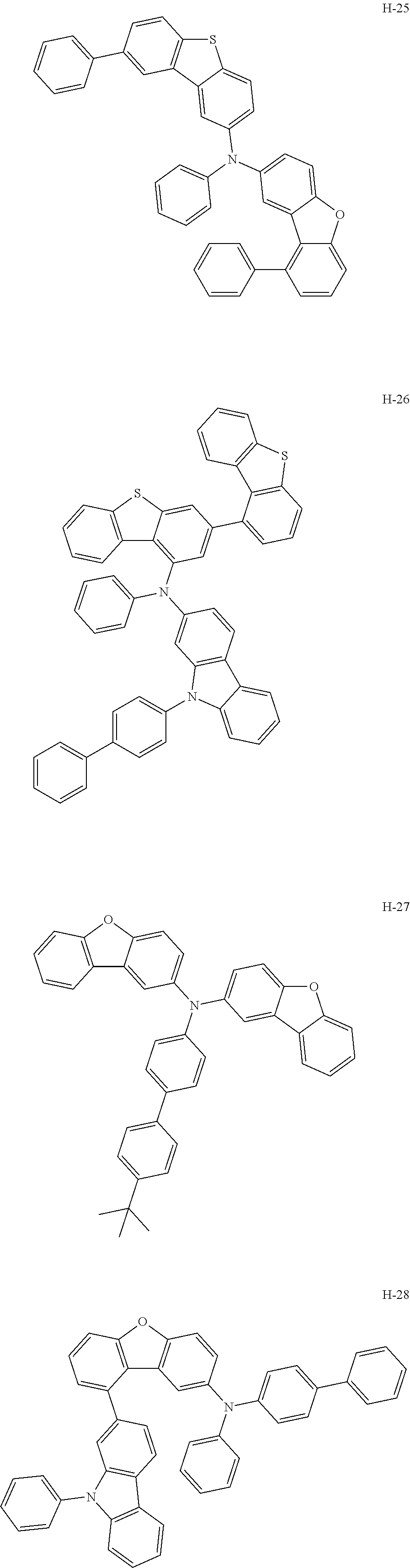 [SMILES: c1ccc(-c2ccc3sc4ccc(N(c5ccccc5)c5ccc6oc7cccc(-c8ccccc8)c7c6c5)cc4c3c2)cc1 ; c1ccc(-c2ccc(-n3c4ccccc4c4ccc(N(c5ccccc5)c5cc(-c6cccc7sc8ccccc8c67)cc6sc7ccccc7c56)cc43)cc2)cc1 ; CC(C)(C)c1ccc(-c2ccc(N(c3ccc4oc5ccccc5c4c3)c3ccc4oc5ccccc5c4c3)cc2)cc1 ; c1ccc(-c2ccc(N(c3ccccc3)c3ccc4oc5cccc(-c6ccc7c8ccccc8n(-c8ccccc8)c7c6)c5c4c3)cc2)cc1]

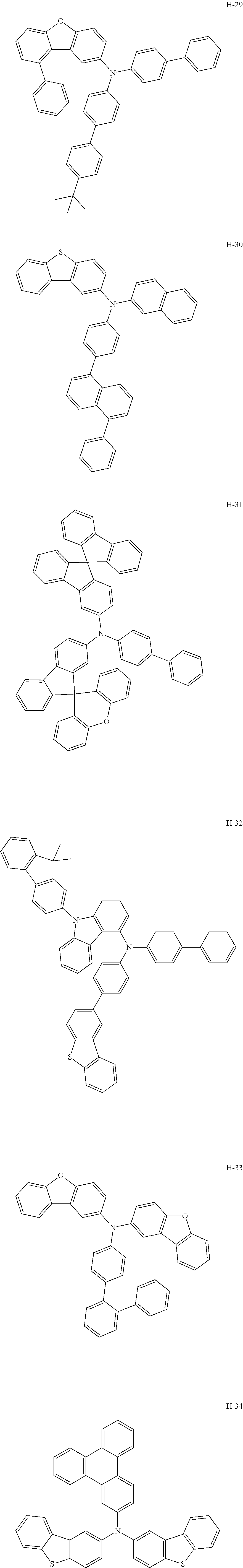

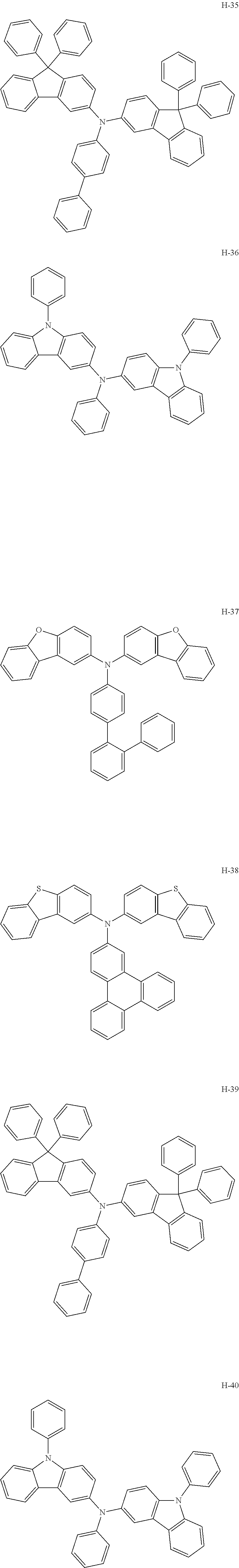

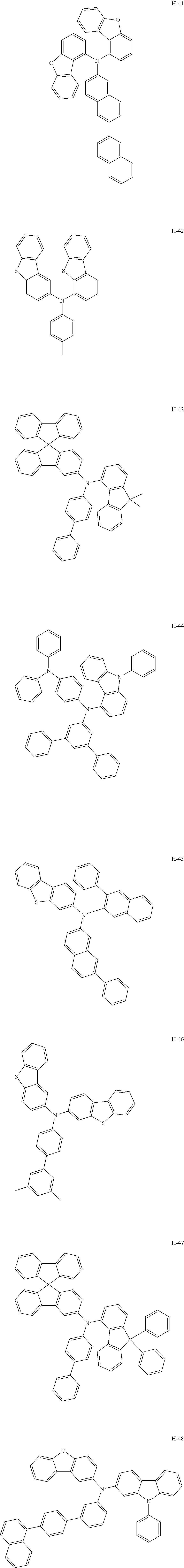

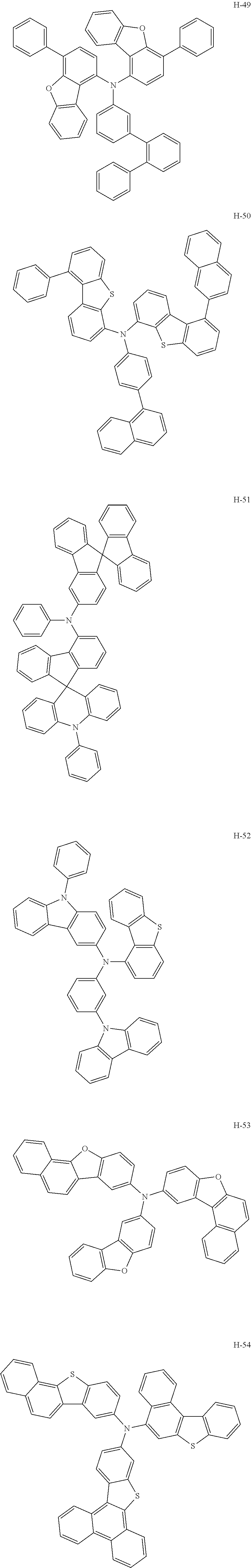

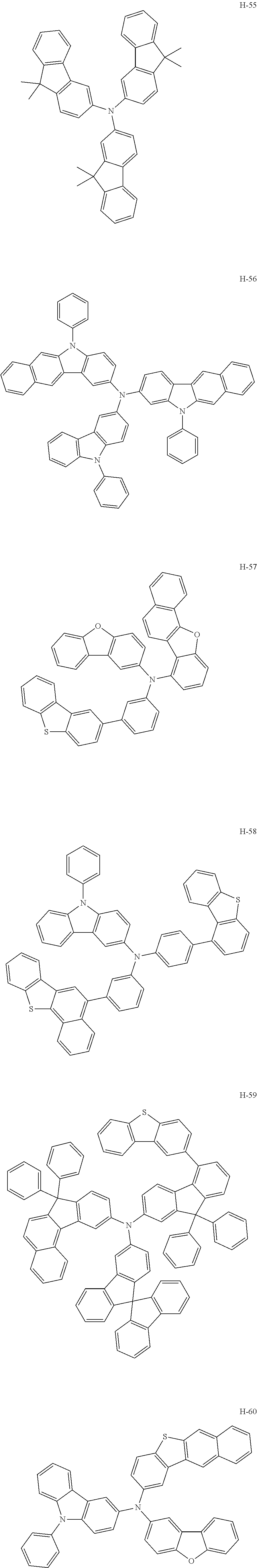

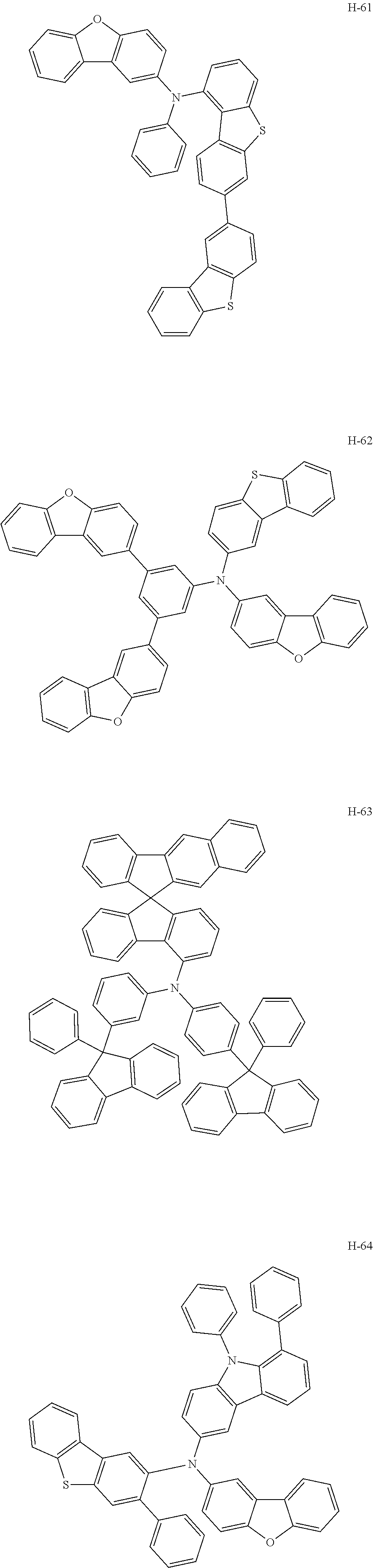 [SMILES: c1ccc(N(c2ccc3oc4ccccc4c3c2)c2cccc3sc4cc(-c5ccc6sc7ccccc7c6c5)ccc4c23)cc1 ; c1ccc2c(c1)oc1ccc(-c3cc(-c4ccc5oc6ccccc6c5c4)cc(N(c4ccc5oc6ccccc6c5c4)c4ccc5sc6ccccc6c5c4)c3)cc12 ; c1ccc(C2(c3ccc(N(c4cccc(C5(c6ccccc6)c6ccccc6-c6ccccc65)c4)c4cccc5c4-c4ccccc4C54c5ccccc5-c5cc6ccccc6cc54)cc3)c3ccccc3-c3ccccc32)cc1 ; c1ccc(-c2cc3sc4ccccc4c3cc2N(c2ccc3oc4ccccc4c3c2)c2ccc3c(c2)c2cccc(-c4ccccc4)c2n3-c2ccccc2)cc1]

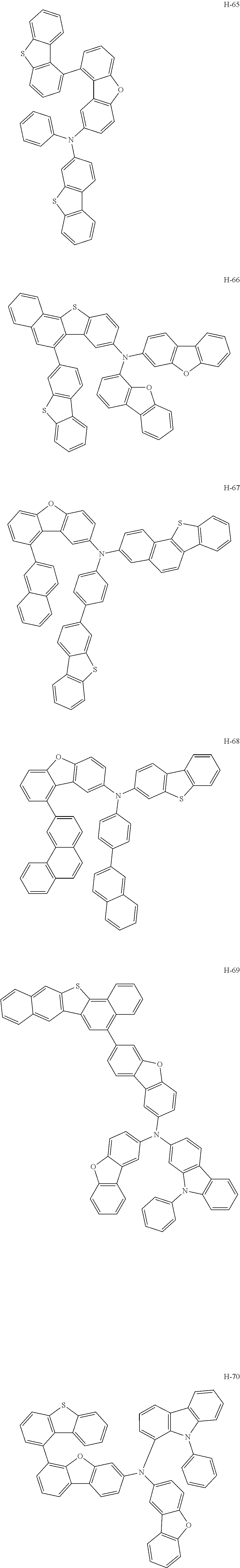

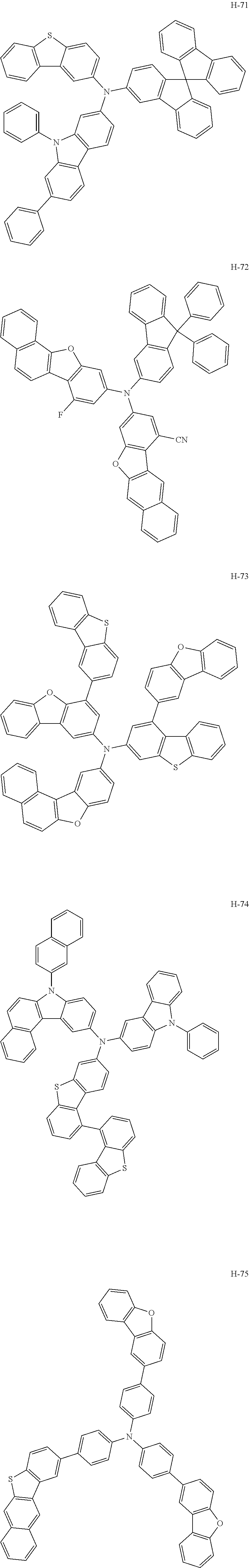

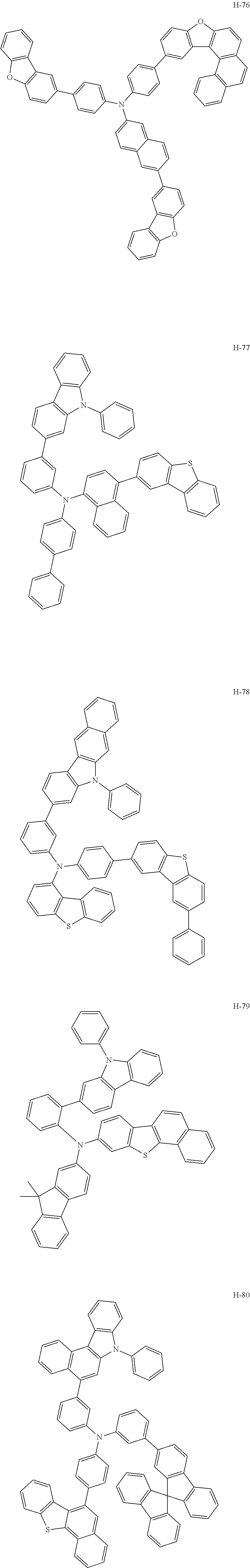

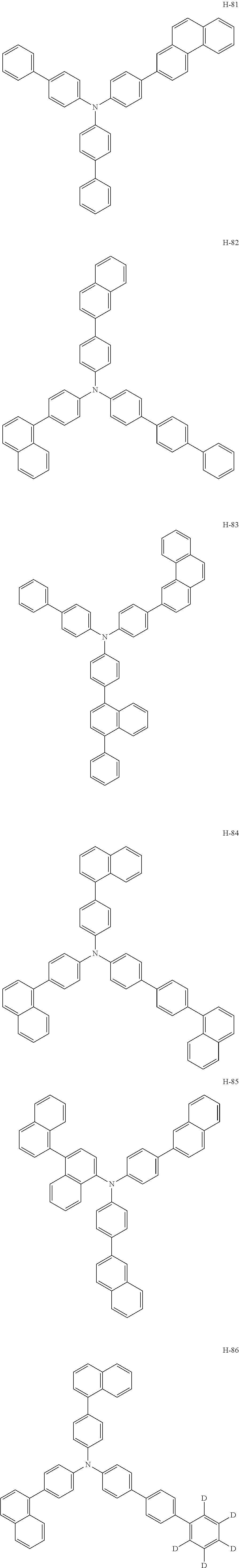

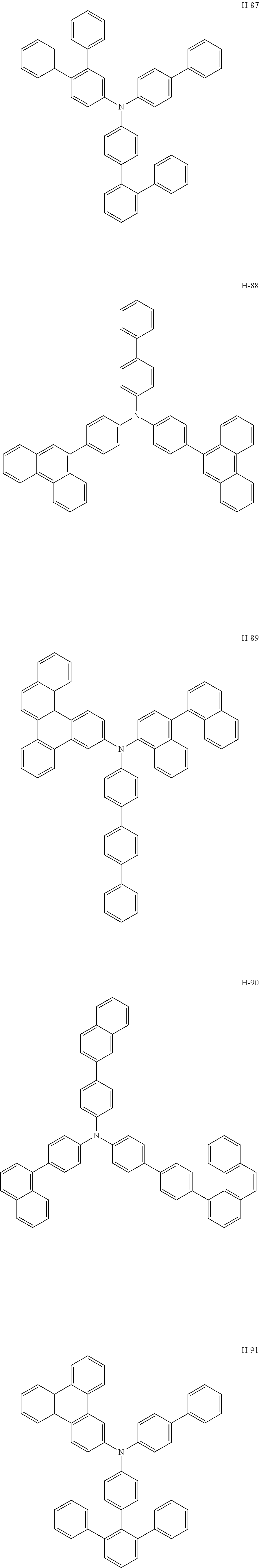 [SMILES: c1ccc(-c2ccc(N(c3ccc(-c4ccccc4-c4ccccc4)cc3)c3ccc(-c4ccccc4)c(-c4ccccc4)c3)cc2)cc1 ; c1ccc(-c2ccc(N(c3ccc(-c4cc5ccccc5c5ccccc45)cc3)c3ccc(-c4cc5ccccc5c5ccccc45)cc3)cc2)cc1 ; c1ccc(-c2ccc(-c3ccc(N(c4ccc5c(c4)c4ccccc4c4ccc6ccccc6c45)c4ccc(-c5cccc6ccccc56)c5ccccc45)cc3)cc2)cc1 ; c1ccc2cc(-c3ccc(N(c4ccc(-c5ccc(-c6cccc7ccc8ccccc8c67)cc5)cc4)c4ccc(-c5cccc6ccccc56)cc4)cc3)ccc2c1 ; c1ccc(-c2ccc(N(c3ccc(-c4c(-c5ccccc5)cccc4-c4ccccc4)cc3)c3ccc4c5ccccc5c5ccccc5c4c3)cc2)cc1]

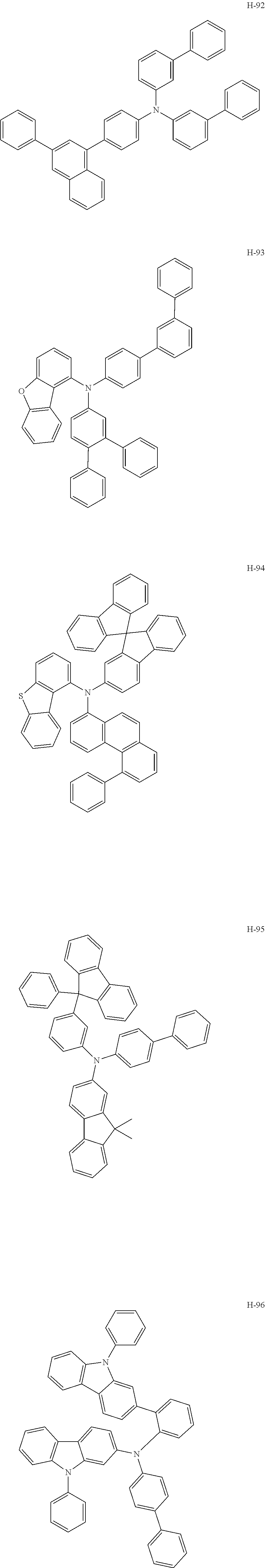 [SMILES: c1ccc(-c2cccc(N(c3ccc(-c4cc(-c5ccccc5)cc5ccccc45)cc3)c3cccc(-c4ccccc4)c3)c2)cc1 ; c1ccc(-c2cccc(-c3ccc(N(c4ccc(-c5ccccc5)c(-c5ccccc5)c4)c4cccc5oc6ccccc6c45)cc3)c2)cc1 ; c1ccc(-c2cccc3ccc4c(N(c5ccc6c(c5)C5(c7ccccc7-c7ccccc75)c5ccccc5-6)c5cccc6sc7ccccc7c56)cccc4c23)cc1 ; CC1(C)c2ccccc2-c2ccc(N(c3ccc(-c4ccccc4)cc3)c3cccc(C4(c5ccccc5)c5ccccc5-c5ccccc54)c3)cc21 ; c1ccc(-c2ccc(N(c3ccc4c5ccccc5n(-c5ccccc5)c4c3)c3ccccc3-c3ccc4c5ccccc5n(-c5ccccc5)c4c3)cc2)cc1]

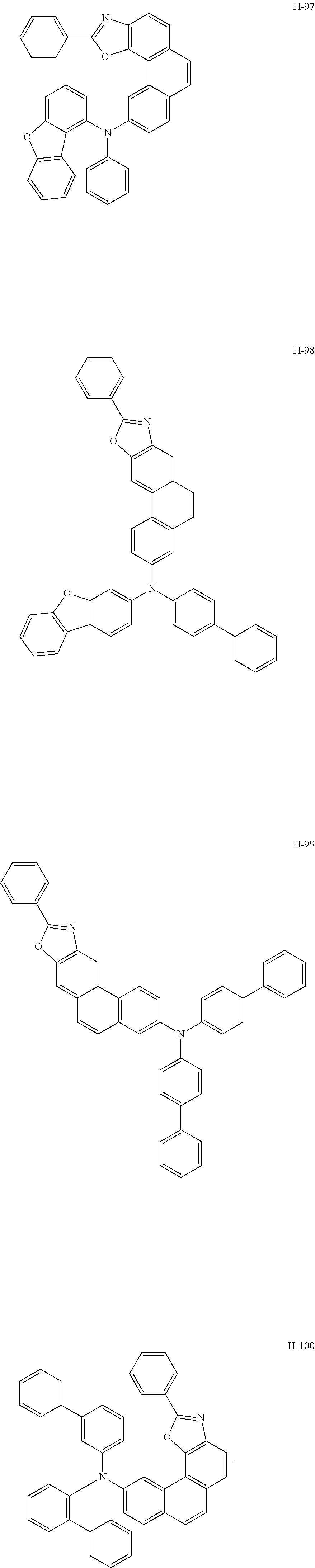 [SMILES: c1ccc(-c2nc3ccc4ccc5ccc(N(c6ccccc6)c6cccc7oc8ccccc8c67)cc5c4c3o2)cc1 ; c1ccc(-c2ccc(N(c3ccc4c(ccc5cc6nc(-c7ccccc7)oc6cc54)c3)c3ccc4c(c3)oc3ccccc34)cc2)cc1 ; c1ccc(-c2ccc(N(c3ccc(-c4ccccc4)cc3)c3ccc4c(ccc5cc6oc(-c7ccccc7)nc6cc54)c3)cc2)cc1 ; c1ccc(-c2cccc(N(c3ccc4ccc5ccc6nc(-c7ccccc7)oc6c5c4c3)c3ccccc3-c3ccccc3)c2)cc1 ; "."]

7. The compound according to claim 5, wherein the compound represented by Formula 5 is any one of the following compounds S-1 to S-108:

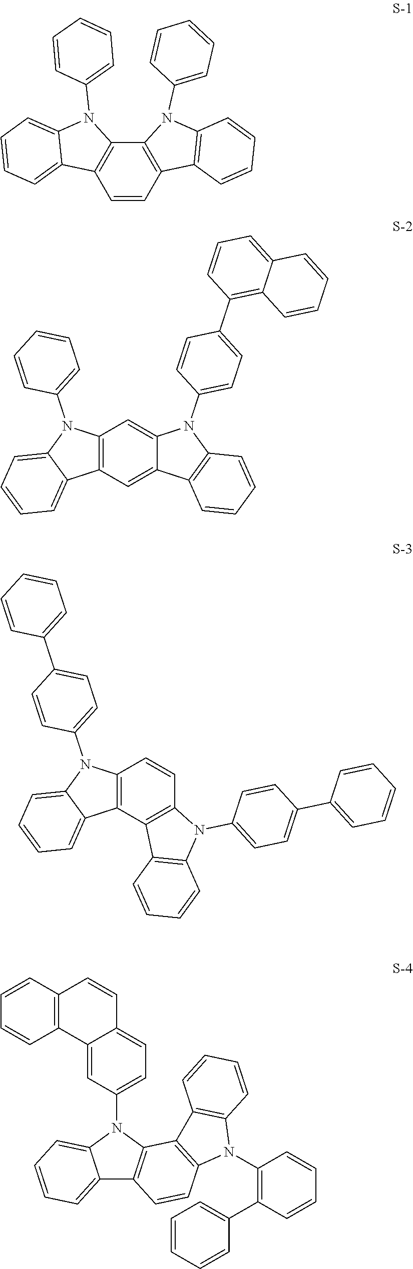 [SMILES: c1ccc(-n2c3ccccc3c3ccc4c5ccccc5n(-c5ccccc5)c4c32)cc1 ; c1ccc(-n2c3ccccc3c3cc4c5ccccc5n(-c5ccc(-c6cccc7ccccc67)cc5)c4cc32)cc1 ; c1ccc(-c2ccc(-n3c4ccccc4c4c5c6ccccc6n(-c6ccc(-c7ccccc7)cc6)c5ccc43)cc2)cc1 ; c1ccc(-c2ccccc2-n2c3ccccc3c3c2ccc2c4ccccc4n(-c4ccc5ccc6ccccc6c5c4)c23)cc1]

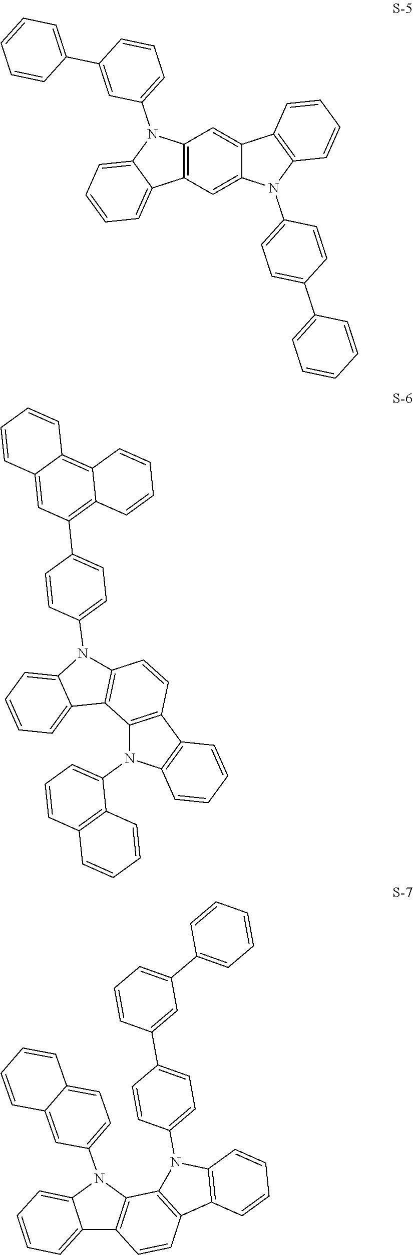 [SMILES: c1ccc(-c2ccc(-n3c4ccccc4c4cc5c(cc43)c3ccccc3n5-c3cccc(-c4ccccc4)c3)cc2)cc1 ; c1ccc2c(-n3c4ccccc4c4ccc5c(c6ccccc6n5-c5ccc(-c6cc7ccccc7c7ccccc67)cc5)c43)cccc2c1 ; c1ccc(-c2cccc(-c3ccc(-n4c5ccccc5c5ccc6c7ccccc7n(-c7ccc8ccccc8c7)c6c54)cc3)c2)cc1]

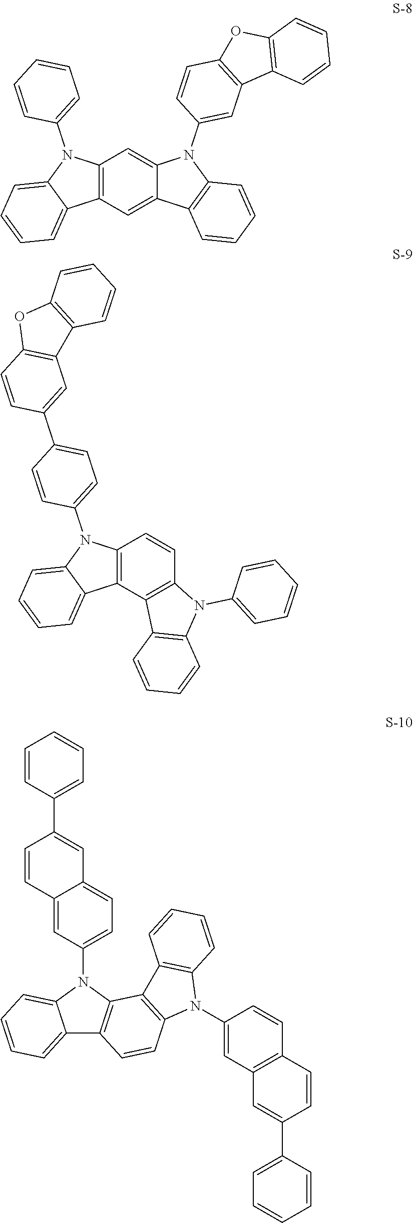

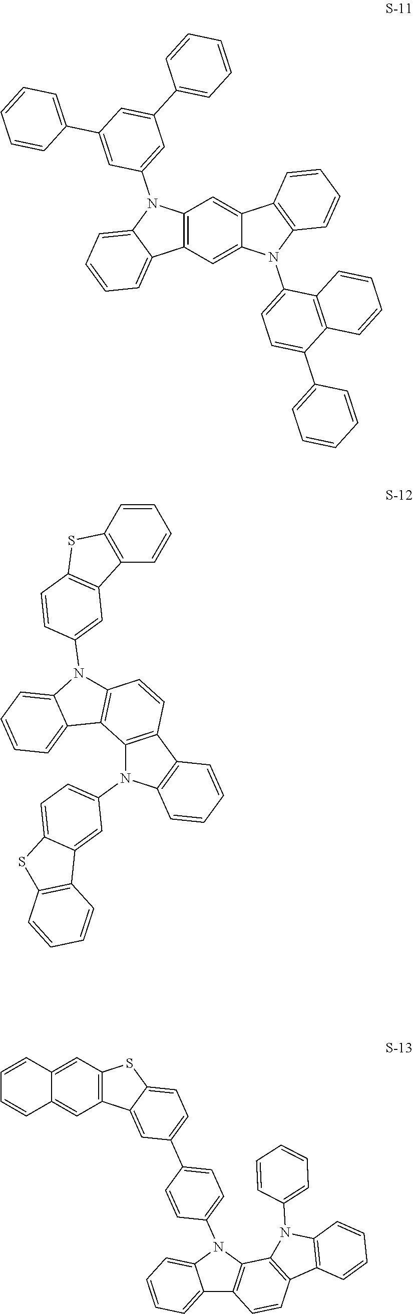 [SMILES: c1ccc(-c2cc(-c3ccccc3)cc(-n3c4ccccc4c4cc5c(cc43)c3ccccc3n5-c3ccc(-c4ccccc4)c4ccccc34)c2)cc1 ; c1ccc2c(c1)sc1ccc(-n3c4ccccc4c4c3ccc3c5ccccc5n(-c5ccc6sc7ccccc7c6c5)c34)cc12 ; c1ccc(-n2c3ccccc3c3ccc4c5ccccc5n(-c5ccc(-c6ccc7sc8cc9ccccc9cc8c7c6)cc5)c4c32)cc1]

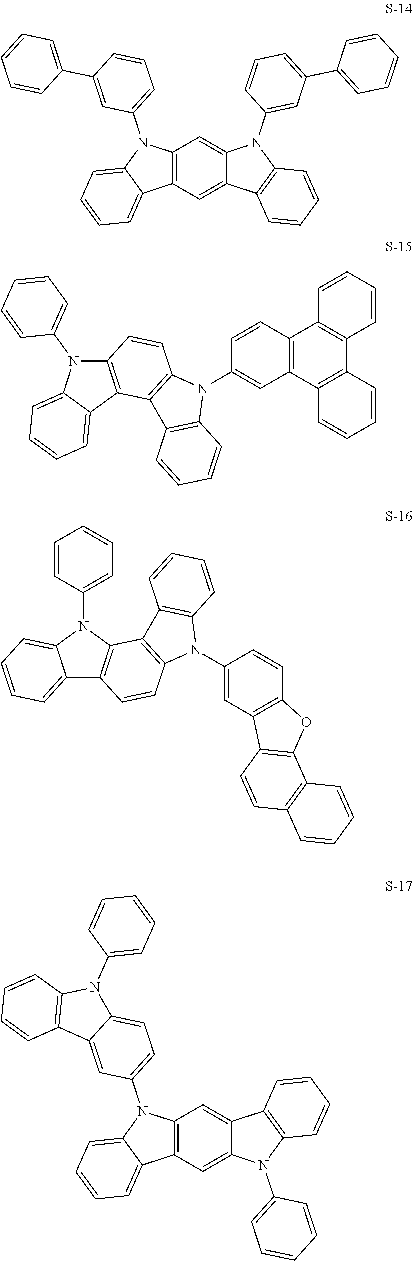 [SMILES: c1ccc(-c2cccc(-n3c4ccccc4c4cc5c6ccccc6n(-c6cccc(-c7ccccc7)c6)c5cc43)c2)cc1 ; c1ccc(-n2c3ccccc3c3c4c5ccccc5n(-c5ccc6c7ccccc7c7ccccc7c6c5)c4ccc32)cc1 ; c1ccc(-n2c3ccccc3c3ccc4c(c5ccccc5n4-c4ccc5oc6c7ccccc7ccc6c5c4)c32)cc1 ; c1ccc(-n2c3ccccc3c3cc(-n4c5ccccc5c5cc6c(cc54)c4ccccc4n6-c4ccccc4)ccc32)cc1]

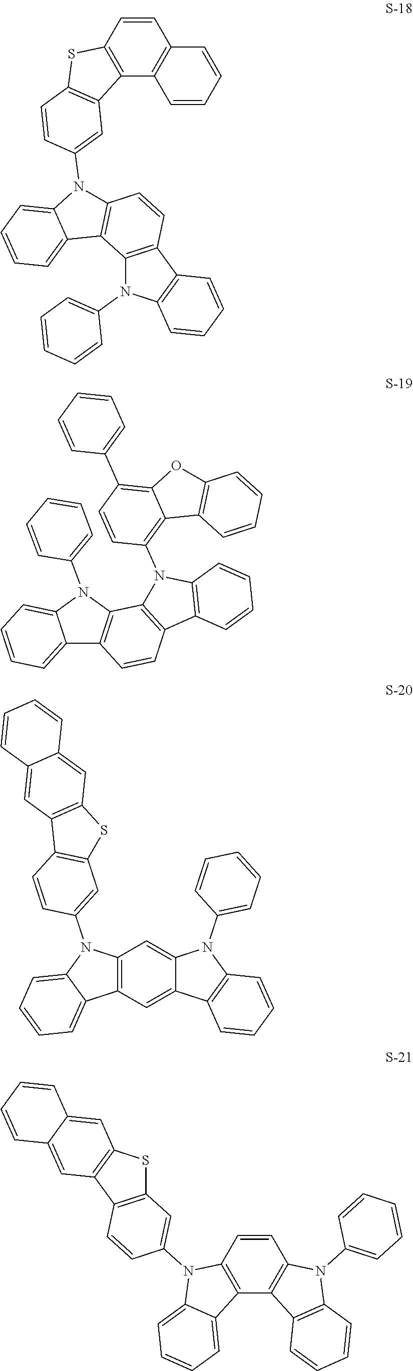

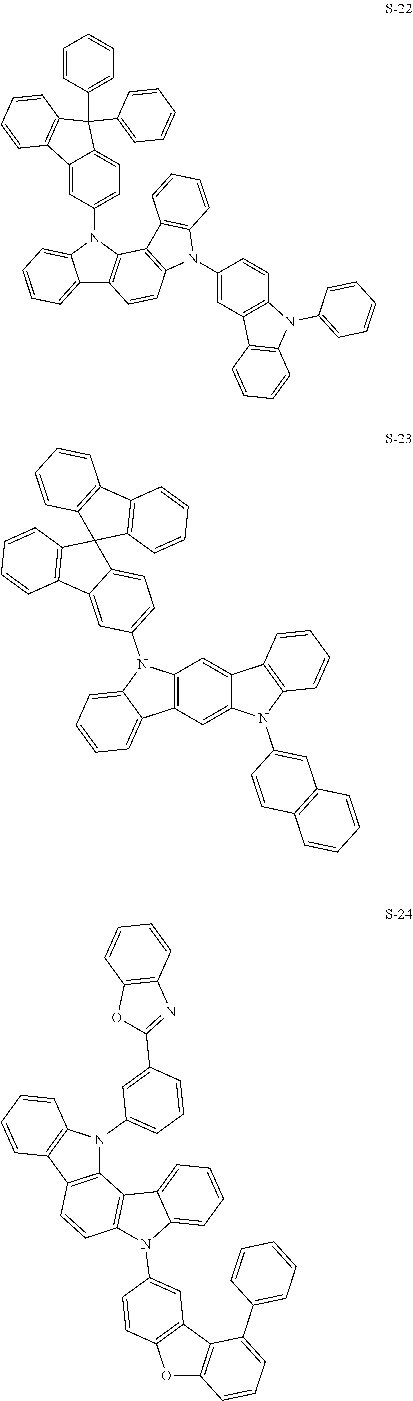 [SMILES: c1ccc(-n2c3ccccc3c3cc(-n4c5ccccc5c5c4ccc4c6ccccc6n(-c6ccc7c(c6)-c6ccccc6C7(c6ccccc6)c6ccccc6)c45)ccc32)cc1 ; c1ccc2c(c1)-c1ccccc1C21c2ccccc2-c2cc(-n3c4ccccc4c4cc5c(cc43)c3ccccc3n5-c3ccc4ccccc4c3)ccc21 ; c1ccc(-c2cccc3oc4ccc(-n5c6ccccc6c6c5ccc5c7ccccc7n(-c7cccc(-c8nc9ccccc9o8)c7)c56)cc4c23)cc1]

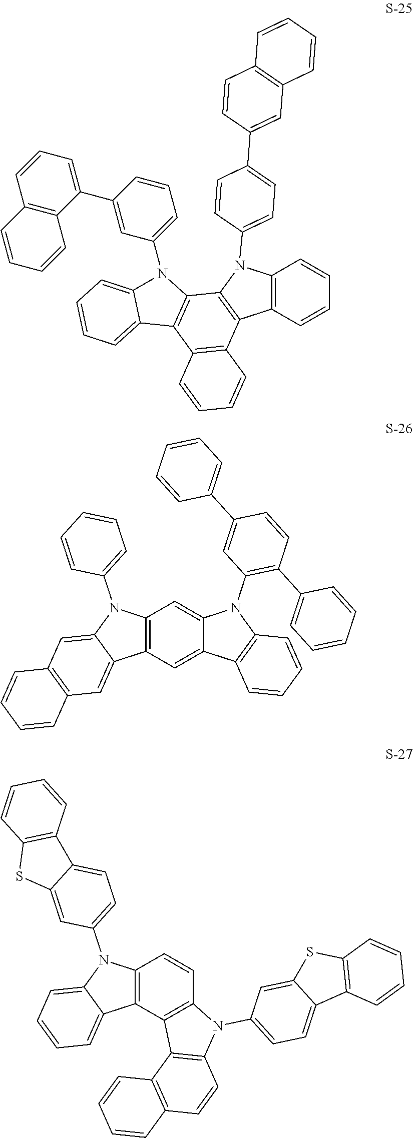

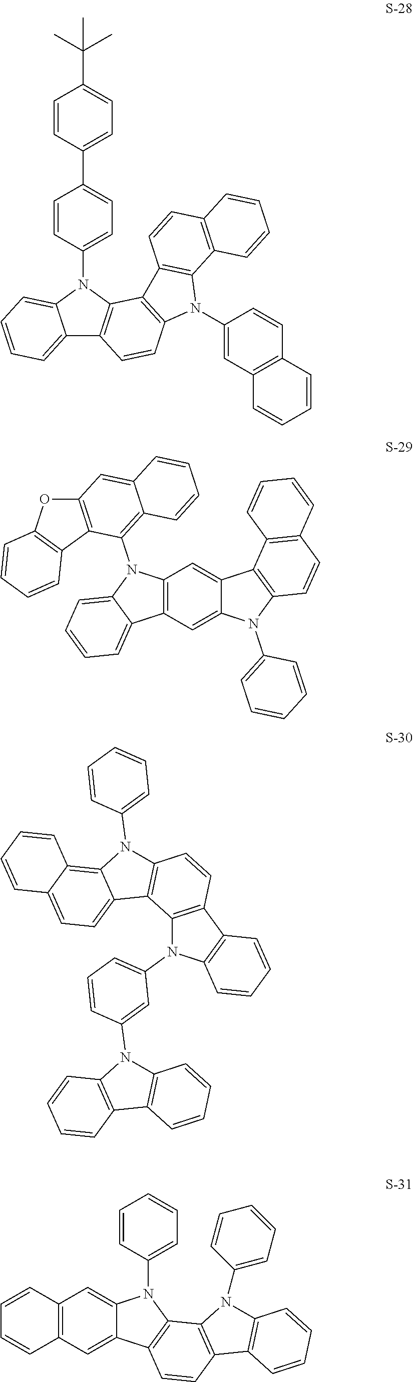 [SMILES: CC(C)(C)c1ccc(-c2ccc(-n3c4ccccc4c4ccc5c(c6ccc7ccccc7c6n5-c5ccc6ccccc6c5)c43)cc2)cc1 ; c1ccc(-n2c3cc4c5ccccc5n(-c5c6ccccc6cc6oc7ccccc7c56)c4cc3c3c4ccccc4ccc32)cc1 ; c1ccc(-n2c3ccc4c5ccccc5n(-c5cccc(-n6c7ccccc7c7ccccc76)c5)c4c3c3ccc4ccccc4c32)cc1 ; c1ccc(-n2c3ccccc3c3ccc4c5cc6ccccc6cc5n(-c5ccccc5)c4c32)cc1]

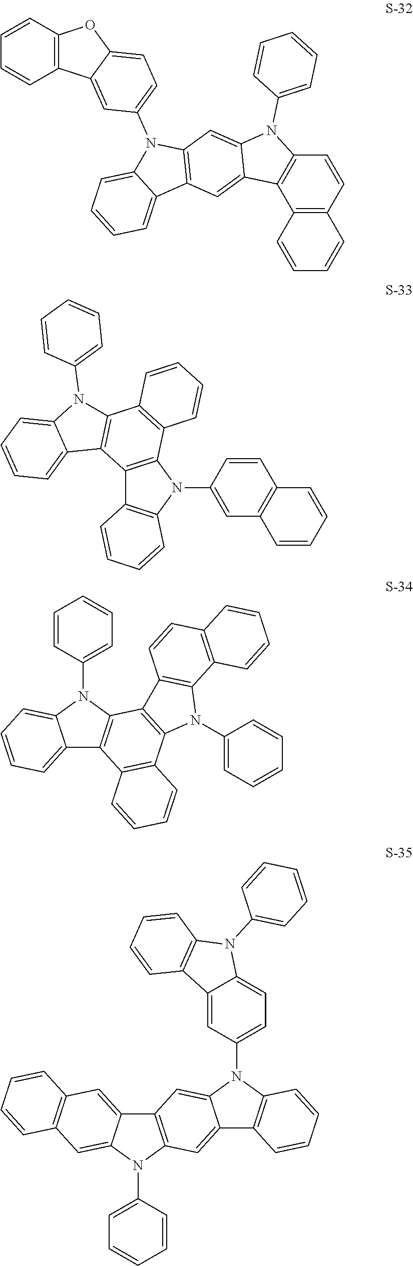

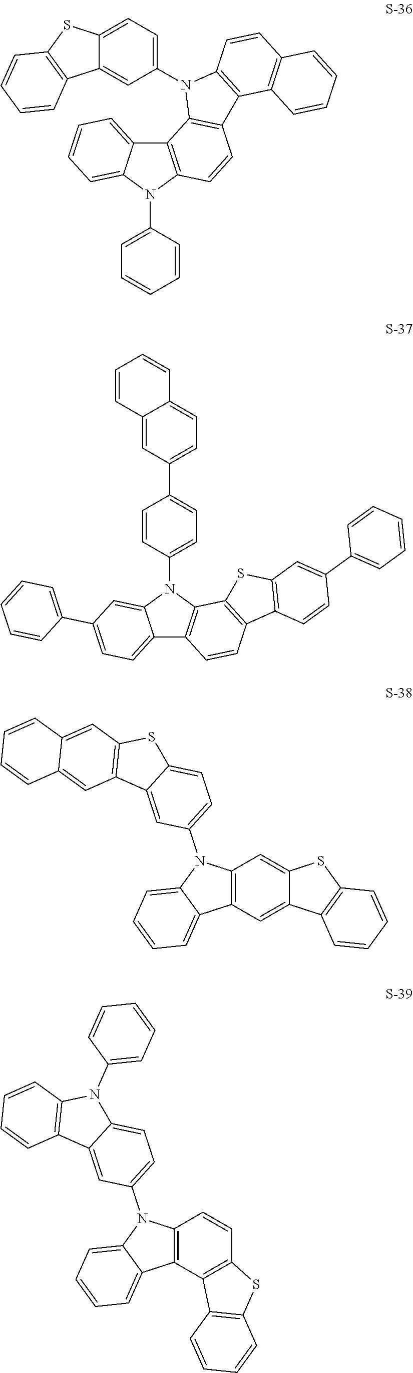 [SMILES: c1ccc(-n2c3ccccc3c3c2ccc2c4c5ccccc5ccc4n(-c4ccc5sc6ccccc6c5c4)c23)cc1 ; c1ccc(-c2ccc3c(c2)sc2c3ccc3c4ccc(-c5ccccc5)cc4n(-c4ccc(-c5ccc6ccccc6c5)cc4)c32)cc1 ; c1ccc2cc3c(cc2c1)sc1ccc(-n2c4ccccc4c4cc5c(cc42)sc2ccccc25)cc13 ; c1ccc(-n2c3ccccc3c3cc(-n4c5ccccc5c5c6c(ccc54)sc4ccccc46)ccc32)cc1]

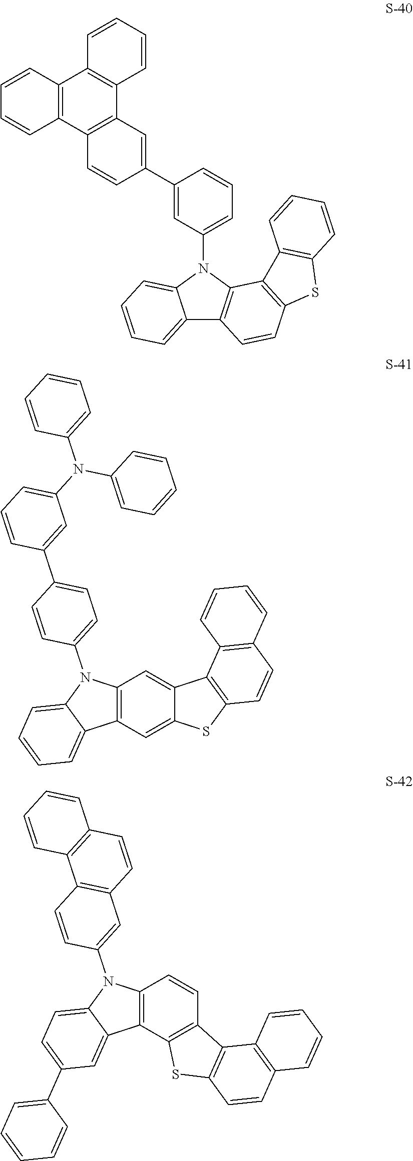

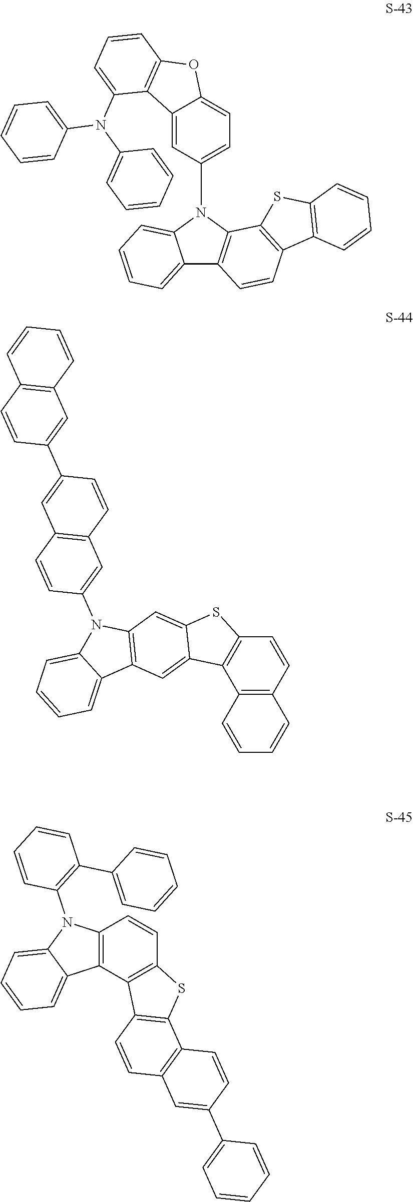 [SMILES: c1ccc(N(c2ccccc2)c2cccc3oc4ccc(-n5c6ccccc6c6ccc7c8ccccc8sc7c65)cc4c23)cc1 ; c1ccc2cc(-c3ccc4cc(-n5c6ccccc6c6cc7c(cc65)sc5ccc6ccccc6c57)ccc4c3)ccc2c1 ; c1ccc(-c2ccc3c(ccc4c3sc3ccc5c(c6ccccc6n5-c5ccccc5-c5ccccc5)c34)c2)cc1]

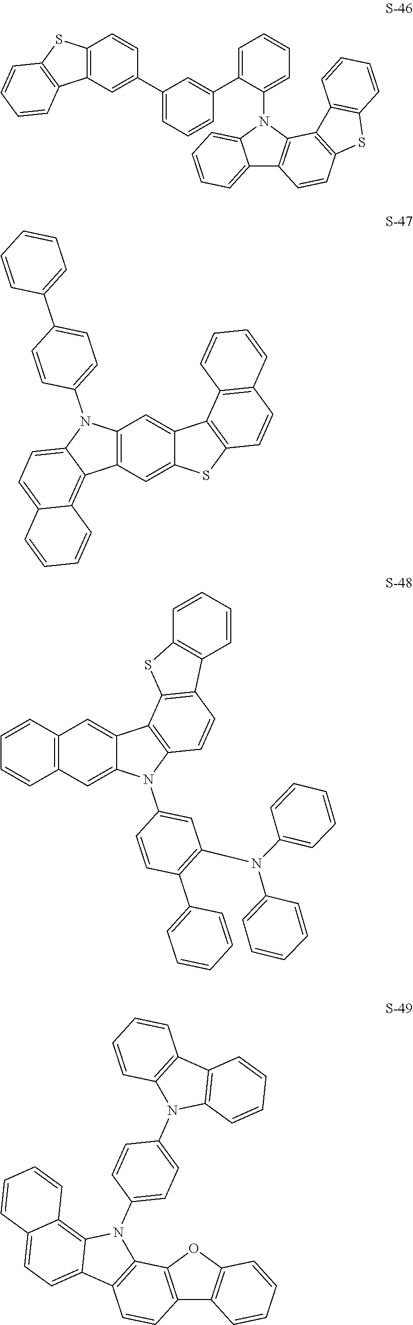 [SMILES: c1cc(-c2ccc3sc4ccccc4c3c2)cc(-c2ccccc2-n2c3ccccc3c3ccc4sc5ccccc5c4c32)c1 ; c1ccc(-c2ccc(-n3c4cc5c(cc4c4c6ccccc6ccc43)sc3ccc4ccccc4c35)cc2)cc1 ; c1ccc(-c2ccc(-n3c4cc5ccccc5cc4c4c5sc6ccccc6c5ccc43)cc2N(c2ccccc2)c2ccccc2)cc1 ; c1ccc2c(c1)ccc1c3ccc4c5ccccc5oc4c3n(-c3ccc(-n4c5ccccc5c5ccccc54)cc3)c21]

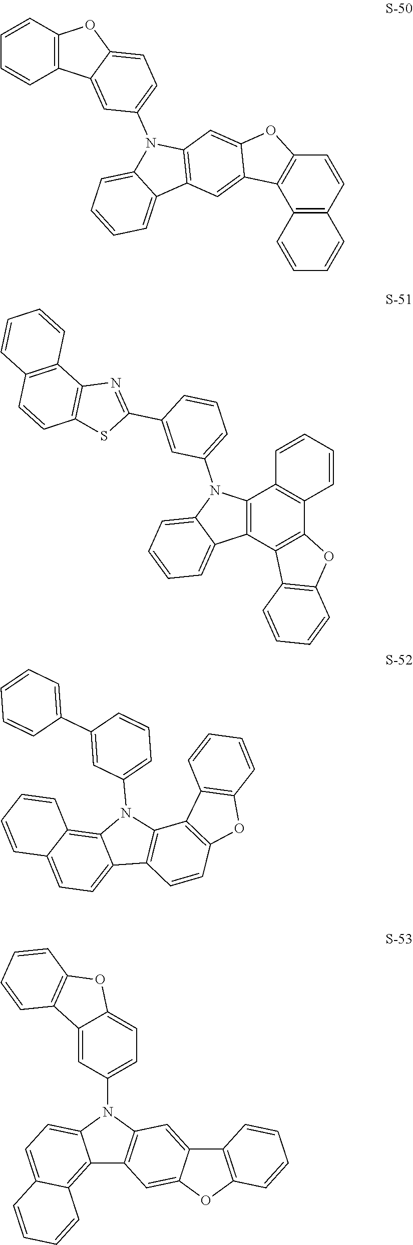 [SMILES: c1ccc2c(c1)ccc1oc3cc4c(cc3c12)c1ccccc1n4-c1ccc2oc3ccccc3c2c1 ; c1cc(-c2nc3c(ccc4ccccc43)s2)cc(-n2c3ccccc3c3c4c5ccccc5oc4c4ccccc4c32)c1 ; c1ccc(-c2cccc(-n3c4c5ccccc5ccc4c4ccc5oc6ccccc6c5c43)c2)cc1 ; c1ccc2c(c1)ccc1c2c2cc3oc4ccccc4c3cc2n1-c1ccc2oc3ccccc3c2c1]

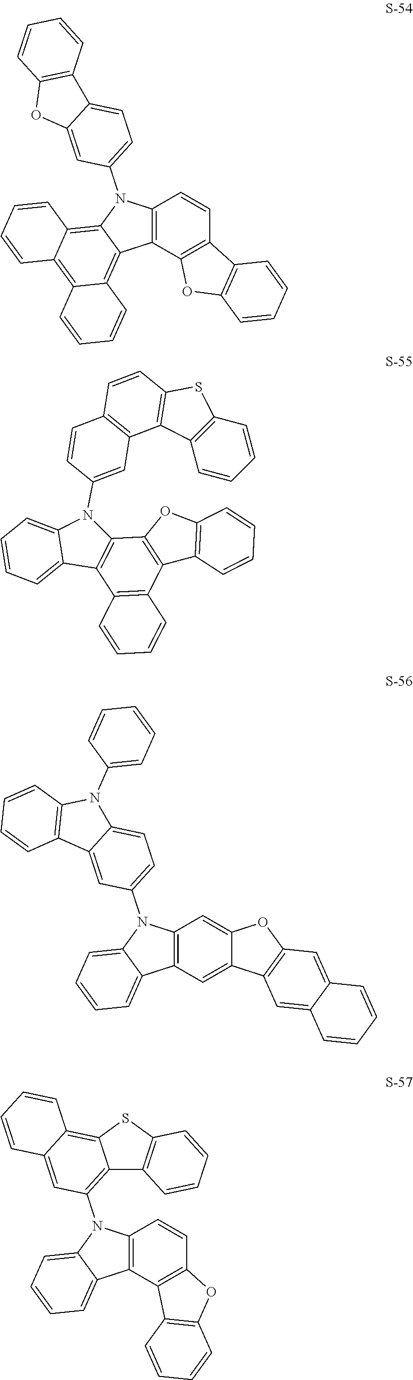

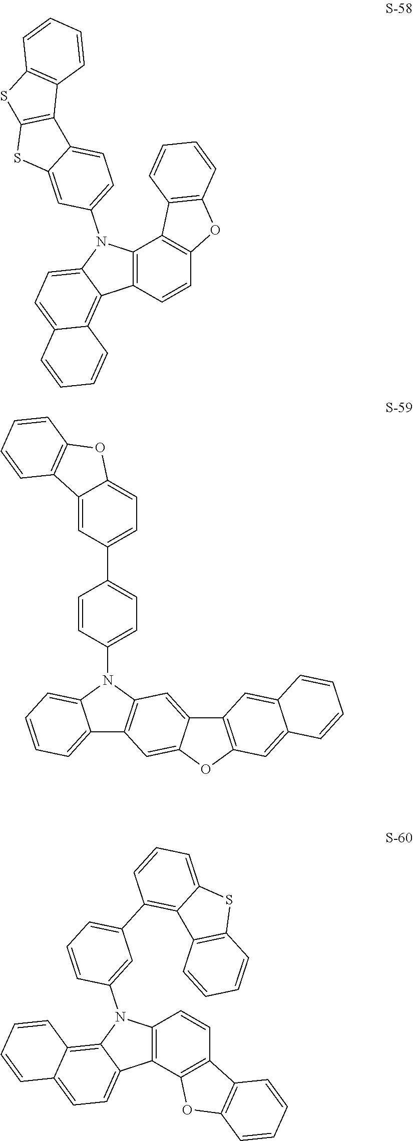

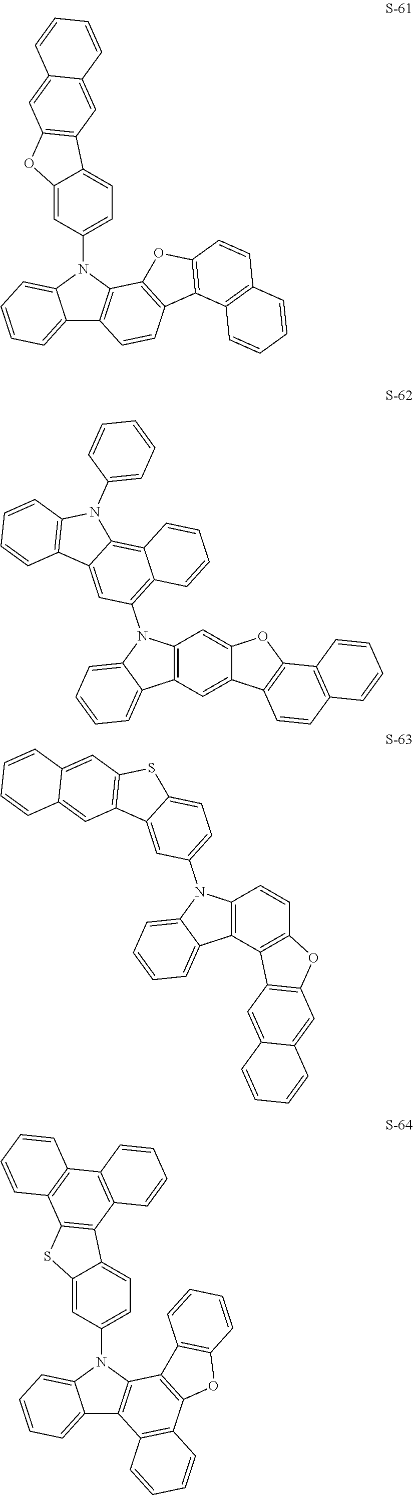

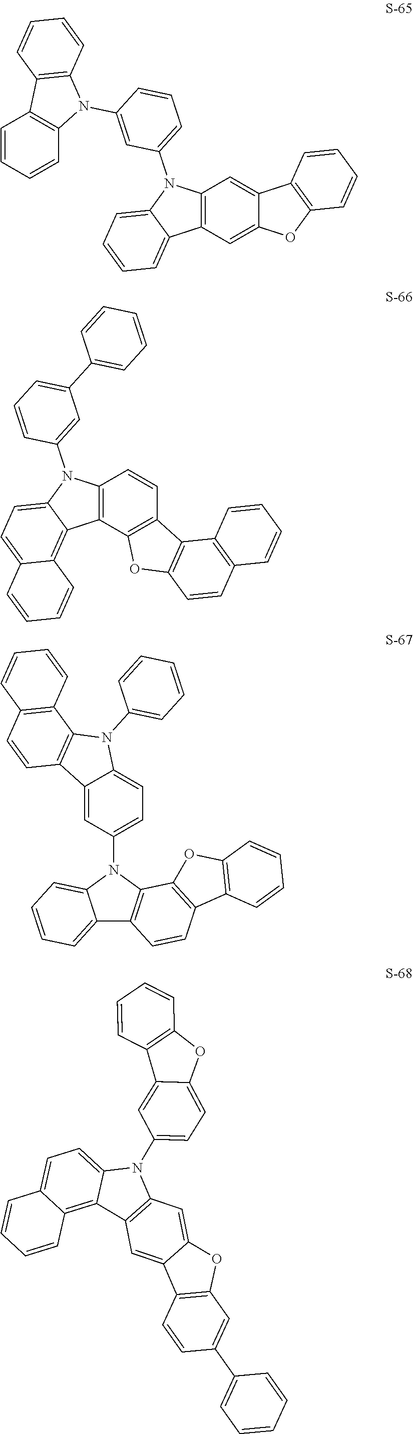

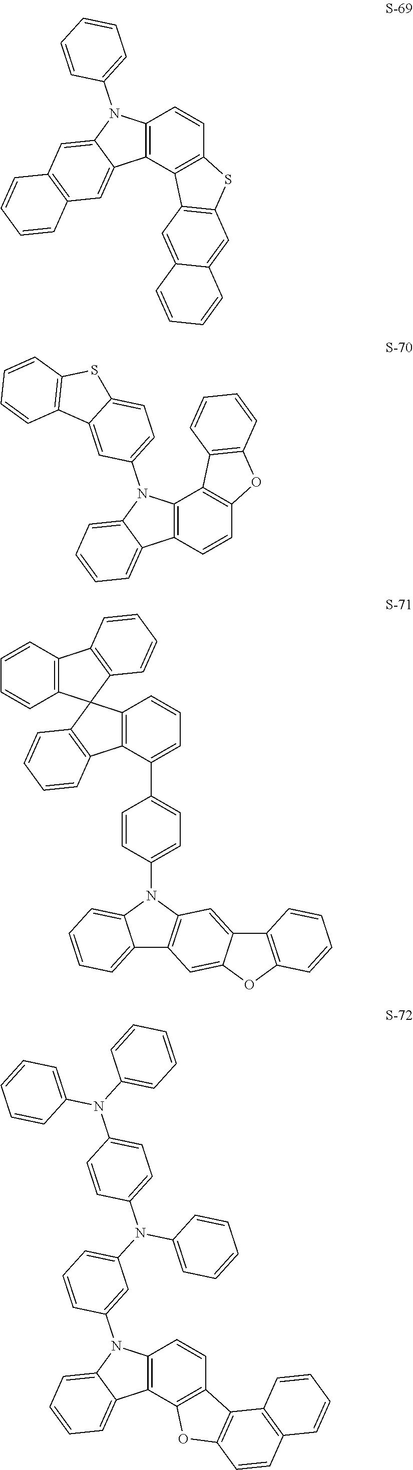

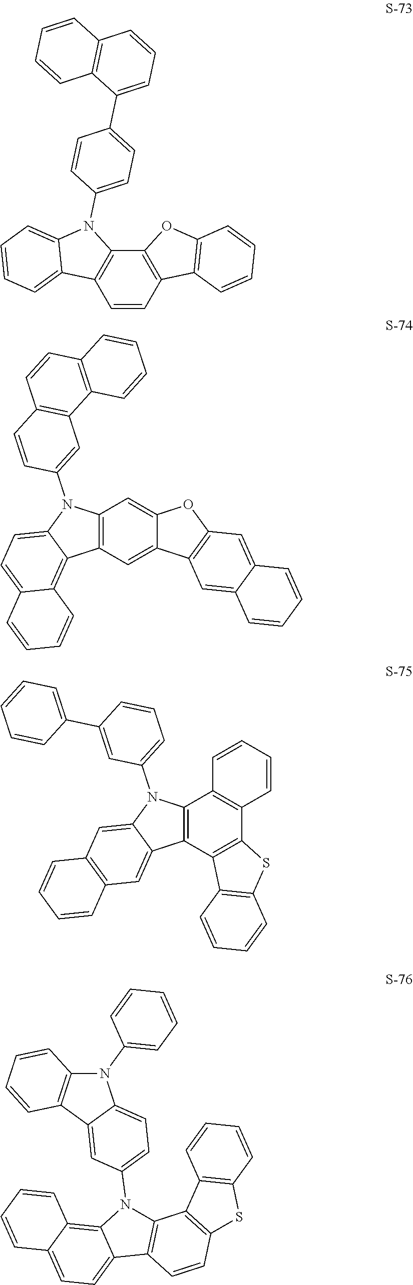

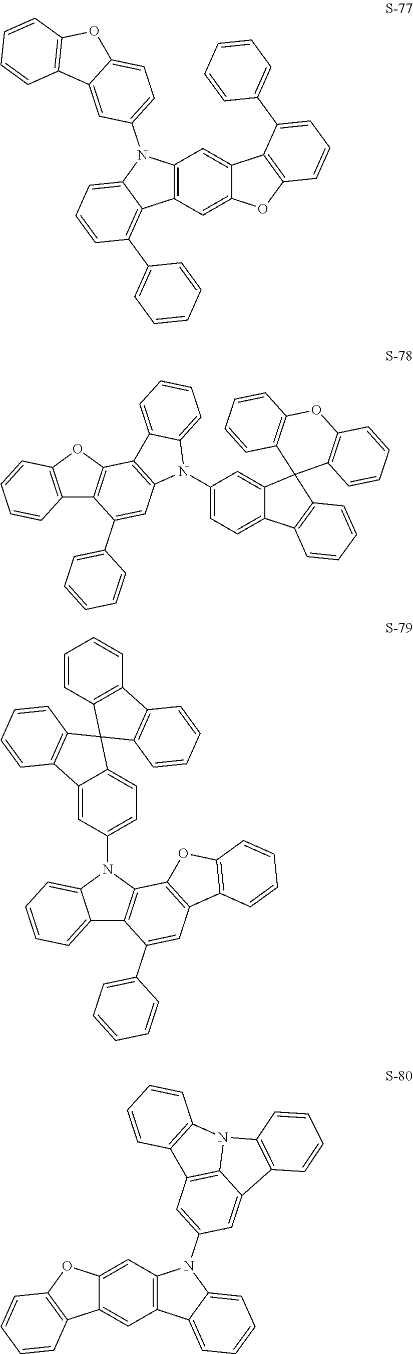

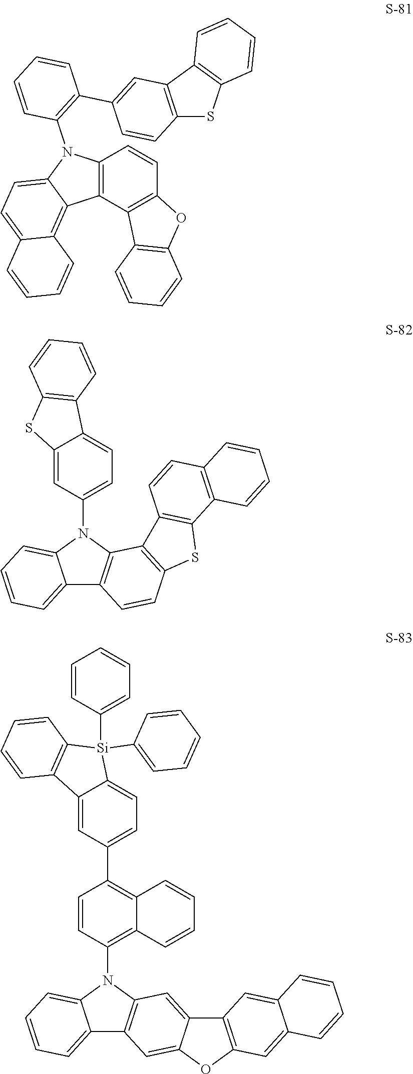

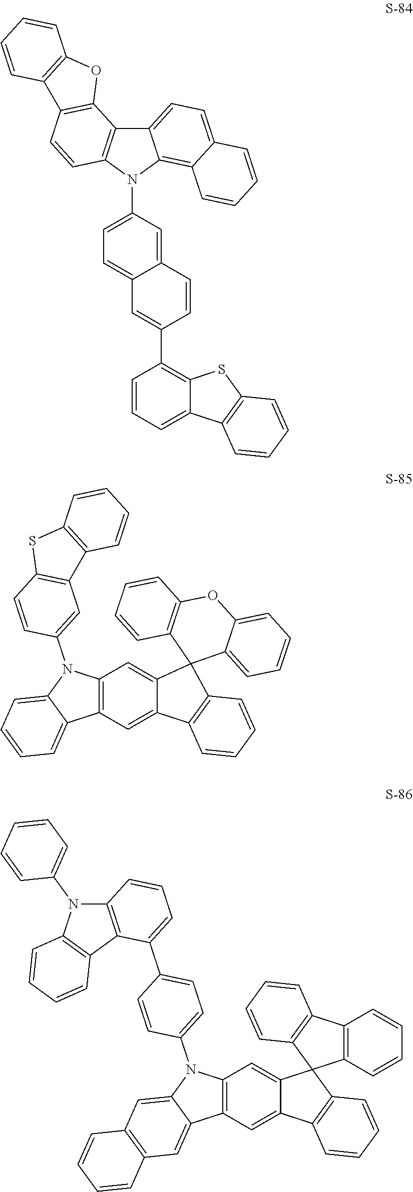 [SMILES: c1ccc2c(c1)ccc1c3c4oc5ccccc5c4ccc3n(-c3ccc4cc(-c5cccc6c5sc5ccccc56)ccc4c3)c21 ; c1ccc2c(c1)Oc1ccccc1C21c2ccccc2-c2cc3c4ccccc4n(-c4ccc5sc6ccccc6c5c4)c3cc21 ; c1ccc(-n2c3ccccc3c3c(-c4ccc(-n5c6cc7c(cc6c6cc8ccccc8cc65)-c5ccccc5C75c6ccccc6-c6ccccc65)cc4)cccc32)cc1]

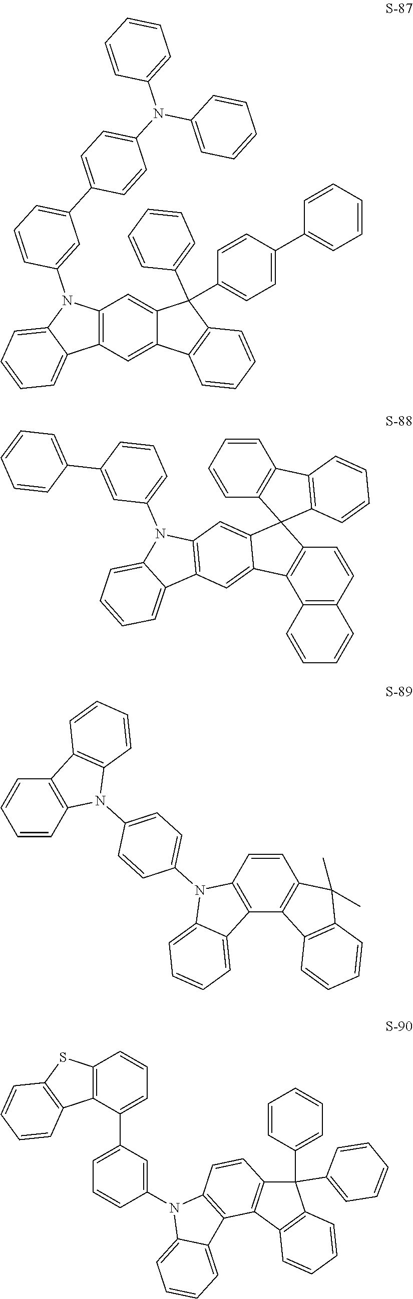

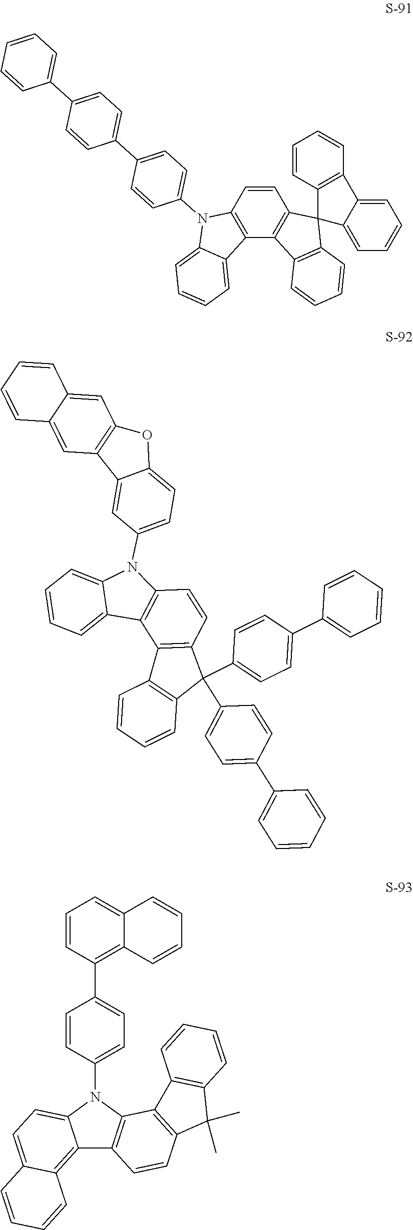 [SMILES: c1ccc(-c2ccc(-c3ccc(-n4c5ccccc5c5c6c(ccc54)C4(c5ccccc5-c5ccccc54)c4ccccc4-6)cc3)cc2)cc1 ; c1ccc(-c2ccc(C3(c4ccc(-c5ccccc5)cc4)c4ccccc4-c4c3ccc3c4c4ccccc4n3-c3ccc4oc5cc6ccccc6cc5c4c3)cc2)cc1 ; CC1(C)c2ccccc2-c2c1ccc1c3c4ccccc4ccc3n(-c3ccc(-c4cccc5ccccc45)cc3)c21]

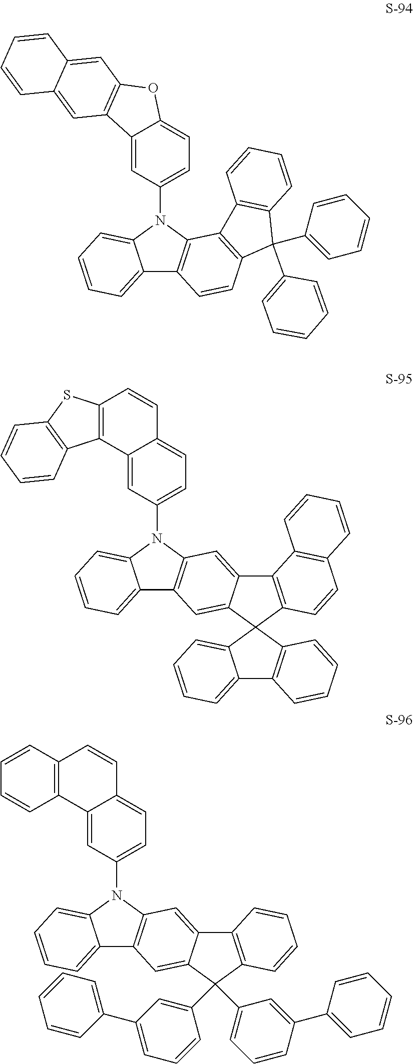 [SMILES: c1ccc(C2(c3ccccc3)c3ccccc3-c3c2ccc2c4ccccc4n(-c4ccc5oc6cc7ccccc7cc6c5c4)c32)cc1 ; c1ccc2c(c1)-c1ccccc1C21c2cc3c4ccccc4n(-c4ccc5ccc6sc7ccccc7c6c5c4)c3cc2-c2c1ccc1ccccc21 ; c1ccc(-c2cccc(C3(c4cccc(-c5ccccc5)c4)c4ccccc4-c4cc5c(cc43)c3ccccc3n5-c3ccc4ccc5ccccc5c4c3)c2)cc1]

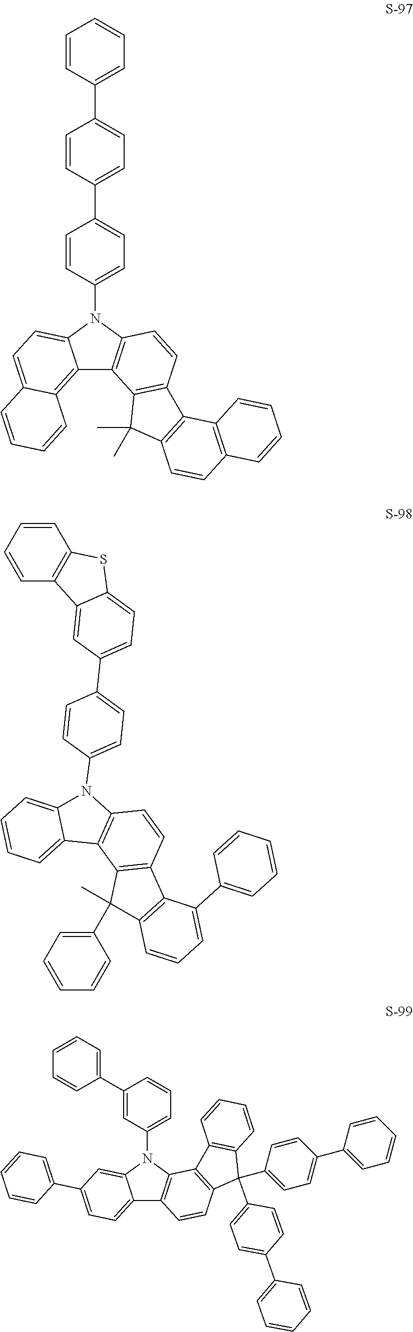 [SMILES: CC1(C)c2ccc3ccccc3c2-c2ccc3c(c21)c1c2ccccc2ccc1n3-c1ccc(-c2ccc(-c3ccccc3)cc2)cc1 ; CC1(c2ccccc2)c2cccc(-c3ccccc3)c2-c2ccc3c(c21)c1ccccc1n3-c1ccc(-c2ccc3sc4ccccc4c3c2)cc1 ; c1ccc(-c2ccc(C3(c4ccc(-c5ccccc5)cc4)c4ccccc4-c4c3ccc3c5ccc(-c6ccccc6)cc5n(-c5cccc(-c6ccccc6)c5)c43)cc2)cc1]

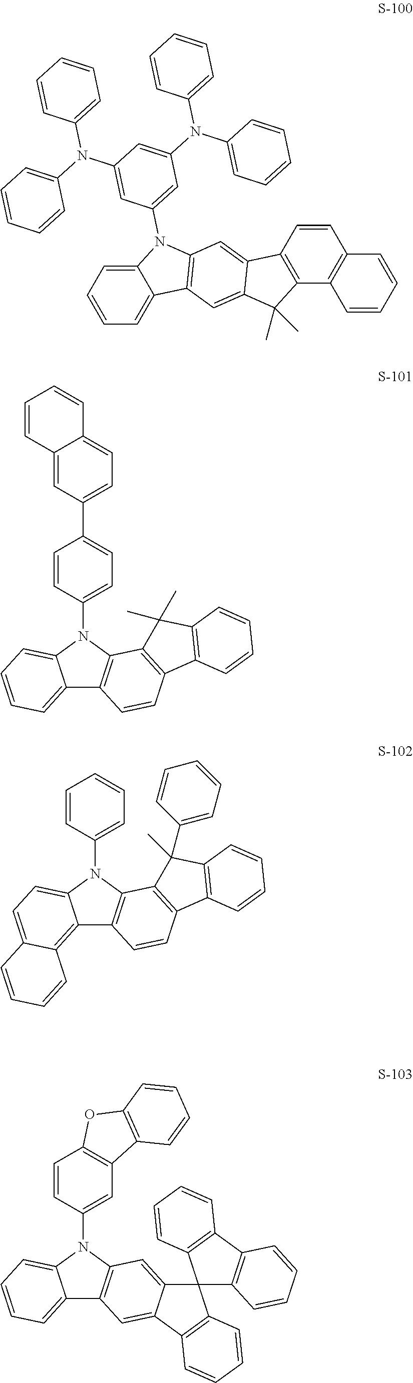 [SMILES: CC1(C)c2cc3c4ccccc4n(-c4cc(N(c5ccccc5)c5ccccc5)cc(N(c5ccccc5)c5ccccc5)c4)c3cc2-c2ccc3ccccc3c21 ; CC1(C)c2ccccc2-c2ccc3c4ccccc4n(-c4ccc(-c5ccc6ccccc6c5)cc4)c3c21 ; CC1(c2ccccc2)c2ccccc2-c2ccc3c4c5ccccc5ccc4n(-c4ccccc4)c3c21 ; c1ccc2c(c1)-c1ccccc1C21c2ccccc2-c2cc3c4ccccc4n(-c4ccc5oc6ccccc6c5c4)c3cc21]

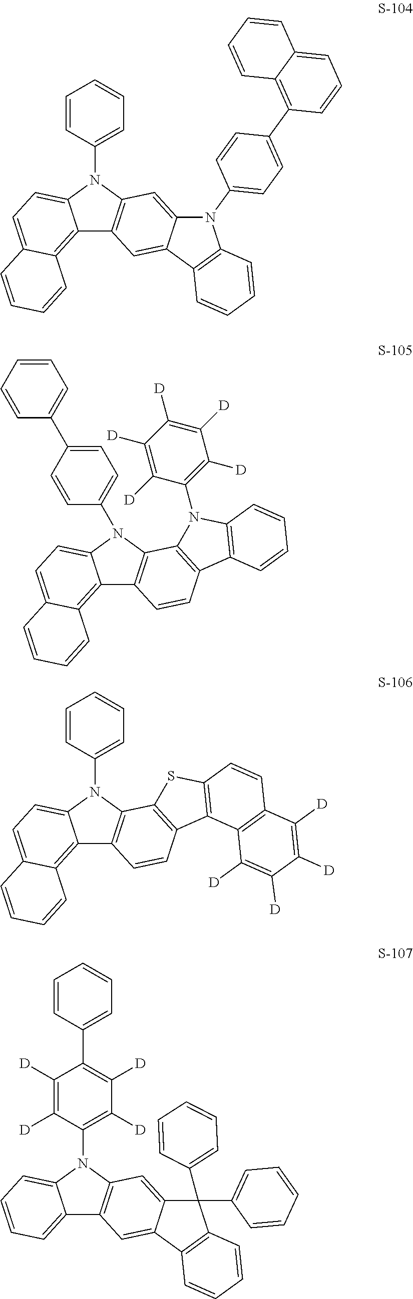

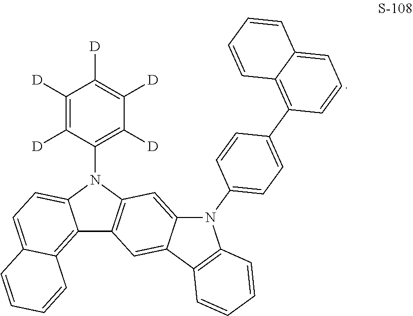 [SMILES: [2H]c1c([2H])c([2H])c(-n2c3cc4c(cc3c3c5ccccc5ccc32)c2ccccc2n4-c2ccc(-c3cccc4ccccc34)cc2)c([2H])c1[2H]]

8. The organic electronic element of claim 5, further comprising a light efficiency enhancing layer formed on at least one surface of the first electrode and the second electrode, the surface being opposite to the organic material layer.

9. The organic electronic element of claim 5, wherein the organic material layer comprises 2 or more stacks comprising a hole transport layer, an emitting layer, and an electron transport layer sequentially formed on the first electrode.

10. The organic electronic element of claim 9, wherein the organic material layer further comprises a charge generation layer formed between the 2 or more stacks.

11. An electronic device comprising a display device comprising the organic electronic element of claim 5; and a control unit for driving the display device.

12. An electronic device according to claim 11, wherein the organic electronic element is at least one of an OLED, an organic solar cell, an organic photo conductor (OPC), organic transistor (organic TFT) and an element for monochromic or white illumination.

13. A method for reusing the compound represented by Formula 3-2 or Formula 3-3 according to claim 1 comprising:
a step of depositing an organic emitting material comprising the compound represented by Formula 3-2 or Formula 3-3 in claim 1 in a manufacturing process of an organic light emitting device;
a step of removing impurities from the crude organic light emitting material recovered from the deposition apparatus;
a step of recovering the removed impurities; and
a step of purifying the recovered impurities to a purity of 99.9% or higher.
